SEQUENCE LISTING (181 sequences)

SEQ ID NO: 1 - DNA, 57 bp, synthetic construct
  misc_feature 1..57: note = Synthetic
  source 1..57: mol_type = other DNA
atggagactgggctgcgctggcttctcctggtcgctgtgctcaaaggtgtccagtgt

SEQ ID NO: 2 - DNA, 2118 bp, synthetic construct
  misc_feature 1..2118: note = Synthetic
  source 1..2118: mol_type = other DNA
cagtcggtggaggagtccgggggtcgcctggtcacgcctgggacacccctgacactcacttgcacagtctctggattctccctcagtagctactacatgacctgggtccgccaggctccagggaaggggctggaatacatcggatacattgcttataatggtaacacatggtacgcgagctgggcgaaaggccgattcaccatctccaaaacctcgaccacggtggatctcaaaatcaccagtccgacaaccgaagacacggccacctatttctgtgccagagatcggggtgatactagtggtatcgactttaacatctggggcccaggcaccctggtcaccgtctcgagcgggcaacctaaggctccatcagttttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacttgcaacgtggcccacccagccaccaacaccaaagtggacaagaccgtggcgccctcgacatgcagcaagcccacgtgcccaccccctgagttcgaggggggaccgtctgtcttcatcttccccccaaaacccaaggacaccctcatgatctcacgcacccccgaggtgacctgcgtggtggtggacgtgagccaggatgaccccgaggtgcagttcacatggtacataaacaacgagcaggtccgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcctccatcgagaagaccatcagcaaggccagagggggcggcggcggcagcggcggcggcggcagcctagcgccctcgacatgcagcaagcccacgtgcccaccccctgagttcgaggggggaccgtctgtcttcatcttccccccaaaacccaaggacaccctcatgatctcacgcacccccgaggtgacctgcgtggtggtggacgtgagccaggatgaccccgaggtgcagttcacatggtacataaacaacgagcaggtccgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcctccatcgagaagaccatcagcaaggccagagggggcggcggcggcagcggcggcggcggcagcctagcgccctcgacatgcagcaagcccacgtgcccaccccctgagttcgaggggggaccgtctgtcttcatcttccccccaaaacccaaggacaccctcatgatctcacgcacccccgaggtgacctgcgtggtggtggacgtgagccaggatgaccccgaggtgcagttcacatggtacataaacaacgagcaggtccgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcctccatcgagaagaccatcagcaaggccagagggcagcccctggagccgaaggtctacaccatgggtcctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacttgctccgtgatgcacgaggccttgcacaaccactacacgcagaagtccatctcccgctctccgggtaaa

SEQ ID NO: 3 - protein, 706 aa, synthetic construct
  REGION 1..706: note = Synthetic
QSVEESGGRLVTPGTPLTLTCTVSGFSLSSYYMTWVRQAPGKGLEYIGYIAYNGNTWYASWAKGRFTISKTSTTVDLKITSPTTEDTATYFCARDRGDTSGIDFNIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPEFEGGPSVFIFPPKPKDTLMISRTPEVTCVVVDVSQDDPEVQFTWYINNEQVRTARPPLREQQFNSTIRVVSTLPIAHQDWLRGKEFKCKVHNKALPASIEKTISKARGGGGGSGGGGSLAPSTCSKPTCPPPEFEGGPSVFIFPPKPKDTLMISRTPEVTCVVVDVSQDDPEVQFTWYINNEQVRTARPPLREQQFNSTIRVVSTLPIAHQDWLRGKEFKCKVHNKALPASIEKTISKARGGGGGSGGGGSLAPSTCSKPTCPPPEFEGGPSVFIFPPKPKDTLMISRTPEVTCVVVDVSQDDPEVQFTWYINNEQVRTARPPLREQQFNSTIRVVSTLPIAHQDWLRGKEFKCKVHNKALPASIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 4 - protein, 8 aa, synthetic construct
  REGION 1..8: note = Synthetic
GFSLSSYY

SEQ ID NO: 5 - protein, 7 aa, synthetic construct
  REGION 1..7: note = Synthetic
IAYNGNT

SEQ ID NO: 6 - protein, 14 aa, synthetic construct
  REGION 1..14: note = Synthetic
ARDRGDTSGIDFNI

SEQ ID NO: 7 - protein, 117 aa, synthetic construct
  REGION 1..117: note = Synthetic
QSVEESGGRLVTPGTPLTLTCTVSGFSLSSYYMTWVRQAPGKGLEYIGYIAYNGNTWYASWAKGRFTISKTSTTVDLKITSPTTEDTATYFCARDRGDTSGIDFNIWGPGTLVTVSS

SEQ ID NO: 8 - DNA, 636 bp, synthetic construct
  misc_feature 1..636: note = Synthetic
  source 1..636: mol_type = other DNA
cagtcggtggaggagtccgggggtcgcctggtcacgcctgggacacccctgacactcacttgcacagtctctggattctccctcagtagctactacatgacctgggtccgccaggctccagggaaggggctggaatacatcggatacattgcttataatggtaacacatggtacgcgagctgggcgaaaggccgattcaccatctccaaaacctcgaccacggtggatctcaaaatcaccagtccgacaaccgaagacacggccacctatttctgtgccagagatcggggtgatactagtggtatcgactttaacatctggggcccaggcaccctggtcaccgtctcgagcgggcaacctaaggctccatcagttttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacttgcaacgtggcccacccagccaccaacaccaaagtggacaagaccgtg

SEQ ID NO: 9 - protein, 212 aa, synthetic construct
  REGION 1..212: note = Synthetic
QSVEESGGRLVTPGTPLTLTCTVSGFSLSSYYMTWVRQAPGKGLEYIGYIAYNGNTWYASWAKGRFTISKTSTTVDLKITSPTTEDTATYFCARDRGDTSGIDFNIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 10 - DNA, 66 bp, synthetic construct
  misc_feature 1..66: note = Synthetic
  source 1..66: mol_type = other DNA
atggacacgagggcccccactcagctgctggggctcctgctgctctggctcccaggtgccacattt

SEQ ID NO: 11 - DNA, 642 bp, synthetic construct
  misc_feature 1..642: note = Synthetic
  source 1..642: mol_type = other DNA
gcctatgatatgacccagactccagcctctgtggaggtagctgtgggaggcacagtcaccatcaagtgccaggccagtgagagcattgatacctacttagcctggtatcagcagaaaccagggcagcctcccaagcaactgatctacagggcatccactctggcatctggggtctcatcgcggttcaaaggcagtggagctgggacacagttcactctcaccatcagcggcgtggagtgtgccgatgctgccacttactactgtcaacaggctactagtagtagtaatattgataatgctttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacttgcaaggtgacccagggcacgacctcagtcgtccagagcttcaataggggtgactgt

SEQ ID NO: 12 - protein, 214 aa, synthetic construct
  REGION 1..214: note = Synthetic
AYDMTQTPASVEVAVGGTVTIKCQASESIDTYLAWYQQKPGQPPKQLIYRASTLASGVSSRFKGSGAGTQFTLTISGVECADAATYYCQQATSSSNIDNAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 13 - protein, 6 aa, synthetic construct
  REGION 1..6: note = Synthetic
ESIDTY

SEQ ID NO: 14 - protein, 4 aa, synthetic construct
  REGION 1..4: note = Synthetic
GGGS

SEQ ID NO: 15 - protein, 12 aa, synthetic construct
  REGION 1..12: note = Synthetic
QQATSSSNIDNA

SEQ ID NO: 16 - protein, 111 aa, synthetic construct
  REGION 1..111: note = Synthetic
AYDMTQTPASVEVAVGGTVTIKCQASESIDTYLAWYQQKPGQPPKQLIYRASTLASGVSSRFKGSGAGTQFTLTISGVECADAATYYCQQATSSSNIDNAFGGGTEVVVKG

SEQ ID NO: 17 - protein, 95 aa, synthetic construct
  REGION 1..95: note = Synthetic
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 18 - protein, 19 aa, synthetic construct
  REGION 1..19: note = Synthetic
APSTCSKPTCPPPELLGGP

SEQ ID NO: 19 - protein, 103 aa, synthetic construct
  REGION 1..103: note = Synthetic
SVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARG

SEQ ID NO: 20 - protein, 106 aa, synthetic construct
  REGION 1..106: note = Synthetic
QPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 21 - DNA, 285 bp, synthetic construct
  misc_feature 1..285: note = Synthetic
  source 1..285: mol_type = other DNA
gggcaacctaaggctccatcagttttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacttgcaacgtggcccacccagccaccaacaccaaagtggacaagaccgtg

SEQ ID NO: 22 - DNA, 57 bp, synthetic construct
  misc_feature 1..57: note = Synthetic
  source 1..57: mol_type = other DNA
gcgccctcgacatgcagcaagcccacgtgcccaccccctgagctcctggggggaccg

SEQ ID NO: 23 - DNA, 309 bp, synthetic construct
  misc_feature 1..309: note = Synthetic
  source 1..309: mol_type = other DNA
tctgtcttcatcttccccccaaaacccaaggacaccctcatgatctcacgcacccccgaggtgacctgcgtggtggtggacgtgagccaggatgaccccgaggtgcagttcacatggtacataaacaacgagcaggtccgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaagaccatcagcaaggccagaggg

SEQ ID NO: 24 - DNA, 318 bp, synthetic construct
  misc_feature 1..318: note = Synthetic
  source 1..318: mol_type = other DNA
cagcccctggagccgaaggtctacaccatgggtcctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacttgctccgtgatgcacgaggccttgcacaaccactacacgcagaagtccatctcccgctctccgggtaaa

SEQ ID NO: 25 - protein, 103 aa, synthetic construct
  REGION 1..103: note = Synthetic
DPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 26 - DNA, 309 bp, synthetic construct
  misc_feature 1..309: note = Synthetic
  source 1..309: mol_type = other DNA
gatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacttgcaaggtgacccagggcacgacctcagtcgtccagagcttcaataggggtgactgt

SEQ ID NO: 27 - DNA, 2103 bp, synthetic construct
  misc_feature 1..2103: note = Synthetic
  source 1..2103: mol_type = other DNA
cagtcggtggaggagtccgggggtcgcctggtcacgcctgggacacccctgacactcacttgcacagtctctggattctccctcagtaactatgcaatgagctgggtccgccaggctccagggaaggggctggaatggatcggaatcgttagtattagtgctggcacatactacgcgagctgggcgaaaggccgattcaccatctccaaaacctcgaccacggtggttctgagagtcaccagtccgacaaccgaggacacggccacctatttctgtgccacagggggttatactggtaacgtctggggcccaggcaccctggtcaccgtctcgagcgggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacttgcaacgtggcccacccagccaccaacaccaaagtggacaagaccgtggcgccctcgacatgcagcaagcccacgtgcccaccccctgagttcgaggggggaccgtctgtcttcatcttccccccaaaacccaaggacaccctcatgatctcacgcacccccgaggtgacctgcgtggtggtggacgtgagccaggatgaccccgaggtgcagttcacatggtacataaacaacgagcaggtccgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcctccatcgagaagaccatcagcaaggccagagggggcggcggcggcagcggcggcggcggcagcctagcgccctcgacatgcagcaagcccacgtgcccaccccctgagttcgaggggggaccgtctgtcttcatcttccccccaaaacccaaggacaccctcatgatctcacgcacccccgaggtgacctgcgtggtggtggacgtgagccaggatgaccccgaggtgcagttcacatggtacataaacaacgagcaggtccgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcctccatcgagaagaccatcagcaaggccagagggggcggcggcggcagcggcggcggcggcagcctagcgccctcgacatgcagcaagcccacgtgcccaccccctgagttcgaggggggaccgtctgtcttcatcttccccccaaaacccaaggacaccctcatgatctcacgcacccccgaggtgacctgcgtggtggtggacgtgagccaggatgaccccgaggtgcagttcacatggtacataaacaacgagcaggtccgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcctccatcgagaagaccatcagcaaggccagagggcagcccctggagccgaaggtctacaccatgggtcctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacttgctccgtgatgcacgaggccttgcacaaccactacacgcagaagtccatctcccgctctccgggtaaa

SEQ ID NO: 28 - protein, 701 aa, synthetic construct
  REGION 1..701: note = Synthetic
QSVEESGGRLVTPGTPLTLTCTVSGFSLSNYAMSWVRQAPGKGLEWIGIVSISAGTYYASWAKGRFTISKTSTTVVLRVTSPTTEDTATYFCATGGYTGNVWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPEFEGGPSVFIFPPKPKDTLMISRTPEVTCVVVDVSQDDPEVQFTWYINNEQVRTARPPLREQQFNSTIRVVSTLPIAHQDWLRGKEFKCKVHNKALPASIEKTISKARGGGGGSGGGGSLAPSTCSKPTCPPPEFEGGPSVFIFPPKPKDTLMISRTPEVTCVVVDVSQDDPEVQFTWYINNEQVRTARPPLREQQFNSTIRVVSTLPIAHQDWLRGKEFKCKVHNKALPASIEKTISKARGGGGGSGGGGSLAPSTCSKPTCPPPEFEGGPSVFIFPPKPKDTLMISRTPEVTCVVVDVSQDDPEVQFTWYINNEQVRTARPPLREQQFNSTIRVVSTLPIAHQDWLRGKEFKCKVHNKALPASIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 29 - protein, 8 aa, synthetic construct
  REGION 1..8: note = Synthetic
GFSLSNYA

SEQ ID NO: 30 - protein, 7 aa, synthetic construct
  REGION 1..7: note = Synthetic
VSISAGT

SEQ ID NO: 31 - protein, 9 aa, synthetic construct
  REGION 1..9: note = Synthetic
ATGGYTGNV

SEQ ID NO: 32 - protein, 112 aa, synthetic construct
  REGION 1..112: note = Synthetic
QSVEESGGRLVTPGTPLTLTCTVSGFSLSNYAMSWVRQAPGKGLEWIGIVSISAGTYYASWAKGRFTISKTSTTVVLRVTSPTTEDTATYFCATGGYTGNVWGPGTLVTVSS

SEQ ID NO: 33 - DNA, 621 bp, synthetic construct
  misc_feature 1..621: note = Synthetic
  source 1..621: mol_type = other DNA
cagtcggtggaggagtccgggggtcgcctggtcacgcctgggacacccctgacactcacttgcacagtctctggattctccctcagtaactatgcaatgagctgggtccgccaggctccagggaaggggctggaatggatcggaatcgttagtattagtgctggcacatactacgcgagctgggcgaaaggccgattcaccatctccaaaacctcgaccacggtggttctgagagtcaccagtccgacaaccgaggacacggccacctatttctgtgccacagggggttatactggtaacgtctggggcccaggcaccctggtcaccgtctcgagcgggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacttgcaacgtggcccacccagccaccaacaccaaagtggacaagaccgtg

SEQ ID NO: 34 - protein, 207 aa, synthetic construct
  REGION 1..207: note = Synthetic
QSVEESGGRLVTPGTPLTLTCTVSGFSLSNYAMSWVRQAPGKGLEWIGIVSISAGTYYASWAKGRFTISKTSTTVVLRVTSPTTEDTATYFCATGGYTGNVWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 35 - DNA, 645 bp, synthetic construct
  misc_feature 1..645: note = Synthetic
  source 1..645: mol_type = other DNA
gcgcaagttctgacccagactgcatcgtccgtgtctgcagctgtgggaggcacagtcaccattagttgccagtccagtcagagtgtttataataagaattggttaggctggtatcagcagaaaccagggcagcctcccaggctcctgatctatggtgcatccactctggcatctggggtcccatcgcggttcagcggcagtggatctgggacacagttcactctcaccatcagcgacgtgcagtgtgacgatgctgccacttactactgtgcaggcggttatagtaataatattgttagtttcggcggaggaaccagggtggtcgtcgaagttgctacattggcacctactgtcctcatcttcccaccatctcctgctgagctggcaactggaacagccaccatcgtgtgcgtggcaaacaaatactttcccgatggcaccgtcacctggcaggtcgatggcaagcccctaacaactggcatcgagaccagtaaaacaccgcagaattctgatgattgtacctacaacctcagcagtactctgacactgaaaagcgacgagtacaacagccacgacgagtacacttgccaggtggcccagggctcaggctcaccggtcgtccagagcttcaataggggtgactgt

SEQ ID NO: 36 - protein, 215 aa, synthetic construct
  REGION 1..215: note = Synthetic
AQVLTQTASSVSAAVGGTVTISCQSSQSVYNKNWLGWYQQKPGQPPRLLIYGASTLASGVPSRFSGSGSGTQFTLTISDVQCDDAATYYCAGGYSNNIVSFGGGTRVVVEVATLAPTVLIFPPSPAELATGTATIVCVANKYFPDGTVTWQVDGKPLTTGIETSKTPQNSDDCTYNLSSTLTLKSDEYNSHDEYTCQVAQGSGSPVVQSFNRGDC

SEQ ID NO: 37 - protein, 8 aa, synthetic construct
  REGION 1..8: note = Synthetic
QSVYNKNW

SEQ ID NO: 38 - protein, 5 aa, synthetic construct
  REGION 1..5: note = Synthetic
GGGGS

SEQ ID NO: 39 - protein, 10 aa, synthetic construct
  REGION 1..10: note = Synthetic
AGGYSNNIVS

SEQ ID NO: 40 - protein, 110 aa, synthetic construct
  REGION 1..110: note = Synthetic
AQVLTQTASSVSAAVGGTVTISCQSSQSVYNKNWLGWYQQKPGQPPRLLIYGASTLASGVPSRFSGSGSGTQFTLTISDVQCDDAATYYCAGGYSNNIVSFGGGTRVVVE

SEQ ID NO: 41 - protein, 105 aa, synthetic construct
  REGION 1..105: note = Synthetic
VATLAPTVLIFPPSPAELATGTATIVCVANKYFPDGTVTWQVDGKPLTTGIETSKTPQNSDDCTYNLSSTLTLKSDEYNSHDEYTCQVAQGSGSPVVQSFNRGDC

SEQ ID NO: 42 - DNA, 315 bp, synthetic construct
  misc_feature 1..315: note = Synthetic
  source 1..315: mol_type = other DNA
gttgctacattggcacctactgtcctcatcttcccaccatctcctgctgagctggcaactggaacagccaccatcgtgtgcgtggcaaacaaatactttcccgatggcaccgtcacctggcaggtcgatggcaagcccctaacaactggcatcgagaccagtaaaacaccgcagaattctgatgattgtacctacaacctcagcagtactctgacactgaaaagcgacgagtacaacagccacgacgagtacacttgccaggtggcccagggctcaggctcaccggtcgtccagagcttcaataggggtgactgt

SEQ ID NO: 43 - protein, 10 aa, synthetic construct
  REGION 1..10: note = Synthetic
GGGGSGGGGS

SEQ ID NO: 44 - protein, 11 aa, synthetic construct
  REGION 1..11: note = Synthetic
GGGGSGGGGSL

SEQ ID NO: 45 - protein, 209 aa, synthetic construct
  REGION 1..209: note = Synthetic
SVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 46 - DNA, 33 bp, synthetic construct
  misc_feature 1..33: note = Synthetic
  source 1..33: mol_type = other DNA
ggcggcggcggcagcggcggcggcggcagccta

SEQ ID NO: 47 - DNA, 627 bp, synthetic construct
  misc_feature 1..627: note = Synthetic
  source 1..627: mol_type = other DNA
tctgtcttcatcttccccccaaaacccaaggacaccctcatgatctcacgcacccccgaggtgacctgcgtggtggtggacgtgagccaggatgaccccgaggtgcagttcacatggtacataaacaacgagcaggtccgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaagaccatcagcaaggccagagggcagcccctggagccgaaggtctacaccatgggtcctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacttgctccgtgatgcacgaggccttgcacaaccactacacgcagaagtccatctcccgctctccgggtaaa

SEQ ID NO: 48 - protein, 19 aa, synthetic construct
  REGION 1..19: note = Synthetic
APSTCSKPTCPPPEFEGGP

SEQ ID NO: 49 - protein, 103 aa, synthetic construct
  REGION 1..103: note = Synthetic
SVFIFPPKPKDTLMISRTPEVTCVVVDVSQDDPEVQFTWYINNEQVRTARPPLREQQFNSTIRVVSTLPIAHQDWLRGKEFKCKVHNKALPASIEKTISKARG

SEQ ID NO: 50 - protein, 209 aa, synthetic construct
  REGION 1..209: note = Synthetic
SVFIFPPKPKDTLMISRTPEVTCVVVDVSQDDPEVQFTWYINNEQVRTARPPLREQQFNSTIRVVSTLPIAHQDWLRGKEFKCKVHNKALPASIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 51 - DNA, 57 bp, synthetic construct
  misc_feature 1..57: note = Synthetic
  source 1..57: mol_type = other DNA
gcgccctcgacatgcagcaagcccacgtgcccaccccctgagttcgaggggggaccg

SEQ ID NO: 52 - protein, 494 aa, synthetic construct
  REGION 1..494: note = Synthetic
APSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGGGGGSGGGGSLAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGGGGGSGGGGSLAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 53 - DNA, 1482 bp, synthetic construct
  misc_feature 1..1482: note = Synthetic
  source 1..1482: mol_type = other DNA
gcgccctcgacatgcagcaagcccacgtgcccaccccctgagctcctggggggaccgtctgtcttcatcttccccccaaaacccaaggacaccctcatgatctcacgcacccccgaggtgacctgcgtggtggtggacgtgagccaggatgaccccgaggtgcagttcacatggtacataaacaacgagcaggtccgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaagaccatcagcaaggccagagggggcggcggcggcagcggcggcggcggcagcctagcgccctcgacatgcagcaagcccacgtgcccaccccctgagctcctggggggaccgtctgtcttcatcttccccccaaaacccaaggacaccctcatgatctcacgcacccccgaggtgacctgcgtggtggtggacgtgagccaggatgaccccgaggtgcagttcacatggtacataaacaacgagcaggtccgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaagaccatcagcaaggccagagggggcggcggcggcagcggcggcggcggcagcctagcgccctcgacatgcagcaagcccacgtgcccaccccctgagctcctggggggaccgtctgtcttcatcttccccccaaaacccaaggacaccctcatgatctcacgcacccccgaggtgacctgcgtggtggtggacgtgagccaggatgaccccgaggtgcagttcacatggtacataaacaacgagcaggtccgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaagaccatcagcaaggccagagggcagcccctggagccgaaggtctacaccatgggtcctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacttgctccgtgatgcacgaggccttgcacaaccactacacgcagaagtccatctcccgctctccgggtaaa

SEQ ID NO: 54 - protein, 494 aa, synthetic construct
  REGION 1..494: note = Synthetic
APSTCSKPTCPPPEFEGGPSVFIFPPKPKDTLMISRTPEVTCVVVDVSQDDPEVQFTWYINNEQVRTARPPLREQQFNSTIRVVSTLPIAHQDWLRGKEFKCKVHNKALPASIEKTISKARGGGGGSGGGGSLAPSTCSKPTCPPPEFEGGPSVFIFPPKPKDTLMISRTPEVTCVVVDVSQDDPEVQFTWYINNEQVRTARPPLREQQFNSTIRVVSTLPIAHQDWLRGKEFKCKVHNKALPASIEKTISKARGGGGGSGGGGSLAPSTCSKPTCPPPEFEGGPSVFIFPPKPKDTLMISRTPEVTCVVVDVSQDDPEVQFTWYINNEQVRTARPPLREQQFNSTIRVVSTLPIAHQDWLRGKEFKCKVHNKALPASIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 55 - DNA, 1482 bp, synthetic construct
  misc_feature 1..1482: note = Synthetic
  source 1..1482: mol_type = other DNA
gcgccctcgacatgcagcaagcccacgtgcccaccccctgagttcgaggggggaccgtctgtcttcatcttccccccaaaacccaaggacaccctcatgatctcacgcacccccgaggtgacctgcgtggtggtggacgtgagccaggatgaccccgaggtgcagttcacatggtacataaacaacgagcaggtccgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcctccatcgagaagaccatcagcaaggccagagggggcggcggcggcagcggcggcggcggcagcctagcgccctcgacatgcagcaagcccacgtgcccaccccctgagttcgaggggggaccgtctgtcttcatcttccccccaaaacccaaggacaccctcatgatctcacgcacccccgaggtgacctgcgtggtggtggacgtgagccaggatgaccccgaggtgcagttcacatggtacataaacaacgagcaggtccgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcctccatcgagaagaccatcagcaaggccagagggggcggcggcggcagcggcggcggcggcagcctagcgccctcgacatgcagcaagcccacgtgcccaccccctgagttcgaggggggaccgtctgtcttcatcttccccccaaaacccaaggacaccctcatgatctcacgcacccccgaggtgacctgcgtggtggtggacgtgagccaggatgaccccgaggtgcagttcacatggtacataaacaacgagcaggtccgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcctccatcgagaagaccatcagcaaggccagagggcagcccctggagccgaaggtctacaccatgggtcctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacttgctccgtgatgcacgaggccttgcacaaccactacacgcagaagtccatctcccgctctccgggtaaa

SEQ ID NO: 56 - protein, 467 aa, synthetic construct
  REGION 1..467: note = Synthetic
APSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGKGGGGSGGGGSLAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 57 - DNA, 1401 bp, synthetic construct
  misc_feature 1..1401: note = Synthetic
  source 1..1401: mol_type = other DNA
gcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaaggcggcggcggcagcggcggcggcggcagcctagcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 58 - DNA, 57 bp, synthetic construct
  misc_feature 1..57: note = Synthetic
  source 1..57: mol_type = other DNA
gcgccctcgacatgcagcaagcccacgtgcccaccccctgaactcctggggggaccg

SEQ ID NO: 59 - DNA, 627 bp, synthetic construct
  misc_feature 1..627: note = Synthetic
  source 1..627: mol_type = other DNA
tctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 60 - protein, 706 aa, synthetic construct
  REGION 1..706: note = Synthetic
APSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGKGGGGSGGGGSLAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGKGGGGSGGGGSLAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 61 - DNA, 2118 bp, synthetic construct
  misc_feature 1..2118: note = Synthetic
  source 1..2118: mol_type = other DNA
gcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaaggcggcggcggcagcggcggcggcggcagcctagcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaaggcggcggcggcagcggcggcggcggcagcctagcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 62 - DNA, 309 bp, synthetic construct
  misc_feature 1..309: note = Synthetic
  source 1..309: mol_type = other DNA
tctgtcttcatcttccccccaaaacccaaggacaccctcatgatctcacgcacccccgaggtgacctgcgtggtggtggacgtgagccaggatgaccccgaggtgcagttcacatggtacataaacaacgagcaggtccgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcctccatcgagaagaccatcagcaaggccagaggg

SEQ ID NO: 63 - protein, 19 aa, synthetic construct
  REGION 1..19: note = Synthetic
METGLRWLLLVAVLKGVQC

SEQ ID NO: 64 - DNA, 339 bp, synthetic construct
  misc_feature 1..339: note = Synthetic
  source 1..339: mol_type = other DNA
cagtcgctggaggagtccgggggtcgcctggtcacgcctgggacacccctgacactcacctgcacagtctctggaatcgacctcagtacctatccaatgggctgggtccgccaggcgacagggaaggggctggaatacatcggaatcattagtagtagtggtagcacatactacgcgagctggacaaaaggccgattcaccatctccaaaacctcgaccacggtggatctgaaaatgaccactctgacaacttcagacacggccacctatttctgtaccagaccatggggatacggcatggacccctggggcccagggaccctcgtcaccgtctcttca

SEQ ID NO: 65 - protein, 113 aa, synthetic construct
  REGION 1..113: note = Synthetic
QSLEESGGRLVTPGTPLTLTCTVSGIDLSTYPMGWVRQATGKGLEYIGIISSSGSTYYASWTKGRFTISKTSTTVDLKMTTLTTSDTATYFCTRPWGYGMDPWGPGTLVTVSS

SEQ ID NO: 66 - protein, 8 aa, synthetic construct
  REGION 1..8: note = Synthetic
GIDLSTYP

SEQ ID NO: 67 - protein, 7 aa, synthetic construct
  REGION 1..7: note = Synthetic
ISSSGST

SEQ ID NO: 68 - protein, 10 aa, synthetic construct
  REGION 1..10: note = Synthetic
TRPWGYGMDP

SEQ ID NO: 69 - protein, 22 aa, synthetic construct
  REGION 1..22: note = Synthetic
MDTRAPTQLLGLLLLWLPGATF

SEQ ID NO: 70 - DNA, 333 bp, synthetic construct
  misc_feature 1..333: note = Synthetic
  source 1..333: mol_type = other DNA
gcgcaagtgctgacccagactccatcccctgtgtctgcagctgtgggaggcacagtcaccatcagttgccaggccagtcagagtgtttataatgacaacgccttatcctggtatcagcagaaaccagggcagcctcccaaagtcctgatctactatgcatccactctggcatctggggtcccatcgcggttcaaaggcagtggatctgggacacagttcactctcaccatcagcgaagtacagtgtgacgatgctgccacttactattgtcaaggctattatagtggtgctatttatgttttcggcggagggaccgaggtggtgctcaaaggt

SEQ ID NO: 71 - protein, 111 aa, synthetic construct
  REGION 1..111: note = Synthetic
AQVLTQTPSPVSAAVGGTVTISCQASQSVYNDNALSWYQQKPGQPPKVLIYYASTLASGVPSRFKGSGSGTQFTLTISEVQCDDAATYYCQGYYSGAIYVFGGGTEVVLKG

SEQ ID NO: 72 - protein, 8 aa, synthetic construct
  REGION 1..8: note = Synthetic
QSVYNDNA

SEQ ID NO: 73 - protein, 10 aa, synthetic construct
  REGION 1..10: note = Synthetic
QGYYSGAIYV

SEQ ID NO: 74 - DNA, 1308 bp, synthetic construct
  misc_feature 1..1308: note = Synthetic
  source 1..1308: mol_type = other DNA
cagtcgctggaggagtccgggggtcgcctggtcacgcctgggacacccctgacactcacctgcacagtctctggaatcgacctcagtacctatccaatgggctgggtccgccaggcgacagggaaggggctggaatacatcggaatcattagtagtagtggtagcacatactacgcgagctggacaaaaggccgattcaccatctccaaaacctcgaccacggtggatctgaaaatgaccactctgacaacttcagacacggccacctatttctgtaccagaccatggggatacggcatggacccctggggcccagggaccctcgtcaccgtctcttcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgtggcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 75 - protein, 436 aa, synthetic construct
  REGION 1..436: note = Synthetic
QSLEESGGRLVTPGTPLTLTCTVSGIDLSTYPMGWVRQATGKGLEYIGIISSSGSTYYASWTKGRFTISKTSTTVDLKMTTLTTSDTATYFCTRPWGYGMDPWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 76 - DNA, 642 bp, synthetic construct
  misc_feature 1..642: note = Synthetic
  source 1..642: mol_type = other DNA
gcgcaagtgctgacccagactccatcccctgtgtctgcagctgtgggaggcacagtcaccatcagttgccaggccagtcagagtgtttataatgacaacgccttatcctggtatcagcagaaaccagggcagcctcccaaagtcctgatctactatgcatccactctggcatctggggtcccatcgcggttcaaaggcagtggatctgggacacagttcactctcaccatcagcgaagtacagtgtgacgatgctgccacttactattgtcaaggctattatagtggtgctatttatgttttcggcggagggaccgaggtggtgctcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgt

SEQ ID NO: 77 - protein, 214 aa, synthetic construct
  REGION 1..214: note = Synthetic
AQVLTQTPSPVSAAVGGTVTISCQASQSVYNDNALSWYQQKPGQPPKVLIYYASTLASGVPSRFKGSGSGTQFTLTISEVQCDDAATYYCQGYYSGAIYVFGGGTEVVLKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 78 - DNA, 624 bp, synthetic construct
  misc_feature 1..624: note = Synthetic
  source 1..624: mol_type = other DNA
cagtcgctggaggagtccgggggtcgcctggtcacgcctgggacacccctgacactcacctgcacagtctctggaatcgacctcagtacctatccaatgggctgggtccgccaggcgacagggaaggggctggaatacatcggaatcattagtagtagtggtagcacatactacgcgagctggacaaaaggccgattcaccatctccaaaacctcgaccacggtggatctgaaaatgaccactctgacaacttcagacacggccacctatttctgtaccagaccatggggatacggcatggacccctggggcccagggaccctcgtcaccgtctcttcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgtg

SEQ ID NO: 79 - protein, 208 aa, synthetic construct
  REGION 1..208: note = Synthetic
QSLEESGGRLVTPGTPLTLTCTVSGIDLSTYPMGWVRQATGKGLEYIGIISSSGSTYYASWTKGRFTISKTSTTVDLKMTTLTTSDTATYFCTRPWGYGMDPWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 80 - DNA, 309 bp, synthetic construct
  misc_feature 1..309: note = Synthetic
  source 1..309: mol_type = other DNA
gatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgt

SEQ ID NO: 81 - DNA, 66 bp, synthetic construct
  misc_feature 1..66: note = Synthetic
  source 1..66: mol_type = other DNA
atggacatgagggtgcccgctcaactgctgggtctgctgcttctttggctgcgcggggcccgatgt

SEQ ID NO: 82 - protein, 22 aa, synthetic construct
  REGION 1..22: note = Synthetic
MDMRVPAQLLGLLLLWLRGARC

SEQ ID NO: 83 - DNA, 357 bp, synthetic construct
  misc_feature 1..357: note = Synthetic
  source 1..357: mol_type = other DNA
gaagtgcacctggtggaaacaggcggaggactggttcagcctaagggcagcctgacactgtcttgtgccgccagcggcttcaccttcaacatcaacgccatgaactgggtccgacaggcccctggcaaaggcctggaatggattgcccggatccgcagcatcatcaacaacaatgccatctactacgccgacagcgtgaaggaccggttcaccatcagcagagatgacagccagagcatggtgtacctgcagatgaacgacgtgaagtccgaggacaccgccatctattactgcgtgcggggactgcctcacgctctggattattggggccagggcacaagcgtgaccgtctcgagc

SEQ ID NO: 84 - protein, 119 aa, synthetic construct
  REGION 1..119: note = Synthetic
EVHLVETGGGLVQPKGSLTLSCAASGFTFNINAMNWVRQAPGKGLEWIARIRSIINNNAIYYADSVKDRFTISRDDSQSMVYLQMNDVKSEDTAIYYCVRGLPHALDYWGQGTSVTVSS

SEQ ID NO: 85 - protein, 8 aa, synthetic construct
  REGION 1..8: note = Synthetic
GFTFNINA

SEQ ID NO: 86 - protein, 10 aa, synthetic construct
  REGION 1..10: note = Synthetic
IRSIINNNAI

SEQ ID NO: 87 - protein, 10 aa, synthetic construct
  REGION 1..10: note = Synthetic
VRGLPHALDY

SEQ ID NO: 88 - DNA, 66 bp, synthetic construct
  misc_feature 1..66: note = Synthetic
  source 1..66: mol_type = other DNA
atggacatgagggtgcccgctcaactgctgggtctgctgcttctttggctgcgcggggcccgatgc

SEQ ID NO: 89 - DNA, 339 bp, synthetic construct
  misc_feature 1..339: note = Synthetic
  source 1..339: mol_type = other DNA
gacgttgtgctgacccagacaccactgagcctgcctgtgtctctgggagatcaggccagcatcagctgcagatctagccagagcctggtgcacagcaacggcaacacctacctgtactggtatctgcagaagcccggacagagccccaagctgctgatctacaaggtgtccaaccggttcagcggcgtgcccgatagattttctggcagcggctctggcaccgacttcaccctgaagatcagctccgtggaagccgaggatctgggcgtgtacttctgcagccagagcacacacgtgccaccttacacatttggcggaggtaccaagctggaaatcaag

SEQ ID NO: 90 - protein, 113 aa, synthetic construct
  REGION 1..113: note = Synthetic
DVVLTQTPLSLPVSLGDQASISCRSSQSLVHSNGNTYLYWYLQKPGQSPKLLIYKVSNRFSGVPDRFSGSGSGTDFTLKISSVEAEDLGVYFCSQSTHVPPYTFGGGTKLEIK

SEQ ID NO: 91 - protein, 11 aa, synthetic construct
  REGION 1..11: note = Synthetic
QSLVHSNGNTY

SEQ ID NO: 92 - protein, 10 aa, synthetic construct
  REGION 1..10: note = Synthetic
SQSTHVPPYT

SEQ ID NO: 93 - DNA, 1335 bp, synthetic construct
  misc_feature 1..1335: note = Synthetic
  source 1..1335: mol_type = other DNA
gaagtgcacctggtggaaacaggcggaggactggttcagcctaagggcagcctgacactgtcttgtgccgccagcggcttcaccttcaacatcaacgccatgaactgggtccgacaggcccctggcaaaggcctggaatggattgcccggatccgcagcatcatcaacaacaatgccatctactacgccgacagcgtgaaggaccggttcaccatcagcagagatgacagccagagcatggtgtacctgcagatgaacgacgtgaagtccgaggacaccgccatctattactgcgtgcggggactgcctcacgctctggattattggggccagggcacaagcgtgaccgtctcgagcgctaagacaacacctcctagcgtgtaccctctggctcctggatctgccgctcagaccaatagcatggtcaccctgggctgtctggtcaagggctactttcctgagcctgtgaccgtgacctggaacagcggatctctgtctagcggcgtgcacacctttccagccgtgctgcagagcgatctgtacaccctgagcagcagcgtgacagtgcctagctctacctggcctagcgagacagtgacctgcaacgtggcccatcctgccagcagcaccaaggtggacaagaaaatcggcgccgtgcccagagactgcggctgcaagccctgcatctgtaccgtgcctgaagtgtccagcgtgttcatcttcccacctaagcctaaggacgtgctgaccatcacactgacccctaaagtgacctgtgtggtggtggacatcagcaaggacgaccccgaggtgcagttcagttggttcgtggacgacgtggaagtgcacacagcccagacacagcctagagaggaacagttcaacagcaccttcagaagcgtgtccgagctgcccatcatgcaccaggattggctgaacggcaaagaattcaagtgcagagtgaacagcgccgcctttcctgctcctatcgagaaaaccatctccaagaccaagggcagacccaaggctccccaggtgtacacaatccctccacctaaagaacagatggccaaggacaaggtgtccctgacctgcatgatcaccgatttcttcccagaggacatcaccgtggaatggcagtggaatggacagcccgccgagaactacaagaacacccagcctatcatggacaccgacggcagctacttcgtgtacagcaagctgaacgtgcagaagtccaactgggaggccggcaacaccttcacctgttctgtgctgcacgagggcctgcacaatcaccacaccgagaagtccctgagccactctcctggaaag

SEQ ID NO: 94 - protein, 445 aa, synthetic construct
  REGION 1..445: note = Synthetic
EVHLVETGGGLVQPKGSLTLSCAASGFTFNINAMNWVRQAPGKGLEWIARIRSIINNNAIYYADSVKDRFTISRDDSQSMVYLQMNDVKSEDTAIYYCVRGLPHALDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIGAVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 95 - DNA, 660 bp, synthetic construct
  misc_feature 1..660: note = Synthetic
  source 1..660: mol_type = other DNA
gacgttgtgctgacccagacaccactgagcctgcctgtgtctctgggagatcaggccagcatcagctgcagatctagccagagcctggtgcacagcaacggcaacacctacctgtactggtatctgcagaagcccggacagagccccaagctgctgatctacaaggtgtccaaccggttcagcggcgtgcccgatagattttctggcagcggctctggcaccgacttcaccctgaagatcagctccgtggaagccgaggatctgggcgtgtacttctgcagccagagcacacacgtgccaccttacacatttggcggaggtaccaagctggaaatcaagcgggccgacgcggcgcccaccgtgagcatcttcccccccagcagcgagcagctgacaagcggcggagctagcgtcgtgtgcttcctgaacaacttctaccccaaggacatcaacgtgaagtggaagatcgacggcagcgagagacagaacggcgtgctgaatagctggaccgaccaggacagcaaggactccacctacagcatgtccagcacactgaccctgaccaaggacgagtacgagcggcacaacagctacacatgcgaggccacacacaagaccagcacaagccccatcgtgaagtccttcaaccggaacgagtgc

SEQ ID NO: 96 - protein, 220 aa, synthetic construct
  REGION 1..220: note = Synthetic
DVVLTQTPLSLPVSLGDQASISCRSSQSLVHSNGNTYLYWYLQKPGQSPKLLIYKVSNRFSGVPDRFSGSGSGTDFTLKISSVEAEDLGVYFCSQSTHVPPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 97 - DNA, 654 bp, synthetic construct
  misc_feature 1..654: note = Synthetic
  source 1..654: mol_type = other DNA
gaagtgcacctggtggaaacaggcggaggactggttcagcctaagggcagcctgacactgtcttgtgccgccagcggcttcaccttcaacatcaacgccatgaactgggtccgacaggcccctggcaaaggcctggaatggattgcccggatccgcagcatcatcaacaacaatgccatctactacgccgacagcgtgaaggaccggttcaccatcagcagagatgacagccagagcatggtgtacctgcagatgaacgacgtgaagtccgaggacaccgccatctattactgcgtgcggggactgcctcacgctctggattattggggccagggcacaagcgtgaccgtctcgagcgctaagacaacacctcctagcgtgtaccctctggctcctggatctgccgctcagaccaatagcatggtcaccctgggctgtctggtcaagggctactttcctgagcctgtgaccgtgacctggaacagcggatctctgtctagcggcgtgcacacctttccagccgtgctgcagagcgatctgtacaccctgagcagcagcgtgacagtgcctagctctacctggcctagcgagacagtgacctgcaacgtggcccatcctgccagcagcaccaaggtggacaagaaaatcggcgcc

SEQ ID NO: 98 - protein, 218 aa, synthetic construct
  REGION 1..218: note = Synthetic
EVHLVETGGGLVQPKGSLTLSCAASGFTFNINAMNWVRQAPGKGLEWIARIRSIINNNAIYYADSVKDRFTISRDDSQSMVYLQMNDVKSEDTAIYYCVRGLPHALDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIGA

SEQ ID NO: 99 - DNA, 297 bp, synthetic construct
  misc_feature 1..297: note = Synthetic
  source 1..297: mol_type = other DNA
gctaagacaacacctcctagcgtgtaccctctggctcctggatctgccgctcagaccaatagcatggtcaccctgggctgtctggtcaagggctactttcctgagcctgtgaccgtgacctggaacagcggatctctgtctagcggcgtgcacacctttccagccgtgctgcagagcgatctgtacaccctgagcagcagcgtgacagtgcctagctctacctggcctagcgagacagtgacctgcaacgtggcccatcctgccagcagcaccaaggtggacaagaaaatcggcgcc

SEQ ID NO: 100 - protein, 99 aa, synthetic construct
  REGION 1..99: note = Synthetic
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIGA

SEQ ID NO: 101 - DNA, 57 bp, synthetic construct
  misc_feature 1..57: note = Synthetic
  source 1..57: mol_type = other DNA
gtgcccagagactgcggctgcaagccctgcatctgtaccgtgcctgaagtgtccagc

SEQ ID NO: 102 - protein, 19 aa, synthetic construct
  REGION 1..19: note = Synthetic
VPRDCGCKPCICTVPEVSS

SEQ ID NO: 103 - DNA, 303 bp, synthetic construct
  misc_feature 1..303: note = Synthetic
  source 1..303: mol_type = other DNA
gtgttcatcttcccacctaagcctaaggacgtgctgaccatcacactgacccctaaagtgacctgtgtggtggtggacatcagcaaggacgaccccgaggtgcagttcagttggttcgtggacgacgtggaagtgcacacagcccagacacagcctagagaggaacagttcaacagcaccttcagaagcgtgtccgagctgcccatcatgcaccaggattggctgaacggcaaagaattcaagtgcagagtgaacagcgccgcctttcctgctcctatcgagaaaaccatctccaagaccaag

SEQ ID NO: 104 - protein, 101 aa, synthetic construct
  REGION 1..101: note = Synthetic
VFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTK

SEQ ID NO: 105 - DNA, 321 bp, synthetic construct
  misc_feature 1..321: note = Synthetic
  source 1..321: mol_type = other DNA
ggcagacccaaggctccccaggtgtacacaatccctccacctaaagaacagatggccaaggacaaggtgtccctgacctgcatgatcaccgatttcttcccagaggacatcaccgtggaatggcagtggaatggacagcccgccgagaactacaagaacacccagcctatcatggacaccgacggcagctacttcgtgtacagcaagctgaacgtgcagaagtccaactgggaggccggcaacaccttcacctgttctgtgctgcacgagggcctgcacaatcaccacaccgagaagtccctgagccactctcctggaaag

SEQ ID NO: 106 - protein, 107 aa, synthetic construct
  REGION 1..107: note = Synthetic
GRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 107 - DNA, 321 bp, synthetic construct
  misc_feature 1..321: note = Synthetic
  source 1..321: mol_type = other DNA
cgggccgacgcggcgcccaccgtgagcatcttcccccccagcagcgagcagctgacaagcggcggagctagcgtcgtgtgcttcctgaacaacttctaccccaaggacatcaacgtgaagtggaagatcgacggcagcgagagacagaacggcgtgctgaatagctggaccgaccaggacagcaaggactccacctacagcatgtccagcacactgaccctgaccaaggacgagtacgagcggcacaacagctacacatgcgaggccacacacaagaccagcacaagccccatcgtgaagtccttcaaccggaacgagtgc

SEQ ID NO: 108 - protein, 107 aa, synthetic construct
  REGION 1..107: note = Synthetic
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 109 - DNA, 351 bp, synthetic construct
  misc_feature 1..351: note = Synthetic
  source 1..351: mol_type = other DNA
gaaggacagctgcagcagtctggacctgagctgcttaaacctggagcctccgtgaagatgagctgcaaggccagcggctacaccttcaccagctacgtgatgcactgggtcaagcagaagccaggccagggccttgagtggatcggctacatcaaccccttcaacgactacaccaactacaacgagaagttccgcggcaaggccacactgaccagcgacaagtctagcaacaccgcctacatggacctgtccagcctgacctctgaggacagcgccgtgtactactgtgccagacaaggtggcagagccctggattattggggccagggcacaagcgttaccgtctcgagc

SEQ ID NO: 110 - protein, 117 aa, synthetic construct
  REGION 1..117: note = Synthetic
EGQLQQSGPELLKPGASVKMSCKASGYTFTSYVMHWVKQKPGQGLEWIGYINPFNDYTNYNEKFRGKATLTSDKSSNTAYMDLSSLTSEDSAVYYCARQGGRALDYWGQGTSVTVSS

SEQ ID NO: 111 - protein, 8 aa, synthetic construct
  REGION 1..8: note = Synthetic
GYTFTSYV

SEQ ID NO: 112 - protein, 8 aa, synthetic construct
  REGION 1..8: note = Synthetic
INPFNDYT

SEQ ID NO: 113 - protein, 10 aa, synthetic construct
  REGION 1..10: note = Synthetic
ARQGGRALDY

SEQ ID NO: 114 - DNA, 318 bp, synthetic construct
  misc_feature 1..318: note = Synthetic
  source 1..318: mol_type = other DNA
cagatcgtgctgacacagagccctgccatcatgtctgctagccctggcgagaaagtgaccatgacctgtagcgccagcagcagcgtgacctacatccactggtatcagctgaagtccggcacaagccccaagcggtggatctacgatacaagcaagctggcctctggcgtgcccgctagattttctggctctggcagcggcaccagctacagcctgacaatcagcagcatggaagccgaggatgccgccacctactactgccagcagtggtccagcaatcccttcacatttggctccggcaccaagctggaaatcaag

SEQ ID NO: 115 - protein, 106 aa, synthetic construct
  REGION 1..106: note = Synthetic
QIVLTQSPAIMSASPGEKVTMTCSASSSVTYIHWYQLKSGTSPKRWIYDTSKLASGVPARFSGSGSGTSYSLTISSMEAEDAATYYCQQWSSNPFTFGSGTKLEIK

SEQ ID NO: 116 - protein, 5 aa, synthetic construct
  REGION 1..5: note = Synthetic
SSVTY

SEQ ID NO: 117 - protein, 9 aa, synthetic construct
  REGION 1..9: note = Synthetic
QQWSSNPFT

SEQ ID NO: 118 - DNA, 1329 bp, synthetic construct
  misc_feature 1..1329: note = Synthetic
  source 1..1329: mol_type = other DNA
gaaggacagctgcagcagtctggacctgagctgcttaaacctggagcctccgtgaagatgagctgcaaggccagcggctacaccttcaccagctacgtgatgcactgggtcaagcagaagccaggccagggccttgagtggatcggctacatcaaccccttcaacgactacaccaactacaacgagaagttccgcggcaaggccacactgaccagcgacaagtctagcaacaccgcctacatggacctgtccagcctgacctctgaggacagcgccgtgtactactgtgccagacaaggtggcagagccctggattattggggccagggcacaagcgttaccgtctcgagcgctaagacaacacctcctagcgtgtaccctctggctcctggatctgccgctcagaccaatagcatggtcaccctgggctgtctggtcaagggctactttcctgagcctgtgaccgtgacctggaacagcggatctctgtctagcggcgtgcacacctttccagccgtgctgcagagcgatctgtacaccctgagcagcagcgtgacagtgcctagctctacctggcctagcgagacagtgacctgcaacgtggcccatcctgccagcagcaccaaggtggacaagaaaatcggcgccgtgcccagagactgcggctgcaagccctgcatctgtaccgtgcctgaagtgtccagcgtgttcatcttcccacctaagcctaaggacgtgctgaccatcacactgacccctaaagtgacctgtgtggtggtggacatcagcaaggacgaccccgaggtgcagttcagttggttcgtggacgacgtggaagtgcacacagcccagacacagcctagagaggaacagttcaacagcaccttcagaagcgtgtccgagctgcccatcatgcaccaggattggctgaacggcaaagaattcaagtgcagagtgaacagcgccgcctttcctgctcctatcgagaaaaccatctccaagaccaagggcagacccaaggctccccaggtgtacacaatccctccacctaaagaacagatggccaaggacaaggtgtccctgacctgcatgatcaccgatttcttcccagaggacatcaccgtggaatggcagtggaatggacagcccgccgagaactacaagaacacccagcctatcatggacaccgacggcagctacttcgtgtacagcaagctgaacgtgcagaagtccaactgggaggccggcaacaccttcacctgttctgtgctgcacgagggcctgcacaatcaccacaccgagaagtccctgagccactctcctggaaag

SEQ ID NO: 119 - protein, 443 aa, synthetic construct
  REGION 1..443: note = Synthetic
EGQLQQSGPELLKPGASVKMSCKASGYTFTSYVMHWVKQKPGQGLEWIGYINPFNDYTNYNEKFRGKATLTSDKSSNTAYMDLSSLTSEDSAVYYCARQGGRALDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIGAVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 120 - DNA, 639 bp, synthetic construct
  misc_feature 1..639: note = Synthetic
  source 1..639: mol_type = other DNA
cagatcgtgctgacacagagccctgccatcatgtctgctagccctggcgagaaagtgaccatgacctgtagcgccagcagcagcgtgacctacatccactggtatcagctgaagtccggcacaagccccaagcggtggatctacgatacaagcaagctggcctctggcgtgcccgctagattttctggctctggcagcggcaccagctacagcctgacaatcagcagcatggaagccgaggatgccgccacctactactgccagcagtggtccagcaatcccttcacatttggctccggcaccaagctggaaatcaagcgggccgacgcggcgcccaccgtgagcatcttcccccccagcagcgagcagctgacaagcggcggagctagcgtcgtgtgcttcctgaacaacttctaccccaaggacatcaacgtgaagtggaagatcgacggcagcgagagacagaacggcgtgctgaatagctggaccgaccaggacagcaaggactccacctacagcatgtccagcacactgaccctgaccaaggacgagtacgagcggcacaacagctacacatgcgaggccacacacaagaccagcacaagccccatcgtgaagtccttcaaccggaacgagtgc

SEQ ID NO: 121 - protein, 213 aa, synthetic construct
  REGION 1..213: note = Synthetic
QIVLTQSPAIMSASPGEKVTMTCSASSSVTYIHWYQLKSGTSPKRWIYDTSKLASGVPARFSGSGSGTSYSLTISSMEAEDAATYYCQQWSSNPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 122 - DNA, 648 bp, synthetic construct
  misc_feature 1..648: note = Synthetic
  source 1..648: mol_type = other DNA
gaaggacagctgcagcagtctggacctgagctgcttaaacctggagcctccgtgaagatgagctgcaaggccagcggctacaccttcaccagctacgtgatgcactgggtcaagcagaagccaggccagggccttgagtggatcggctacatcaaccccttcaacgactacaccaactacaacgagaagttccgcggcaaggccacactgaccagcgacaagtctagcaacaccgcctacatggacctgtccagcctgacctctgaggacagcgccgtgtactactgtgccagacaaggtggcagagccctggattattggggccagggcacaagcgttaccgtctcgagcgctaagacaacacctcctagcgtgtaccctctggctcctggatctgccgctcagaccaatagcatggtcaccctgggctgtctggtcaagggctactttcctgagcctgtgaccgtgacctggaacagcggatctctgtctagcggcgtgcacacctttccagccgtgctgcagagcgatctgtacaccctgagcagcagcgtgacagtgcctagctctacctggcctagcgagacagtgacctgcaacgtggcccatcctgccagcagcaccaaggtggacaagaaaatcggcgcc

SEQ ID NO: 123 - protein, 216 aa, synthetic construct
  REGION 1..216: note = Synthetic
EGQLQQSGPELLKPGASVKMSCKASGYTFTSYVMHWVKQKPGQGLEWIGYINPFNDYTNYNEKFRGKATLTSDKSSNTAYMDLSSLTSEDSAVYYCARQGGRALDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIGA

SEQ ID NO: 124 - DNA, 57 bp, synthetic construct
  misc_feature 1..57: note = Synthetic
  source 1..57: mol_type = other DNA
atggagactgggctgcgctggctgctcctggtcgctgtgctcaaaggtgtccagtgt

SEQ ID NO: 125 - DNA, 351 bp, synthetic construct
  misc_feature 1..351: note = Synthetic
  source 1..351: mol_type = other DNA
cagtcgctggaggagtccgggggtcgcctggtggcgcctgggacacccctgacactcacctgcacagtctctggaatcgacctcagtagctatgcaatgggctgggtccgccaggctccagggaaggggctggaatacatcggatacataagcagtagtggtagcacagactacgcgagctgggcgagaggccgattcaccatctccaaaacctcgtcgaccacggtggatctggaaatgaccagtctgacaaccgaggacacggccacctatttctgtgccagggacacgaattataatggttatcgatttaagatctggggcccaggcaccctggtcaccgtctcctca

SEQ ID NO: 126 - protein, 117 aa, synthetic construct
  REGION 1..117: note = Synthetic
QSLEESGGRLVAPGTPLTLTCTVSGIDLSSYAMGWVRQAPGKGLEYIGYISSSGSTDYASWARGRFTISKTSSTTVDLEMTSLTTEDTATYFCARDTNYNGYRFKIWGPGTLVTVSS

SEQ ID NO: 127 - protein, 8 aa, synthetic construct
  REGION 1..8: note = Synthetic
GIDLSSYA

SEQ ID NO: 128 - protein, 7 aa, synthetic construct
  REGION 1..7: note = Synthetic
ISSSGST

SEQ ID NO: 129 - protein, 13 aa, synthetic construct
  REGION 1..13: note = Synthetic
ARDTNYNGYRFKI

SEQ ID NO: 130 - DNA, 336 bp, synthetic construct
  misc_feature 1..336: note = Synthetic
  source 1..336: mol_type = other DNA
gccgccgtgctgacccagactccagcctccgtgtctgcagctgtgggaggcacagtcaccatcaattgccagcccagtcagagtgtttacaataatcactacttagcctggtatcaacagaaatcagggcagcctcccaagctcctgatctatggtgcatcttatttggaatctggggtcccatcaaggttcaaaggcagtggatctgggacacagttcactctcaccatcagcgacgtacagtgtgacgatgctgccacttactactgtgtgggcgcttatgatagttatagtgatactgctttcggcggcgggaccgaggtggtggtcaaaggt

SEQ ID NO: 131 - protein, 112 aa, synthetic construct
  REGION 1..112: note = Synthetic
AAVLTQTPASVSAAVGGTVTINCQPSQSVYNNHYLAWYQQKSGQPPKLLIYGASYLESGVPSRFKGSGSGTQFTLTISDVQCDDAATYYCVGAYDSYSDTAFGGGTEVVVKG

SEQ ID NO: 132 - protein, 8 aa, synthetic construct
  REGION 1..8: note = Synthetic
QSVYNNHY

SEQ ID NO: 133 - protein, 11 aa, synthetic construct
  REGION 1..11: note = Synthetic
VGAYDSYSDTA

SEQ ID NO: 134 - DNA, 1002 bp, synthetic construct
  misc_feature 1..1002: note = Synthetic
  source 1..1002: mol_type = other DNA
cagtcgctggaggagtccgggggtcgcctggtggcgcctgggacacccctgacactcacctgcacagtctctggaatcgacctcagtagctatgcaatgggctgggtccgccaggctccagggaaggggctggaatacatcggatacataagcagtagtggtagcacagactacgcgagctgggcgagaggccgattcaccatctccaaaacctcgtcgaccacggtggatctggaaatgaccagtctgacaaccgaggacacggccacctatttctgtgccagggacacgaattataatggttatcgatttaagatctggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgtggcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagaggg

SEQ ID NO: 135 - protein, 440 aa, synthetic construct
  REGION 1..440: note = Synthetic
QSLEESGGRLVAPGTPLTLTCTVSGIDLSSYAMGWVRQAPGKGLEYIGYISSSGSTDYASWARGRFTISKTSSTTVDLEMTSLTTEDTATYFCARDTNYNGYRFKIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 136 - DNA, 645 bp, synthetic construct
  misc_feature 1..645: note = Synthetic
  source 1..645: mol_type = other DNA
gccgccgtgctgacccagactccagcctccgtgtctgcagctgtgggaggcacagtcaccatcaattgccagcccagtcagagtgtttacaataatcactacttagcctggtatcaacagaaatcagggcagcctcccaagctcctgatctatggtgcatcttatttggaatctggggtcccatcaaggttcaaaggcagtggatctgggacacagttcactctcaccatcagcgacgtacagtgtgacgatgctgccacttactactgtgtgggcgcttatgatagttatagtgatactgctttcggcggcgggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgt

SEQ ID NO: 137 - protein, 215 aa, synthetic construct
  REGION 1..215: note = Synthetic
AAVLTQTPASVSAAVGGTVTINCQPSQSVYNNHYLAWYQQKSGQPPKLLIYGASYLESGVPSRFKGSGSGTQFTLTISDVQCDDAATYYCVGAYDSYSDTAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 138 - DNA, 954 bp, synthetic construct
  misc_feature 1..954: note = Synthetic
  source 1..954: mol_type = other DNA
cagtcgctggaggagtccgggggtcgcctggtggcgcctgggacacccctgacactcacctgcacagtctctggaatcgacctcagtagctatgcaatgggctgggtccgccaggctccagggaaggggctggaatacatcggatacataagcagtagtggtagcacagactacgcgagctgggcgagaggccgattcaccatctccaaaacctcgtcgaccacggtggatctggaaatgaccagtctgacaaccgaggacacggccacctatttctgtgccagggacacgaattataatggttatcgatttaagatctggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgtgcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 139 - protein, 212 aa, synthetic construct
  REGION 1..212: note = Synthetic
QSLEESGGRLVAPGTPLTLTCTVSGIDLSSYAMGWVRQAPGKGLEYIGYISSSGSTDYASWARGRFTISKTSSTTVDLEMTSLTTEDTATYFCARDTNYNGYRFKIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 140 - DNA, 318 bp, synthetic construct
  misc_feature 1..318: note = Synthetic
  source 1..318: mol_type = other DNA
cagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 141 - protein, 106 aa, synthetic construct
  REGION 1..106: note = Synthetic
QPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 142 - DNA, 624 bp, synthetic construct
  misc_feature 1..624: note = Synthetic
  source 1..624: mol_type = other DNA
gtgttcatcttcccacctaagcctaaggacgtgctgaccatcacactgacccctaaagtgacctgtgtggtggtggacatcagcaaggacgaccccgaggtgcagttcagttggttcgtggacgacgtggaagtgcacacagcccagacacagcctagagaggaacagttcaacagcaccttcagaagcgtgtccgagctgcccatcatgcaccaggattggctgaacggcaaagaattcaagtgcagagtgaacagcgccgcctttcctgctcctatcgagaaaaccatctccaagaccaagggcagacccaaggctccccaggtgtacacaatccctccacctaaagaacagatggccaaggacaaggtgtccctgacctgcatgatcaccgatttcttcccagaggacatcaccgtggaatggcagtggaatggacagcccgccgagaactacaagaacacccagcctatcatggacaccgacggcagctacttcgtgtacagcaagctgaacgtgcagaagtccaactgggaggccggcaacaccttcacctgttctgtgctgcacgagggcctgcacaatcaccacaccgagaagtccctgagccactctcctggaaag

SEQ ID NO: 143 - protein, 208 aa, synthetic construct
  REGION 1..208: note = Synthetic
VFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 144 - DNA, 357 bp, synthetic construct
  misc_feature 1..357: note = Synthetic
  source 1..357: mol_type = other DNA
gatgtgcagattcaggagtcgggacctggcctggtgaaaccttctcagtctctgtccctcacttgcactgtcactggctactcaatcaccagtgattatgcctggaactggatacggcagtttccaggaaacaaactggagtggatgggctacataagctacagtggtagcacttactacaacccatctctcaaaagtcgaatctcattcactcgggacacatccaagaatcagttcttcctgcagctgaactctgtgactactgaggacacagccacatattactgtgcaagatgggagagggacttctatgctatggactactggggtcaaggaacctcagtcaccgtctcgagc

SEQ ID NO: 145 - protein, 119 aa, synthetic construct
  REGION 1..119: note = Synthetic
DVQIQESGPGLVKPSQSLSLTCTVTGYSITSDYAWNWIRQFPGNKLEWMGYISYSGSTYYNPSLKSRISFTRDTSKNQFFLQLNSVTTEDTATYYCARWERDFYAMDYWGQGTSVTVSS

SEQ ID NO: 146 - protein, 9 aa, synthetic construct
  REGION 1..9: note = Synthetic
GYSITSDYA

SEQ ID NO: 147 - protein, 7 aa, synthetic construct
  REGION 1..7: note = Synthetic
ISYSGST

SEQ ID NO: 148 - protein, 12 aa, synthetic construct
  REGION 1..12: note = Synthetic
ARWERDFYAMDY

SEQ ID NO: 149 - DNA, 321 bp, synthetic construct
  misc_feature 1..321: note = Synthetic
  source 1..321: mol_type = other DNA
gacattgtgatgacccagtctcacaaattcatgtccacatcaataggagacagggtcagcatcacttgcaatgccagtcagggtgtgactactgctgtagcctggtatcaacagaaaccaggacagtctcctaaactactgatttcctcggcatcctaccggtacactggagtccctgatcgcttcactggcagtggatctgggacggatttcactttcaccatcagcagtgtgcaggctgaagacctggcactttattactgtcagcaatattatagtcctacgtggacgttcggtggaggcaccaagctggaaatcaaa

SEQ ID NO: 150 - protein, 107 aa, synthetic construct
  REGION 1..107: note = Synthetic
DIVMTQSHKFMSTSIGDRVSITCNASQGVTTAVAWYQQKPGQSPKLLISSASYRYTGVPDRFTGSGSGTDFTFTISSVQAEDLALYYCQQYYSPTWTFGGGTKLEIK

SEQ ID NO: 151 - protein, 6 aa, synthetic construct
  REGION 1..6: note = Synthetic
QGVTTA

SEQ ID NO: 152 - protein, 9 aa, synthetic construct
  REGION 1..9: note = Synthetic
QQYYSPTWT

SEQ ID NO: 153 - DNA, 1332 bp, synthetic construct
  misc_feature 1..1332: note = Synthetic
  source 1..1332: mol_type = other DNA
gatgtgcagattcaggagtcgggacctggcctggtgaaaccttctcagtctctgtccctcacttgcactgtcactggctactcaatcaccagtgattatgcctggaactggatacggcagtttccaggaaacaaactggagtggatgggctacataagctacagtggtagcacttactacaacccatctctcaaaagtcgaatctcattcactcgggacacatccaagaatcagttcttcctgcagctgaactctgtgactactgaggacacagccacatattactgtgcaagatgggagagggacttctatgctatggactactggggtcaaggaacctcagtcaccgtctcgagcgctaagaccaccccccccagcgtgtaccccctggcccccggcagcgccgcccagaccaacagcatggtgaccctgggctgcctggtgaagggctacttccccgagcccgtgaccgtgacctggaacagcggcagcctgagcagcggcgtgcacaccttccccgccgtgctgcagagcgacctgtacaccctgagcagcagcgtgaccgtgcccagcagcacctggcccagcgagaccgtgacctgcaacgtggcccaccccgccagcagcaccaaggtggacaagaagatcgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 154 - protein, 444 aa, synthetic construct
  REGION 1..444: note = Synthetic
DVQIQESGPGLVKPSQSLSLTCTVTGYSITSDYAWNWIRQFPGNKLEWMGYISYSGSTYYNPSLKSRISFTRDTSKNQFFLQLNSVTTEDTATYYCARWERDFYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 155 - DNA, 642 bp, synthetic construct
  misc_feature 1..642: note = Synthetic
  source 1..642: mol_type = other DNA
gacattgtgatgacccagtctcacaaattcatgtccacatcaataggagacagggtcagcatcacttgcaatgccagtcagggtgtgactactgctgtagcctggtatcaacagaaaccaggacagtctcctaaactactgatttcctcggcatcctaccggtacactggagtccctgatcgcttcactggcagtggatctgggacggatttcactttcaccatcagcagtgtgcaggctgaagacctggcactttattactgtcagcaatattatagtcctacgtggacgttcggtggaggcaccaagctggaaatcaaacgggccgacgcggcgcccaccgtgagcatcttcccccccagcagcgagcagctgaccagcggcggcgcgagcgtggtgtgcttcctgaacaacttctaccccaaggacatcaacgtgaagtggaagatcgacggcagcgagaggcagaacggcgtgctgaacagctggaccgaccaggacagcaaggacagcacctacagcatgagcagcaccctgaccctgaccaaggacgagtacgagaggcacaacagctacacatgtgaggccacccacaagaccagcaccagccccatcgtgaagagcttcaacaggaacgagtgc

SEQ ID NO: 156 - protein, 214 aa, synthetic construct
  REGION 1..214: note = Synthetic
DIVMTQSHKFMSTSIGDRVSITCNASQGVTTAVAWYQQKPGQSPKLLISSASYRYTGVPDRFTGSGSGTDFTFTISSVQAEDLALYYCQQYYSPTWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 157 - DNA, 648 bp, synthetic construct
  misc_feature 1..648: note = Synthetic
  source 1..648: mol_type = other DNA
gatgtgcagattcaggagtcgggacctggcctggtgaaaccttctcagtctctgtccctcacttgcactgtcactggctactcaatcaccagtgattatgcctggaactggatacggcagtttccaggaaacaaactggagtggatgggctacataagctacagtggtagcacttactacaacccatctctcaaaagtcgaatctcattcactcgggacacatccaagaatcagttcttcctgcagctgaactctgtgactactgaggacacagccacatattactgtgcaagatgggagagggacttctatgctatggactactggggtcaaggaacctcagtcaccgtctcgagcgctaagaccaccccccccagcgtgtaccccctggcccccggcagcgccgcccagaccaacagcatggtgaccctgggctgcctggtgaagggctacttccccgagcccgtgaccgtgacctggaacagcggcagcctgagcagcggcgtgcacaccttccccgccgtgctgcagagcgacctgtacaccctgagcagcagcgtgaccgtgcccagcagcacctggcccagcgagaccgtgacctgcaacgtggcccaccccgccagcagcaccaaggtggacaagaagatc

SEQ ID NO: 158 - protein, 216 aa, synthetic construct
  REGION 1..216: note = Synthetic
DVQIQESGPGLVKPSQSLSLTCTVTGYSITSDYAWNWIRQFPGNKLEWMGYISYSGSTYYNPSLKSRISFTRDTSKNQFFLQLNSVTTEDTATYYCARWERDFYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKI

SEQ ID NO: 159 - DNA, 291 bp, synthetic construct
  misc_feature 1..291: note = Synthetic
  source 1..291: mol_type = other DNA
gctaagaccaccccccccagcgtgtaccccctggcccccggcagcgccgcccagaccaacagcatggtgaccctgggctgcctggtgaagggctacttccccgagcccgtgaccgtgacctggaacagcggcagcctgagcagcggcgtgcacaccttccccgccgtgctgcagagcgacctgtacaccctgagcagcagcgtgaccgtgcccagcagcacctggcccagcgagaccgtgacctgcaacgtggcccaccccgccagcagcaccaaggtggacaagaagatc

SEQ ID NO: 160 - protein, 97 aa, synthetic construct
  REGION 1..97: note = Synthetic
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKI

SEQ ID NO: 161 - DNA, 321 bp, synthetic construct
  misc_feature 1..321: note = Synthetic
  source 1..321: mol_type = other DNA
cgggccgacgcggcgcccaccgtgagcatcttcccccccagcagcgagcagctgaccagcggcggcgcgagcgtggtgtgcttcctgaacaacttctaccccaaggacatcaacgtgaagtggaagatcgacggcagcgagaggcagaacggcgtgctgaacagctggaccgaccaggacagcaaggacagcacctacagcatgagcagcaccctgaccctgaccaaggacgagtacgagaggcacaacagctacacatgtgaggccacccacaagaccagcaccagccccatcgtgaagagcttcaacaggaacgagtgc

SEQ ID NO: 162 - protein, 114 aa, synthetic construct
  REGION 1..114: note = Synthetic
QSVEESGGRLVTPGTPLTLTCTVSGFSLNNHAMGWVRQAPRKGLEYIGYISISGTTYYASWAKGRFTISKTSTTVDLKITSPTTEDTGTYFCGRGWASDNLYLWGQGTLVTVSS

SEQ ID NO: 163 - protein, 8 aa, synthetic construct
  REGION 1..8: note = Synthetic
GFSLNNHA

SEQ ID NO: 164 - protein, 7 aa, synthetic construct
  REGION 1..7: note = Synthetic
ISISGTT

SEQ ID NO: 165 - protein, 11 aa, synthetic construct
  REGION 1..11: note = Synthetic
GRGWASDNLYL

SEQ ID NO: 166 - DNA, 345 bp, synthetic construct
  misc_feature 1..345: note = Synthetic
  source 1..345: mol_type = other DNA
gcccaagttctgacaacccagactccatcgtccgtgtctgcagctgtgggaggcacagtcaccatcaactgccaggccagtcagagtctttataataagaaaaatttagcctggtttcagcagaaaccagggcagcctcccaagcgcctgatctattatgcatccactctggcatctggggtctcatcgcggttcaaaggcagtggatctgggacacagttcactctcaccatcagtgacgtgcagtgtgacgatactgccacttactactgtctaggcgactttgattgtagtagtgctgattgttatagtttcggcggagggaccgaggtggtcgtcaaaggt

SEQ ID NO: 167 - protein, 115 aa, synthetic construct
  REGION 1..115: note = Synthetic
AQVLTTQTPSSVSAAVGGTVTINCQASQSLYNKKNLAWFQQKPGQPPKRLIYYASTLASGVSSRFKGSGSGTQFTLTISDVQCDDTATYYCLGDFDCSSADCYSFGGGTEVVVKG

SEQ ID NO: 168 - protein, 8 aa, synthetic construct
  REGION 1..8: note = Synthetic
QSLYNKKN

SEQ ID NO: 169 - protein, 13 aa, synthetic construct
  REGION 1..13: note = Synthetic
LGDFDCSSADCYS

SEQ ID NO: 170 - DNA, 1311 bp, synthetic construct
  misc_feature 1..1311: note = Synthetic
  source 1..1311: mol_type = other DNA
cagtcggtggaggagtccgggggtcgcctggtcacgcctgggacacccctgacactcacctgcaccgtctctggattctccctcaataaccatgcaatgggctgggtccgccaggctcctaggaaggggctggaatatatcggatacattagtattagtggtaccacatactacgcgagctgggcgaaaggccgattcaccatctccaagacctcgaccacggtggatctgaaaatcaccagtccgacaaccgaggacacgggcacctatttctgtggcagaggttgggctagtgataatttgtacttgtggggccaaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgtggcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 171 - protein, 437 aa, synthetic construct
  REGION 1..437: note = Synthetic
QSVEESGGRLVTPGTPLTLTCTVSGFSLNNHAMGWVRQAPRKGLEYIGYISISGTTYYASWAKGRFTISKTSTTVDLKITSPTTEDTGTYFCGRGWASDNLYLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 172 - DNA, 654 bp, synthetic construct
  misc_feature 1..654: note = Synthetic
  source 1..654: mol_type = other DNA
gcccaagttctgacaacccagactccatcgtccgtgtctgcagctgtgggaggcacagtcaccatcaactgccaggccagtcagagtctttataataagaaaaatttagcctggtttcagcagaaaccagggcagcctcccaagcgcctgatctattatgcatccactctggcatctggggtctcatcgcggttcaaaggcagtggatctgggacacagttcactctcaccatcagtgacgtgcagtgtgacgatactgccacttactactgtctaggcgactttgattgtagtagtgctgattgttatagtttcggcggagggaccgaggtggtcgtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgt

SEQ ID NO: 173 - protein, 218 aa, synthetic construct
  REGION 1..218: note = Synthetic
AQVLTTQTPSSVSAAVGGTVTINCQASQSLYNKKNLAWFQQKPGQPPKRLIYYASTLASGVSSRFKGSGSGTQFTLTISDVQCDDTATYYCLGDFDCSSADCYSFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 174 - DNA, 627 bp, synthetic construct
  misc_feature 1..627: note = Synthetic
  source 1..627: mol_type = other DNA
cagtcggtggaggagtccgggggtcgcctggtcacgcctgggacacccctgacactcacctgcaccgtctctggattctccctcaataaccatgcaatgggctgggtccgccaggctcctaggaaggggctggaatatatcggatacattagtattagtggtaccacatactacgcgagctgggcgaaaggccgattcaccatctccaagacctcgaccacggtggatctgaaaatcaccagtccgacaaccgaggacacgggcacctatttctgtggcagaggttgggctagtgataatttgtacttgtggggccaaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgtg

SEQ ID NO: 175 - protein, 209 aa, synthetic construct
  REGION 1..209: note = Synthetic
QSVEESGGRLVTPGTPLTLTCTVSGFSLNNHAMGWVRQAPRKGLEYIGYISISGTTYYASWAKGRFTISKTSTTVDLKITSPTTEDTGTYFCGRGWASDNLYLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTV

SEQ ID NO: 176 - DNA, 285 bp, synthetic construct
  misc_feature 1..285: note = Synthetic
  source 1..285: mol_type = other DNA
gggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacttgcaacgtggcccacccagccaccaacaccaaagtggacaagaccgtg

SEQ ID NO: 177 - DNA, 285 bp, synthetic construct
  misc_feature 1..285: note = Synthetic
  source 1..285: mol_type = other DNA
gggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgtg

SEQ ID NO: 178 - DNA, 309 bp, synthetic construct
  misc_feature 1..309: note = Synthetic
  source 1..309: mol_type = other DNA
tctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagaggg

SEQ ID NO: 179 - DNA, 318 bp, synthetic construct
  misc_feature 1..318: note = Synthetic
  source 1..318: mol_type = other DNA
cagcccctggagccgaaggtctacaccatgggccctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaa

SEQ ID NO: 180 - DNA, 627 bp, synthetic construct
  misc_feature 1..627: note = Synthetic
  source 1..627: mol_type = other DNA
tctgtcttcatcttccccccaaaacccaaggacaccctcatgatctcacgcacccccgaggtgacctgcgtggtggtggacgtgagccaggatgaccccgaggtgcagttcacatggtacataaacaacgagcaggtccgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcctccatcgagaagaccatcagcaaggccagagggcagcccctggagccgaaggtctacaccatgggtcctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacttgctccgtgatgcacgaggccttgcacaaccactacacgcagaagtccatctcccgctctccgggtaaa

SEQ ID NO: 181 - DNA, 342 bp, synthetic construct
  misc_feature 1..342: note = Synthetic
  source 1..342: mol_type = other DNA
cagtcggtggaggagtccgggggtcgcctggtcacgcctgggacacccctgacactcacctgcaccgtctctggattctccctcaataaccatgcaatgggctgggtccgccaggctcctaggaaggggctggaatatatcggatacattagtattagtggtaccacatactacgcgagctgggcgaaaggccgattcaccatctccaagacctcgaccacggtggatctgaaaatcaccagtccgacaaccgaggacacgggcacctatttctgtggcagaggttgggctagtgataatttgtacttgtggggccaaggcaccctggtcaccgtctcctca

(19) United States
(12) Patent Application Publication
(10) Pub. No.: US 2026/0308277 A1
(43) Pub. Date: Oct. 8, 2026

(54) Engineered Antibodies and Uses Thereof

(71) Applicant: Invitrogen BioServices India Private Limited, Bangalore (IN)

(72) Inventors: Haripriya Sridharan, Bangalore (IN); Swathi SundarRaj, Bangalore (IN); Pavitra Bimal Desai, Greensboro, NC (US); Thangaraj Karuppuchamy, Bangalore (IN); Sharad Vashisht, Chicago, IL (US)

(21) Appl. No.: 19/482,219

(22) PCT Filed: Sep. 13, 2024
(86) PCT No.: PCT/IN2024/051736

§ 371 Date: Nov. 6, 2025

(30) Foreign Application Priority Data
Sep. 14, 2023 (IN) IN202341061835
Sep. 14, 2023 (IN) IN202341061917
Sep. 3, 2024 (IN) IN202441066590

(51) Int. Cl. C07K 16/40; C07K 16/18; C07K 16/44; G01N 33/58; G01N 33/68

(52) U.S. Cl. CPC C07K 16/40; C07K 16/18; C07K 16/44; G01N 33/582; G01N 33/6854; C07K 2317/524; C07K 2317/526; C07K 2317/53; C07K 2317/55; C07K 2317/565; C07K 2317/624; C07K 2317/92; G01N 2333/47

(57) ABSTRACT

Disclosed herein are isolated polypeptides and antibodies with improved sensitivity and methods of using the same.

### CROSS-REFERENCE TO RELATED APPLICATIONS

This application is a 35 U.S.C 371 national phase of PCT International Application No. PCT/IN2024/051736, filed on Sep. 13, 2024, which claims the benefit under 35 U.S.C. § 119(e) and priority to Indian Provisional Application no. IN202441066590, filed Sep. 3, 2024, Indian Provisional Application no. IN202341061917, filed Sep. 14, 2023, and Indian Provisional Application no. IN202341061835, filed Sep. 14, 2023. The entire contents of the aforementioned applications are incorporated by reference herein.

### REFERENCE TO ELECTRONIC SEQUENCE LISTING

This application contains a Sequence Listing which has been submitted electronically herewith in .XML format and is hereby incorporated by reference in its entirety. Said .XML copy, created on Sep. 10, 2024, is named “TP385350WO1.xml” and is 279,164 bytes in size. The sequence listing contained in this .XML file is part of the specification and is hereby incorporated by reference herein in its entirety.

### TECHNICAL FIELD

This invention relates to isolated polypeptides and antibodies with improved sensitivity and methods of use thereof.

### BACKGROUND

Recombinant antibodies offer the ability to rationally engineer beneficial properties. One such beneficial property is antibody sensitivity, i.e., the strength of the signal produced in a particular immunoassay. A highly sensitive antibody has several advantages such as the detection of low abundance or rare targets and the ability to detect the target protein in difficult imaging assays. Antibody sensitivity can be increased by affinity maturation of the antibody clone, which involves mutation of the antigen binding site or paratope. However, affinity maturation has to be tailored for each antibody under consideration and is therefore not a scalable solution to address a large number of antibodies. In addition, introduction of mutations to the paratope changes the clone of the antibody and essentially produces a new antibody. There is a need for improved approaches for increasing antibody sensitivity by, for example, rational modification of the antibody Fc region. The disclosed peptides, antibodies, compositions and uses thereof are directed to these and other important needs.

### SUMMARY

Disclosed herein are isolated polypeptides comprising the amino acid sequence of Formula I: Z-(X-Yₙ-Zᵣ)ₚ-X, wherein Z is a hinge region, X is a CH2 domain, Y is a linker, n is an integer of 1 or greater, p is an integer of 2 or greater, and r is an integer of 0 or 1. In certain embodiments, the isolated polypeptides have at least a 1.5-fold increase in signal enhancement or sensitivity. In certain embodiments, the isolated polypeptides have at least a 1.5-fold reduction in background signal.

Disclosed herein are antibodies comprising a polypeptide comprising the amino acid sequence of Formula I: Z-(X-Yₙ-Zᵣ)ₚ-X, wherein Z is a hinge region, X is a CH2 domain, Y is a linker, n is an integer of 1 or greater, p is an integer of 2 or greater, and r is an integer of 0 or 1 linked to a Fab domain. In certain embodiments, the antibodies have at least a 1.5-fold increase in signal enhancement or sensitivity compared to a corresponding wild type antibody. In certain embodiments, the antibodies have at least a 1.5-fold reduction in background signal compared to a corresponding wild type antibody.

Disclosed herein are antibodies that immunospecifically bind to Parkin, wherein the antibodies comprise a heavy chain (HC) and a light chain (LC) that pair to form a binding domain comprising a heavy chain complementarity determining region 1 (HCDR1) comprising the amino acid sequence of SEQ ID NO: 4, a HCDR2 comprising the amino acid sequence of SEQ ID NO: 5, a HCDR3 comprising the amino acid sequence of SEQ ID NO: 6, a light chain complementarity determining region 1 (LCDR1) comprising the amino acid sequence of SEQ ID NO: 13, a LCDR2 comprising the amino acid sequence RAS, and a LCDR3 comprising the amino acid sequence of SEQ ID NO: 15, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

Disclosed herein are antibodies that immunospecifically bind to OCT4, wherein the antibodies comprise a heavy chain (HC) and a light chain (LC) that pair to form a binding domain comprising a heavy chain complementarity determining region 1 (HCDR1) comprising the amino acid sequence of SEQ ID NO: 29, a HCDR2 comprising the amino acid sequence of SEQ ID NO: 30, and a HCDR3 comprising the amino acid sequence of SEQ ID NO: 31, a light chain complementarity determining region 1 (LCDR1) comprising the amino acid sequence of SEQ ID NO: 37, a LCDR2 comprising the amino acid sequence GAS, and a LCDR3 comprising the amino acid sequence of SEQ ID NO: 39, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

Disclosed herein are antibodies that immunospecifically bind to IDO1, wherein the antibodies comprise a heavy chain (HC) and a light chain (LC) that pair to form a binding domain comprising a heavy chain complementarity determining region 1 (HCDR1) comprising the amino acid sequence of SEQ ID NO: 66, a HCDR2 comprising the amino acid sequence of SEQ ID NO: 67, and a HCDR3 comprising the amino acid sequence of SEQ ID NO: 68, a light chain complementarity determining region 1 (LCDR1) comprising the amino acid sequence of SEQ ID NO: 72, a LCDR2 comprising the amino acid sequence of YAS, and a LCDR3 comprising the amino acid sequence of SEQ ID NO: 73, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

Disclosed herein are antibodies that immunospecifically bind to FLAG-tag, wherein the antibodies comprise a heavy chain (HC) and a light chain (LC) that pair to form a binding domain comprising a heavy chain complementarity determining region 1 (HCDR1) comprising the amino acid sequence of SEQ ID NO: 163, a HCDR2 comprising the amino acid sequence of SEQ ID NO: 164, and a HCDR3 comprising the amino acid sequence of SEQ ID NO: 165, a light chain complementarity determining region 1 (LCDR1) comprising the amino acid sequence of SEQ ID NO: 168, a LCDR2 comprising the amino acid sequence of YAS, and a LCDR3 comprising the amino acid sequence of SEQ ID NO: 169, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

Disclosed herein are rabbit anti-mouse (RAM) secondary antibodies, wherein the antibodies comprise a heavy chain (HC) and a light chain (LC) that pair to form a binding domain comprising a heavy chain complementarity determining region 1 (HCDR1) comprising the amino acid sequence of SEQ ID NO: 127, a HCDR2 comprising the amino acid sequence of SEQ ID NO: 128, and a HCDR3 comprising the amino acid sequence of SEQ ID NO: 129, a light chain complementarity determining region 1 (LCDR1) comprising the amino acid sequence of SEQ ID NO: 132, a LCDR2 comprising the amino acid sequence of GAS, and a LCDR3 comprising the amino acid sequence of SEQ ID NO: 133, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

Disclosed herein are antibodies that immunospecifically bind to pERK1/2, wherein the antibodies comprise a heavy chain (HC) and a light chain (LC) that pair to form a binding domain comprising a heavy chain complementarity determining region 1 (HCDR1) comprising the amino acid sequence of SEQ ID NO: 85, a HCDR2 comprising the amino acid sequence of SEQ ID NO: 86, and a HCDR3 comprising the amino acid sequence of SEQ ID NO: 87, a light chain complementarity determining region 1 (LCDR1) comprising the amino acid sequence of SEQ ID NO: 91, a LCDR2 comprising the amino acid sequence of KVS, and a LCDR3 comprising the amino acid sequence of SEQ ID NO: 92, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

Disclosed herein are antibodies that immunospecifically bind to PSD-95, wherein the antibodies comprise a heavy chain (HC) and a light chain (LC) that pair to form a binding domain comprising a heavy chain complementarity determining region 1 (HCDR1) comprising the amino acid sequence of SEQ ID NO: 111, a HCDR2 comprising the amino acid sequence of SEQ ID NO: 112, and a HCDR3 comprising the amino acid sequence of SEQ ID NO: 113, a light chain complementarity determining region 1 (LCDR1) comprising the amino acid sequence of SEQ ID NO: 116, a LCDR2 comprising the amino acid sequence of DTS, and a LCDR3 comprising the amino acid sequence of SEQ ID NO: 117, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

Disclosed herein are antibodies that immunospecifically bind to TIGIT, wherein the antibodies comprise a heavy chain (HC) and a light chain (LC) that pair to form a binding domain comprising a heavy chain complementarity determining region 1 (HCDR1) comprising the amino acid sequence of SEQ ID NO: 146, a HCDR2 comprising the amino acid sequence of SEQ ID NO: 147, and a HCDR3 comprising the amino acid sequence of SEQ ID NO: 148, a light chain complementarity determining region 1 (LCDR1) comprising the amino acid sequence of SEQ ID NO: 151, a LCDR2 comprising the amino acid sequence of SAS, and a LCDR3 comprising the amino acid sequence of SEQ ID NO: 152, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

Disclosed herein are methods of detecting the presence of a target protein in a sample, said methods comprising applying the herein disclosed isolated polypeptides or the herein disclosed antibodies to a sample containing a target protein, incubating the isolated polypeptide or the antibody in the sample under conditions permitting the isolated polypeptides or the polypeptide or the antibody to bind to the target protein and to form a bound isolated polypeptide or a bound polypeptide or the antibody, and determining the presence of the bound isolated polypeptide or the bound polypeptide or the antibody, thereby detecting the presence of the target protein.

Disclosed herein are methods of increasing the sensitivity of an antibody, comprising introducing into a heavy chain of the antibody a polypeptide comprising the amino acid sequence of Formula I: Z-(X-Yₙ-Zᵣ)ₚ-X, wherein Z is a hinge, X is a CH2 domain, Y is a linker, n is an integer of 1 or greater, p is an integer of 2 or greater, and r is an integer of 0 or 1, wherein the sensitivity of the antibody is increased at least 1.5-fold compared to a corresponding wild type antibody. In certain embodiments, the method further comprises introducing LFLEPS mutations into the amino acid sequence of X, wherein the LFLEPS mutations comprise L234F, L235E and P331S substitutions.

Disclosed herein are methods of reducing the background signal of an antibody, comprising introducing into a heavy chain of the antibody a polypeptide comprising the amino acid sequence of Formula I: Z-(X-Yₙ-Zᵣ)ₚ-X, wherein Z is a hinge, X is a CH2 domain, Y is a linker, n is an integer of 1 or greater, p is an integer of 2 or greater, and r is an integer of 0 or 1, wherein the background signal of the antibody is reduced at least 1.5-fold compared to a corresponding wild type antibody. In certain embodiments, the method further comprises introducing LFLEPS mutations into the amino acid sequence of X, wherein the LFLEPS mutations comprise L234F, L235E and P331S substitutions.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1A is a schematic diagram of Parent wild type antibody (WT IgG, ~150 kDa)

FIG. 1B is a schematic diagram of the 1TdFc design (IgG-2Fc, ~200 kDa). The 1TdFc design has one additional Fc domain connected by a linker and hinge.

FIG. 1C is a schematic diagram of the 2TdFc design (IgG-3Fc, ~250 kDa). The 2TdFc design has two additional Fc domains connected by a linker and hinge.

FIG. 1D is a schematic diagram of the 2TdCH2 design (~200 kDa). The 2TdCH2 design has two additional CH2 domains connected by linker and optional hinge.

FIG. 1E is a schematic diagram of the 2TdCH2-LFLEPS design (~200 kDa). The 2TdCH2-LFLEPS design has two additional CH2 domains connected by a linker and optional hinge, and FcR mutations (stars) represented by the acronym “LFLEPS”.

FIG. 2A is a plasmid map showing that the polynucleotide sequence of the anti-OCT4 antibody heavy chain (HC) with the 2TdCH2-LFLEPS configuration was synthesized and cloned between XbaI-NotI restriction sites in pcDNA3.4 Vector. XbaI-KasI restriction enzyme can be used for subcloning of another target HC “Fab” region.

FIG. 2B is a plasmid map showing that the polynucleotide sequence of the anti-OCT4 antibody light chain (LC) was synthesized and cloned between XbaI-NotI restriction sites in pcDNA3.4 Vector. XbaI-NotI restriction enzyme can be used for subcloning of another target LC “Fab” region.

FIG. 2C shows a restriction digestion analysis of the anti-OCT4 antibody HC with the 2TdCH2-LFLEPS configuration and anti-OCT4 antibody LC. Anti-OCT4 antibody HC 2TdCH2-LFLEPS shows a 2203 bp insert released upon XbaI-NotI digestion. Anti-OCT4 antibody LC shows a 746 bp insert released upon XbaI-NotI digestion.

FIG. 3A is a plasmid map showing that the polynucleotide sequence of the anti-Parkin antibody heavy chain (HC) with 2TdCH2-LFLEPS configuration was synthesized and cloned between XbaI-NotI restriction sites in pcDNA3.4 Vector. XbaI-KasI restriction enzyme can be used for subcloning of another target HC “Fab” region.

FIG. 3B is a plasmid map showing that the polynucleotide sequence of the anti-Parkin antibody light chain (LC) was synthesized and cloned between XbaI-NotI restriction sites in pcDNA3.4 Vector. XbaI-NotI restriction enzyme can be used for subcloning of another target LC “Fab” region.

FIG. 3C shows a restriction digestion analysis of anti-Parkin antibody HC with the 2TdCH2-LFLEPS configuration and anti-Parkin antibody LC. Anti-Parkin antibody HC 2TdCH2-LFLEPS shows a 2218 bp insert released upon XbaI-NotI digestion. Anti-Parkin antibody LC shows a 743 bp insert released upon XbaI-NotI digestion.

FIG. 4A shows a representative HPLC profile of parent anti-Parkin wild type (WT) antibody having purity of 95% monomer.

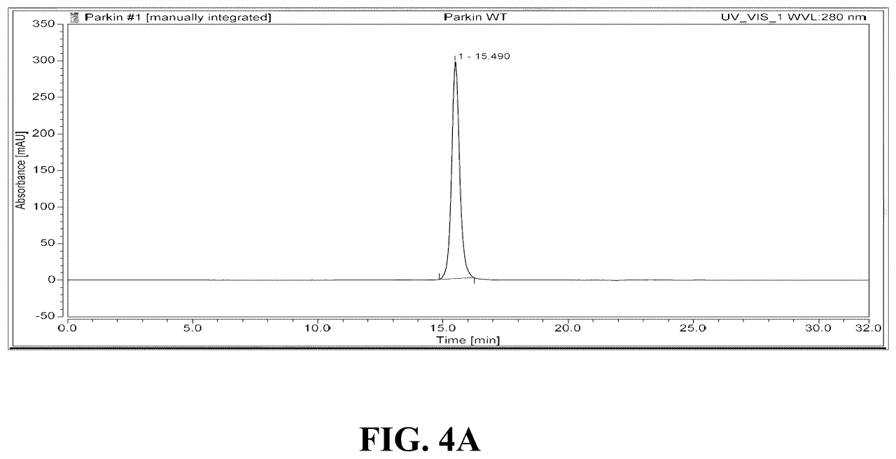

FIG. 4B shows a representative HPLC profile of anti-OCT4 1TdFc antibody having purity of 63% monomer.

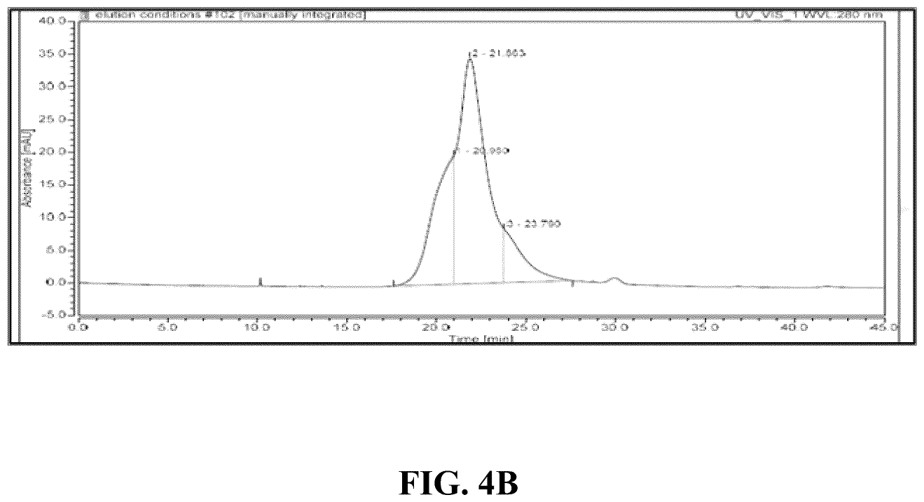

FIG. 4C shows a representative HPLC profile of anti-Parkin 2TdFc antibody having purity of 33% monomer.

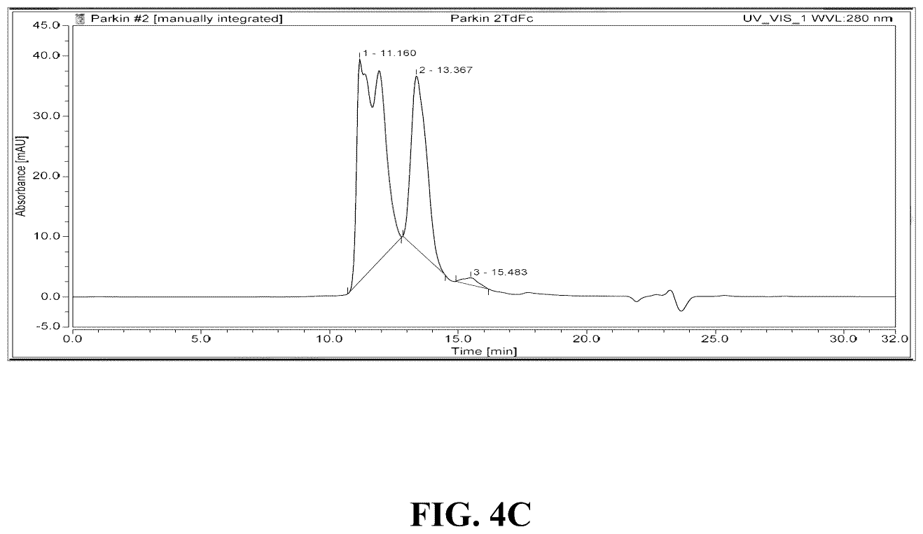

FIG. 4D shows a representative HPLC profile of anti-Parkin 2TdCH2-LFLEPS antibody having purity of 87% monomer.

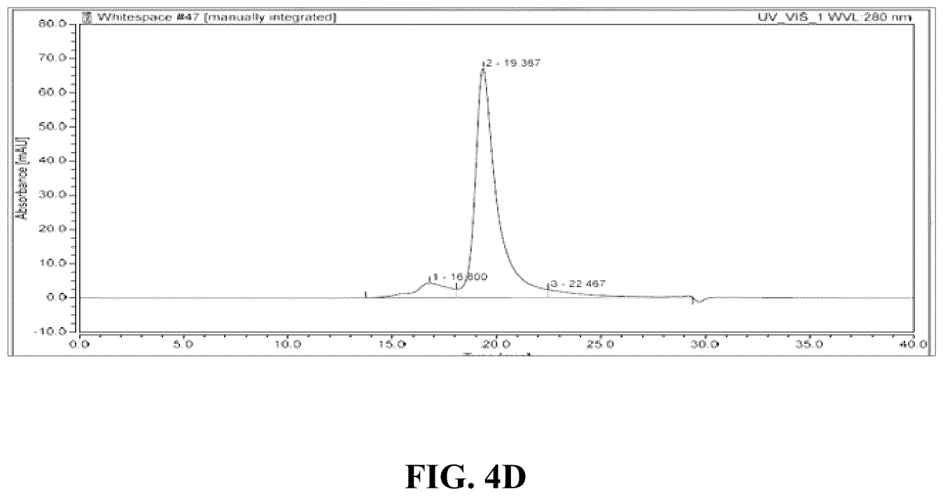

FIG. 5A is a western blot (WB) analysis performed on whole cell extracts of rat brain tissue (left panel) and PC-12 cells (right panel). The blots were probed with wild type (WT) and engineered anti-Parkin antibodies (1TdFC, 2TdFc, 2TdCH2). A ~48 kDa band corresponding to Parkin was observed in rat brain and PC-12 cell lysates tested.

FIG. 5B is a western blot (top panel) showing how antibody specificity was demonstrated by siRNA-mediated knockdown of the target protein. SH-SY5Y cells were transfected with Parkin siRNA and a decrease in signal intensity was observed in western blot application using anti-Parkin 2TdCH2-LFLEPS antibody. The bottom panel is a graphical representation of the western blot analysis.

FIG. 5C is a western blot analysis that was performed on whole cell extracts of PC-12 cells. The blots were probed with wild type (WT) and engineered anti-Parkin antibodies (2TdCH2, 2TdCH2-LFLEPS). A similar intensity of ~48 kDa band corresponding to Parkin was observed in PC-12 cell lysates with 2TdCH2 vs 2TdCH2-LFLEPS.

FIG. 5D is a scatter plot analysis of the WB data: anti-Parkin 2TdCH2 antibody shows significant fold enhancement (**p<0.01) compared to WT antibody.

FIG. 6A is a western blot analysis that was performed on whole cell extracts of NTERA-2 (left panel) and F9 cells (right panel). The blots were probed with wild type (WT) and engineered anti-OCT4 antibodies (1TdFC, 2TdFc, 2TdCH2). A ~38 kDa band corresponding to OCT4 was observed in the cell lysates tested.

FIG. 6B is a western blot analysis performed on whole cell extracts of NTERA-2 cells. The blots were probed with wild type (WT) and engineered anti-OCT4 antibodies (2TdCH2, 2TdCH2-LFLEPS). A similar intensity of ~38 kDa band corresponding to OCT4 was observed in NTERA-2 cell lysates with anti-OCT4 2TdCH2 vs 2TdCH2-LFLEPS antibodies.

FIG. 6C shows antibody specificity was demonstrated by relative expression (top panel) with BOI in NTERA-2, NCCIT and F9 cells. HEK293, A431 and HELA were negative cell lines. siRNA-mediated knockdown (bottom panel) of the target protein. NTERA-2 cells were transfected with OCT4 siRNA and a decrease in signal intensity was observed in western blot application using anti-OCT4 2TdCH2-LFLEPS antibody.

FIG. 6D is a scatter plot analysis of WB data: anti-OCT4 2TdCH2 antibody show significant fold enhancement (****p<0.0001) compared to WT antibody.

FIG. 7A is a bar graph analysis of western blot data obtained from anti-Parkin 2TdCH2 antibody with various secondary antibodies as indicated. The anti-Parkin 2TdCH2 antibody showed fold enhancement across different secondaries compared to wild type (WT) antibody.

FIG. 7B is a bar graph analysis of western blot data obtained from anti-OCT4 2TdCH2 antibody with various secondary antibodies as indicated. The anti-OCT4 2TdCH2 antibody showed fold enhancement across different secondaries compared to wild type (WT) antibody.

FIG. 8A is an immunofluorescent analysis of OCT4 performed using 70% confluent NTERA-2 cells. The cells were stained with different concentrations of anti-OCT4 wild type (WT) antibody (upper panel) or anti-OCT4 2TdCH2-LFLEPS antibody (1:1,000) (lower panel).

FIG. 8B shows a knockdown of OCT4 was achieved by transfecting NTERA-2 cells with specific validated siRNA (Silencer® select Product #s10871, s10873). Immunofluorescence analysis was performed on NTERA-2 cells (untransfected, panels a-d), transfected with OCT4 specific siRNA (panel i-1) or non-specific scrambled siRNA (panels e-h).

FIG. 9A is an immunohistochemical analysis of Parkin performed using formalin-fixed paraffin-embedded mouse kidney tissue sections. The sections were probed with anti-Parkin wild type (WT) (lower panel) or anti-Parkin 2TdCH2-LFLEPS (upper panel) (1:100 dilution) antibody.

FIG. 9B is an immunohistochemical analysis of Parkin performed using formalin-fixed paraffin-embedded mouse kidney tissue sections. The sections were probed with anti-Parkin polyclonal antibody (Benchmark (BM) or positive control) (lower panel) or Parkin 2TdCH2-LFLEPS (upper panel) (1:100 dilution) antibody.

FIG. 10 is a flow cytometry analysis of Parkin performed in PC-12 cells. Cells were fixed and permeabilized using the Intracellular Fixation & Permeabilization Buffer Set (Product #88-8824-00) and then stained intracellularly with 2 μg of anti-Parkin wild type (WT) antibody or anti-Parkin 2TdCH2-LFLEPS antibody or positive control (PC) antibody (PA5-1339) antibody. Unstained samples were used as a control. The top panel shows the flow cytometry spectra and the bottom panel shows a graphical representation of the flow data.

FIG. 11 is a graphical representation of an immunofluorescent analysis of IDO1 that was performed using 70% confluent CD64-GFP (FcγR1) transfected HEK-293E cells demonstrating the amount of FcγR1 binding by the antibodies. The cells were stained with 0.5 μg/ml and 2.5 μg/ml of IDO1 wild type (WT) ±LFLEPS antibody or anti-IDO1 2TdCH2±LFLEPS antibody followed by Goat anti-Rabbit Alexa Fluor™ 647 secondary antibody (Product #A21244).

FIG. 12A is a flow cytometry analysis of IDO1 that was performed using 70% confluent CD64-GFP (FcγR1) transfected HEK-293E cells. The cells were stained with 0.5 μg/ml (lower panel) and 2.5 μg/ml (upper panel) with anti-IDO1 wild type ±LFLEPS (WT) antibody or anti-IDO1 2TdCH2±LFLEPS antibody followed by Goat anti-Rabbit Alexa Fluor™ 647 secondary antibody (Product #A21244) Viable cells were used for analysis and data was acquired using Attune™ N×T Flow Cytometer (Product #A29004). A shift in the histogram towards the left represents the background signal reduction upon LFLEPS mutation.

FIG. 12B is a graphical representation of the flow cytometry analysis shown in FIG. 12A demonstrating the amount of FcγR1 binding by the antibodies. The asterisk (*) indicates statistical significance as determined by p<0.05 using 2-tailed Student's T test.

FIG. 13A is a graphical representation of an immunofluorescent analysis of IDO1 that was performed using 70% confluent CD64-GFP (FcγR1) transfected HEK-293E cells with/without treatment of Interferon-gamma (INFT). The cells were stained with 2.5 μg/ml of anti-IDO1 wild type (WT) antibody ±LFLEPS or anti-IDO1 2TdCH2±LFLEPS antibody, followed by Goat anti-Rabbit Alexa Fluor™ 647 secondary antibody (Product #A21244).

FIG. 13B is a graphical representation of an immunofluorescent analysis of IDO1 that was performed using 70% confluent CD64-GFP (FcγR1) transfected HEK-293E cells with/without treatment of INFγ. The cells were stained with 0.5 μg/ml of anti-IDO1 wild type (WT) antibody ±LFLEPS or anti-IDO1 2TdCH2±LFLEPS antibody, followed by Goat anti-Rabbit Alexa Fluor™ 647 secondary antibody (Product #A21244).

FIG. 14A is a flow cytometry analysis demonstrating that the 2TdCH2-LFLEPS design in a mouse-rabbit chimeric antibody (Fab-Fc Chimera) enhances TIGIT signal detection in human PBMCs compared to wild type design. Human PBMCs were stained with CD4 FITC (Product #11-0049-42) and 0.25 μg of anti-TIGIT wild type antibody, or anti-TIGIT+LFLEPS antibody, or anti-TIGIT+2TdCH2-LFLEPS antibody, followed by Goat anti-Rabbit IgG (H+L) Alexa Fluor™ Plus 647 secondary antibody (Product #A55060). Viable cells in the lymphocyte gate were used for analysis and data was acquired using Attune™ N×T Flow Cytometer (Product #A29004).

FIG. 14B is a graphical representation of the flow cytometry analysis shown in FIG. 14A showing the staining index of the various antibodies. The asterisk (*) indicates statistical significance as determined by p<0.05 and asterisk (**) indicates statistical significance as determined by p<0.01 using 2-tailed Student's T test.

FIG. 15 is a graphical representation of an immunofluorescent analysis of the effect of the 2TdCH2 LFLEPS design on a rabbit anti-mouse (RAM) IgG secondary antibody and showing the staining index of the various antibodies. The analysis was performed using 70% confluent CD64-GFP (FcγR1) transfected HEK-293E cells. The cells were stained with 0.5 μg/ml and 2.5 μg/ml of a RAM wild type (WT) antibody ±LFLEPS or a RAM 2TdCH2-LFLEPS ±LFLEPS antibody followed by Donkey anti-Rabbit Alexa Fluor™ 647 secondary antibody d(A32795) to detect the RAM antibody. Increased staining with the RAM WT antibody and the RAM 2TdCH2 antibody (without LFLEPS) indicates the non-specific binding of the secondary antibody as there is no primary antibody present in the sample. The asterisk (*) indicates statistical significance as determined by p<0.05 using 2-tailed Student's T test.

FIG. 16A shows a western blot (left panel) that was performed using 0.5 μg/ml of anti-pERK1/2 WT antibody and anti-pERK1/2 2TdCH2 recombinant mouse monoclonal antibody followed by Goat anti-Mouse IgG (H+L) Horseradish Peroxidase (HRP) Superclonal™ secondary antibody (Product #A28177) by loading whole cell extracts of Jurkat cells with and without PMA treatment (200 ng/ml). The right panel shows a graphical representation of the western blot analysis.

FIG. 16B is an immunofluorescent analysis was performed using 2.5 μg/ml of anti-pERK1/2 WT antibody and anti-pERK1/2 2TdCH2 recombinant mouse monoclonal antibody followed by Goat anti-Mouse IgG (H+L) Alexa Fluor™ Plus 488 secondary antibody in serum starved A431 cells with EGF treatment (200 ng/ml). The left panel shows the immunofluorescence images and the right panel shows a graphical representation of the immunofluorescent analysis.

FIG. 17 shows a western blot that was performed using 4.0 to 0.5 μg/ml of anti-PSD-95 WT antibody and anti-PSD-95 2TdCH2 antibody followed by Goat anti-Mouse IgG (H+L) HRP Superclonal™ Secondary antibody (Product #A28177) by loading whole cell extracts of Neuro2A cells.

FIG. 18A is an immunofluorescent analysis that was performed using 1 μg/ml of wild type or 2TdCH2 LFLEPS anti-OCT4-Alexa Fluor™ Plus 555 antibody conjugate in NTERA2 cells.

FIG. 18B is a graphical representation of the immunofluorescent analysis shown in FIG. 18A. The asterisk (**) indicates statistical significance as determined by p<0.01 using 2-tailed Student's T test.

FIG. 19A is an immunofluorescent analysis that was performed using 1 μg/ml of wild type or 2TdCH2 LFLEPS anti-OCT4-Alexa Fluor™ Plus 647 conjugate in NTERA2 cells.

FIG. 19B is a graphical representation of the immunofluorescent analysis shown in FIG. 19A. The asterisk (*) indicates statistical significance as determined by p<0.05 using 2-tailed Student's T test.

FIG. 20A is western blot analysis that was performed using 1 μg/ml of wild type or 2TdCH2 LFLEPS anti-Parkin HRP conjugate at 4 and 8 molar ratio in PC-12 cell whole cell lysate (WCL).

FIG. 20B is western blot analysis that was performed using 1 μg/ml of wild type or 2TdCH2 LFLEPS anti-FLAG-tag HRP conjugate at 4 and 8 molar ratio in EXPI293 cell transfected with FLAG-Histone H4 construct.

FIG. 20C is western blot analysis that was performed using 1 μg/ml of wild type or 2TdCH2 LFLEPS anti-FLAG-tag Biotin conjugate at 25 and 50 molar ratio in EXPI293 cell transfected with FLAG-Histone H4 construct.

FIG. 21A indicates degree of labeling (DOL) from SiteClick™ conjugation of Alexa Fluor™ 488 dyes with anti-Parkin WT, 2TdCH2 and 2TdCH2 LFLEPS antibodies. The asterisk (*) indicates statistical significance as determined by p<0.05 and asterisk (**) indicates statistical significance as determined by p<0.01 using 2-tailed Student's T test.

FIG. 21B indicates degree of labeling (DOL) from SiteClick™ conjugation of Alexa Fluor™ 488 dyes with anti-OCT4 WT, 2TdCH2 and 2TdCH2 LFLEPS antibodies. The asterisk (*) indicates statistical significance as determined by p<0.05 using 2-tailed Student's T test.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

The disclosed polypeptides, antibodies and methods may be understood more readily by reference to the following detailed description taken in connection with the accompanying figures, which form a part of this disclosure. It is to be understood that the disclosed polypeptides, antibodies, and methods are not limited to the specific polypeptides, antibodies, and methods described and/or shown herein, and that the terminology used herein is for the purpose of describing particular embodiments by way of example only and is not intended to be limiting of the claimed polypeptides, antibodies, and methods.

Unless specifically stated otherwise, any description as to a possible mechanism or mode of action or reason for improvement is meant to be illustrative only, and the disclosed polypeptides, antibodies, and methods are not to be constrained by the correctness or incorrectness of any such suggested mechanism or mode of action or reason for improvement.

Throughout this text, the descriptions refer to polypeptides, antibodies, and methods of producing the polypeptides and antibodies. Where the disclosure describes or claims a feature or embodiment associated with polypeptides or antibodies, such a feature or embodiment is equally applicable to the methods of producing the polypeptides or antibodies. Likewise, where the disclosure describes or claims a feature or embodiment associated with methods of producing the polypeptide or antibodies, such a feature or embodiment is equally applicable to the polypeptides or antibodies.

It is to be appreciated that certain features of the disclosed polypeptides, antibodies, and methods which are, for clarity, described herein in the context of separate embodiments, may also be provided in combination in a single embodiment. Conversely, various features of the disclosed polypeptides, antibodies, and methods that are, for brevity, described in the context of a single embodiment, may also be provided separately or in any subcombination.

As used herein, the singular forms “a,” “an,” and “the” include the plural.

When a list is presented, unless stated otherwise, it is to be understood that each individual element of that list, and every combination of that list, is a separate embodiment. For example, a list of embodiments presented as “A, B, or C” is to be interpreted as including the embodiments: “A;” “B;” “C;” “A or B;” “A or C;” “B or C;” or “A, B, or C.”

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “comprising” is intended to include examples encompassed by the terms “consisting essentially of” and “consisting of”; similarly, the term “consisting essentially of” is intended to include examples encompassed by the term “consisting of.”

### Certain Terminology

“Antibody” as used herein means an antibody of classes IgG, IgM, IgA, IgD, or IgE, or fragments, or derivatives thereof, derived from any animal including, but not limited to, sheep, rabbits, goats, mice, camelids, and egg yolk.

Antibodies include but are not limited to monoclonal or polyclonal antibodies, bispecific antibodies, bifunctional antibodies, fragments of antibodies, humanized antibodies, chimeric antibodies, engineered antibodies, antibody constructs, and altered antibody-like structures. Engineered antibodies or antibody constructs include antibody-like structures having the herein described polypeptides comprising the amino acid sequence of Formula I: Z-(X-Yₙ-Zᵣ)ₚ-X, wherein Z is a hinge comprising an amino acid sequence at least 90% identical to SEQ ID NO: 18, SEQ ID NO: 48, or SEQ ID NO: 102, X is a CH2 domain comprising an amino acid sequence at least 90% identical to SEQ ID NO: 19, SEQ ID NO: 49, or SEQ ID NO: 104, Y is a linker, n is an integer of 1 or greater, p is an integer of 2 or greater, and r is an integer of 0 or 1. Antibodies may be produced or modified by recombinant DNA or other synthetic techniques. The antibody can be an antibody isolated from the serum sample of a mammal, an affinity purified antibody, or any mixture thereof which exhibits sufficient binding specificity to a desired epitope or a sequence derived therefrom. Also included are functional derivatives or “equivalents” of antibodies, e.g., single chain antibodies, CDR-grafted antibodies, etc. Also included are VHH antibodies that may be monovalent or bivalent.

“Biotin” as used herein means biotin or any biotin derivative, including without limitation, substituted and unsubstituted biotin, and analogs and derivatives thereof, as well as substituted and unsubstituted derivatives of caproylamidobiotin, biocytin, desthiobiotin, desthiobiocytin, iminobiotin, and biotin sulfone.

“Coding sequence” or “polynucleotide encoding” as used herein means RNA or DNA molecules that comprise a nucleotide sequence which encodes a protein. The coding sequence can further include initiation and termination signals operably linked to regulatory elements including a promoter and polyadenylation signal capable of directing expression in the cells of a subject or mammal to which the nucleic acid molecule is administered.

“Identity” or “percent identity” as used herein in the context of two or more nucleic acid molecules or polypeptide sequences means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Fluorophore” as used herein means a chemical compound that, when excited by exposure to a particular wavelength of light, emits light (fluoresces), for example at a different wavelength than that to which it was exposed. Fluorophores may contain substituents that alter the solubility, spectral properties or physical properties of the fluorophore.

Exemplary fluorophores include, but are not limited to, xanthene, fluorescein, rhodamine, silicon rhodamine, rhodol, roseamine, carbopyranone, indole, indacene, borapolyazaindacene, furan, benzofuran, cyanine, benzocyanine, benzopyrilium, pyrene, coumarin, styryl, squarine, resorufin, anthraquinone, acridine, benzophenoxazine, quinoline, quinazolinone, oligonucleotide-based fluorescent dyes, conjugated polymer dyes, and tandem dyes.

Examples of fluorophores suitable for use with the disclosed assays, include, but are not limited to, 6-carboxyfluorescein (FAM), tetrachlorofluorescein (TET), tetramethylrhodamine (TMR), hexachlorofluorescein (HEX), JOE, 6-carboxy-X-rhodamine (ROX), CAL Fluor™, Pulsar™, Quasar™, Texas Red™, Texas Red-X, Cy™3, Cy™5, BODIPY™ (boron-dipyrromethene), Alexa Fluor™ dyes (such as Alexa Fluor™ 488), Alexa Fluor Plus dyes (such as Alexa Fluor™ Plus 488), DyLight™ dyes (such as DyLight™ 488), Oregon Green™, and NovaFluor™ dyes (such as NovaFluor™ Red 710), as well as other fluorophores described in Johnson, I. and Spence, M. T. Z, (Eds.). (2010) The Molecular Probes Handbook: A Guide to Fluorescent Probes and Labeling Technologies, 1 1 h edition.

“Immunospecifically binds” as used herein refers to the ability of the disclosed polypeptides or antibodies to preferentially bind to their target(s) without preferentially binding other molecules in a sample or environment containing a mixed population of molecules.

“Label” as used herein means an agent capable of detection, for example by ELISA, spectrophotometry, flow cytometry, or microscopy. For example, a label can be attached (e.g., covalently linked or conjugated) to a nucleic acid molecule or protein (such as an antibody or antigen binding fragment thereof), thereby permitting detection of the nucleic acid molecule or protein. Examples of labels include, but are not limited to, radioactive isotopes, enzyme substrates, co-factors, ligands, chemiluminescent agents, fluorophores, haptens, enzymes, and combinations thereof. Specific non-limiting examples of labels include fluorescent and fluorogenic moieties (e.g., fluorophores).

Illustrative labels include labels that can be directly observed or measured or indirectly observed or measured. Such labels include, but are not limited to, radiolabels that can be measured with radiation-counting devices; pigments, dyes or other chromogens that can be visually observed or measured with a spectrophotometer; spin labels that can be measured with a spin label analyzer; and fluorescent moieties, where the output signal is generated by the excitation of a suitable molecular adduct and that can be visualized by excitation with light that is absorbed by the dye or can be measured with standard fluorometers or imaging systems, for example.

The label can be a luminescent substance such as a phosphor or fluorogen; a bioluminescent substance; a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The label may also take the form of a chemical or biochemical, or an inert particle, including but not limited to colloidal gold, microspheres, quantum dots, or inorganic crystals such as nanocrystals or phosphors (see, e.g., Beverloo, et al., Anal. Biochem. 203, 326-34 (1992)).

The term label can also refer to a “tag” or hapten that can bind selectively to a labeled molecule such that the labeled molecule, when added subsequently, is used to generate a detectable signal. For instance, one can use biotin, iminobiotin or desthiobiotin as a tag and then use an avidin or streptavidin conjugate of horseradish peroxidase (HRP) to bind to the tag, and then use a chromogenic substrate (e.g., tetramethylbenzidine) or a fluorogenic substrate such as Amplex™ Red or Amplex™ Gold (Thermo Fisher Scientific, Waltham, MA) to detect the presence of HRP. In a similar fashion, the tag can be a hapten or antigen (e.g., digoxigenin), and an enzymatically, fluorescently, or radioactively labeled antibody can be used to bind to the tag.

Numerous labels are known by those of skill in the art and include, but are not limited to, particles, fluorescent dyes, haptens, enzymes and their chromogenic, fluorogenic, and chemiluminescent substrates, and other labels that are described in Johnson, I. and Spence, M. T. Z, (Eds.). (2010) The Molecular Probes Handbook: A Guide to Fluorescent Probes and Labeling Technologies, 11 h edition.

“Nucleic acid molecule,” “oligonucleotide,” or “polynucleotide” as used herein means at least two nucleotides covalently linked together. The depiction of a single strand also defines the sequence of the complementary strand. Thus, a nucleic acid molecule also encompasses the complementary strand of a depicted single strand. Many variants of a nucleic acid molecule can be used for the same purpose as a given nucleic acid molecule. Thus, a nucleic acid molecule also encompasses substantially identical nucleic acid molecules and complements thereof.

Nucleic acid molecules can be single stranded or double stranded, or can contain portions of double stranded and single stranded sequence. The nucleic acid molecule can be DNA (both genomic and cDNA), RNA, or a hybrid, where the nucleic acid molecule can contain combinations of deoxyribo- and ribo-nucleotides, and combinations of bases including uracil, adenine, thymine, cytosine, guanine, inosine, xanthine hypoxanthine, isocytosine and isoguanine. Nucleic acid molecules can have post-transcriptional chemical modifications. Nucleic acid molecules can be obtained by chemical synthesis methods or by recombinant methods.

“Oligonucleotide-based fluorescent dye” as used herein means a nucleic acid nanostructure composed of a DNA-based scaffold that precisely arranges fluorophores in order to engineer their interactions and the overall fluorescent properties of the structure. Examples of oligonucleotide-based fluorescent dyes are described in International Patent Application Publication No. WO 2018/231805 and include, but are not limited to, NovaFluor™ dyes (Thermo Fisher Scientific, Waltham, MA).

A “peptide,” “protein,” or “polypeptide” as used herein can mean a linked sequence of amino acids and can be natural, synthetic, or a modification or combination of natural and synthetic. A peptide, protein, or polypeptide can have post-translational modifications.

“Vector” as used herein means a polynucleotide sequence containing an origin of replication. A vector can be a viral vector, bacteriophage, bacterial artificial chromosome, or yeast artificial chromosome. A vector can be a DNA or RNA vector. A vector can be a self-replicating extrachromosomal vector, and preferably, is a DNA plasmid. The vector can contain or include one or more heterologous polynucleotide sequences.

### Engineered Antibodies

Recombinant antibodies offer the ability to engineer benefits in a rational method. One of the most important characteristics of an antibody is its sensitivity, i.e. the strength of the signal produced in a particular immunoassay. A highly sensitive antibody has several benefits, such as the detection of low abundance or rare targets and the ability to detect the protein of interest in complex assays such as imaging. Antibody sensitivity is influenced both by increased signal and decreased background. Antibody sensitivity can be increased by affinity maturation of the antibody clone, which involves mutations of the antigen binding site or paratope. However, affinity maturation has to be tailored for each antibody and therefore is not a scalable solution to address a large number of antibodies. Furthermore, mutations to the paratope change the clone of the antibody and essentially produces a new antibody.

The instant disclosure provides an approach for increasing antibody sensitivity by rational modification of the antibody Fc region. Amplification or expansion of the entire Fc domain or portions of the Fc domain provides additional binding sites for secondary antibodies, resulting in an enhanced signal. Prior studies have utilized Fc amplification for other purposes such as increased antibody effects in a therapeutic application by enhanced binding to Fc receptors, in the case of a humanized antibody. It was found that antibody modification by adding one additional Fc domain (1TdFc) or two additional Fc domains (2TdFc) to the antibody resulted in poor expression resulting in a low yield and have aggregation and purity challenges. To overcome the above limitations, the instant disclosure provides a rational design to circumvent the disadvantages of the prior studies by selectively adding additional CH2 domains (2TdCH2) instead of the entire Fc region. The 2TdCH2 design of the instant polypeptides and antibodies provided herein overcame the above challenges and it was unexpectedly found that the 2TdCH2 engineered antibodies provided herein had comparable yields, monomer purity, and stability to wildtype antibodies. The two additional CH2 domains, joined by a hinge and a linker, offer additional epitopes for secondary antibody binding and additional lysine residues for conjugation. In addition, the 2TdCH2 engineered antibodies of the present disclosure provide a significant increase in sensitivity in multiple applications such as western blotting and immunocytochemistry and allow for broader applicability of the antibodies. In addition to the benefits for the unconjugated antibodies, 2TdCH2 engineering also provides additional binding sites for different conjugation chemistries. It was also unexpectedly found that introduction of specific mutations, L234F, L235E and P331S, herein described as “LFLEPS mutations”, into the engineered backbone reduced background binding of the antibody to Fc gamma receptors present on selected cells. Thus, the advantages of the engineered antibodies of the present disclosure include: i) increased sensitivity of at least 1.5-fold; ii) reduced background by at least 1.5-fold; ii) detection of low abundance targets that are otherwise difficult to detect with standard antibodies; iii) increased application coverage through the use of multiplexing; iv) increased conjugation sites for primary conjugates; and v) addresses design challenges of expression, purity and stability found with other recombinant antibodies.

Disclosed herein are isolated polypeptides comprising, consisting essentially of, or consisting of the amino acid sequence of Formula I: Z-(X-Yₙ-Zᵣ)ₚ-X, wherein: Z is a hinge region, X is a CH2 domain, Y is a linker, n is an integer of 1 or greater, p is an integer of 2 or greater, and r is an integer of 0 or 1. In certain embodiments the elements of the amino acid sequence of Formula I defined herein as Z-(X-Yₙ-Zᵣ)ₚ-X are linked directly to one another with no intervening amino acid sequences.

The hinge (Z) region can comprise the amino acid sequence of SEQ ID NO: 18. The hinge (Z) can comprise an amino acid sequence with at least 90% sequence identity to SEQ ID NO: 18. The hinge (Z) can comprise an amino acid sequence with at least 95% sequence identity to SEQ ID NO: 18. The hinge (Z) can comprise the amino acid sequence of SEQ ID NO: 48. The hinge (Z) can comprise an amino acid sequence with at least 90% sequence identity to SEQ ID NO: 48. The hinge (Z) can comprise an amino acid sequence with at least 95% sequence identity to SEQ ID NO: 48. The hinge (Z) can comprise the amino acid sequence of SEQ ID NO: 102. The hinge (Z) can comprise an amino acid sequence with at least 90% sequence identity to SEQ ID NO: 102. The hinge (Z) can comprise an amino acid sequence with at least 95% sequence identity to SEQ ID NO: 102.

The CH2 domain (X) can comprise the amino acid sequence of SEQ ID NO: 19. The CH2 domain (X) can comprise an amino acid sequence with at least 90% sequence identity to SEQ ID NO: 19. The CH2 domain (X) can comprise an amino acid sequence with at least 95% sequence identity to SEQ ID NO: 19. The CH2 domain (X) can comprise the amino acid sequence of SEQ ID NO: 49. The CH2 domain (X) can comprise an amino acid sequence with at least 90% sequence identity to SEQ ID NO: 49. The CH2 domain (X) can comprise an amino acid sequence with at least 95% sequence identity to SEQ ID NO: 49. The CH2 domain (X) can comprise the amino acid sequence of SEQ ID NO: 104. The CH2 domain (X) can comprise an amino acid sequence with at least 90% sequence identity to SEQ ID NO: 104. The CH2 domain (X) can comprise an amino acid sequence with at least 95% sequence identity to SEQ ID NO: 104.

The isolated polypeptide can comprise one or more linkers (Y) comprising the amino acid sequence of GS, GGS, SEQ ID NO: 14, SEQ ID NO: 38, SEQ ID NO: 43, or SEQ ID NO: 44. The one or more linkers (Y) can comprise GS. The one or more linkers (Y) can comprise GGS. The one or more linkers (Y) can comprise SEQ ID NO: 14. The one or more linkers (Y) can comprise SEQ ID NO: 38. The one or more linkers can comprise SEQ ID NO: 43. The one or more linkers (Y) can comprise a sequence with at least 90% sequence identity to SEQ ID NO: 43. The one or more linkers (Y) can comprise SEQ ID NO: 44. The one or more linkers (Y) can comprise a sequence with at least 90% sequence identity to SEQ ID NO: 44.

The isolated polypeptide can comprise one linker (Y), such that n is 1. The isolated polypeptide can comprise two linkers (Y), such that n is 2. The isolated polypeptide can comprise three linkers (Y), such that n is 3. The isolated polypeptide can comprise four linkers (Y), such that n is 4. The isolated polypeptide can comprise five linkers (Y), such that n is 5.

The isolated polypeptide can comprise two or more regions comprising a CH2-linker region ((X-Yₙ)ₚ), such that p is 2 or greater. The isolated polypeptide can comprise two CH2-linker regions ((X-Yₙ)ₚ), such that p is 2. The isolated polypeptide can comprise three CH2-linker regions ((X-Yₙ)ₚ), such that p is 3. The isolated polypeptide can comprise four CH2-linker regions ((X-Yₙ)ₚ), such that p is 4. The isolated polypeptide can comprise five CH2-linker regions ((X—Y₁)ₚ), such that p is 5.

The isolated polypeptide can comprise two or more regions comprising a CH2-linker-hinge region ((X-Yₙ-Zᵣ)ₚ), such that p is 2 or greater. The isolated polypeptide can comprise two CH2-linker-hinge regions ((X-Yₙ-Zᵣ)ₚ), such that p is 2. The isolated polypeptide can comprise three CH2-linker-hinge regions ((X-Yₙ-Zᵣ)ₚ), such that p is 3. The isolated polypeptide can comprise four CH2-linker-hinge regions ((X-Yₙ-Zᵣ)ₚ), such that p is 4. The isolated polypeptide can comprise five CH2-linker-hinge regions ((X-Yₙ-Zᵣ)ₚ), such that p is 5.

In some embodiments, the isolated polypeptide comprises, consists essentially of, or consists of (X-Yₙ-Zᵣ)ₚ, wherein Z (hinge) comprises the amino acid sequence of SEQ ID NO: 18, X(CH2 domain) comprises the amino acid sequence of SEQ ID NO: 19, Y (linker) comprises the amino acid sequence of SEQ ID NO: 44, wherein n is 1, r is 0 or 1, and p is 2.

In some embodiments, the isolated polypeptide comprises, consists essentially of, or consists of (X-Yₙ-Zᵣ)ₚ, wherein Z (hinge) comprises the amino acid sequence of SEQ ID NO: 48, X(CH2 domain) comprises the amino acid sequence of SEQ ID NO: 49, Y (linker) comprises the amino acid sequence of SEQ ID NO: 44, wherein n is 1, r is 0 or 1, and p is 2.

In some embodiments, the isolated polypeptide comprises, consists essentially of, or consists of (X-Yₙ-Zᵣ)ₚ, wherein Z (hinge) comprises the amino acid sequence of SEQ ID NO: 102, X(CH2 domain) comprises the amino acid sequence of SEQ ID NO: 104, Y (linker) comprises the amino acid sequence of SEQ ID NO: 44, wherein n is 1, r is 0 or 1, and p is 2.

In some embodiments, the isolated polypeptide comprises, consists essentially of, or consists of (X-Yₙ-Zᵣ)ₚ, wherein Z (hinge) comprises the amino acid sequence of SEQ ID NO: 102, X(CH2 domain) comprises the amino acid sequence of SEQ ID NO: 104, Y (linker) comprises the amino acid sequence of SEQ ID NO: 45, wherein n is 1, r is 0 or 1, and p is 2.

In some embodiments, the isolated polypeptide comprises two additional CH2 domains N-terminal to the Fc domain connected via a hinge and optionally, one or more linkers. The isolated polypeptide comprising two additional CH2 domains N-terminal to the Fc domain connected via a hinge and optionally one or more linkers can comprise the amino acid sequence of SEQ ID NO: 52 or SEQ ID NO: 54. In some embodiments, the isolated polypeptide comprising a CH3 domain comprises SEQ ID NO: 52. The isolated polypeptide comprising two additional CH2 domains N-terminal to the Fc domain connected via a hinge and optionally one or more linkers can comprise an amino acid sequence with at least 90% identity to SEQ ID NO: 52. The isolated polypeptide comprising two additional CH2 domains N-terminal to the Fc domain connected via a hinge and optionally one or more linkers can comprise an amino acid sequence with at least 95% identity to SEQ ID NO: 52.

In some embodiments, the isolated polypeptide comprises two additional CH2 domains N-terminal to the Fc domain connected via a hinge and optionally one or more linkers and comprises “LFLEPS” mutations within the lower hinge and CH2 domains. The “LFLEPS” mutations can comprise the following amino acid substitutions: L234F, L235E and P331S. The isolated polypeptide comprising two additional CH2 domains N-terminal to the Fc domain connected via a hinge and optionally one or more linkers and comprises “LFLEPS” mutations within the lower hinge and CH2 domains comprises SEQ ID NO: 54. The isolated polypeptide comprising two additional CH2 domains N-terminal to the Fc domain connected via a hinge and optionally one or more linkers and comprises “LFLEPS” mutations within the lower hinge and CH2 domains can comprise an amino acid sequence with at least 90% identity to SEQ ID NO: 54. The isolated polypeptide comprising two additional CH2 domains N-terminal to the Fc domain connected via a hinge and optionally one or more linkers and comprises “LFLEPS” mutations within the lower hinge and CH2 domains can comprise an amino acid sequence with at least 95% identity to SEQ ID NO: 54.

Any isolated polypeptide disclosed herein can be linked to a second polypeptide by one or more disulfide bonds, wherein the isolated polypeptide and the second polypeptide share the same amino acid sequence. The one or more disulfide bonds can occur between a hinge of the isolated polypeptide and a hinge of the second polypeptide.

Any isolated polypeptide disclosed herein can further comprise a CH3 domain. In certain embodiments, isolated polypeptides disclosed herein further comprises no more than one CH3 domain. In some embodiments the CH3 domain comprises the amino acid sequence of SEQ ID NO: 20. The CH3 domain can comprise an amino acid sequence with at least 90% sequence identity to SEQ ID NO: 20. The CH3 domain can comprise an amino acid sequence with at least 95% sequence identity to SEQ ID NO: 20.

In some embodiments, the isolated polypeptide comprising a CH3 domain comprises SEQ ID NO: 106. The isolated polypeptide comprising a CH3 domain can comprise an amino acid sequence with at least 90% identity to SEQ ID NO: 106. The isolated polypeptide comprising a CH3 domain can comprise an amino acid sequence with at least 95% identity to SEQ ID NO: 106.

In some embodiments, the isolated polypeptide comprising a CH3 domain comprises SEQ ID NO: 141. The isolated polypeptide comprising a CH3 domain can comprise an amino acid sequence with at least 90% identity to SEQ ID NO: 141. The isolated polypeptide comprising a CH3 domain can comprise an amino acid sequence with at least 95% identity to SEQ ID NO: 141.

Also provided herein are antibodies comprising any isolated peptide disclosed herein linked to a Fab domain. The Fab can comprise a CH1 domain. The CH1 domain can comprise the amino acid sequence of SEQ ID NO: 17. The CH1 domain can comprise an amino acid sequence with at least 90% sequence identity to SEQ ID NO: 17. The CH1 domain can comprise an amino acid sequence with at least 95% sequence identity to SEQ ID NO: 17. The Fab can comprise a Fab heavy chain and a Fab light chain that pair to form a binding domain.

In some embodiments, the Fab can comprise a CH1 domain. The CH1 domain can comprise the amino acid sequence of SEQ ID NO: 100. The CH1 domain can comprise an amino acid sequence with at least 90% sequence identity to SEQ ID NO: 100. The CH1 domain can comprise an amino acid sequence with at least 95% sequence identity to SEQ ID NO: 100. The Fab can comprise a Fab heavy chain and a Fab light chain that pair to form a binding domain.

In some embodiments, the Fab can comprise a CH1 domain. The CH1 domain can comprise the amino acid sequence of SEQ ID NO: 160. The CH1 domain can comprise an amino acid sequence with at least 90% sequence identity to SEQ ID NO: 160. The CH1 domain can comprise an amino acid sequence with at least 95% sequence identity to SEQ ID NO: 160. The Fab can comprise a Fab heavy chain and a Fab light chain that pair to form a binding domain.

In some embodiments, the antibodies comprising any isolated peptide disclosed herein linked to a Fab domain have a reduction of background signal of at least 1.5-fold, compared to a corresponding wildtype antibody. In some embodiments, the reduction of background signal is at least 1.5-fold, at least 2-fold, at least 2.5-fold, at least 3-fold, at least 3.5-fold, at least 4-fold, at least 4.5-fold or at least 5-fold compared to a corresponding wild type antibody.

In some embodiments, the antibodies comprising any isolated peptide disclosed herein linked to a Fab domain have an increase in sensitivity of at least 1.5-fold compared to a corresponding wild type antibody. In some embodiments, the increase in sensitivity is at least 1.5-fold, at least 2-fold, at least 2.5-fold, at least 3-fold, at least 3.5-fold, at least 4-fold, at least 4.5-fold, at least 5-fold, at least 5.5-fold, at least 6-fold, at least 6.5-fold, at least 7-fold, at least 7.5-fold, at least 8-fold, at least 8.5-fold, at least 9-fold, at least 9.5-fold, or at least 10-fold compared to a corresponding wild type antibody.

In some embodiments, the antibodies comprising any isolated peptide disclosed herein linked to a Fab domain has an increase in signal enhancement of at least 1.5-fold compared to a corresponding wild type antibody. In some embodiments, the increase in signal enhancement is at least 1.5-fold, at least 2-fold, at least 2.5-fold, at least 3-fold, at least 3.5-fold, at least 4-fold, at least 4.5-fold, at least 5-fold, at least 5.5-fold, at least 6-fold, at least 6.5-fold, at least 7-fold, at least 7.5-fold, at least 8-fold, at least 8.5-fold, at least 9-fold, at least 9.5-fold, or at least 10-fold compared to a corresponding wild type antibody.

The Fab of the antibody can immunospecifically bind to one or more target molecules. The Fab can immunospecifically bind to Parkin, OCT4, IDO1, FLAG-tag, pERK1/2, PSD-95, or TIGIT, or the Fab can be a rabbit anti-mouse (RAM) IgG secondary antibody.

In some embodiments, the Fab immunospecifically binds to Parkin. The Fab that immunospecifically binds to Parkin can comprise a Fab heavy chain comprising a heavy chain complementarity determining region 1 (HCDR1) comprising the amino acid sequence of SEQ ID NO: 4, a HCDR2 comprising the amino acid sequence of SEQ ID NO: 5, and a HCDR3 comprising the amino acid sequence of SEQ ID NO: 6. In certain embodiments throughout this disclosure, the CDRs are defined according to the IMGT system.

The Fab that immunospecifically binds to Parkin can comprise a Fab heavy chain comprising a variable region comprising the amino acid sequence of SEQ ID NO: 7. The Fab that immunospecifically binds to Parkin can comprise a Fab heavy chain comprising a variable region comprising an amino acid sequence with at least 90% sequence identity to SEQ ID NO: 7. The Fab that immunospecifically binds to Parkin can comprise a Fab heavy chain comprising a variable region comprising an amino acid sequence with at least 95% sequence identity to SEQ ID NO: 7.

The Fab that immunospecifically binds to Parkin can comprise a Fab heavy chain comprising a variable region comprising the amino acid sequence of SEQ ID NO: 9. The Fab that immunospecifically binds to Parkin can comprise a Fab heavy chain comprising a variable region comprising an amino acid sequence with at least 90% sequence identity to SEQ ID NO: 9. The Fab that immunospecifically binds to Parkin can comprise a Fab heavy chain comprising a variable region comprising an amino acid sequence with at least 95% sequence identity to SEQ ID NO: 9.

The Fab that immunospecifically binds to Parkin can comprise a Fab light chain comprising a light chain complementarity determining region 1 (LCDR1) comprising the amino acid sequence of SEQ ID NO: 13, a LCDR2 comprising the amino acid sequence RAS, and a LCDR3 comprising the amino acid sequence of SEQ ID NO: 15.

The Fab that immunospecifically binds to Parkin can comprise a Fab light chain comprising a variable region comprising the amino acid sequence of SEQ ID NO: 16. The Fab that immunospecifically binds to Parkin can comprise a Fab light chain comprising a variable region comprising an amino acid sequence with at least 90% sequence identity to SEQ ID NO: 16. The Fab that immunospecifically binds to Parkin can comprise a Fab light chain comprising a variable region comprising an amino acid sequence with at least 95% sequence identity to SEQ ID NO: 16.

The Fab that immunospecifically binds to Parkin can comprise a Fab light chain comprising a variable region comprising the amino acid sequence of SEQ ID NO: 12. The Fab that immunospecifically binds to Parkin can comprise a Fab light chain comprising a variable region comprising an amino acid sequence with at least 90% sequence identity to SEQ ID NO: 12. The Fab that immunospecifically binds to Parkin can comprise a Fab light chain comprising a variable region comprising an amino acid sequence with at least 95% sequence identity to SEQ ID NO: 12.

The Fab that immunospecifically binds to Parkin can comprise a Fab heavy chain comprising a heavy chain complementarity determining region 1 (HCDR1) comprising the amino acid sequence of SEQ ID NO: 4, a HCDR2 comprising the amino acid sequence of SEQ ID NO: 5, and a HCDR3 comprising the amino acid sequence of SEQ ID NO: 6, and a Fab light chain comprising a light chain complementarity determining region 1 (LCDR1) comprising the amino acid sequence of SEQ ID NO: 13, a LCDR2 comprising the amino acid sequence RAS, and a LCDR3 comprising the amino acid sequence of SEQ ID NO: 15.

The Fab that immunospecifically binds to Parkin can comprise a Fab heavy chain comprising a variable region comprising the amino acid sequence of SEQ ID NO: 7 and a Fab light chain comprising a variable region comprising the amino acid sequence of SEQ ID NO: 16. The Fab that immunospecifically binds to Parkin can comprise a Fab heavy chain comprising a variable region comprising an amino acid sequence with at least 90% sequence identity to SEQ ID NO: 7 and a Fab light chain comprising a variable region comprising an amino acid sequence with at least 90% sequence identity to the amino acid sequence of SEQ ID NO: 16. The Fab that immunospecifically binds to Parkin can comprise a Fab heavy chain comprising a variable region comprising an amino acid sequence with at least 95% sequence identity to SEQ ID NO: 7 and a Fab light chain comprising a variable region comprising an amino acid sequence with at least 95% sequence identity to the amino acid sequence of SEQ ID NO: 16.

The Fab that immunospecifically binds to Parkin can comprise a Fab heavy chain comprising a variable region comprising the amino acid sequence of SEQ ID NO: 9 and a Fab light chain comprising a variable region comprising the amino acid sequence of SEQ ID NO: 12. The Fab that immunospecifically binds to Parkin can comprise a Fab heavy chain comprising a variable region comprising an amino acid sequence with at least 90% sequence identity to the amino acid sequence of SEQ ID NO: 9 and a Fab light chain comprising a variable region comprising an amino acid sequence with at least 90% sequence identity to the amino acid sequence of SEQ ID NO: 12. The Fab that immunospecifically binds to Parkin can comprise a Fab heavy chain comprising a variable region comprising an amino acid sequence with at least 95% sequence identity to the amino acid sequence of SEQ ID NO: 9 and a Fab light chain comprising a variable region comprising an amino acid sequence with at least 95% sequence identity to the amino acid sequence of SEQ ID NO: 12.

In some embodiments, the antibody comprising a Fab that immunospecifically binds to Parkin comprises the amino acid sequence of SEQ ID NO: 3. The antibody comprising a Fab that immunospecifically binds to Parkin can comprise an amino acid sequence with at least 90% sequence identity to SEQ ID NO: 3. The antibody comprising a Fab that immunospecifically binds to Parkin can comprise an amino acid sequence with at least 95% sequence identity to SEQ ID NO: 3.

In some embodiments, the Fab immunospecifically binds to OCT4. The Fab that immunospecifically binds to OCT4 can comprise a heavy chain complementarity determining region 1 (HCDR1) comprising the amino acid sequence of SEQ ID NO: 29, a HCDR2 comprising the amino acid sequence of SEQ ID NO: 30, and a HCDR3 comprising the amino acid sequence of SEQ ID NO: 31.

The Fab that immunospecifically binds to OCT4 can comprise a Fab heavy chain comprising a variable region comprising the amino acid sequence of SEQ ID NO: 32. The Fab that immunospecifically binds to OCT4 can comprise a Fab heavy chain comprising a variable region comprising an amino acid sequence with at least 90% sequence identity to SEQ ID NO: 32. The Fab that immunospecifically binds to OCT4 can comprise a Fab heavy chain comprising a variable region comprising an amino acid sequence with at least 95% sequence identity to SEQ ID NO: 32.

The Fab that immunospecifically binds to OCT4 can comprise a Fab heavy chain comprising a variable region comprising the amino acid sequence of SEQ ID NO: 34. The Fab that immunospecifically binds to OCT4 can comprise a Fab heavy chain comprising a variable region comprising an amino acid sequence with at least 90% sequence identity to SEQ ID NO: 34. The Fab that immunospecifically binds to OCT4 can comprise a Fab heavy chain comprising a variable region comprising an amino acid sequence with at least 95% sequence identity to SEQ ID NO: 34.

The Fab that immunospecifically binds OCT4 can comprise a Fab light chain comprising a light chain complementarity determining region 1 (LCDR1) comprising the amino acid sequence of SEQ ID NO: 37, a LCDR2 comprising the amino acid sequence GAS, and a LCDR3 comprising the amino acid sequence of SEQ ID NO: 39.

The Fab that immunospecifically binds OCT4 can comprise a Fab light chain comprising a variable region comprising the amino acid sequence of SEQ ID NO: 40. The Fab that immunospecifically binds OCT4 can comprise a Fab light chain comprising a variable region comprising an amino acid sequence with at least 90% sequence identity to SEQ ID NO: 40. The Fab that immunospecifically binds OCT4 can comprise a Fab light chain comprising a variable region comprising an amino acid sequence with at least 95% sequence identity to SEQ ID NO: 40.

The Fab that immunospecifically binds to OCT4 can comprise a Fab light chain comprising a variable region comprising the amino acid sequence of SEQ ID NO: 36. The Fab that immunospecifically binds OCT4 can comprise a Fab light chain comprising a variable region comprising an amino acid sequence with at least 90% sequence identity to SEQ ID NO: 36. The Fab that immunospecifically binds OCT4 can comprise a Fab light chain comprising a variable region comprising an amino acid sequence with at least 95% sequence identity to SEQ ID NO: 36.

The Fab that immunospecifically binds to OCT4 can comprise a Fab heavy chain comprising a heavy chain complementarity determining region 1 (HCDR1) comprising the amino acid sequence of SEQ ID NO: 29, a HCDR2 comprising the amino acid sequence of SEQ ID NO: 30, and a HCDR3 comprising the amino acid sequence of SEQ ID NO: 31, and a Fab light chain comprising a light chain complementarity determining region 1 (LCDR1) comprising the amino acid sequence of SEQ ID NO: 37, a LCDR2 comprising the amino acid sequence GAS, and a LCDR3 comprising the amino acid sequence of SEQ ID NO: 39.

The Fab that immunospecifically binds to OCT4 can comprise a Fab heavy chain comprising a variable region comprising the amino acid sequence of SEQ ID NO: 32 and a Fab light chain comprising a variable region comprising the amino acid sequence of SEQ ID NO: 40. The Fab that immunospecifically binds to OCT4 can comprise a Fab heavy chain comprising a variable region comprising an amino acid sequence with at least 90% sequence identity to SEQ ID NO: 32 and a Fab light chain comprising a variable region comprising an amino acid sequence with at least 90% sequence identity to the amino acid sequence of SEQ ID NO: 40. The Fab that immunospecifically binds to OCT4 can comprise a Fab heavy chain comprising a variable region comprising an amino acid sequence with at least 95% sequence identity to SEQ ID NO: 32 and a Fab light chain comprising a variable region comprising an amino acid sequence with at least 95% sequence identity to the amino acid sequence of SEQ ID NO: 40.

The Fab that immunospecifically binds to OCT4 can comprise a Fab heavy chain comprising a variable region comprising the amino acid sequence of SEQ ID NO: 34 and a Fab light chain comprising a variable region comprising the amino acid sequence of SEQ ID NO: 36. The Fab that immunospecifically binds to OCT4 can comprise a Fab heavy chain comprising a variable region comprising an amino acid sequence with at least 90% sequence identity to the amino acid sequence of SEQ ID NO: 34 and a Fab light chain comprising a variable region comprising an amino acid sequence with at least 90% sequence identity to the amino acid sequence of SEQ ID NO: 36. The Fab that immunospecifically binds to OCT4 can comprise a Fab heavy chain comprising a variable region comprising an amino acid sequence with at least 95% sequence identity to the amino acid sequence of SEQ ID NO: 34 and a Fab light chain comprising a variable region comprising an amino acid sequence with at least 95% sequence identity to the amino acid sequence of SEQ ID NO: 36.

In some embodiments, the antibody comprising a Fab that immunospecifically binds to OCT4 comprises the amino acid sequence of SEQ ID NO: 28. The isolated polypeptide comprising a Fab that immunospecifically binds to OCT4 can comprise an amino acid sequence with at least 90% sequence identity to SEQ ID NO: 28. The isolated polypeptide comprising a Fab that immunospecifically binds to OCT4 can comprise an amino acid sequence with at least 95% sequence identity to SEQ ID NO: 28.

In some embodiments, the Fab immunospecifically binds to IDO1. The Fab that immunospecifically binds to IDO1 can comprise a heavy chain complementarity determining region 1 (HCDR1) comprising the amino acid sequence of SEQ ID NO: 66, a HCDR2 comprising the amino acid sequence of SEQ ID NO: 67, and a HCDR3 comprising the amino acid sequence of SEQ ID NO: 68.

The Fab that immunospecifically binds to IDO1 can comprise a Fab heavy chain comprising a variable region comprising the amino acid sequence of SEQ ID NO: 65. The Fab that immunospecifically binds to IDO1 can comprise a Fab heavy chain comprising a variable region comprising an amino acid sequence with at least 90% sequence identity to SEQ ID NO: 65. The Fab that immunospecifically binds to IDO1 can comprise a Fab heavy chain comprising a variable region comprising an amino acid sequence with at least 95% sequence identity to SEQ ID NO: 65.

The Fab that immunospecifically binds IDO1 can comprise a Fab light chain comprising a light chain complementarity determining region 1 (LCDR1) comprising the amino acid sequence of SEQ ID NO: 72, a LCDR2 comprising the amino acid sequence of YAS, and a LCDR3 comprising the amino acid sequence of SEQ ID NO: 73.

The Fab that immunospecifically binds IDO1 can comprise a Fab light chain comprising a variable region comprising the amino acid sequence of SEQ ID NO: 71. The Fab that immunospecifically binds IDO1 can comprise a Fab light chain comprising a variable region comprising an amino acid sequence with at least 90% sequence identity to SEQ ID NO: 71. The Fab that immunospecifically binds IDO1 can comprise a Fab light chain comprising a variable domain comprising an amino acid sequence with at least 95% sequence identity to SEQ ID NO: 71.

The Fab that immunospecifically binds to IDO1 can comprise a Fab heavy chain comprising a heavy chain complementarity determining region 1 (HCDR1) comprising the amino acid sequence of SEQ ID NO: 66, a HCDR2 comprising the amino acid sequence of SEQ ID NO: 67, and a HCDR3 comprising the amino acid sequence of SEQ ID NO: 68, and a Fab light chain comprising a light chain complementarity determining region 1 (LCDR1) comprising the amino acid sequence of SEQ ID NO: 72, a LCDR2 comprising the amino acid sequence of YAS, and a LCDR3 comprising the amino acid sequence of SEQ ID NO: 73.

The Fab that immunospecifically binds to IDO1 can comprise a Fab heavy chain comprising a variable region comprising the amino acid sequence of SEQ ID NO: 65 and a Fab light chain comprising a variable region comprising the amino acid sequence of SEQ ID NO: 71. The Fab that immunospecifically binds to IDO1 can comprise a Fab heavy chain comprising a variable region comprising an amino acid sequence with at least 90% sequence identity to SEQ ID NO: 65 and a Fab light chain comprising a variable region comprising an amino acid sequence with at least 90% sequence identity to the amino acid sequence of SEQ ID NO: 71. The Fab that immunospecifically binds to IDO1 can comprise a Fab heavy chain comprising a variable region comprising an amino acid sequence with at least 95% sequence identity to SEQ ID NO: 65 and a Fab light chain comprising a variable region comprising an amino acid sequence with at least 95% sequence identity to the amino acid sequence of SEQ ID NO: 71.

In some embodiments, the antibody comprising a Fab that immunospecifically binds to IDO1 comprises the amino acid sequence of SEQ ID NO: 79. The isolated polypeptide comprising a Fab that immunospecifically binds to IDO1 can comprise an amino acid sequence with at least 90% sequence identity to SEQ ID NO: 79. The isolated polypeptide comprising a Fab that immunospecifically binds to IDO1 can comprise an amino acid sequence with at least 95% sequence identity to SEQ ID NO: 79.

In some embodiments, the Fab immunospecifically binds to FLAG-tag. The Fab that immunospecifically binds to FLAG-tag can comprise a heavy chain complementarity determining region 1 (HCDR1) comprising the amino acid sequence of SEQ ID NO: 163, a HCDR2 comprising the amino acid sequence of SEQ ID NO: 164, and a HCDR3 comprising the amino acid sequence of SEQ ID NO: 165.

The Fab that immunospecifically binds to FLAG-tag can comprise a Fab heavy chain comprising a variable region comprising the amino acid sequence of SEQ ID NO: 162. The Fab that immunospecifically binds to FLAG-tag can comprise a Fab heavy chain comprising a variable region comprising an amino acid sequence with at least 90% sequence identity to SEQ ID NO: 162. The Fab that immunospecifically binds to FLAG-tag can comprise a Fab heavy chain comprising a variable region comprising an amino acid sequence with at least 95% sequence identity to SEQ ID NO: 162.

The Fab that immunospecifically binds FLAG-tag can comprise a Fab light chain comprising a light chain complementarity determining region 1 (LCDR1) comprising the amino acid sequence of SEQ ID NO: 168, a LCDR2 comprising the amino acid sequence of YAS, and a LCDR3 comprising the amino acid sequence of SEQ ID NO: 169.

The Fab that immunospecifically binds FLAG-tag can comprise a Fab light chain comprising a variable region comprising the amino acid sequence of SEQ ID NO: 167. The Fab that immunospecifically binds FLAG-tag can comprise a Fab light chain comprising a variable region comprising an amino acid sequence with at least 90% sequence identity to SEQ ID NO: 167. The Fab that immunospecifically binds FLAG-tag can comprise a Fab light chain comprising a variable domain comprising an amino acid sequence with at least 95% sequence identity to SEQ ID NO: 167.

The Fab that immunospecifically binds to FLAG-tag can comprise a Fab heavy chain comprising a heavy chain complementarity determining region 1 (HCDR1) comprising the amino acid sequence of SEQ ID NO: 163, a HCDR2 comprising the amino acid sequence of SEQ ID NO: 164, and a HCDR3 comprising the amino acid sequence of SEQ ID NO: 165, and a Fab light chain comprising a light chain complementarity determining region 1 (LCDR1) comprising the amino acid sequence of SEQ ID NO: 168, a LCDR2 comprising the amino acid sequence of YAS, and a LCDR3 comprising the amino acid sequence of SEQ ID NO: 169.

The Fab that immunospecifically binds to FLAG-tag can comprise a Fab heavy chain comprising a variable region comprising the amino acid sequence of SEQ ID NO: 162 and a Fab light chain comprising a variable region comprising the amino acid sequence of SEQ ID NO: 167. The Fab that immunospecifically binds to FLAG-tag can comprise a Fab heavy chain comprising a variable region comprising an amino acid sequence with at least 90% sequence identity to SEQ ID NO: 162 and a Fab light chain comprising a variable region comprising an amino acid sequence with at least 90% sequence identity to the amino acid sequence of SEQ ID NO: 167. The Fab that immunospecifically binds to FLAG-tag can comprise a Fab heavy chain comprising a variable region comprising an amino acid sequence with at least 95% sequence identity to SEQ ID NO: 162 and a Fab light chain comprising a variable region comprising an amino acid sequence with at least 95% sequence identity to the amino acid sequence of SEQ ID NO: 167.

In some embodiments, the antibody comprising a Fab that immunospecifically binds to FLAG-tag comprises the amino acid sequence of SEQ ID NO: 175. The isolated polypeptide comprising a Fab that immunospecifically binds to FLAG-tag can comprise an amino acid sequence with at least 90% sequence identity to SEQ ID NO: 175. The isolated polypeptide comprising a Fab that immunospecifically binds to FLAG-tag can comprise an amino acid sequence with at least 95% sequence identity to SEQ ID NO: 175.

In some embodiments, the Fab is a rabbit anti-mouse IgG secondary antibody (also referred to herein as “rabbit anti-mouse IgG antibody,” “rabbit anti-mouse IgG,” or “RAM”). The Fab that is a RAM can comprise a heavy chain complementarity determining region 1 (HCDR1) comprising the amino acid sequence of SEQ ID NO: 127, a HCDR2 comprising the amino acid sequence of SEQ ID NO: 128, and a HCDR3 comprising the amino acid sequence of SEQ ID NO: 129.

The Fab that is a RAM can comprise a Fab heavy chain comprising a variable region comprising the amino acid sequence of SEQ ID NO: 126. The Fab that is a RAM can comprise a Fab heavy chain comprising a variable region comprising an amino acid sequence with at least 90% sequence identity to SEQ ID NO: 126. The Fab that is a RAM can comprise a Fab heavy chain comprising a variable region comprising an amino acid sequence with at least 95% sequence identity to SEQ ID NO: 126.

The Fab that is a RAM can comprise a Fab light chain comprising a light chain complementarity determining region 1 (LCDR1) comprising the amino acid sequence of SEQ ID NO: 132, a LCDR2 comprising the amino acid sequence of GAS, and a LCDR3 comprising the amino acid sequence of SEQ ID NO: 133.

The Fab that is a RAM can comprise a Fab light chain comprising a variable region comprising the amino acid sequence of SEQ ID NO: 131. The Fab that is a RAM can comprise a Fab light chain comprising a variable region comprising an amino acid sequence with at least 90% sequence identity to SEQ ID NO: 131. The Fab that is a RAM can comprise a Fab light chain comprising a variable region comprising an amino acid sequence with at least 95% sequence identity to SEQ ID NO: 131.

The Fab that is a RAM can comprise a Fab heavy chain comprising a heavy chain complementarity determining region 1 (HCDR1) comprising the amino acid sequence of SEQ ID NO: 127, a HCDR2 comprising the amino acid sequence of SEQ ID NO: 128, and a HCDR3 comprising the amino acid sequence of SEQ ID NO: 129, and a Fab light chain comprising a light chain complementarity determining region 1 (LCDR1) comprising the amino acid sequence of SEQ ID NO: 132, a LCDR2 comprising the amino acid sequence of GAS, and a LCDR3 comprising the amino acid sequence of SEQ ID NO: 133.

The Fab that is a RAM can comprise a Fab heavy chain comprising a variable region comprising the amino acid sequence of SEQ ID NO: 126 and a Fab light chain comprising a variable region comprising the amino acid sequence of SEQ ID NO: 131. The Fab that is a RAM can comprise a Fab heavy chain comprising a variable region comprising an amino acid sequence with at least 90% sequence identity to SEQ ID NO: 126 and a Fab light chain comprising a variable region comprising an amino acid sequence with at least 90% sequence identity to the amino acid sequence of SEQ ID NO: 131. The Fab that is a RAM can comprise a Fab heavy chain comprising a variable region comprising an amino acid sequence with at least 95% sequence identity to SEQ ID NO: 126 and a Fab light chain comprising a variable region comprising an amino acid sequence with at least 95% sequence identity to the amino acid sequence of SEQ ID NO: 131.

In some embodiments, the antibody comprising a Fab that is a RAM comprises the amino acid sequence of SEQ ID NO: 139. The isolated polypeptide comprising a RAM can comprise an amino acid sequence with at least 90% sequence identity to SEQ ID NO: 139. The isolated polypeptide comprising a RAM can comprise an amino acid sequence with at least 95% sequence identity to SEQ ID NO: 139.

In some embodiments, the Fab immunospecifically binds to pERK1/2. The Fab that immunospecifically binds to pERK1/2 can comprise a heavy chain complementarity determining region 1 (HCDR1) comprising the amino acid sequence of SEQ ID NO: 85, a HCDR2 comprising the amino acid sequence of SEQ ID NO: 86, and a HCDR3 comprising the amino acid sequence of SEQ ID NO: 87.

The Fab that immunospecifically binds to pERK1/2 can comprise a Fab heavy chain comprising a variable region comprising the amino acid sequence of SEQ ID NO: 84. The Fab that immunospecifically binds to pERK1/2 can comprise a Fab heavy chain comprising a variable region comprising an amino acid sequence with at least 90% sequence identity to SEQ ID NO: 84. The Fab that immunospecifically binds to pERK1/2 can comprise a Fab heavy chain comprising a variable region comprising an amino acid sequence with at least 95% sequence identity to SEQ ID NO: 84.

The Fab that immunospecifically binds pERK1/2 can comprise a Fab light chain comprising a light chain complementarity determining region 1 (LCDR1) comprising the amino acid sequence of SEQ ID NO: 91, a LCDR2 comprising the amino acid sequence of KVS, and a LCDR3 comprising the amino acid sequence of SEQ ID NO: 92.

The Fab that immunospecifically binds pERK1/2 can comprise a Fab light chain comprising a variable region comprising the amino acid sequence of SEQ ID NO: 90. The Fab that immunospecifically binds pERK1/2 can comprise a Fab light chain comprising a variable region comprising an amino acid sequence with at least 90% sequence identity to SEQ ID NO: 90. The Fab that immunospecifically binds pERK1/2 can comprise a Fab light chain comprising a variable region comprising an amino acid sequence with at least 95% sequence identity to SEQ ID NO: 90.

The Fab that immunospecifically binds to pERK1/2 can comprise a Fab heavy chain comprising a heavy chain complementarity determining region 1 (HCDR1) comprising the amino acid sequence of SEQ ID NO: 85, a HCDR2 comprising the amino acid sequence of SEQ ID NO: 86, and a HCDR3 comprising the amino acid sequence of SEQ ID NO: 87, and a Fab light chain comprising a light chain complementarity determining region 1 (LCDR1) comprising the amino acid sequence of SEQ ID NO: 91, a LCDR2 comprising the amino acid sequence of KVS, and a LCDR3 comprising the amino acid sequence of SEQ ID NO: 92.

The Fab that immunospecifically binds to pERK1/2 can comprise a Fab heavy chain comprising a variable region comprising the amino acid sequence of SEQ ID NO: 84 and a Fab light chain comprising a variable region comprising the amino acid sequence of SEQ ID NO: 90. The Fab that immunospecifically binds to pERK1/2 can comprise a Fab heavy chain comprising a variable region comprising an amino acid sequence with at least 90% sequence identity to SEQ ID NO: 84 and a Fab light chain comprising a variable region comprising an amino acid sequence with at least 90% sequence identity to the amino acid sequence of SEQ ID NO: 90. The Fab that immunospecifically binds to pERK1/2 can comprise a Fab heavy chain comprising a variable region comprising an amino acid sequence with at least 95% sequence identity to SEQ ID NO: 84 and a Fab light chain comprising a variable region comprising an amino acid sequence with at least 95% sequence identity to the amino acid sequence of SEQ ID NO: 90.

In some embodiments, the antibody comprising a Fab that immunospecifically binds to pERK1/2 comprises the amino acid sequence of SEQ ID NO: 98. The isolated polypeptide comprising a Fab that immunospecifically binds to pERK1/2 can comprise an amino acid sequence with at least 90% sequence identity to SEQ ID NO: 98. The isolated polypeptide comprising a Fab that immunospecifically binds to pERK1/2 can comprise an amino acid sequence with at least 95% sequence identity to SEQ ID NO: 98.

In some embodiments, the Fab immunospecifically binds to PSD-95. The Fab that immunospecifically binds to PSD-95 can comprise a heavy chain complementarity determining region 1 (HCDR1) comprising the amino acid sequence of SEQ ID NO: 111, a HCDR2 comprising the amino acid sequence of SEQ ID NO: 112, and a HCDR3 comprising the amino acid sequence of SEQ ID NO: 113.

The Fab that immunospecifically binds to PSD-95 can comprise a Fab heavy chain comprising a variable region comprising the amino acid sequence of SEQ ID NO: 110. The Fab that immunospecifically binds to PSD-95 can comprise a Fab heavy chain comprising a variable region comprising an amino acid sequence with at least 90% sequence identity to SEQ ID NO: 110. The Fab that immunospecifically binds to PSD-95 can comprise a Fab heavy chain comprising a variable region comprising an amino acid sequence with at least 95% sequence identity to SEQ ID NO: 110.

The Fab that immunospecifically binds PSD-95 can comprise a Fab light chain comprising a light chain complementarity determining region 1 (LCDR1) comprising the amino acid sequence of SEQ ID NO: 116, a LCDR2 comprising the amino acid sequence of DTS, and a LCDR3 comprising the amino acid sequence of SEQ ID NO: 117.

The Fab that immunospecifically binds PSD-95 can comprise a Fab light chain comprising a variable region comprising the amino acid sequence of SEQ ID NO: 115. The Fab that immunospecifically binds PSD-95 can comprise a Fab light chain comprising a variable region comprising an amino acid sequence with at least 90% sequence identity to SEQ ID NO: 115. The Fab that immunospecifically binds PSD-95 can comprise a Fab light chain comprising a variable region comprising an amino acid sequence with at least 95% sequence identity to SEQ ID NO: 115.

The Fab that immunospecifically binds to PSD-95 can comprise a Fab heavy chain comprising a heavy chain complementarity determining region 1 (HCDR1) comprising the amino acid sequence of SEQ ID NO: 111, a HCDR2 comprising the amino acid sequence of SEQ ID NO: 112, and a HCDR3 comprising the amino acid sequence of SEQ ID NO: 113, and a Fab light chain comprising a light chain complementarity determining region 1 (LCDR1) comprising the amino acid sequence of SEQ ID NO: 116, a LCDR2 comprising the amino acid sequence of DTS, and a LCDR3 comprising the amino acid sequence of SEQ ID NO: 117.

The Fab that immunospecifically binds to PSD-95 can comprise a Fab heavy chain comprising a variable region comprising the amino acid sequence of SEQ ID NO: 110 and a Fab light chain comprising a variable region comprising the amino acid sequence of SEQ ID NO: 115. The Fab that immunospecifically binds to PSD-95 can comprise a Fab heavy chain comprising a variable region comprising an amino acid sequence with at least 90% sequence identity to SEQ ID NO: 110 and a Fab light chain comprising a variable region comprising an amino acid sequence with at least 90% sequence identity to the amino acid sequence of SEQ ID NO: 115. The Fab that immunospecifically binds to PSD-95 can comprise a Fab heavy chain comprising a variable region comprising an amino acid sequence with at least 95% sequence identity to SEQ ID NO: 110 and a Fab light chain comprising a variable region comprising an amino acid sequence with at least 95% sequence identity to the amino acid sequence of SEQ ID NO: 115.

In some embodiments, the antibody comprising a Fab that immunospecifically binds to PSD-95 comprises the amino acid sequence of SEQ ID NO: 123. The isolated polypeptide comprising a Fab that immunospecifically binds to PSD-95 can comprise an amino acid sequence with at least 90% sequence identity to SEQ ID NO: 123. The isolated polypeptide comprising a Fab that immunospecifically binds to PSD-95 can comprise an amino acid sequence with at least 95% sequence identity to SEQ ID NO: 123.

In some embodiments, the Fab immunospecifically binds to TIGIT. The Fab that immunospecifically binds to TIGIT can comprise a heavy chain complementarity determining region 1 (HCDR1) comprising the amino acid sequence of SEQ ID NO: 146, a HCDR2 comprising the amino acid sequence of SEQ ID NO: 147, and a HCDR3 comprising the amino acid sequence of SEQ ID NO: 148.

The Fab that immunospecifically binds to TIGIT can comprise a Fab heavy chain comprising a variable region comprising the amino acid sequence of SEQ ID NO: 145. The Fab that immunospecifically binds to TIGIT can comprise a Fab heavy chain comprising a variable region comprising an amino acid sequence with at least 90% sequence identity to SEQ ID NO: 145. The Fab that immunospecifically binds to TIGIT can comprise a Fab heavy chain comprising a variable region comprising an amino acid sequence with at least 95% sequence identity to SEQ ID NO: 145.

The Fab that immunospecifically binds to TIGIT can comprise a Fab light chain comprising a light chain complementarity determining region 1 (LCDR1) comprising the amino acid sequence of SEQ ID NO: 151, a LCDR2 comprising the amino acid sequence of SAS, and a LCDR3 comprising the amino acid sequence of SEQ ID NO: 152.

The Fab that immunospecifically binds to TIGIT can comprise a Fab light chain comprising a variable region comprising the amino acid sequence of SEQ ID NO: 150. The Fab that immunospecifically binds to TIGIT can comprise a Fab light chain comprising a variable region comprising an amino acid sequence with at least 90% sequence identity to SEQ ID NO: 150. The Fab that immunospecifically binds to TIGIT can comprise a Fab light chain comprising a variable region comprising an amino acid sequence with at least 95% sequence identity to SEQ ID NO: 150.

The Fab that immunospecifically binds to TIGIT can comprise a Fab heavy chain comprising a heavy chain complementarity determining region 1 (HCDR1) comprising the amino acid sequence of SEQ ID NO: 146, a HCDR2 comprising the amino acid sequence of SEQ ID NO: 147, and a HCDR3 comprising the amino acid sequence of SEQ ID NO: 148, and a Fab light chain comprising a light chain complementarity determining region 1 (LCDR1) comprising the amino acid sequence of SEQ ID NO: 151, a LCDR2 comprising the amino acid sequence of SAS, and a LCDR3 comprising the amino acid sequence of SEQ ID NO: 152.

The Fab that immunospecifically binds to TIGIT can comprise a Fab heavy chain comprising a variable region comprising the amino acid sequence of SEQ ID NO: 145 and a Fab light chain comprising a variable region comprising the amino acid sequence of SEQ ID NO: 150. The Fab that immunospecifically binds to TIGIT can comprise a Fab heavy chain comprising a variable region comprising an amino acid sequence with at least 90% sequence identity to SEQ ID NO: 145 and a Fab light chain comprising a variable region comprising an amino acid sequence with at least 90% sequence identity to the amino acid sequence of SEQ ID NO: 150. The Fab that immunospecifically binds to TIGIT can comprise a Fab heavy chain comprising a variable region comprising an amino acid sequence with at least 95% sequence identity to SEQ ID NO: 145 and a Fab light chain comprising a variable region comprising an amino acid sequence with at least 95% sequence identity to the amino acid sequence of SEQ ID NO: 150.

In some embodiments, the Fab that immunospecifically binds to TIGIT comprises the amino acid sequence of SEQ ID NO: 157. The isolated polypeptide comprising a Fab that immunospecifically binds to TIGIT can comprise an amino acid sequence with at least 90% sequence identity to SEQ ID NO: 157. The isolated polypeptide comprising a Fab that immunospecifically binds to TIGIT can comprise an amino acid sequence with at least 95% sequence identity to SEQ ID NO: 157.

Any antibody disclosed herein can be an antibody with the isotype IgG.

In some embodiments, the isolated polypeptide or the antibody further comprises a label.

Disclosed herein are nucleic acid molecules comprising, consisting essentially of, or consisting of polynucleotides encoding isolated polypeptides comprising, consisting essentially of, or consisting of the amino acid sequence of Formula I: Z-(X-Yₙ-Zᵣ)ₚ-X, wherein Z is a hinge, X is a CH2 domain, Y is a linker, n is an integer of 1 or greater, p is an integer of 2 or greater, and r is an integer of 0 or 1.

The nucleic acid molecules can comprise polynucleotides that encode a hinge (Z) comprising the amino acid sequence of SEQ ID NO: 18. The polynucleotides can encode a hinge (Z) comprising an amino acid sequence with at least 90% sequence identity to SEQ ID NO: 18. The polynucleotides can encode a hinge (Z) comprising an amino acid sequence with at least 95% sequence identity to SEQ ID NO: 18. In some embodiments, the polynucleotides encoding the hinge (Z) comprise SEQ ID NO: 22.

The nucleic acid molecules can comprise polynucleotides that encode a polypeptide that comprises, consists essentially of, or consists of (X-Yₙ-Zᵣ)ₚ, wherein the hinge (Z) comprises the amino acid sequence of SEQ ID NO: 48. The polynucleotides can encode a hinge (Z) comprising an amino acid sequence with at least 90% sequence identity to SEQ ID NO: 48. The polynucleotides can encode a hinge (Z) comprising an amino acid sequence with at least 95% sequence identity to SEQ ID NO: 48. In some embodiments, the polynucleotides encoding the hinge (Z) comprise SEQ ID NO: 51.

The nucleic acid molecules can comprise polynucleotides that encode a polypeptide that comprises, consists essentially of, or consists of (X-Yₙ-Zᵣ), wherein the hinge (Z) comprises the amino acid sequence of SEQ ID NO: 102. The polynucleotides can encode a hinge (Z) comprising an amino acid sequence with at least 90% sequence identity to SEQ ID NO: 102. The polynucleotides can encode a hinge (Z) comprising an amino acid sequence with at least 95% sequence identity to SEQ ID NO: 102. In some embodiments, the polynucleotides encoding the hinge (Z) comprise SEQ ID NO: 101.

The polynucleotides encoding a polypeptide comprising, consisting essentially of, or consisting of (X-Yₙ-Zᵣ)ₚ can encode a CH2 domain (X) comprising the amino acid sequence of SEQ ID NO: 19. The polynucleotides can encode a CH2 domain (X) comprising an amino acid sequence with at least 90% sequence identity to SEQ ID NO: 19. The polynucleotides can encode a CH2 domain (X) comprising an amino acid sequence with at least 95% sequence identity to SEQ ID NO: 19. In some embodiments, the polynucleotides encoding the CH2 domain (X) comprise SEQ ID NO: 23 or SEQ ID NO: 178.

The nucleic acid molecules encoding a polypeptide comprising, consisting essentially of, or consisting of (X-Yₙ-Zᵣ)ₚ, can comprise polynucleotides that encode a CH2 domain (X) comprising the amino acid sequence of SEQ ID NO: 49. The polynucleotides can encode a CH2 domain (X) comprising an amino acid sequence with at least 90% sequence identity to SEQ ID NO: 49. The polynucleotides can encode a CH2 domain (X) comprising an amino acid sequence with at least 95% sequence identity to SEQ ID NO: 49. In some embodiments, the polynucleotides encoding the CH2 domain (X) comprise SEQ ID NO: 62.

The polynucleotides encoding a polypeptide comprising, consisting essentially of, or consisting of (X-Yₙ-Zᵣ)ₚ can encode a CH2 domain (X) comprising the amino acid sequence of SEQ ID NO: 102. The polynucleotides can encode a CH2 domain (X) comprising an amino acid sequence with at least 90% sequence identity to SEQ ID NO: 102. The polynucleotides can encode a CH2 domain (X) comprising an amino acid sequence with at least 95% sequence identity to SEQ ID NO: 102. In some embodiments, the polynucleotides encoding the CH2 domain (X) comprise SEQ ID NO: 101.

The nucleic acid molecules encoding a polypeptide comprising, consisting essentially of, or consisting of (X-Yₙ-Zᵣ)ₚ can comprise polynucleotides that encode an isolated polypeptide comprising one or more linkers (Y) comprising the amino acid sequence of GS, GGS, SEQ ID NO: 14, SEQ ID NO: 38, SEQ ID NO: 43, or SEQ ID NO: 44. The polynucleotide can encode one or more linkers (Y) comprising GS. The polynucleotide can encode one or more linkers (Y) comprising GGS. The polynucleotide can encode one or more linkers (Y) comprising SEQ ID NO: 14. The polynucleotide can encode one or more linkers (Y) comprising SEQ ID NO: 38. The polynucleotide can encode one or more linkers (Y) comprising SEQ ID NO: 43. The polynucleotide can encode one or more linkers (Y) comprising an amino acid sequence with at least 90% sequence identity to SEQ ID NO: 43. The polynucleotide can encode one or more linkers (Y) comprising SEQ ID NO: 44. The polynucleotide can encode one or more linkers (Y) comprising an amino acid sequence with at least 90% sequence identity to SEQ ID NO: 44. In some embodiments, the polynucleotide encoding the one or more linkers (Y) comprise SEQ ID NO: 46.

The polynucleotide can encode an isolated polypeptide comprising one linker (Y), such that n is 1. The polynucleotide can encode an isolated polypeptide comprising two linkers (Y), such that n is 2. The polynucleotide can encode an isolated polypeptide comprising three linkers (Y), such that n is 3. The polynucleotide can encode an isolated polypeptide comprising four linkers (Y), such that n is 4. The polynucleotide can encode an isolated polypeptide comprising five linkers (Y), such that n is 5.

The polynucleotide can encode an isolated polypeptide comprising two or more regions comprising a CH2-linker region ((X-Yₙ)ₚ), such that p is 2 or greater. The polynucleotide can encode an isolated polypeptide comprising two CH2-linker regions ((X-Yₙ)ₚ), such that p is 2. The polynucleotide can encode an isolated polypeptide comprising three CH2-linker regions ((X-Yₙ)ₚ), such that p is 3. The polynucleotide can encode an isolated polypeptide comprising four CH2-linker regions ((X-Yₙ)ₚ), such that p is 4. The polynucleotide can encode an isolated polypeptide comprising five CH2-linker regions ((X-Yₙ)ₚ), such that p is 5.

The polynucleotide can encode an isolated polypeptide comprising two or more regions comprising a CH2-linker-hinge region ((X-Yₙ-Zᵣ)ₚ), such that p is 2 or greater.

The polynucleotide can encode an isolated polypeptide comprising two CH2-linker-hinge regions ((X-Yₙ-Zᵣ)ₚ), such that p is 2. The polynucleotide can encode an isolated polypeptide comprising three CH2-linker-hinge regions ((X-Yₙ-Zᵣ)ₚ), such that p is 3. The polynucleotide can encode an isolated polypeptide comprising four CH2-linker-hinge regions ((X-Yₙ-Zᵣ)ₚ), such that p is 4. The polynucleotide can encode an isolated polypeptide comprising five CH2-linker-hinge regions ((X-Yₙ-Zᵣ)ₚ), such that p is 5.

In some embodiments, the nucleic acid molecule comprises a polynucleotide encoding an isolated polypeptide comprising, consisting essentially of, or consisting of: Z (hinge) comprising the amino acid sequence of SEQ ID NO: 18, X(CH2 domain) comprising the amino acid sequence of SEQ ID NO: 19, Y (linker) comprising the amino acid sequence of SEQ ID NO: 44, wherein n is 1, and p is 2. In some embodiments, the polynucleotide encoding the hinge (X) comprises SEQ ID NO: 22 and the polynucleotide encoding the CH2 domain (X) comprises SEQ ID NO: 23 or SEQ ID NO: 178.

In some embodiments, the nucleic acid molecule comprises a polynucleotide encoding an isolated polypeptide comprising, consisting essentially of, or consisting of: Z (hinge) comprising the amino acid sequence of SEQ ID NO: 48, X(CH2 domain) comprising the amino acid sequence of SEQ ID NO: 49, Y (linker) comprising the amino acid sequence of SEQ ID NO: 44, wherein n is 1, and p is 2. In some embodiments, the polynucleotide encoding the hinge (X) comprises SEQ ID NO: 51 and the polynucleotide encoding the CH2 domain (X) comprises SEQ ID NO: 62.

In some embodiments, the nucleic acid molecule comprises a polynucleotide encoding an isolated polypeptide comprising, consisting essentially of, or consisting of: Z (hinge) comprising the amino acid sequence of SEQ ID NO: 102, X(CH2 domain) comprising the amino acid sequence of SEQ ID NO: 104, Y (linker) comprising the amino acid sequence of SEQ ID NO: 44 wherein n is 1, r is 0 or 1, and p is 2. In some embodiments, the polynucleotide encoding the hinge (X) comprises SEQ ID NO: 101 and the polynucleotide encoding the CH2 domain (X) comprises SEQ ID NO: 103.

The polynucleotide can encode an isolated polypeptide comprising two additional CH2 domains N-terminal to the Fc domain connected via a hinge and optionally one or more linkers, wherein the isolated polypeptide comprises the amino acid sequence of SEQ ID NO: 52. In some embodiments, the polynucleotide encodes an isolated polypeptide comprising two additional CH2 domains N-terminal to the Fc domain connected via a hinge and optionally one or more linkers, the isolated polypeptide comprising SEQ ID NO: 52. The polynucleotide can encode an isolated polypeptide comprising two additional CH2 domains N-terminal to the Fc domain connected via a hinge and optionally one or more linkers, the isolated polypeptide comprising an amino acid sequence with at least 90% identity to SEQ ID NO: 52. The polynucleotide can encode an isolated polypeptide comprising two additional CH2 domains N-terminal to the Fc domain connected via a hinge and optionally one or more linkers, the isolated polypeptide comprising an amino acid sequence with at least 95% identity to SEQ ID NO: 52. In some embodiments, in the polynucleotide encoding an isolated polypeptide comprising two additional CH2 domains N-terminal to the Fc domain connected via a hinge and optionally one or more linkers comprises SEQ ID NO: 53.

In some embodiments, the polynucleotide encodes an isolated polypeptide comprising two additional CH2 domains N-terminal to the Fc domain connected via a hinge and optionally one or more linkers and comprises “LFLEPS” mutations within the lower hinge and CH2 domains. In some embodiments, the “LFLEPS” mutations can comprise the following amino acid substitutions: L234F, L235E and P331S. In some embodiments, the polynucleotide encodes an isolated polypeptide comprising two additional CH2 domains N-terminal to the Fc domain connected via a hinge and optionally one or more linkers and comprises “LFLEPS” mutations within the lower hinge and CH2 domains, the isolated polypeptide comprising SEQ ID NO: 54. The polynucleotide can encode an isolated polypeptide comprising two additional CH2 domains N-terminal to the Fc domain connected via a hinge and optionally one or more linkers and comprises “LFLEPS” mutations within the lower hinge and CH2 domains, the isolated polypeptide comprising an amino acid sequence with at least 90% identity to SEQ ID NO: 54. The polynucleotide can encode an isolated polypeptide comprising two additional CH2 domains N-terminal to the Fc domain connected via a hinge and optionally one or more linkers and comprises “LFLEPS” mutations within the lower hinge and CH2 domains, the isolated polypeptide comprising an amino acid sequence with at least 95% identity to SEQ ID NO: 54. In some embodiments, in the polynucleotide encoding an isolated polypeptide comprising two additional CH2 domains N-terminal to the Fc domain connected via a hinge and optionally one or more linkers and comprises “LFLEPS” mutations within the lower hinge and CH2 domains comprises SEQ ID NO: 55.

The polynucleotide can encode any isolated polypeptide disclosed herein and can further encode a second polypeptide, wherein the isolated polypeptide and the second polypeptide share the same amino acid sequence.

The polynucleotide can encode any isolated polypeptide disclosed herein that further comprises a CH3 domain. In some embodiments the polynucleotide encodes a CH3 domain comprising the amino acid sequence of SEQ ID NO: 20. The polynucleotide can encode a CH3 domain comprising an amino acid sequence with at least 90% sequence identity to SEQ ID NO: 20. The polynucleotide can encode a CH3 domain comprising an amino acid sequence with at least 95% sequence identity to SEQ ID NO: 20. In some embodiments, the polynucleotide encoding the CH3 domain comprises SEQ ID NO: 24 or SEQ ID NO: 179.

In some embodiments, the polynucleotide encodes an isolated polypeptide comprising a CH3 domain, the isolated polypeptide comprising SEQ ID NO: 106. The polynucleotide can encode an isolated polypeptide comprising a CH3 domain, the isolated polypeptide comprising an amino acid sequence with at least 90% identity to SEQ ID NO: 106. The polynucleotide can encode an isolated polypeptide comprising a CH3 domain, the isolated polypeptide comprising an amino acid sequence with at least 95% identity to SEQ ID NO: 106. In some embodiments, in the polynucleotide encoding an isolated polypeptide comprising a CH3 domain comprises SEQ ID NO: 105.

In some embodiments, the polynucleotide encodes an isolated polypeptide comprising a CH3 domain, the isolated polypeptide comprising SEQ ID NO: 141. The polynucleotide can encode an isolated polypeptide comprising a CH3 domain, the isolated polypeptide comprising an amino acid sequence with at least 90% identity to SEQ ID NO: 141. The polynucleotide can encode an isolated polypeptide comprising a CH3 domain, the isolated polypeptide comprising an amino acid sequence with at least 95% identity to SEQ ID NO: 141. In some embodiments, in the polynucleotide encoding an isolated polypeptide comprising a CH3 domain comprises SEQ ID NO: 140.

The polynucleotide can encode any antibody comprising any isolated polypeptide disclosed herein that further comprises a Fab. The polynucleotide can encode a Fab comprising a CH1 domain. The polynucleotide can encode a CH1 domain comprising the amino acid sequence of SEQ ID NO: 17. The polynucleotide can encode a CH1 domain comprising an amino acid sequence with at least 90% sequence identity to SEQ ID NO: 17. The polynucleotide can encode a CH1 domain comprising amino acid sequence with at least 95% sequence identity to SEQ ID NO: 17. The polynucleotide encoding a CH1 domain can comprise SEQ ID NO: 21, SEQ ID NO: 176, or SEQ ID NO: 177. The polynucleotide can encode a Fab comprising a Fab heavy chain and a Fab light chain that pair to form a binding domain.

The polynucleotide can encode any antibody comprising any isolated polypeptide disclosed herein that further comprises a Fab. The polynucleotide can encode a Fab comprising a CH1 domain. The polynucleotide can encode a CH1 domain comprising the amino acid sequence of SEQ ID NO: 100. The polynucleotide can encode a CH1 domain comprising an amino acid sequence with at least 90% sequence identity to SEQ ID NO: 100. The polynucleotide can encode a CH1 domain comprising amino acid sequence with at least 95% sequence identity to SEQ ID NO: 100. The polynucleotide encoding a CH1 domain can comprise SEQ ID NO: 99. The polynucleotide can encode a Fab comprising a Fab heavy chain and a Fab light chain that pair to form a binding domain.

The polynucleotide can encode any antibody comprising any isolated polypeptide disclosed herein that further comprises a Fab. The polynucleotide can encode a Fab comprising a CH1 domain. The polynucleotide can encode a CH1 domain comprising the amino acid sequence of SEQ ID NO: 160. The polynucleotide can encode a CH1 domain comprising an amino acid sequence with at least 90% sequence identity to SEQ ID NO: 160.

The polynucleotide can encode a CH1 domain comprising amino acid sequence with at least 95% sequence identity to SEQ ID NO: 160. The polynucleotide encoding a CH1 domain can comprise SEQ ID NO: 159. The polynucleotide can encode a Fab comprising a Fab heavy chain and a Fab light chain that pair to form a binding domain.

The polynucleotide can encode a Fab that immunospecifically binds to one or more target molecules. The polynucleotide can encode a Fab that immunospecifically binds to Parkin, OCT4, IDO1, FLAG-tag, pERK1/2, PSD-95, or TIGIT, or the Fab can be a rabbit anti-mouse (RAM) IgG secondary antibody.

In some embodiments, the polynucleotide can encode a Fab that immunospecifically binds to Parkin. The polynucleotide can encode a Fab that immunospecifically binds to Parkin, the Fab comprising a Fab heavy chain comprising a heavy chain complementarity determining region 1 (HCDR1) comprising the amino acid sequence of SEQ ID NO: 4, a HCDR2 comprising the amino acid sequence of SEQ ID NO: 5, and a HCDR3 comprising the amino acid sequence of SEQ ID NO: 6.

The polynucleotide can encode a Fab that immunospecifically binds to Parkin, the Fab comprising a Fab heavy chain comprising a variable region comprising the amino acid sequence of SEQ ID NO: 7. The polynucleotide can encode a Fab heavy chain comprising a variable region comprising an amino acid sequence with at least 90% sequence identity to SEQ ID NO: 7. The polynucleotide can encode a Fab heavy chain comprising a variable region comprising an amino acid sequence with at least 95% sequence identity to SEQ ID NO: 7.

The polynucleotide can encode a Fab that immunospecifically binds to Parkin, the Fab comprising a Fab heavy chain comprising a variable region comprising the amino acid sequence of SEQ ID NO: 9. The polynucleotide can encode a Fab heavy chain comprising a variable region comprising an amino acid sequence with at least 90% sequence identity to SEQ ID NO: 9. The polynucleotide can encode a Fab heavy chain comprising a variable region comprising an amino acid sequence with at least 95% sequence identity to SEQ ID NO: 9. In some embodiments, in the polynucleotide encoding a Fab that immunospecifically binds to Parkin, the polynucleotide comprises SEQ ID NO: 8.

The polynucleotide can encode a Fab that immunospecifically binds to Parkin, the Fab comprising a Fab light chain comprising a light chain complementarity determining region 1 (LCDR1) comprising the amino acid sequence of SEQ ID NO: 13, a LCDR2 comprising the amino acid sequence RAS, and a LCDR3 comprising the amino acid sequence of SEQ ID NO: 15.

The polynucleotide can encode a Fab that immunospecifically binds to Parkin, the Fab comprising a Fab light chain comprising a variable region comprising the amino acid sequence of SEQ ID NO: 16. The polynucleotide can encode a Fab light chain comprising a variable region comprising an amino acid sequence with at least 90% sequence identity to SEQ ID NO: 16. The polynucleotide can encode a Fab light chain comprising a variable region comprising an amino acid sequence with at least 95% sequence identity to SEQ ID NO: 16.

The polynucleotide can encode a Fab that immunospecifically binds to Parkin, the Fab comprising a Fab light chain comprising a variable region comprising the amino acid sequence of SEQ ID NO: 12. The polynucleotide can encode a Fab light chain comprising a variable region comprising an amino acid sequence with at least 90% sequence identity to SEQ ID NO: 12. The polynucleotide can encode a Fab light chain comprising a variable region comprising an amino acid sequence with at least 95% sequence identity to SEQ ID NO: 12. In some embodiments, in the polynucleotide encoding a Fab that immunospecifically binds to Parkin, the polynucleotide comprises SEQ ID NO: 11.

The polynucleotide can encode a Fab that immunospecifically binds to Parkin, the Fab comprising a Fab heavy chain comprising a heavy chain complementarity determining region 1 (HCDR1) comprising the amino acid sequence of SEQ ID NO: 4, a HCDR2 comprising the amino acid sequence of SEQ ID NO: 5, and a HCDR3 comprising the amino acid sequence of SEQ ID NO: 6, and a Fab light chain comprising a light chain complementarity determining region 1 (LCDR1) comprising the amino acid sequence of SEQ ID NO: 13, a LCDR2 comprising the amino acid sequence RAS, and a LCDR3 comprising the amino acid sequence of SEQ ID NO: 15.

The polynucleotide can encode a Fab that immunospecifically binds to Parkin, the Fab comprising a Fab heavy chain comprising a variable region comprising the amino acid sequence of SEQ ID NO: 7 and Fab light chain comprising a variable region comprising the amino acid sequence of SEQ ID NO: 16. The polynucleotide can encode a Fab heavy chain comprising a variable region comprising an amino acid sequence with at least 90% sequence identity to SEQ ID NO: 7 and a Fab light chain comprising a variable region comprising an amino acid sequence with at least 90% sequence identity to the amino acid sequence of SEQ ID NO: 16. The polynucleotide can encode a Fab heavy chain comprising a variable region comprising an amino acid sequence with at least 95% sequence identity to SEQ ID NO: 7 and a Fab light chain comprising a variable region comprising an amino acid sequence with at least 95% sequence identity to the amino acid sequence of SEQ ID NO: 16.

The polynucleotide can encode a Fab that immunospecifically binds to Parkin, the Fab comprising a Fab heavy chain comprising a variable region comprising the amino acid sequence of SEQ ID NO: 9 and a Fab light chain comprising a variable region comprising the amino acid sequence of SEQ ID NO: 12. The polynucleotide can encode a Fab heavy chain comprising a variable region comprising an amino acid sequence with at least 90% sequence identity to the amino acid sequence of SEQ ID NO: 9 and Fab light chain comprising a variable region comprising an amino acid sequence with at least 90% sequence identity to the amino acid sequence of SEQ ID NO: 12. The polynucleotide can encode a Fab heavy chain comprising a variable region comprising an amino acid sequence with at least 95% sequence identity to the amino acid sequence of SEQ ID NO: 9 and Fab light chain comprising a variable region comprising an amino acid sequence with at least 95% sequence identity to the amino acid sequence of SEQ ID NO: 12. In some embodiments, in the polynucleotide encoding a Fab that immunospecifically binds to Parkin, the polynucleotide comprises SEQ ID NO: 8 and SEQ ID NO: 11.

In some embodiments, the polynucleotide can encode an antibody comprising a Fab that immunospecifically binds to Parkin, the Fab comprising the amino acid sequence of SEQ ID NO: 3. The polynucleotide can encode a Fab comprising an amino acid sequence with at least 90% sequence identity to SEQ ID NO: 3. The polynucleotide can encode a Fab comprising an amino acid sequence with at least 95% sequence identity to SEQ ID NO: 3. In some embodiments, in the polynucleotide encoding a Fab that immunospecifically binds to Parkin, the polynucleotide comprises SEQ ID NO: 2.

In some embodiments, the polynucleotide can encode a Fab that immunospecifically binds to OCT4. The polynucleotide can encode a Fab that immunospecifically binds to OCT4, the Fab comprising a heavy chain complementarity determining region 1 (HCDR1) comprising the amino acid sequence of SEQ ID NO: 29, a HCDR2 comprising the amino acid sequence of SEQ ID NO: 30, and a HCDR3 comprising the amino acid sequence of SEQ ID NO: 31.

The polynucleotide can encode a Fab that immunospecifically binds to OCT4, the Fab comprising a Fab heavy chain comprising a variable region comprising the amino acid sequence of SEQ ID NO: 32. The polynucleotide can encode a Fab heavy chain comprising a variable region comprising an amino acid sequence with at least 90% sequence identity to SEQ ID NO: 32. The polynucleotide can encode a Fab heavy chain comprising a variable region comprising an amino acid sequence with at least 95% sequence identity to SEQ ID NO: 32.

The polynucleotide can encode a Fab that immunospecifically binds to OCT4, the Fab comprising a Fab heavy chain comprising a variable region comprising the amino acid sequence of SEQ ID NO: 34. The polynucleotide can encode a Fab heavy chain comprising a variable region comprising an amino acid sequence with at least 90% sequence identity to SEQ ID NO: 34. The polynucleotide can encode a Fab heavy chain comprising a variable region comprising an amino acid sequence with at least 95% sequence identity to SEQ ID NO: 34. In some embodiments, in the polynucleotide encoding a Fab that immunospecifically binds to OCT4, the polynucleotide comprises SEQ ID NO: 33.

The polynucleotide can encode a Fab that immunospecifically binds to OCT4, the Fab comprising a Fab light chain comprising a light chain complementarity determining region 1 (LCDR1) comprising the amino acid sequence of SEQ ID NO: 37, a LCDR2 comprising the amino acid sequence GAS, and a LCDR3 comprising the amino acid sequence of SEQ ID NO: 39.

The polynucleotide can encode a Fab that immunospecifically binds to OCT4, the Fab comprising a Fab light chain comprising a variable region comprising the amino acid sequence of SEQ ID NO: 40. The polynucleotide can encode a Fab light chain comprising a variable region comprising an amino acid sequence with at least 90% sequence identity to SEQ ID NO: 40. The polynucleotide can encode a Fab light chain comprising a variable region comprising an amino acid sequence with at least 95% sequence identity to SEQ ID NO: 40.

The polynucleotide can encode a Fab that immunospecifically binds to OCT4, the Fab comprising a Fab light chain comprising the amino acid sequence of SEQ ID NO: 36. The polynucleotide can encode a Fab light chain comprising a variable region comprising an amino acid sequence with at least 90% sequence identity to SEQ ID NO: 36.

The polynucleotide can encode a Fab light chain comprising a variable region comprising an amino acid sequence with at least 95% sequence identity to SEQ ID NO: 36. In some embodiments, in the polynucleotide encoding a Fab that immunospecifically binds to OCT4, the polynucleotide comprises SEQ ID NO: 35.

The polynucleotide can encode a Fab that immunospecifically binds to OCT4, the Fab comprising a Fab heavy chain comprising a heavy chain complementarity determining region 1 (HCDR1) comprising the amino acid sequence of SEQ ID NO: 29, a HCDR2 comprising the amino acid sequence of SEQ ID NO: 30, and a HCDR3 comprising the amino acid sequence of SEQ ID NO: 31, and a Fab light chain comprising a light chain complementarity determining region 1 (LCDR1) comprising the amino acid sequence of SEQ ID NO: 37, a LCDR2 comprising the amino acid sequence GAS, and a LCDR3 comprising the amino acid sequence of SEQ ID NO: 39.

The polynucleotide can encode a Fab that immunospecifically binds to OCT4, the Fab comprising a Fab heavy chain comprising a variable region comprising the amino acid sequence of SEQ ID NO: 32 and Fab light chain comprising a variable region comprising the amino acid sequence of SEQ ID NO: 40. The polynucleotide can encode a Fab heavy chain comprising a variable region comprising an amino acid sequence with at least 90% sequence identity to SEQ ID NO: 32 and a Fab light chain comprising a variable region comprising an amino acid sequence with at least 90% sequence identity to the amino acid sequence of SEQ ID NO: 40. The polynucleotide can encode a Fab heavy chain comprising a variable region comprising an amino acid sequence with at least 95% sequence identity to SEQ ID NO: 32 and a Fab light chain comprising a variable region comprising an amino acid sequence with at least 95% sequence identity to the amino acid sequence of SEQ ID NO: 40.

The polynucleotide can encode a Fab that immunospecifically binds to OCT4, the Fab comprising a Fab heavy chain comprising a variable region comprising the amino acid sequence of SEQ ID NO: 34 and a Fab light chain comprising a variable region comprising the amino acid sequence of SEQ ID NO: 36. The polynucleotide can encode a Fab heavy chain comprising a variable region comprising an amino acid sequence with at least 90% sequence identity to the amino acid sequence of SEQ ID NO: 34 and Fab light chain comprising a variable region comprising an amino acid sequence with at least 90% sequence identity to the amino acid sequence of SEQ ID NO: 36. The polynucleotide can encode a Fab heavy chain comprising a variable region comprising an amino acid sequence with at least 95% sequence identity to the amino acid sequence of SEQ ID NO: 34 and Fab light chain comprising a variable region comprising an amino acid sequence with at least 95% sequence identity to the amino acid sequence of SEQ ID NO: 36. In some embodiments, in the polynucleotide encoding a Fab that immunospecifically binds to OCT4, the polynucleotide comprises SEQ ID NO: 33 and SEQ ID NO: 35.

In some embodiments, the polynucleotide can encode an antibody comprising a Fab that immunospecifically binds to OCT4, the Fab comprising the amino acid sequence of SEQ ID NO: 28. The polynucleotide can encode a Fab comprising an amino acid sequence with at least 90% sequence identity to SEQ ID NO: 28. The polynucleotide can encode a Fab comprising an amino acid sequence with at least 95% sequence identity to SEQ ID NO: 28. In some embodiments, in the polynucleotide encoding a Fab that immunospecifically binds to OCT4, the polynucleotide comprises SEQ ID NO: 27.

In some embodiments, the polynucleotide can encode a Fab that immunospecifically binds to IDO1. The polynucleotide can encode a Fab that immunospecifically binds to IDO1, the Fab comprising a Fab heavy chain comprising a heavy chain complementarity determining region 1 (HCDR1) comprising the amino acid sequence of SEQ ID NO: 66, a HCDR2 comprising the amino acid sequence of SEQ ID NO: 67, and a HCDR3 comprising the amino acid sequence of SEQ ID NO: 68.

The polynucleotide can encode a Fab that immunospecifically binds to IDO1, the Fab comprising a Fab heavy chain comprising a variable region comprising the amino acid sequence of SEQ ID NO: 64. The polynucleotide can encode a Fab heavy chain comprising a variable region comprising an amino acid sequence with at least 90% sequence identity to SEQ ID NO: 64. The polynucleotide can encode a Fab heavy chain comprising a variable region comprising an amino acid sequence with at least 95% sequence identity to SEQ ID NO: 64.

The polynucleotide can encode a Fab that immunospecifically binds IDO1, the Fab comprising a Fab light chain comprising a light chain complementarity determining region 1 (LCDR1) comprising the amino acid sequence of SEQ ID NO: 72, a LCDR2 comprising the amino acid sequence of YAS, and a LCDR3 comprising the amino acid sequence of SEQ ID NO: 73.

The polynucleotide can encode a Fab that immunospecifically binds IDO1, the Fab comprising a Fab light chain comprising a variable region comprising the amino acid sequence of SEQ ID NO: 71. The polynucleotide can encode a Fab light chain comprising a variable region comprising an amino acid sequence with at least 90% sequence identity to SEQ ID NO: 71. The polynucleotide can encode a Fab light chain comprising a variable region comprising an amino acid sequence with at least 95% sequence identity to SEQ ID NO: 71.

The polynucleotide can encode a Fab that immunospecifically binds to IDO1, the Fab comprising a Fab heavy chain comprising a heavy chain complementarity determining region 1 (HCDR1) comprising the amino acid sequence of SEQ ID NO: 66, a HCDR2 comprising the amino acid sequence of SEQ ID NO: 67, and a HCDR3 comprising the amino acid sequence of SEQ ID NO: 68, and a Fab light chain comprising a light chain complementarity determining region 1 (LCDR1) comprising the amino acid sequence of SEQ ID NO: 72, a LCDR2 comprising the amino acid sequence of YAS, and a LCDR3 comprising the amino acid sequence of SEQ ID NO: 73.

The polynucleotide can encode a Fab that immunospecifically binds to IDO1, the Fab comprising a Fab heavy chain comprising a variable region comprising the amino acid sequence of SEQ ID NO: 65 and a Fab light chain comprising a variable region comprising the amino acid sequence of SEQ ID NO: 71. The polynucleotide can encode a Fab heavy chain comprising a variable region comprising an amino acid sequence with at least 90% sequence identity to SEQ ID NO: 65 and a Fab light chain comprising a variable region comprising an amino acid sequence with at least 90% sequence identity to the amino acid sequence of SEQ ID NO: 71. The polynucleotide can encode a Fab heavy chain comprising a variable region comprising an amino acid sequence with at least 95% sequence identity to SEQ ID NO: 65 and a Fab light chain comprising a variable region comprising an amino acid sequence with at least 95% sequence identity to the amino acid sequence of SEQ ID NO: 71. In some embodiments, in the polynucleotide encoding a Fab that immunospecifically binds to IDO1, the polynucleotide comprises SEQ ID NO: 64 and SEQ ID NO: 70.

In some embodiments, the polynucleotide can encode an antibody comprising a Fab that immunospecifically binds to IDO1, the Fab comprising the amino acid sequence of SEQ ID NO: 79. The polynucleotide can encode a Fab comprising an amino acid sequence with at least 90% sequence identity to SEQ ID NO: 79. The polynucleotide can encode a Fab comprising an amino acid sequence with at least 95% sequence identity to SEQ ID NO: 79. In some embodiments, in the polynucleotide encoding a Fab that immunospecifically binds to IDO1, the polynucleotide comprises SEQ ID NO: 78.

The polynucleotide can encode a Fab that immunospecifically binds to FLAG-tag, the Fab comprising a Fab heavy chain comprising a variable region comprising the amino acid sequence of SEQ ID NO: 162. The polynucleotide can encode a Fab heavy chain comprising a variable region comprising an amino acid sequence with at least 90% sequence identity to SEQ ID NO: 162. The polynucleotide can encode a Fab heavy chain comprising a variable region comprising an amino acid sequence with at least 95% sequence identity to SEQ ID NO: 162.

The polynucleotide can encode a Fab that immunospecifically binds FLAG-tag, the Fab comprising a Fab light chain comprising a light chain complementarity determining region 1 (LCDR1) comprising the amino acid sequence of SEQ ID NO: 168, a LCDR2 comprising the amino acid sequence of YAS, and a LCDR3 comprising the amino acid sequence of SEQ ID NO: 169.

The polynucleotide can encode a Fab that immunospecifically binds FLAG-tag, the Fab comprising a Fab light chain comprising a variable region comprising the amino acid sequence of SEQ ID NO: 167. The polynucleotide can encode a Fab light chain comprising a variable region comprising an amino acid sequence with at least 90% sequence identity to SEQ ID NO: 167. The polynucleotide can encode a Fab light chain comprising a variable region comprising an amino acid sequence with at least 95% sequence identity to SEQ ID NO: 167.

The polynucleotide can encode a Fab that immunospecifically binds to FLAG-tag, the Fab comprising a Fab heavy chain comprising a heavy chain complementarity determining region 1 (HCDR1) comprising the amino acid sequence of SEQ ID NO: 163, a HCDR2 comprising the amino acid sequence of SEQ ID NO: 164, and a HCDR3 comprising the amino acid sequence of SEQ ID NO: 165, and a Fab light chain comprising a light chain complementarity determining region 1 (LCDR1) comprising the amino acid sequence of SEQ ID NO: 168, a LCDR2 comprising the amino acid sequence of YAS, and a LCDR3 comprising the amino acid sequence of SEQ ID NO: 169.

The polynucleotide can encode a Fab that immunospecifically binds to FLAG-tag, the Fab comprising a Fab heavy chain comprising a variable region comprising the amino acid sequence of SEQ ID NO: 162 and a Fab light chain comprising a variable region comprising the amino acid sequence of SEQ ID NO: 167. The polynucleotide can encode a Fab heavy chain comprising a variable region comprising an amino acid sequence with at least 90% sequence identity to SEQ ID NO: 162 and a Fab light chain comprising a variable region comprising an amino acid sequence with at least 90% sequence identity to the amino acid sequence of SEQ ID NO: 167. The polynucleotide can encode a Fab heavy chain comprising a variable region comprising an amino acid sequence with at least 95% sequence identity to SEQ ID NO: 162 and a Fab light chain comprising a variable region comprising an amino acid sequence with at least 95% sequence identity to the amino acid sequence of SEQ ID NO: 167. In some embodiments, in the polynucleotide encoding a Fab that immunospecifically binds to FLAG-tag, the polynucleotide comprises SEQ ID NO: 181 and SEQ ID NO: 166.

In some embodiments, the polynucleotide can encode an antibody comprising a Fab that immunospecifically binds to FLAG-tag, the Fab comprising the amino acid sequence of SEQ ID NO: 175. The polynucleotide can encode a Fab comprising an amino acid sequence with at least 90% sequence identity to SEQ ID NO: 175. The polynucleotide can encode a Fab comprising an amino acid sequence with at least 95% sequence identity to SEQ ID NO: 175. In some embodiments, in the polynucleotide encoding a Fab that immunospecifically binds to FLAG-tag, the polynucleotide comprises SEQ ID NO: 174.

In some embodiments, the polynucleotide can encode a Fab that is a rabbit anti-mouse IgG secondary antibody (RAM). The polynucleotide can encode a Fab that is a RAM, the Fab comprising a Fab heavy chain comprising a heavy chain complementarity determining region 1 (HCDR1) comprising the amino acid sequence of SEQ ID NO: 127, a HCDR2 comprising the amino acid sequence of SEQ ID NO: 128, and a HCDR3 comprising the amino acid sequence of SEQ ID NO: 129.

The polynucleotide can encode a Fab that is a RAM, the Fab comprising a Fab heavy chain comprising a variable region comprising the amino acid sequence of SEQ ID NO: 126. The polynucleotide can encode a Fab heavy chain comprising a variable region comprising an amino acid sequence with at least 90% sequence identity to SEQ ID NO: 126. The polynucleotide can encode a Fab heavy chain comprising a variable region comprising an amino acid sequence with at least 95% sequence identity to SEQ ID NO: 126.

The polynucleotide can encode a Fab that is a RAM, the Fab comprising a Fab light chain comprising a light chain complementarity determining region 1 (LCDR1) comprising the amino acid sequence of SEQ ID NO: 132, a LCDR2 comprising the amino acid sequence of GAS, and a LCDR3 comprising the amino acid sequence of SEQ ID NO: 133.

The polynucleotide can encode a Fab that is a RAM, the Fab comprising a Fab light chain comprising a variable region comprising the amino acid sequence of SEQ ID NO: 131. The polynucleotide can encode a Fab light chain comprising a variable region comprising an amino acid sequence with at least 90% sequence identity to SEQ ID NO: 131.

The polynucleotide can encode a Fab light chain comprising a variable region comprising an amino acid sequence with at least 95% sequence identity to SEQ ID NO: 131. The polynucleotide can encode a Fab that is a RAM, the Fab comprising a Fab heavy chain comprising a heavy chain complementarity determining region 1 (HCDR1) comprising the amino acid sequence of SEQ ID NO: 127, a HCDR2 comprising the amino acid sequence of SEQ ID NO: 128, and a HCDR3 comprising the amino acid sequence of SEQ ID NO: 129, and a Fab light chain comprising a light chain complementarity determining region 1 (LCDR1) comprising the amino acid sequence of SEQ ID NO: 132, a LCDR2 comprising the amino acid sequence of GAS, and a LCDR3 comprising the amino acid sequence of SEQ ID NO: 133.

The polynucleotide can encode a Fab that is a RAM, the Fab comprising a Fab heavy chain comprising a variable region comprising the amino acid sequence of SEQ ID NO: 126 and a Fab light chain comprising a variable region comprising the amino acid sequence of SEQ ID NO: 131. The polynucleotide can encode a Fab heavy chain comprising a variable region comprising an amino acid sequence with at least 90% sequence identity to SEQ ID NO: 126 and a Fab light chain comprising a variable region comprising an amino acid sequence with at least 90% sequence identity to the amino acid sequence of SEQ ID NO: 131. The polynucleotide can encode a Fab heavy chain comprising a variable region comprising an amino acid sequence with at least 95% sequence identity to SEQ ID NO: 126 and a Fab light chain comprising a variable region comprising an amino acid sequence with at least 95% sequence identity to the amino acid sequence of SEQ ID NO: 131. In some embodiments, in the polynucleotide encoding a Fab that immunospecifically binds is a RAM, the polynucleotide comprises SEQ ID NO: 125 and SEQ ID NO: 130.

In some embodiments, the polynucleotide can encode an antibody comprising a Fab that is a RAM, the Fab comprising the amino acid sequence of SEQ ID NO: 139. The polynucleotide can encode a Fab comprising an amino acid sequence with at least 90% sequence identity to SEQ ID NO: 139. The polynucleotide can encode a Fab comprising an amino acid sequence with at least 95% sequence identity to SEQ ID NO: 139. In some embodiments, in the polynucleotide encoding a Fab that is a RAM, the polynucleotide comprises SEQ ID NO: 138.

In some embodiments, the polynucleotide can encode a Fab that immunospecifically binds to pERK1/2. The polynucleotide can encode a Fab that immunospecifically binds to pERK1/2, the Fab comprising a Fab heavy chain comprising a heavy chain complementarity determining region 1 (HCDR1) comprising the amino acid sequence of SEQ ID NO: 85, a HCDR2 comprising the amino acid sequence of SEQ ID NO: 86, and a HCDR3 comprising the amino acid sequence of SEQ ID NO: 87.

The polynucleotide can encode a Fab that immunospecifically binds to pERK1/2, the Fab comprising a Fab heavy chain comprising a variable region comprising the amino acid sequence of SEQ ID NO: 84. The polynucleotide can encode a Fab heavy chain comprising a variable region comprising an amino acid sequence with at least 90% sequence identity to SEQ ID NO: 84. The polynucleotide can encode a Fab heavy chain comprising a variable region comprising an amino acid sequence with at least 95% sequence identity to SEQ ID NO: 84.

The polynucleotide can encode a Fab that immunospecifically binds pERK1/2, the Fab comprising a Fab light chain comprising a light chain complementarity determining region 1 (LCDR1) comprising the amino acid sequence of SEQ ID NO: 91, a LCDR2 comprising the amino acid sequence of KVS, and a LCDR3 comprising the amino acid sequence of SEQ ID NO: 92.

The polynucleotide can encode a Fab that immunospecifically binds pERK1/2, the Fab comprising a Fab light chain comprising a variable region comprising the amino acid sequence of SEQ ID NO: 90. The polynucleotide can encode a Fab light chain comprising a variable region comprising an amino acid sequence with at least 90% sequence identity to SEQ ID NO: 90. The polynucleotide can encode a Fab light chain comprising a variable region comprising an amino acid sequence with at least 95% sequence identity to SEQ ID NO: 90.

The polynucleotide can encode a Fab that immunospecifically binds to pERK1/2, the Fab comprising a Fab heavy chain comprising a heavy chain complementarity determining region 1 (HCDR1) comprising the amino acid sequence of SEQ ID NO: 85, a HCDR2 comprising the amino acid sequence of SEQ ID NO: 86, and a HCDR3 comprising the amino acid sequence of SEQ ID NO: 87, and a Fab light chain comprising a light chain complementarity determining region 1 (LCDR1) comprising the amino acid sequence of SEQ ID NO: 91, a LCDR2 comprising the amino acid sequence of KVS, and a LCDR3 comprising the amino acid sequence of SEQ ID NO: 92.

The polynucleotide can encode a Fab that immunospecifically binds to pERK1/2, the Fab comprising a Fab heavy chain comprising a variable region comprising the amino acid sequence of SEQ ID NO: 84 and a Fab light chain comprising a variable region comprising the amino acid sequence of SEQ ID NO: 90. The polynucleotide can encode a Fab heavy chain comprising a variable region comprising an amino acid sequence with at least 90% sequence identity to SEQ ID NO: 84 and a Fab light chain comprising a variable region comprising an amino acid sequence with at least 90% sequence identity to the amino acid sequence of SEQ ID NO: 90. The polynucleotide can encode a Fab heavy chain comprising a variable region comprising an amino acid sequence with at least 95% sequence identity to SEQ ID NO: 84 and a Fab light chain comprising a variable region comprising an amino acid sequence with at least 95% sequence identity to the amino acid sequence of SEQ ID NO: 90. In some embodiments, in the polynucleotide encoding a Fab that immunospecifically binds to pERK1/2, the polynucleotide comprises SEQ ID NO: 83 and SEQ ID NO: 89.

In some embodiments, the polynucleotide can encode an antibody comprising a Fab that immunospecifically binds to pERK1/2, the Fab comprising the amino acid sequence of SEQ ID NO: 98. The polynucleotide can encode a Fab comprising an amino acid sequence with at least 90% sequence identity to SEQ ID NO: 98. The polynucleotide can encode a Fab comprising an amino acid sequence with at least 95% sequence identity to SEQ ID NO: 98. In some embodiments, in the polynucleotide encoding a Fab that immunospecifically binds to pERK1/2, the polynucleotide comprises SEQ ID NO: 97.

In some embodiments, the polynucleotide can encode a Fab that immunospecifically binds to PSD-95. The polynucleotide can encode a Fab that immunospecifically binds to PSD-95, the Fab comprising a Fab heavy chain comprising a heavy chain complementarity determining region 1 (HCDR1) comprising the amino acid sequence of SEQ ID NO: 111, a HCDR2 comprising the amino acid sequence of SEQ ID NO: 112, and a HCDR3 comprising the amino acid sequence of SEQ ID NO: 113.

The polynucleotide can encode a Fab that immunospecifically binds to PSD-95, the Fab comprising a Fab heavy chain comprising a variable region comprising the amino acid sequence of SEQ ID NO: 110. The polynucleotide can encode a Fab heavy chain comprising a variable region comprising an amino acid sequence with at least 90% sequence identity to SEQ ID NO: 110. The polynucleotide can encode a Fab heavy chain comprising a variable region comprising an amino acid sequence with at least 95% sequence identity to SEQ ID NO: 110.

The polynucleotide can encode a Fab that immunospecifically binds PSD-95, the Fab comprising a Fab light chain comprising a light chain complementarity determining region 1 (LCDR1) comprising the amino acid sequence of SEQ ID NO: 116, a LCDR2 comprising the amino acid sequence of DTS, and a LCDR3 comprising the amino acid sequence of SEQ ID NO: 117.

The polynucleotide can encode a Fab that immunospecifically binds PSD-95, the Fab comprising a Fab light chain comprising a variable region comprising the amino acid sequence of SEQ ID NO: 115. The polynucleotide can encode a Fab light chain comprising a variable region comprising an amino acid sequence with at least 90% sequence identity to SEQ ID NO: 115. The polynucleotide can encode a Fab light chain comprising a variable region comprising an amino acid sequence with at least 95% sequence identity to SEQ ID NO: 115.

The polynucleotide can encode a Fab that immunospecifically binds to PSD-95, the Fab comprising a Fab heavy chain comprising a heavy chain complementarity determining region 1 (HCDR1) comprising the amino acid sequence of SEQ ID NO: 111, a HCDR2 comprising the amino acid sequence of SEQ ID NO: 112, and a HCDR3 comprising the amino acid sequence of SEQ ID NO: 113, and a Fab light chain comprising a light chain complementarity determining region 1 (LCDR1) comprising the amino acid sequence of SEQ ID NO: 116, a LCDR2 comprising the amino acid sequence of DTS, and a LCDR3 comprising the amino acid sequence of SEQ ID NO: 117.

The polynucleotide can encode a Fab that immunospecifically binds to PSD-95, the Fab comprising a Fab heavy chain comprising a variable region comprising the amino acid sequence of SEQ ID NO: 110 and a Fab light chain comprising a variable region comprising the amino acid sequence of SEQ ID NO: 115. The polynucleotide can encode a Fab heavy chain comprising a variable region comprising an amino acid sequence with at least 90% sequence identity to SEQ ID NO: 110 and a Fab light chain comprising a variable region comprising an amino acid sequence with at least 90% sequence identity to the amino acid sequence of SEQ ID NO: 115. The polynucleotide can encode a Fab heavy chain comprising a variable region comprising an amino acid sequence with at least 95% sequence identity to SEQ ID NO: 110 and a Fab light chain comprising a variable region comprising an amino acid sequence with at least 95% sequence identity to the amino acid sequence of SEQ ID NO: 115. In some embodiments, in the polynucleotide encoding a Fab that immunospecifically binds to PSD-95, the polynucleotide comprises SEQ ID NO: 125 and SEQ ID NO: 130.

In some embodiments, the polynucleotide can encode an antibody comprising a Fab that immunospecifically binds to PSD-95, the Fab comprising the amino acid sequence of SEQ ID NO: 123. The polynucleotide can encode a Fab comprising an amino acid sequence with at least 90% sequence identity to SEQ ID NO: 123. The polynucleotide can encode a Fab comprising an amino acid sequence with at least 95% sequence identity to SEQ ID NO: 123. In some embodiments, in the polynucleotide encoding a Fab that immunospecifically binds to PSD-95, the polynucleotide comprises SEQ ID NO: 122.

In some embodiments, the polynucleotide can encode a Fab that immunospecifically binds to TIGIT. The polynucleotide can encode a Fab that immunospecifically binds to TIGIT, the Fab comprising a heavy chain complementarity determining region 1 (HCDR1) comprising the amino acid sequence of SEQ ID NO: 146, a HCDR2 comprising the amino acid sequence of SEQ ID NO: 147, and a HCDR3 comprising the amino acid sequence of SEQ ID NO: 148

The polynucleotide can encode a Fab that immunospecifically binds to TIGIT, the Fab comprising a Fab heavy chain comprising a variable region comprising the amino acid sequence of SEQ ID NO: 145. The polynucleotide can encode a Fab heavy chain comprising a variable region comprising an amino acid sequence with at least 90% sequence identity to SEQ ID NO: 145. The polynucleotide can encode a Fab heavy chain comprising a variable region comprising an amino acid sequence with at least 95% sequence identity to SEQ ID NO: 145.

The polynucleotide can encode a Fab that immunospecifically binds to TIGIT, the Fab comprising a Fab light chain comprising a light chain complementarity determining region 1 (LCDR1) comprising the amino acid sequence of SEQ ID NO: 151, a LCDR2 comprising the amino acid sequence of SAS, and a LCDR3 comprising the amino acid sequence of SEQ ID NO: 152.

The polynucleotide can encode a Fab that immunospecifically binds to TIGIT, the Fab comprising a Fab light chain comprising a variable region comprising the amino acid sequence of SEQ ID NO: 150. The polynucleotide can encode a Fab light chain comprising a variable region comprising an amino acid sequence with at least 90% sequence identity to SEQ ID NO: 150. The polynucleotide can encode a Fab light chain comprising a variable region comprising an amino acid sequence with at least 95% sequence identity to SEQ ID NO: 150.

The polynucleotide can encode a Fab that immunospecifically binds to TIGIT, the Fab comprising a Fab heavy chain comprising a heavy chain complementarity determining region 1 (HCDR1) comprising the amino acid sequence of SEQ ID NO: 146, a HCDR2 comprising the amino acid sequence of SEQ ID NO: 147, and a HCDR3 comprising the amino acid sequence of SEQ ID NO: 148, and a Fab light chain comprising a light chain complementarity determining region 1 (LCDR1) comprising the amino acid sequence of SEQ ID NO: 151, a LCDR2 comprising the amino acid sequence of SAS, and a LCDR3 comprising the amino acid sequence of SEQ ID NO: 152.

The polynucleotide can encode a Fab that immunospecifically binds to TIGIT, the Fab comprising a Fab heavy chain comprising a variable region comprising the amino acid sequence of SEQ ID NO: 145 and a Fab light chain comprising a variable region comprising the amino acid sequence of SEQ ID NO: 150. The polynucleotide can encode a Fab heavy chain comprising a variable region comprising an amino acid sequence with at least 90% sequence identity to SEQ ID NO: 145 and a Fab light chain comprising a variable region comprising an amino acid sequence with at least 90% sequence identity to the amino acid sequence of SEQ ID NO: 150. The polynucleotide can encode a Fab heavy chain comprising a variable region comprising an amino acid sequence with at least 95% sequence identity to SEQ ID NO: 145 and a Fab light chain comprising a variable region comprising an amino acid sequence with at least 95% sequence identity to the amino acid sequence of SEQ ID NO: 150. In some embodiments, in the polynucleotide encoding a Fab that immunospecifically binds to TIGIT, the polynucleotide comprises SEQ ID NO: 144 and SEQ ID NO: 149.

In some embodiments, the polynucleotide can encode a Fab that immunospecifically binds to TIGIT, the Fab comprising the amino acid sequence of SEQ ID NO: 158. The polynucleotide can encode a Fab comprising an amino acid sequence with at least 90% sequence identity to SEQ ID NO: 158. The polynucleotide can encode a Fab comprising an amino acid sequence with at least 95% sequence identity to SEQ ID NO: 158. In some embodiments, in the polynucleotide encoding a Fab that immunospecifically binds to TIGIT, the polynucleotide comprises SEQ ID NO: 157.

The polynucleotide can encode any antibody disclosed herein wherein the antibody has the isotype IgG.

Disclosed herein are vectors comprising any of the herein described nucleic acid molecules that comprise polynucleotides encoding isolated polypeptides or antibodies. Disclosed herein are also host cells comprising any of the herein disclosed vectors. Suitable host cells include eukaryotic cells and prokaryotic cells.

Disclosed herein are methods of producing an isolated polypeptide or antibody, the methods comprising culturing a host cell comprising any of the herein disclosed vectors under conditions suitable for expression of the isolated polypeptide or antibody and purifying the isolated polypeptide or antibody.

Disclosed herein are antibodies comprising the isolated polypeptide comprising the amino acid sequence of Formula I: Z-(X-Yₙ-Zᵣ)ₚ-X, wherein Z is a hinge region, X is a CH2 domain, Y is a linker, n is an integer of 1 or greater, p is an integer of 2 or greater, and r is an integer of 0 or 1 linked to a Fab domain.

The hinge (Z) region can comprise the amino acid sequence of SEQ ID NO: 18. The hinge (Z) can comprise an amino acid sequence with at least 90% sequence identity to SEQ ID NO: 18. The hinge (Z) can comprise an amino acid sequence with at least 95% sequence identity to SEQ ID NO: 18. The hinge (Z) can comprise the amino acid sequence of SEQ ID NO: 48. The hinge (Z) can comprise an amino acid sequence with at least 90% sequence identity to SEQ ID NO: 48. The hinge (Z) can comprise an amino acid sequence with at least 95% sequence identity to SEQ ID NO: 48. The hinge (Z) can comprise the amino acid sequence of SEQ ID NO: 102. The hinge (Z) can comprise an amino acid sequence with at least 90% sequence identity to SEQ ID NO: 102. The hinge (Z) can comprise an amino acid sequence with at least 95% sequence identity to SEQ ID NO: 102.

The CH2 domain (X) can comprise the amino acid sequence of SEQ ID NO: 19. The CH2 domain (X) can comprise an amino acid sequence with at least 90% sequence identity to SEQ ID NO: 19. The CH2 domain (X) can comprise an amino acid sequence with at least 95% sequence identity to SEQ ID NO: 19. The CH2 domain (X) can comprise the amino acid sequence of SEQ ID NO: 49. The CH2 domain (X) can comprise an amino acid sequence with at least 90% sequence identity to SEQ ID NO: 49. The CH2 domain (X) can comprise an amino acid sequence with at least 95% sequence identity to SEQ ID NO: 49. The CH2 domain (X) can comprise the amino acid sequence of SEQ ID NO: 104. The CH2 domain (X) can comprise an amino acid sequence with at least 90% sequence identity to SEQ ID NO: 104. The CH2 domain (X) can comprise an amino acid sequence with at least 95% sequence identity to SEQ ID NO: 104.

In some embodiments, the antibody comprising the isolated polypeptide can comprise one or more linkers (Y) comprising the amino acid sequence of GS, GGS, SEQ ID NO: 14, SEQ ID NO: 38, SEQ ID NO: 43, or SEQ ID NO: 44. The one or more linkers (Y) can comprise GS. The one or more linkers (Y) can comprise GGS. The one or more linkers (Y) can comprise SEQ ID NO: 14. The one or more linkers (Y) can comprise SEQ ID NO: 38. The one or more linkers can comprise SEQ ID NO: 43. The one or more linkers (Y) can comprise a sequence with at least 90% sequence identity to SEQ ID NO: 43. The one or more linkers (Y) can comprise SEQ ID NO: 44. The one or more linkers (Y) can comprise a sequence with at least 90% sequence identity to SEQ ID NO: 44.

The isolated polypeptide can comprise one linker (Y), such that n is 1. The isolated polypeptide can comprise two linkers (Y), such that n is 2. The isolated polypeptide can comprise three linkers (Y), such that n is 3. The isolated polypeptide can comprise four linkers (Y), such that n is 4. The isolated polypeptide can comprise five linkers (Y), such that n is 5.

In some embodiments, the antibody comprising the isolated polypeptide can comprise two or more regions comprising a CH2-linker region ((X-Yₙ)ₚ), such that p is 2 or greater. The isolated polypeptide can comprise two CH2-linker regions ((X-Yₙ)ₚ), such that p is 2. The isolated polypeptide can comprise three CH2-linker regions ((X-Yₙ)ₚ), such that p is 3. The isolated polypeptide can comprise four CH2-linker regions ((X-Yₙ)ₚ), such that p is 4. The isolated polypeptide can comprise five CH2-linker regions ((X-Yₙ)ₚ), such that p is 5.

In some embodiments, the antibody comprising the isolated polypeptide can comprise two or more regions comprising a CH2-linker-hinge region ((X-Yₙ-Zᵣ)ₚ), such that p is 2 or greater. The isolated polypeptide can comprise two CH2-linker-hinge regions ((X-Yₙ-Zᵣ)ₚ), such that p is 2. The isolated polypeptide can comprise three CH2-linker-hinge regions ((X-Yₙ-Zᵣ)ₚ), such that p is 3. The isolated polypeptide can comprise four CH2-linker-hinge regions ((X-Yₙ-Zᵣ)ₚ), such that p is 4. The isolated polypeptide can comprise five CH2-linker-hinge regions ((X-Yₙ-Zᵣ)ₚ), such that p is 5.

In some embodiments, the antibody comprising the isolated polypeptide comprises, consists essentially of, or consists of (X-Yₙ-Zᵣ)ₚ, wherein Z (hinge) comprises the amino acid sequence of SEQ ID NO: 18, X(CH2 domain) comprises the amino acid sequence of SEQ ID NO: 19, Y (linker) comprises the amino acid sequence of SEQ ID NO: 44, wherein n is 1, r is 0 or 1, and p is 2.

In some embodiments, the antibody comprising the isolated polypeptide comprises, consists essentially of, or consists of (X-Yₙ-Zᵣ)ₚ, wherein Z (hinge) comprises the amino acid sequence of SEQ ID NO: 48, X(CH2 domain) comprises the amino acid sequence of SEQ ID NO: 49, Y (linker) comprises the amino acid sequence of SEQ ID NO: 44, wherein n is 1, r is 0 or 1, and p is 2.

In some embodiments, the antibody comprising the isolated polypeptide comprises, consists essentially of, or consists of (X-Yₙ-Zᵣ)ₚ, wherein Z (hinge) comprises the amino acid sequence of SEQ ID NO: 102, X(CH2 domain) comprises the amino acid sequence of SEQ ID NO: 104, Y (linker) comprises the amino acid sequence of SEQ ID NO: 44, wherein n is 1, r is 0 or 1, and p is 2.

In some embodiments, the antibody comprising the isolated polypeptide comprises, consists essentially of, or consists of (X-Yₙ-Zᵣ)ₚ, wherein Z (hinge) comprises the amino acid sequence of SEQ ID NO: 102, X(CH2 domain) comprises the amino acid sequence of SEQ ID NO: 104, Y (linker) comprises the amino acid sequence of SEQ ID NO: 45, wherein n is 1, r is 0 or 1, and p is 2.

In some embodiments, the antibody comprising the isolated polypeptide comprises two additional CH2 domains N-terminal to the Fc domain connected via a hinge and optionally, one or more linkers. The isolated polypeptide comprising two additional CH2 domains N-terminal to the Fc domain connected via a hinge and optionally one or more linkers can comprise the amino acid sequence of SEQ ID NO: 52 or SEQ ID NO: 54. In some embodiments, the isolated polypeptide comprising a CH3 domain comprises SEQ ID NO: 52.

The isolated polypeptide comprising two additional CH2 domains N-terminal to the Fc domain connected via a hinge and optionally one or more linkers can comprise an amino acid sequence with at least 90% identity to SEQ ID NO: 52. The isolated polypeptide comprising two additional CH2 domains N-terminal to the Fc domain connected via a hinge and optionally one or more linkers can comprise an amino acid sequence with at least 95% identity to SEQ ID NO: 52.

In some embodiments, the antibody comprising the isolated polypeptide comprises two additional CH2 domains N-terminal to the Fc domain connected via a hinge and optionally one or more linkers and comprises “LFLEPS” mutations within the lower hinge and CH2 domains. The “LFLEPS” mutations can comprise the following amino acid substitutions: L234F, L235E and P331S. The isolated polypeptide comprising two additional CH2 domains N-terminal to the Fc domain connected via a hinge and optionally one or more linkers and comprises “LFLEPS” mutations within the lower hinge and CH2 domains comprises SEQ ID NO: 54. The isolated polypeptide comprising two additional CH2 domains N-terminal to the Fc domain connected via a hinge and optionally one or more linkers and comprises “LFLEPS” mutations within the lower hinge and CH2 domains can comprise an amino acid sequence with at least 90% identity to SEQ ID NO: 54. The isolated polypeptide comprising two additional CH2 domains N-terminal to the Fc domain connected via a hinge and optionally one or more linkers and comprises “LFLEPS” mutations within the lower hinge and CH2 domains can comprise an amino acid sequence with at least 95% identity to SEQ ID NO: 54.

In some embodiments, the antibodies comprising the isolated peptide disclosed herein have a reduction of background signal of at least 1.5-fold, compared to a corresponding wild type antibody. In some embodiments, the reduction of background signal is at least 1.5-fold, at least 2-fold, at least 2.5-fold, at least 3-fold, at least 3.5-fold, at least 4-fold, at least 4.5-fold or at least 5-fold compared to a corresponding wild type antibody.

In some embodiments, the antibodies comprising any isolated peptide disclosed herein have an increase in sensitivity or signal enhancement of at least 1.5-fold compared to a corresponding wild type antibody. In some embodiments, the increase in sensitivity is at least 1.5-fold, at least 2-fold, at least 2.5-fold, at least 3-fold, at least 3.5-fold, at least 4-fold, at least 4.5-fold, at least 5-fold, at least 5.5-fold, at least 6-fold, at least 6.5-fold, at least 7-fold, at least 7.5-fold, at least 8-fold, at least 8.5-fold, at least 9-fold, at least 9.5-fold, or at least 10-fold compared to a corresponding wild type antibody.

Disclosed herein are antibodies that immunospecifically bind to Parkin, wherein the antibodies comprise, consist essentially of, or consist of a heavy chain (HC) and a light chain (LC) that pair to form a binding domain comprising a heavy chain complementarity determining region 1 (HCDR1) comprising the amino acid sequence of SEQ ID NO: 4, a HCDR2 comprising the amino acid sequence of SEQ ID NO: 5, a HCDR3 comprising the amino acid sequence of SEQ ID NO: 6, a light chain complementarity determining region 1 (LCDR1) comprising the amino acid sequence of SEQ ID NO: 13, a LCDR2 comprising the amino acid sequence RAS, and a LCDR3 comprising the amino acid sequence of SEQ ID NO: 15, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge. In some embodiments, the antibodies that immunospecifically bind to Parkin further comprise a CH3 domain linked to the C-terminus of the third CH2 domain.

The first CH2 domain, the second CH2 domain, and the third CH2 domain can each comprise the amino acid sequence of SEQ ID NO: 19 or SEQ ID NO: 50. In some embodiments, the first CH2 domain, the second CH2 domain, and the third CH2 domain each comprise the amino acid sequence of SEQ ID NO: 19. The first CH2 domain, the second CH2 domain, and the third CH2 domain can each comprise an amino acid sequence with at least 90% sequence identity to SEQ ID NO: 19. The first CH2 domain, the second CH2 domain, and the third CH2 domain can each comprise an amino acid sequence with at least 95% sequence identity to SEQ ID NO: 19.

In some embodiments, the first CH2 domain, the second CH2 domain, and the third CH2 domain can each comprise the amino acid sequence of SEQ ID NO: 49. The first CH2 domain, the second CH2 domain, and the third CH2 domain can each comprise an amino acid sequence with at least 90% sequence identity to SEQ ID NO: 49. The first CH2 domain, the second CH2 domain, and the third CH2 domain can each comprise an amino acid sequence with at least 95% sequence identity to SEQ ID NO: 49.

The first hinge and the second hinge can each comprise the amino acid sequence of SEQ ID NO: 18 or SEQ ID NO: 48. In some embodiments, the first hinge and the second hinge can each comprise the amino acid sequence of SEQ ID NO: 18. The first hinge and the second hinge can each comprise an amino acid sequence with at least 90% sequence identity to SEQ ID NO: 18. The first hinge and the second hinge can each comprise an amino acid sequence with at least 95% sequence identity to SEQ ID NO: 18.

In some embodiments, the first hinge and the second hinge can each comprise the amino acid sequence of SEQ ID NO: 48. The first hinge and the second hinge can each comprise an amino acid sequence with at least 90% sequence identity to SEQ ID NO: 48. The first hinge and the second hinge can each comprise an amino acid sequence with at least 95% sequence identity to SEQ ID NO: 48.

The first linker and the second linker can each comprise the amino acid sequence of GS, GGS, SEQ ID NO: 14, SEQ ID NO: 38, SEQ ID NO: 43, or SEQ ID NO: 44. The first and the second linkers can comprise GS. The first and the second linkers can comprise GGS. The first and the second linkers can comprise SEQ ID NO: 14. The first and the second linkers can comprise SEQ ID NO: 38. The first and the second linkers can comprise SEQ ID NO: 43. The first and the second linkers can comprise a sequence with at least 90% sequence identity to SEQ ID NO: 43. The first and the second linkers can comprise SEQ ID NO: 44. The first and the second linkers can comprise a sequence with at least 90% sequence identity to SEQ ID NO: 44.

In some embodiments, the antibody comprises a CH3 domain comprising the amino acid sequence of SEQ ID NO: 20. The antibody can comprise a CH3 domain having an amino acid sequence with at least 90% sequence identity to SEQ ID NO: 20. The antibody can comprise a CH3 domain having an amino acid sequence with at least 95% sequence identity to SEQ ID NO: 20.

In some embodiments, the antibody comprises a heavy chain variable region having the polypeptide sequence of SEQ ID NO: 7 and a light chain variable region having the polypeptide sequence of SEQ ID NO: 16. The antibody can comprise a heavy chain variable region having an amino acid sequence with at least 90% sequence identity to SEQ ID NO: 7 and a light chain variable region having an amino acid sequence with at least 90% sequence identity to SEQ ID NO: 16. The antibody can comprise a heavy chain variable region having an amino acid sequence with at least 95% sequence identity to SEQ ID NO: 7 and a light chain variable region having an amino acid sequence with at least 95% sequence identity to SEQ ID NO: 16.

In some embodiments, the antibody comprises a heavy chain having a polypeptide sequence of SEQ ID NO: 3 and a light chain having the polypeptide sequence of SEQ ID NO: 12. The antibody can comprise a heavy chain having an amino acid sequence with at least 90% sequence identity to SEQ ID NO: 3 and a light chain having an amino acid sequence with at least 90% sequence identity to SEQ ID NO: 12. The antibody can comprise a heavy chain having an amino acid sequence with at least 95% sequence identity to SEQ ID NO: 3 and a light chain having an amino acid sequence with at least 95% sequence identity to SEQ ID NO: 12.

In some embodiments, the antibody comprises a heavy chain that is encoded by a polynucleotide sequence of SEQ ID NO: 2 and a light chain that is encoded by a polynucleotide sequence of SEQ ID NO: 11.

Disclosed herein are antibodies that immunospecifically bind to OCT4, wherein the antibodies comprise, consist essentially of, or consist a heavy chain (HC) and a light chain (LC) that pair to form a binding domain comprising a heavy chain complementarity determining region 1 (HCDR1) comprising the amino acid sequence of SEQ ID NO: 29, a HCDR2 comprising the amino acid sequence of SEQ ID NO: 30, and a HCDR3 comprising the amino acid sequence of SEQ ID NO: 31, a light chain complementarity determining region 1 (LCDR1) comprising the amino acid sequence of SEQ ID NO: 37, a LCDR2 comprising the amino acid sequence GAS, and a LCDR3 comprising the amino acid sequence of SEQ ID NO: 39, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge. In some embodiments, the antibodies that immunospecifically bind to OCT4 further comprise a CH3 domain linked to the C-terminus of the third CH2 domain.

The first CH2 domain, the second CH2 domain, and the third CH2 domain can each comprise the amino acid sequence of SEQ ID NO: 19 or SEQ ID NO: 50. In some embodiments, the first CH2 domain, the second CH2 domain, and the third CH2 domain can each comprise the amino acid sequence of SEQ ID NO: 19. The first CH2 domain, the second CH2 domain, and the third CH2 domain can each comprise an amino acid sequence with at least 90% sequence identity to SEQ ID NO: 19. The first CH2 domain, the second CH2 domain, and the third CH2 domain can each comprise an amino acid sequence with at least 95% sequence identity to SEQ ID NO: 19.

In some embodiments, the first CH2 domain, the second CH2 domain, and the third CH2 domain can each comprise the amino acid sequence of SEQ ID NO: 49. The first CH2 domain, the second CH2 domain, and the third CH2 domain can each comprise an amino acid sequence with at least 90% sequence identity to SEQ ID NO: 49. The first CH2 domain, the second CH2 domain, and the third CH2 domain can each comprise an amino acid sequence with at least 95% sequence identity to SEQ ID NO: 49.

The first hinge and the second hinge can each comprise the amino acid sequence of SEQ ID NO: 18 or SEQ ID NO: 48. In some embodiments, the first hinge and the second hinge each comprise the amino acid sequence of SEQ ID NO: 18. The first hinge and the second hinge can each comprise an amino acid sequence with at least 90% sequence identity to SEQ ID NO: 18. The first hinge and the second hinge can each comprise an amino acid sequence with at least 95% sequence identity to SEQ ID NO: 18.

In some embodiments, the first hinge and the second hinge can each comprise the amino acid sequence of SEQ ID NO: 48. The first hinge and the second hinge can each comprise an amino acid sequence with at least 90% sequence identity to SEQ ID NO: 48. The first hinge and the second hinge can each comprise an amino acid sequence with at least 95% sequence identity to SEQ ID NO: 48.

The first linker and the second linker can each comprise the amino acid sequence of GS, GGS, SEQ ID NO: 14, SEQ ID NO: 38, SEQ ID NO: 43, or SEQ ID NO: 44. The first and the second linkers can comprise GS. The first and the second linkers can comprise GGS. The first and the second linkers can comprise SEQ ID NO: 14. The first and the second linkers can comprise SEQ ID NO: 38. The first and the second linkers can comprise SEQ ID NO: 43. The first and the second linkers can comprise a sequence with at least 90% sequence identity to SEQ ID NO: 43. The first and the second linkers can comprise SEQ ID NO: 44. The first and the second linkers can comprise a sequence with at least 90% sequence identity to SEQ ID NO: 44.

In some embodiments, the antibody comprises a CH3 domain comprising the amino acid sequence of SEQ ID NO: 20. The antibody can comprise a CH3 domain having an amino acid sequence with at least 90% sequence identity to SEQ ID NO: 20. The antibody can comprise a CH3 domain having an amino acid sequence with at least 95% sequence identity to SEQ ID NO: 20.

In some embodiments, the antibody comprises a heavy chain variable region having the polypeptide sequence of SEQ ID NO: 32 and a light chain variable region having the polypeptide sequence of SEQ ID NO: 40. The antibody can comprise a heavy chain variable region having an amino acid sequence with at least 90% sequence identity to SEQ ID NO: 32 and a light chain variable region having an amino acid sequence with at least 90% sequence identity to SEQ ID NO: 40. The antibody can comprise a heavy chain variable region having an amino acid sequence with at least 95% sequence identity to SEQ ID NO: 32 and a light chain variable region having an amino acid sequence with at least 95% sequence identity to SEQ ID NO: 40.

In some embodiments, the antibody comprises a heavy chain having a polypeptide sequence of SEQ ID NO: 28 and a light chain having the polypeptide sequence of SEQ ID NO: 36. The antibody can comprise a heavy chain having an amino acid sequence with at least 90% sequence identity to SEQ ID NO: 28 and a light chain having an amino acid sequence with at least 90% sequence identity to SEQ ID NO: 36. The antibody can comprise a heavy chain having an amino acid sequence with at least 95% sequence identity to SEQ ID NO: 28 and a light chain having an amino acid sequence with at least 95% sequence identity to SEQ ID NO: 36.

In some embodiments, the antibody comprises a heavy chain that is encoded by a polynucleotide sequence of SEQ ID NO: 27 and a light chain that is encoded by a polynucleotide sequence of SEQ ID NO: 35.

Disclosed herein are antibodies that immunospecifically bind to IDO1, wherein the antibodies comprise, consist essentially of, or consist of a heavy chain (HC) and a light chain (LC) that pair to form a binding domain comprising a heavy chain complementarity determining region 1 (HCDR1) comprising the amino acid sequence of SEQ ID NO: 66, a HCDR2 comprising the amino acid sequence of SEQ ID NO: 67, a HCDR3 comprising the amino acid sequence of SEQ ID NO: 68, a light chain complementarity determining region 1 (LCDR1) comprising the amino acid sequence of SEQ ID NO: 72, a LCDR2 comprising the amino acid sequence of YAS, and a LCDR3 comprising the amino acid sequence of SEQ ID NO: 73, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge. In some embodiments, the antibodies that immunospecifically bind to IDO1 further comprise a CH3 domain linked to the C-terminus of the third CH2 domain.

The first CH2 domain, the second CH2 domain, and the third CH2 domain can each comprise the amino acid sequence of SEQ ID NO: 19 or SEQ ID NO: 49. In some embodiments, the first CH2 domain, the second CH2 domain, and the third CH2 domain each comprise the amino acid sequence of SEQ ID NO: 19. The first CH2 domain, the second CH2 domain, and the third CH2 domain can each comprise an amino acid sequence with at least 90% sequence identity to SEQ ID NO: 19. The first CH2 domain, the second CH2 domain, and the third CH2 domain can each comprise an amino acid sequence with at least 95% sequence identity to SEQ ID NO: 19.

In some embodiments, the first CH2 domain, the second CH2 domain, and the third CH2 domain can each comprise the amino acid sequence of SEQ ID NO: 49. The first CH2 domain, the second CH2 domain, and the third CH2 domain can each comprise an amino acid sequence with at least 90% sequence identity to SEQ ID NO: 49. The first CH2 domain, the second CH2 domain, and the third CH2 domain can each comprise an amino acid sequence with at least 95% sequence identity to SEQ ID NO: 49.

The first hinge and the second hinge can each comprise the amino acid sequence of SEQ ID NO: 18 or SEQ ID NO: 48. In some embodiments, the first hinge and the second hinge can each comprise the amino acid sequence of SEQ ID NO: 18. The first hinge and the second hinge can each comprise an amino acid sequence with at least 90% sequence identity to SEQ ID NO: 18. The first hinge and the second hinge can each comprise an amino acid sequence with at least 95% sequence identity to SEQ ID NO: 18.

In some embodiments, the first hinge and the second hinge can each comprise the amino acid sequence of SEQ ID NO: 48. The first hinge and the second hinge can each comprise an amino acid sequence with at least 90% sequence identity to SEQ ID NO: 48. The first hinge and the second hinge can each comprise an amino acid sequence with at least 95% sequence identity to SEQ ID NO: 48.

The first linker and the second linker can each comprise the amino acid sequence of GS, GGS, SEQ ID NO: 14, SEQ ID NO: 38, SEQ ID NO: 43, or SEQ ID NO: 44. The first and the second linkers can comprise GS. The first and the second linkers can comprise GGS. The first and the second linkers can comprise SEQ ID NO: 14. The first and the second linkers can comprise SEQ ID NO: 38. The first and the second linkers can comprise SEQ ID NO: 43. The first and the second linkers can comprise a sequence with at least 90% sequence identity to SEQ ID NO: 43. The first and the second linkers can comprise SEQ ID NO: 44. The first and the second linkers can comprise a sequence with at least 90% sequence identity to SEQ ID NO: 44.

In some embodiments, the antibody comprises a CH3 domain comprising the amino acid sequence of SEQ ID NO: 20. The antibody can comprise a CH3 domain having an amino acid sequence with at least 90% sequence identity to SEQ ID NO: 20. The antibody can comprise a CH3 domain having an amino acid sequence with at least 95% sequence identity to SEQ ID NO: 20.

In some embodiments, the antibody comprises a heavy chain variable region having the polypeptide sequence of SEQ ID NO: 65 and a light chain variable region having the polypeptide sequence of SEQ ID NO: 71. The antibody can comprise a heavy chain variable region having an amino acid sequence with at least 90% sequence identity to SEQ ID NO: 65 and a light chain variable region having an amino acid sequence with at least 90% sequence identity to SEQ ID NO: 71. The antibody can comprise a heavy chain variable region having an amino acid sequence with at least 95% sequence identity to SEQ ID NO: 65 and a light chain variable region having an amino acid sequence with at least 95% sequence identity to SEQ ID NO: 71.

In some embodiments, the antibody comprises a heavy chain having a polypeptide sequence of SEQ ID NO: 75 and a light chain having the polypeptide sequence of SEQ ID NO: 77. The antibody can comprise a heavy chain having an amino acid sequence with at least 90% sequence identity to SEQ ID NO: 75 and a light chain having an amino acid sequence with at least 90% sequence identity to SEQ ID NO: 77. The antibody can comprise a heavy chain having an amino acid sequence with at least 95% sequence identity to SEQ ID NO: 75 and a light chain having an amino acid sequence with at least 95% sequence identity to SEQ ID NO: 77.

In some embodiments, the antibody comprises a heavy chain that is encoded by a polynucleotide sequence of SEQ ID NO: 74 and a light chain that is encoded by a polynucleotide sequence of SEQ ID NO: 76.

Disclosed herein are antibodies that immunospecifically bind to FLAG-tag, wherein the antibodies comprise, consist essentially of, or consist of a heavy chain (HC) and a light chain (LC) that pair to form a binding domain comprising a heavy chain complementarity determining region 1 (HCDR1) comprising the amino acid sequence of SEQ ID NO: 163, a HCDR2 comprising the amino acid sequence of SEQ ID NO: 164, a HCDR3 comprising the amino acid sequence of SEQ ID NO: 165, a light chain complementarity determining region 1 (LCDR1) comprising the amino acid sequence of SEQ ID NO: 168, a LCDR2 comprising the amino acid sequence of YAS, and a LCDR3 comprising the amino acid sequence of SEQ ID NO: 169, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge. In some embodiments, the antibodies that immunospecifically bind to FLAG-tag further comprise a CH3 domain linked to the C-terminus of the third CH2 domain.

The first CH2 domain, the second CH2 domain, and the third CH2 domain can each comprise the amino acid sequence of SEQ ID NO: 19 or SEQ ID NO: 49. In some embodiments, the first CH2 domain, the second CH2 domain, and the third CH2 domain each comprise the amino acid sequence of SEQ ID NO: 19. The first CH2 domain, the second CH2 domain, and the third CH2 domain can each comprise an amino acid sequence with at least 90% sequence identity to SEQ ID NO: 19. The first CH2 domain, the second CH2 domain, and the third CH2 domain can each comprise an amino acid sequence with at least 95% sequence identity to SEQ ID NO: 19.

In some embodiments, the first CH2 domain, the second CH2 domain, and the third CH2 domain can each comprise the amino acid sequence of SEQ ID NO: 49. The first CH2 domain, the second CH2 domain, and the third CH2 domain can each comprise an amino acid sequence with at least 90% sequence identity to SEQ ID NO: 49. The first CH2 domain, the second CH2 domain, and the third CH2 domain can each comprise an amino acid sequence with at least 95% sequence identity to SEQ ID NO: 49.

The first hinge and the second hinge can each comprise the amino acid sequence of SEQ ID NO: 18 or SEQ ID NO: 48. In some embodiments, the first hinge and the second hinge can each comprise the amino acid sequence of SEQ ID NO: 18. The first hinge and the second hinge can each comprise an amino acid sequence with at least 90% sequence identity to SEQ ID NO: 18. The first hinge and the second hinge can each comprise an amino acid sequence with at least 95% sequence identity to SEQ ID NO: 18.

In some embodiments, the first hinge and the second hinge can each comprise the amino acid sequence of SEQ ID NO: 48. The first hinge and the second hinge can each comprise an amino acid sequence with at least 90% sequence identity to SEQ ID NO: 48. The first hinge and the second hinge can each comprise an amino acid sequence with at least 95% sequence identity to SEQ ID NO: 48.

The first linker and the second linker can each comprise the amino acid sequence of GS, GGS, SEQ ID NO: 14, SEQ ID NO: 38, SEQ ID NO: 43, or SEQ ID NO: 44. The first and the second linkers can comprise GS. The first and the second linkers can comprise GGS. The first and the second linkers can comprise SEQ ID NO: 14. The first and the second linkers can comprise SEQ ID NO: 38. The first and the second linkers can comprise SEQ ID NO: 43. The first and the second linkers can comprise a sequence with at least 90% sequence identity to SEQ ID NO: 43. The first and the second linkers can comprise SEQ ID NO: 44. The first and the second linkers can comprise a sequence with at least 90% sequence identity to SEQ ID NO: 44.

In some embodiments, the antibody comprises a CH3 domain comprising the amino acid sequence of SEQ ID NO: 20. The antibody can comprise a CH3 domain having an amino acid sequence with at least 90% sequence identity to SEQ ID NO: 20. The antibody can comprise a CH3 domain having an amino acid sequence with at least 95% sequence identity to SEQ ID NO: 20.

In some embodiments, the antibody comprises a heavy chain variable region having the polypeptide sequence of SEQ ID NO: 162 and a light chain variable region having the polypeptide sequence of SEQ ID NO: 167. The antibody can comprise a heavy chain variable region having an amino acid sequence with at least 90% sequence identity to SEQ ID NO: 162 and a light chain variable region having an amino acid sequence with at least 90% sequence identity to SEQ ID NO: 167. The antibody can comprise a heavy chain variable region having an amino acid sequence with at least 95% sequence identity to SEQ ID NO: 162 and a light chain variable region having an amino acid sequence with at least 95% sequence identity to SEQ ID NO: 167.

In some embodiments, the antibody comprises a heavy chain having a polypeptide sequence of SEQ ID NO: 171 and a light chain having the polypeptide sequence of SEQ ID NO: 173. The antibody can comprise a heavy chain having an amino acid sequence with at least 90% sequence identity to SEQ ID NO: 171 and a light chain having an amino acid sequence with at least 90% sequence identity to SEQ ID NO: 173. The antibody can comprise a heavy chain having an amino acid sequence with at least 95% sequence identity to SEQ ID NO: 171 and a light chain having an amino acid sequence with at least 95% sequence identity to SEQ ID NO: 173.

In some embodiments, the antibody comprises a heavy chain that is encoded by a polynucleotide sequence of SEQ ID NO: 170 and a light chain that is encoded by a polynucleotide sequence of SEQ ID NO: 172.

Disclosed herein are rabbit anti-mouse IgG secondary antibodies (RAM), wherein the antibodies comprise, consist essentially of, or consist of a heavy chain (HC) and a light chain (LC) that pair to form a binding domain comprising a heavy chain complementarity determining region 1 (HCDR1) comprising the amino acid sequence of SEQ ID NO: 127, a HCDR2 comprising the amino acid sequence of SEQ ID NO: 128, a HCDR3 comprising the amino acid sequence of SEQ ID NO: 129, a light chain complementarity determining region 1 (LCDR1) comprising the amino acid sequence of SEQ ID NO: 132, a LCDR2 comprising the amino acid sequence of GAS, and a LCDR3 comprising the amino acid sequence of SEQ ID NO: 133, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge. In some embodiments, the rabbit anti-mouse IgG secondary antibodies further comprise a CH3 domain linked to the C-terminus of the third CH2 domain.

The first CH2 domain, the second CH2 domain, and the third CH2 domain can each comprise the amino acid sequence of SEQ ID NO: 19 or SEQ ID NO: 49. In some embodiments, the first CH2 domain, the second CH2 domain, and the third CH2 domain each comprise the amino acid sequence of SEQ ID NO: 19. The first CH2 domain, the second CH2 domain, and the third CH2 domain can each comprise an amino acid sequence with at least 90% sequence identity to SEQ ID NO: 19. The first CH2 domain, the second CH2 domain, and the third CH2 domain can each comprise an amino acid sequence with at least 95% sequence identity to SEQ ID NO: 19.

In some embodiments, the first CH2 domain, the second CH2 domain, and the third CH2 domain can each comprise the amino acid sequence of SEQ ID NO: 49. The first CH2 domain, the second CH2 domain, and the third CH2 domain can each comprise an amino acid sequence with at least 90% sequence identity to SEQ ID NO: 49. The first CH2 domain, the second CH2 domain, and the third CH2 domain can each comprise an amino acid sequence with at least 95% sequence identity to SEQ ID NO: 49.

The first hinge and the second hinge can each comprise the amino acid sequence of SEQ ID NO: 18 or SEQ ID NO: 48. In some embodiments, the first hinge and the second hinge can each comprise the amino acid sequence of SEQ ID NO: 18 The first hinge and the second hinge can each comprise an amino acid sequence with at least 90% sequence identity to SEQ ID NO: 18. The first hinge and the second hinge can each comprise an amino acid sequence with at least 95% sequence identity to SEQ ID NO: 18.

In some embodiments, the first hinge and the second hinge can each comprise the amino acid sequence of SEQ ID NO: 48. The first hinge and the second hinge can each comprise an amino acid sequence with at least 90% sequence identity to SEQ ID NO: 48. The first hinge and the second hinge can each comprise an amino acid sequence with at least 95% sequence identity to SEQ ID NO: 48.

The first linker and the second linker can each comprise the amino acid sequence of GS, GGS, SEQ ID NO: 14, SEQ ID NO: 38, SEQ ID NO: 43, or SEQ ID NO: 44. The first and the second linkers can comprise GS. The first and the second linkers can comprise GGS. The first and the second linkers can comprise SEQ ID NO: 14. The first and the second linkers can comprise SEQ ID NO: 38. The first and the second linkers can comprise SEQ ID NO: 43. The first and the second linkers can comprise a sequence with at least 90% sequence identity to SEQ ID NO: 43. The first and the second linkers can comprise SEQ ID NO: 44. The first and the second linkers can comprise a sequence with at least 90% sequence identity to SEQ ID NO: 44.

In some embodiments, the antibody comprises a CH3 domain comprising the amino acid sequence of SEQ ID NO: 141. The antibody can comprise a CH3 domain having an amino acid sequence with at least 90% sequence identity to SEQ ID NO: 141. The antibody can comprise a CH3 domain having an amino acid sequence with at least 95% sequence identity to SEQ ID NO: 141.

In some embodiments, the antibody comprises a heavy chain variable region having the polypeptide sequence of SEQ ID NO: 126 and a light chain variable region having the polypeptide sequence of SEQ ID NO: 131. The antibody can comprise a heavy chain variable region having an amino acid sequence with at least 90% sequence identity to SEQ ID NO: 126 and a light chain variable region having an amino acid sequence with at least 90% sequence identity to SEQ ID NO: 131. The antibody can comprise a heavy chain variable region having an amino acid sequence with at least 95% sequence identity to SEQ ID NO: 126 and a light chain variable region having an amino acid sequence with at least 95% sequence identity to SEQ ID NO: 131.

In some embodiments, the antibody comprises a heavy chain having a polypeptide sequence of SEQ ID NO: 135 and a light chain having the polypeptide sequence of SEQ ID NO: 137. The antibody can comprise a heavy chain having an amino acid sequence with at least 90% sequence identity to SEQ ID NO: 135 and a light chain having an amino acid sequence with at least 90% sequence identity to SEQ ID NO: 137. The antibody can comprise a heavy chain having an amino acid sequence with at least 95% sequence identity to SEQ ID NO: 135 and a light chain having an amino acid sequence with at least 95% sequence identity to SEQ ID NO: 137.

In some embodiments, the antibody comprises a heavy chain that is encoded by a polynucleotide sequence of SEQ ID NO: 134 and a light chain that is encoded by a polynucleotide sequence of SEQ ID NO: 136.

Disclosed herein are antibodies that immunospecifically bind to pERK1/2, wherein the antibodies comprise, consist essentially of, or consist of a heavy chain (HC) and a light chain (LC) that pair to form a binding domain comprising a heavy chain complementarity determining region 1 (HCDR1) comprising the amino acid sequence of SEQ ID NO: 85, a HCDR2 comprising the amino acid sequence of SEQ ID NO: 86, a HCDR3 comprising the amino acid sequence of SEQ ID NO: 87, a light chain complementarity determining region 1 (LCDR1) comprising the amino acid sequence of SEQ ID NO: 91, a LCDR2 comprising the amino acid sequence of KVS, and a LCDR3 comprising the amino acid sequence of SEQ ID NO: 92, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge. In some embodiments, the antibodies that immunospecifically bind to pERK1/2 further comprise a CH3 domain linked to the C-terminus of the third CH2 domain.

The first CH2 domain, the second CH2 domain, and the third CH2 domain can each comprise the amino acid sequence of SEQ ID NO: 104. In some embodiments, the first CH2 domain, the second CH2 domain, and the third CH2 domain each comprise the amino acid sequence of SEQ ID NO: 104. The first CH2 domain, the second CH2 domain, and the third CH2 domain can each comprise an amino acid sequence with at least 90% sequence identity to SEQ ID NO: 104. The first CH2 domain, the second CH2 domain, and the third CH2 domain can each comprise an amino acid sequence with at least 95% sequence identity to SEQ ID NO: 104.

The first hinge and the second hinge can each comprise the amino acid sequence of SEQ ID NO: 102. In some embodiments, the first hinge and the second hinge can each comprise the amino acid sequence of SEQ ID NO: 102. The first hinge and the second hinge can each comprise an amino acid sequence with at least 90% sequence identity to SEQ ID NO: 102. The first hinge and the second hinge can each comprise an amino acid sequence with at least 95% sequence identity to SEQ ID NO: 102.

The first linker and the second linker can each comprise the amino acid sequence of GS, GGS, SEQ ID NO: 14, SEQ ID NO: 38, SEQ ID NO: 43, or SEQ ID NO: 44. The first and the second linkers can comprise GS. The first and the second linkers can comprise GGS. The first and the second linkers can comprise SEQ ID NO: 14. The first and the second linkers can comprise SEQ ID NO: 38. The first and the second linkers can comprise SEQ ID NO: 43. The first and the second linkers can comprise a sequence with at least 90% sequence identity to SEQ ID NO: 43. The first and the second linkers can comprise SEQ ID NO: 44. The first and the second linkers can comprise a sequence with at least 90% sequence identity to SEQ ID NO: 44.

In some embodiments, the antibody comprises a CH3 domain comprising the amino acid sequence of SEQ ID NO: 106. The antibody can comprise a CH3 domain having an amino acid sequence with at least 90% sequence identity to SEQ ID NO: 106. The antibody can comprise a CH3 domain having an amino acid sequence with at least 95% sequence identity to SEQ ID NO: 106.

In some embodiments, the antibody comprises a heavy chain variable region having the polypeptide sequence of SEQ ID NO: 84 and a light chain variable region having the polypeptide sequence of SEQ ID NO: 90. The antibody can comprise a heavy chain variable region having an amino acid sequence with at least 90% sequence identity to SEQ ID NO: 84 and a light chain variable region having an amino acid sequence with at least 90% sequence identity to SEQ ID NO: 90. The antibody can comprise a heavy chain variable region having an amino acid sequence with at least 95% sequence identity to SEQ ID NO: 84 and a light chain variable region having an amino acid sequence with at least 95% sequence identity to SEQ ID NO: 90.

In some embodiments, the antibody comprises a heavy chain having a polypeptide sequence of SEQ ID NO: 94 and a light chain having the polypeptide sequence of SEQ ID NO: 96. The antibody can comprise a heavy chain having an amino acid sequence with at least 90% sequence identity to SEQ ID NO: 94 and a light chain having an amino acid sequence with at least 90% sequence identity to SEQ ID NO: 96. The antibody can comprise a heavy chain having an amino acid sequence with at least 95% sequence identity to SEQ ID NO: 94 and a light chain having an amino acid sequence with at least 95% sequence identity to SEQ ID NO: 96.

In some embodiments, the antibody comprises a heavy chain that is encoded by a polynucleotide sequence of SEQ ID NO: 93 and a light chain that is encoded by a polynucleotide sequence of SEQ ID NO: 95.

Disclosed herein are antibodies that immunospecifically bind to PSD-95, wherein the antibodies comprise, consist essentially of, or consist of a heavy chain (HC) and a light chain (LC) that pair to form a binding domain comprising a heavy chain complementarity determining region 1 (HCDR1) comprising the amino acid sequence of SEQ ID NO: 111, a HCDR2 comprising the amino acid sequence of SEQ ID NO: 112, a HCDR3 comprising the amino acid sequence of SEQ ID NO: 113, a light chain complementarity determining region 1 (LCDR1) comprising the amino acid sequence of SEQ ID NO: 116, a LCDR2 comprising the amino acid sequence of DTS, and a LCDR3 comprising the amino acid sequence of SEQ ID NO: 117, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge. In some embodiments, the antibodies that immunospecifically bind to PSD-95 further comprise a CH3 domain linked to the C-terminus of the third CH2 domain.

The first CH2 domain, the second CH2 domain, and the third CH2 domain can each comprise the amino acid sequence of SEQ ID NO: 104. In some embodiments, the first CH2 domain, the second CH2 domain, and the third CH2 domain each comprise the amino acid sequence of SEQ ID NO: 104. The first CH2 domain, the second CH2 domain, and the third CH2 domain can each comprise an amino acid sequence with at least 90% sequence identity to SEQ ID NO: 104. The first CH2 domain, the second CH2 domain, and the third CH2 domain can each comprise an amino acid sequence with at least 95% sequence identity to SEQ ID NO: 104.

The first hinge and the second hinge can each comprise the amino acid sequence of SEQ ID NO: 102. In some embodiments, the first hinge and the second hinge can each comprise the amino acid sequence of SEQ ID NO: 102. The first hinge and the second hinge can each comprise an amino acid sequence with at least 90% sequence identity to SEQ ID NO: 102. The first hinge and the second hinge can each comprise an amino acid sequence with at least 95% sequence identity to SEQ ID NO: 102.

The first linker and the second linker can each comprise the amino acid sequence of GS, GGS, SEQ ID NO: 14, SEQ ID NO: 38, SEQ ID NO: 43, or SEQ ID NO: 44. The first and the second linkers can comprise GS. The first and the second linkers can comprise GGS. The first and the second linkers can comprise SEQ ID NO: 14. The first and the second linkers can comprise SEQ ID NO: 38. The first and the second linkers can comprise SEQ ID NO: 43. The first and the second linkers can comprise a sequence with at least 90% sequence identity to SEQ ID NO: 43. The first and the second linkers can comprise SEQ ID NO: 44. The first and the second linkers can comprise a sequence with at least 90% sequence identity to SEQ ID NO: 44.

In some embodiments, the antibody comprises a CH3 domain comprising the amino acid sequence of SEQ ID NO: 106. The antibody can comprise a CH3 domain having an amino acid sequence with at least 90% sequence identity to SEQ ID NO: 106. The antibody can comprise a CH3 domain having an amino acid sequence with at least 95% sequence identity to SEQ ID NO: 106.

In some embodiments, the antibody comprises a heavy chain variable region having the polypeptide sequence of SEQ ID NO: 110 and a light chain variable region having the polypeptide sequence of SEQ ID NO: 115. The antibody can comprise a heavy chain variable region having an amino acid sequence with at least 90% sequence identity to SEQ ID NO: 110 and a light chain variable region having an amino acid sequence with at least 90% sequence identity to SEQ ID NO: 115. The antibody can comprise a heavy chain variable region having an amino acid sequence with at least 95% sequence identity to SEQ ID NO: 110 and a light chain variable region having an amino acid sequence with at least 95% sequence identity to SEQ ID NO: 115.

In some embodiments, the antibody comprises a heavy chain having a polypeptide sequence of SEQ ID NO: 119 and a light chain having the polypeptide sequence of SEQ ID NO: 121. The antibody can comprise a heavy chain having an amino acid sequence with at least 90% sequence identity to SEQ ID NO: 119 and a light chain having an amino acid sequence with at least 90% sequence identity to SEQ ID NO: 121. The antibody can comprise a heavy chain having an amino acid sequence with at least 95% sequence identity to SEQ ID NO: 119 and a light chain having an amino acid sequence with at least 95% sequence identity to SEQ ID NO: 121.

In some embodiments, the antibody comprises a heavy chain that is encoded by a polynucleotide sequence of SEQ ID NO: 118 and a light chain that is encoded by a polynucleotide sequence of SEQ ID NO: 120.

Disclosed herein are antibodies that immunospecifically bind to TIGIT, wherein the antibodies comprise, consist essentially of, or consist of a heavy chain (HC) and a light chain (LC) that pair to form a binding domain comprising a heavy chain complementarity determining region 1 (HCDR1) comprising the amino acid sequence of SEQ ID NO: 146, a HCDR2 comprising the amino acid sequence of SEQ ID NO: 147, a HCDR3 comprising the amino acid sequence of SEQ ID NO: 148, a light chain complementarity determining region 1 (LCDR1) comprising the amino acid sequence of SEQ ID NO: 151, a LCDR2 comprising the amino acid sequence of SAS, and a LCDR3 comprising the amino acid sequence of SEQ ID NO: 152, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge. In some embodiments, the antibodies that immunospecifically bind to TIGIT further comprise a CH3 domain linked to the C-terminus of the third CH2 domain.

The first CH2 domain, the second CH2 domain, and the third CH2 domain can each comprise the amino acid sequence of SEQ ID NO: 19. In some embodiments, the first CH2 domain, the second CH2 domain, and the third CH2 domain each comprise the amino acid sequence of SEQ ID NO: 19. The first CH2 domain, the second CH2 domain, and the third CH2 domain can each comprise an amino acid sequence with at least 90% sequence identity to SEQ ID NO: 19. The first CH2 domain, the second CH2 domain, and the third CH2 domain can each comprise an amino acid sequence with at least 95% sequence identity to SEQ ID NO: 19.

The first hinge and the second hinge can each comprise the amino acid sequence of SEQ ID NO: 18. In some embodiments, the first hinge and the second hinge can each comprise the amino acid sequence of SEQ ID NO: 18. The first hinge and the second hinge can each comprise an amino acid sequence with at least 90% sequence identity to SEQ ID NO: 18. The first hinge and the second hinge can each comprise an amino acid sequence with at least 95% sequence identity to SEQ ID NO: 18.

The first linker and the second linker can each comprise the amino acid sequence of GS, GGS, SEQ ID NO: 14, SEQ ID NO: 38, SEQ ID NO: 43, or SEQ ID NO: 44. The first and the second linkers can comprise GS. The first and the second linkers can comprise GGS. The first and the second linkers can comprise SEQ ID NO: 14. The first and the second linkers can comprise SEQ ID NO: 38. The first and the second linkers can comprise SEQ ID NO: 43. The first and the second linkers can comprise a sequence with at least 90% sequence identity to SEQ ID NO: 43. The first and the second linkers can comprise SEQ ID NO: 44. The first and the second linkers can comprise a sequence with at least 90% sequence identity to SEQ ID NO: 44.

In some embodiments, the antibody comprises a CH3 domain comprising the amino acid sequence of SEQ ID NO: 20. The antibody can comprise a CH3 domain having an amino acid sequence with at least 90% sequence identity to SEQ ID NO: 20. The antibody can comprise a CH3 domain having an amino acid sequence with at least 95% sequence identity to SEQ ID NO: 20.

In some embodiments, the antibody comprises a heavy chain variable region having the polypeptide sequence of SEQ ID NO: 145 and a light chain variable region having the polypeptide sequence of SEQ ID NO: 150. The antibody can comprise a heavy chain variable region having an amino acid sequence with at least 90% sequence identity to SEQ ID NO: 145 and a light chain variable region having an amino acid sequence with at least 90% sequence identity to SEQ ID NO: 150. The antibody can comprise a heavy chain variable region having an amino acid sequence with at least 95% sequence identity to SEQ ID NO: 145 and a light chain variable region having an amino acid sequence with at least 95% sequence identity to SEQ ID NO: 150.

In some embodiments, the antibody comprises a heavy chain having a polypeptide sequence of SEQ ID NO: 154 and a light chain having the polypeptide sequence of SEQ ID NO: 156. The antibody can comprise a heavy chain having an amino acid sequence with at least 90% sequence identity to SEQ ID NO: 154 and a light chain having an amino acid sequence with at least 90% sequence identity to SEQ ID NO: 156. The antibody can comprise a heavy chain having an amino acid sequence with at least 95% sequence identity to SEQ ID NO: 154 and a light chain having an amino acid sequence with at least 95% sequence identity to SEQ ID NO: 156.

In some embodiments, the antibody comprises a heavy chain that is encoded by a polynucleotide sequence of SEQ ID NO: 153 and a light chain that is encoded by a polynucleotide sequence of SEQ ID NO: 155.

In some embodiments, the antibody is labeled. The label can be selected from a fluorophore, a fluorescent protein, a phosphorescent dye, a tandem dye, a polymer dye, a particle, an electron transfer agent, a hapten, an enzyme, a radioisotope, a chromophore, and an oligonucleotide-based fluorescent dye. The label can be a fluorophore.

The label can be a fluorescent protein. The label can be a phosphorescent dye. The label can be a tandem dye. The label can be a polymer dye. The label can be a particle. The label can be an electron transfer agent. The label can be a hapten. The label can be an enzyme. The label can be a radioisotope. The label can be a chromophore. The label can be an oligonucleotide-based fluorescent dye.

In some embodiments, the label is a fluorophore. The fluorophore can be a pyrene, an anthracene, a naphthalene, an acridine, a stilbene, an indole or benzindole, an oxazole or benzoxazole, a thiazole or benzothiazole, an indacene, a 4-amino-7-nitrobenz-2-oxa-1,3-diazole (NBD), a cyanine, a carbocyanine, a benzocyanine, a carbostyryl, a porphyrin, a salicylate, an anthranilate, an azulene, a perylene, a pyridine, a quinoline, a borapolyazaindacene, a xanthene, a rhodamine, a silicon rhodamine, a fluorescein, a rhodol, a rosamine, an oxazine or a benzoxazine, a benzophenoxazine, a carbazine, a phenalenone, a coumarin, a furan or a benzofuran, a benzophenalenone, a benzopyrilium, a styryl, a squarine, a resorufin, an anthraquinone, a quinazolinone, an oligonucleotide-based fluorescent dyes, a conjugated polymer dye, or a tandem dye. The fluorophore can be a pyrene. The fluorophore can be an anthracene. The fluorophore can be a naphthalene. The fluorophore can be an acridine. The fluorophore can be a stilbene. The fluorophore can be an indole. The fluorophore can be a benzindole. The fluorophore can be an oxazole. The fluorophore can be a benzoxazole. The fluorophore can be a thiazole. The fluorophore can be a benzothiazole.

The fluorophore can be an indacene. The fluorophore can be a 4-amino-7-nitrobenz-2-oxa-1, 3-diazole (NBD). The fluorophore can be a cyanine. The fluorophore can be a carbocyanine.

The fluorophore can be a benzocyanine. The fluorophore can be a carbostyryl. The fluorophore can be a porphyrin. The fluorophore can be a salicylate. The fluorophore can be an anthranilate. The fluorophore can be an azulene. The fluorophore can be a perylene. The fluorophore can be a pyridine. The fluorophore can be a quinoline. The fluorophore can be a borapolyazaindacene. The fluorophore can be a xanthene. The fluorophore can be a rhodamine. The fluorophore can be a silicon rhodamine. The fluorophore can be a fluorescein. The fluorophore can be a rhodol. The fluorophore can be a rosamine. The fluorophore can be an oxazine. The fluorophore can be a benzoxazine. The fluorophore can be a benzophenoxazine. The fluorophore can be a carbazine. The fluorophore can be a phenalenone. The fluorophore can be a coumarin. The fluorophore can be a furan. The fluorophore can be a benzofuran. The fluorophore can be a benzophenalenone. The fluorophore can be a benzopyrilium. The fluorophore can be a styryl. The fluorophore can be a squarine. The fluorophore can be a resorufin. The fluorophore can be an anthrquinone.

The fluorophore can be a quinazolinone. The fluorophore can be an oligonucleotide-based fluorescent dye. The fluorophore can be a conjugated polymer dye. The fluorophore can be a tandem dye.

In some embodiments, the label is a particle. The particle can be a semiconductor nanocrystal.

In some embodiments, the label is a hapten. The hapten can be a biotin or a biotin derivative. The hapten can be biotin. The hapten can be a biotin derivative.

In some embodiments, the label is an enzyme. The enzyme can be a peroxidase, a phosphatase, a glycosidase, and a luciferase. The enzyme can be a peroxidase.

The enzyme can be a phosphatase. The enzyme can be a glycosidase. The enzyme can be a luciferase.

Disclosed herein are nucleic acid molecules comprising, consisting essentially of, or consisting of polynucleotides encoding antibodies that immunospecifically binds to Parkin, wherein the antibodies comprise a heavy chain (HC) and a light chain (LC) that pair to form a binding domain comprising a heavy chain complementarity determining region 1 (HCDR1) comprising the amino acid sequence of SEQ ID NO: 4, a HCDR2 comprising the amino acid sequence of SEQ ID NO: 5, a HCDR3 comprising the amino acid sequence of SEQ ID NO: 6, a light chain complementarity determining region 1 (LCDR1) comprising the amino acid sequence of SEQ ID NO: 13, a LCDR2 comprising the amino acid sequence RAS, and a LCDR3 comprising the amino acid sequence of SEQ ID NO: 15, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge. In some embodiments, polynucleotides further encode a CH3 domain linked to the C-terminus of the third CH2 domain.

The polynucleotide can encode a first CH2 domain, a second CH2 domain, and a third CH2 domain each comprising the amino acid sequence of SEQ ID NO: 19 or SEQ ID NO: 49. In some embodiments, the polynucleotide encodes a first CH2 domain, a second CH2 domain, and a third CH2 domain each comprising the amino acid sequence of SEQ ID NO: 19. The polynucleotide can encode a first CH2 domain, a second CH2 domain, and a third CH2 domain each comprising an amino acid sequence with at least 90% sequence identity to SEQ ID NO: 19. The polynucleotide can encode a first CH2 domain, a second CH2 domain, and a third CH2 domain each comprising an amino acid sequence with at least 95% sequence identity to SEQ ID NO: 19. In some embodiments, in the polynucleotide encoding the first CH2 domain, the second CH2 domain, and the third CH2 domain, the polynucleotide comprises SEQ ID NO: 23.

In some embodiments, the polynucleotide encodes a first CH2 domain, a second CH2 domain, and a third CH2 domain each comprising the amino acid sequence of SEQ ID NO: 49. The polynucleotide can encode a first CH2 domain, a second CH2 domain, and a third CH2 domain each comprising an amino acid sequence with at least 90% sequence identity to SEQ ID NO: 49. The polynucleotide can encode a first CH2 domain, a second CH2 domain, and a third CH2 domain each comprising an amino acid sequence with at least 95% sequence identity to SEQ ID NO: 49. In some embodiments, in the polynucleotide encoding the first CH2 domain, the second CH2 domain, and the third CH2 domain, the polynucleotide encoding each CH2 domain comprises SEQ ID NO: 62.

The polynucleotide can encode a first hinge and a second hinge each comprising the amino acid sequence of SEQ ID NO: 18 or SEQ ID NO: 48. In some embodiments, the polynucleotide encodes a first hinge and a second hinge each comprising the amino acid sequence of SEQ ID NO: 18. The polynucleotide can encode a first hinge and a second hinge each comprising an amino acid sequence with at least 90% sequence identity to SEQ ID NO: 18. The polynucleotide can encode a first hinge and a second hinge each comprising an amino acid sequence with at least 95% sequence identity to SEQ ID NO: 18.

In some embodiments, in the polynucleotide encoding the first hinge and the second hinge, the polynucleotide encoding each hinge comprises SEQ ID NO: 22.

In some embodiments, the polynucleotide encodes a first hinge and a second hinge each comprising the amino acid sequence of SEQ ID NO: 48. The polynucleotide can encode a first hinge and a second hinge each comprising an amino acid sequence with at least 90% sequence identity to SEQ ID NO: 48. The polynucleotide can encode a first hinge and a second hinge each comprising an amino acid sequence with at least 95% sequence identity to SEQ ID NO: 48. In some embodiments, in the polynucleotide encoding the first hinge and the second hinge, the polynucleotide encoding each hinge comprises SEQ ID NO: 51.

The polynucleotide can encode a first linker and a second linker comprising the amino acid sequence of GS, GGS, SEQ ID NO: 14, SEQ ID NO: 38, SEQ ID NO: 43, or SEQ ID NO: 44. The polynucleotide can encode the first and the second linkers comprising GS. The first and the second linkers can comprise GGS. The polynucleotide can encode the first and the second linkers comprising SEQ ID NO: 14. The polynucleotide can encode the first and the second linkers comprising SEQ ID NO: 38. The polynucleotide can encode the first and the second linkers comprising SEQ ID NO: 43. The polynucleotide can encode a first and a second linker comprising a sequence with at least 90% sequence identity to SEQ ID NO: 43. The polynucleotide can encode the first and the second linkers comprising SEQ ID NO: 44. The polynucleotide can encode a first and a second linker comprising a sequence with at least 90% sequence identity to SEQ ID NO: 44.

In some embodiments, the polynucleotide encodes an antibody that comprises a CH3 domain comprising the amino acid sequence of SEQ ID NO: 20. In some embodiments, the polynucleotide encodes an antibody that comprises a CH3 domain having an amino acid sequence with at least 90% sequence identity to SEQ ID NO: 20. In some embodiments, the polynucleotide encodes an antibody that comprises a CH3 domain having an amino acid sequence with at least 95% sequence identity to SEQ ID NO: 20.

In some embodiments, the polynucleotide encodes an antibody comprising a heavy chain variable region having the polypeptide sequence of SEQ ID NO: 7 and a light chain variable region having the polypeptide sequence of SEQ ID NO: 16. The polynucleotide can encode an antibody comprising a heavy chain variable region having an amino acid sequence with at least 90% sequence identity to SEQ ID NO: 7 and a light chain variable region having an amino acid sequence with at least 90% sequence identity to SEQ ID NO: 16. The polynucleotide can encode an antibody comprising a heavy chain variable region having an amino acid sequence with at least 95% sequence identity to SEQ ID NO: 7 and a light chain variable region having an amino acid sequence with at least 95% sequence identity to SEQ ID NO: 16.

In some embodiments, the polynucleotide encodes an antibody comprising a heavy chain having a polypeptide sequence of SEQ ID NO: 3 and a light chain having the polypeptide sequence of SEQ ID NO: 12. The polynucleotide can encode an antibody comprising a heavy chain having an amino acid sequence with at least 90% sequence identity to SEQ ID NO: 3 and a light chain having an amino acid sequence with at least 90% sequence identity to SEQ ID NO: 12. The polynucleotide can encode an antibody comprising a heavy chain having an amino acid sequence with at least 95% sequence identity to SEQ ID NO: 3 and a light chain having an amino acid sequence with at least 95% sequence identity to SEQ ID NO: 12. In some embodiments, in the polynucleotide encoding the heavy chain and the light chain, the polynucleotide comprises SEQ ID NO: 2 and SEQ ID NO: 11.

Disclosed herein are nucleic acid molecules comprising, consisting essentially of, or consisting of polynucleotides encoding antibodies that immunospecifically binds to OCT4, wherein the antibody comprises a heavy chain (HC) and a light chain (LC) that pair to form a binding domain comprising a heavy chain complementarity determining region 1 (HCDR1) comprising the amino acid sequence of SEQ ID NO: 29, a HCDR2 comprising the amino acid sequence of SEQ ID NO: 30, and a HCDR3 comprising the amino acid sequence of SEQ ID NO: 31, a light chain complementarity determining region 1 (LCDR1) comprising the amino acid sequence of SEQ ID NO: 37, a LCDR2 comprising the amino acid sequence GAS, and a LCDR3 comprising the amino acid sequence of SEQ ID NO: 39, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge. In some embodiments, polynucleotides further encode a CH3 domain linked to the C-terminus of the third CH2 domain.

The polynucleotide can encode a first CH2 domain, a second CH2 domain, and a third CH2 domain each comprising the amino acid sequence of SEQ ID NO: 19 or SEQ ID NO: 49. In some embodiments, the polynucleotide encodes a first CH2 domain, a second CH2 domain, and a third CH2 domain each comprising the amino acid sequence of SEQ ID NO: 19. The polynucleotide can encode a first CH2 domain, a second CH2 domain, and a third CH2 domain each comprising an amino acid sequence with at least 90% sequence identity to SEQ ID NO: 19. The polynucleotide can encode a first CH2 domain, a second CH2 domain, and a third CH2 domain each comprising an amino acid sequence with at least 95% sequence identity to SEQ ID NO: 19. In some embodiments, in the polynucleotide encoding the first CH2 domain, the second CH2 domain, and the third CH2 domain, the polynucleotide comprises SEQ ID NO: 23.

In some embodiments, the polynucleotide encodes a first CH2 domain, a second CH2 domain, and a third CH2 domain each comprising the amino acid sequence of SEQ ID NO: 49. The polynucleotide can encode a first CH2 domain, a second CH2 domain, and a third CH2 domain each comprising an amino acid sequence with at least 90% sequence identity to SEQ ID NO: 49. The polynucleotide can encode a first CH2 domain, a second CH2 domain, and a third CH2 domain each comprising an amino acid sequence with at least 95% sequence identity to SEQ ID NO: 49. In some embodiments, in the polynucleotide encoding the first CH2 domain, the second CH2 domain, and the third CH2 domain, the polynucleotide encoding each CH2 domain comprises SEQ ID NO: 62.

The polynucleotide can encode a first hinge and a second hinge each comprising the amino acid sequence of SEQ ID NO: 18 or SEQ ID NO: 48. In some embodiments, the polynucleotide encodes a first hinge and a second hinge each comprising the amino acid sequence of SEQ ID NO: 18. The polynucleotide can encode a first hinge and a second hinge each comprising an amino acid sequence with at least 90% sequence identity to SEQ ID NO: 18. The polynucleotide can encode a first hinge and a second hinge each comprising an amino acid sequence with at least 95% sequence identity to SEQ ID NO: 18.

In some embodiments, in the polynucleotide encoding the first hinge and the second hinge, the polynucleotide encoding each hinge comprises SEQ ID NO: 22.

In some embodiments, the polynucleotide encodes a first hinge and a second hinge each comprising the amino acid sequence of SEQ ID NO: 48. The polynucleotide can encode a first hinge and a second hinge each comprising an amino acid sequence with at least 90% sequence identity to SEQ ID NO: 48. The polynucleotide can encode a first hinge and a second hinge each comprising an amino acid sequence with at least 95% sequence identity to SEQ ID NO: 48. In some embodiments, in the polynucleotide encoding the first hinge and the second hinge, the polynucleotide encoding each hinge comprises SEQ ID NO: 51.

The polynucleotide can encode a first linker and a second linker comprising the amino acid sequence of GS, GGS, SEQ ID NO: 14, SEQ ID NO: 38, SEQ ID NO: 43, or SEQ ID NO: 44. The polynucleotide can encode the first and the second linkers comprising GS. The first and the second linkers can comprise GGS. The polynucleotide can encode the first and the second linkers comprising SEQ ID NO: 14. The polynucleotide can encode the first and the second linkers comprising SEQ ID NO: 38. The polynucleotide can encode the first and the second linkers comprising SEQ ID NO: 43. The polynucleotide can encode a first and a second linker comprising a sequence with at least 90% sequence identity to SEQ ID NO: 43. The polynucleotide can encode the first and the second linkers comprising SEQ ID NO: 44. The polynucleotide can encode a first and a second linker comprising a sequence with at least 90% sequence identity to SEQ ID NO: 44.

In some embodiments, the polynucleotide encodes an antibody comprising a heavy chain variable region having the polypeptide sequence of SEQ ID NO: 32 and a light chain variable region having the polypeptide sequence of SEQ ID NO: 40. The polynucleotide can encode an antibody comprising a heavy chain variable region having an amino acid sequence with at least 90% sequence identity to SEQ ID NO: 32 and a light chain variable region having an amino acid sequence with at least 90% sequence identity to SEQ ID NO: 40. The polynucleotide can encode an antibody comprising a heavy chain variable region having an amino acid sequence with at least 95% sequence identity to SEQ ID NO: 32 and a light chain variable region having an amino acid sequence with at least 95% sequence identity to SEQ ID NO: 40.

In some embodiments, the polynucleotide encodes an antibody that comprises a CH3 domain comprising the amino acid sequence of SEQ ID NO: 20. In some embodiments, the polynucleotide encodes an antibody that comprises a CH3 domain having an amino acid sequence with at least 90% sequence identity to SEQ ID NO: 20. In some embodiments, the polynucleotide encodes an antibody that comprises a CH3 domain having an amino acid sequence with at least 95% sequence identity to SEQ ID NO: 20.

In some embodiments, the polynucleotide encodes an antibody comprising a heavy chain having a polypeptide sequence of SEQ ID NO: 28 and a light chain having the polypeptide sequence of SEQ ID NO: 36. The polynucleotide can encode an antibody comprising a heavy chain having an amino acid sequence with at least 90% sequence identity to SEQ ID NO: 28 and a light chain having an amino acid sequence with at least 90% sequence identity to SEQ ID NO: 36. The polynucleotide can encode an antibody comprising a heavy chain having an amino acid sequence with at least 95% sequence identity to SEQ ID NO: 28 and a light chain having an amino acid sequence with at least 95% sequence identity to SEQ ID NO: 36. In some embodiments, in the polynucleotide encoding the heavy chain and the light chain, the polynucleotide comprises SEQ ID NO: 27 and SEQ ID NO: 35.

Disclosed herein are nucleic acid molecules comprising, consisting essentially of, or consisting of polynucleotides encoding antibodies that immunospecifically binds to IDO1, wherein the antibodies comprise a heavy chain (HC) and a light chain (LC) that pair to form a binding domain comprising a heavy chain complementarity determining region 1 (HCDR1) comprising the amino acid sequence of SEQ ID NO: 66, a HCDR2 comprising the amino acid sequence of SEQ ID NO: 67, a HCDR3 comprising the amino acid sequence of SEQ ID NO: 68, a light chain complementarity determining region 1 (LCDR1) comprising the amino acid sequence of SEQ ID NO: 72, a LCDR2 comprising the amino acid sequence of YAS, and a LCDR3 comprising the amino acid sequence of SEQ ID NO: 73, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge. In some embodiments, polynucleotides further encode a CH3 domain linked to the C-terminus of the third CH2 domain.

The polynucleotide can encode a first CH2 domain, a second CH2 domain, and a third CH2 domain each comprising the amino acid sequence of SEQ ID NO: 19 or SEQ ID NO: 49. In some embodiments, the polynucleotide encodes a first CH2 domain, a second CH2 domain, and a third CH2 domain each comprising the amino acid sequence of SEQ ID NO: 19. The polynucleotide can encode a first CH2 domain, a second CH2 domain, and a third CH2 domain each comprising an amino acid sequence with at least 90% sequence identity to SEQ ID NO: 19. The polynucleotide can encode a first CH2 domain, a second CH2 domain, and a third CH2 domain each comprising an amino acid sequence with at least 95% sequence identity to SEQ ID NO: 19. In some embodiments, in the polynucleotide encoding the first CH2 domain, the second CH2 domain, and the third CH2 domain, the polynucleotide comprises SEQ ID NO: 178.

In some embodiments, the polynucleotide encodes a first CH2 domain, a second CH2 domain, and a third CH2 domain each comprising the amino acid sequence of SEQ ID NO: 49. The polynucleotide can encode a first CH2 domain, a second CH2 domain, and a third CH2 domain each comprising an amino acid sequence with at least 90% sequence identity to SEQ ID NO: 49. The polynucleotide can encode a first CH2 domain, a second CH2 domain, and a third CH2 domain each comprising an amino acid sequence with at least 95% sequence identity to SEQ ID NO: 49. In some embodiments, in the polynucleotide encoding the first CH2 domain, the second CH2 domain, and the third CH2 domain, the polynucleotide encoding each CH2 domain comprises SEQ ID NO: 62.

The polynucleotide can encode a first hinge and a second hinge each comprising the amino acid sequence of SEQ ID NO: 18 or SEQ ID NO: 48. In some embodiments, the polynucleotide encodes a first hinge and a second hinge each comprising the amino acid sequence of SEQ ID NO: 18. The polynucleotide can encode a first hinge and a second hinge each comprising an amino acid sequence with at least 90% sequence identity to SEQ ID NO: 18. The polynucleotide can encode a first hinge and a second hinge each comprising an amino acid sequence with at least 95% sequence identity to SEQ ID NO: 18.

In some embodiments, in the polynucleotide encoding the first hinge and the second hinge, the polynucleotide encoding each hinge comprises SEQ ID NO: 22.

In some embodiments, the polynucleotide encodes a first hinge and a second hinge each comprising the amino acid sequence of SEQ ID NO: 48. The polynucleotide can encode a first hinge and a second hinge each comprising an amino acid sequence with at least 90% sequence identity to SEQ ID NO: 48. The polynucleotide can encode a first hinge and a second hinge each comprising an amino acid sequence with at least 95% sequence identity to SEQ ID NO: 48. In some embodiments, in the polynucleotide encoding the first hinge and the second hinge, the polynucleotide encoding each hinge comprises SEQ ID NO: 51.

The polynucleotide can encode a first linker and a second linker comprising the amino acid sequence of GS, GGS, SEQ ID NO: 14, SEQ ID NO: 38, SEQ ID NO: 43, or SEQ ID NO: 44. The polynucleotide can encode the first and the second linkers comprising GS. The first and the second linkers can comprise GGS. The polynucleotide can encode the first and the second linkers comprising SEQ ID NO: 14. The polynucleotide can encode the first and the second linkers comprising SEQ ID NO: 38. The polynucleotide can encode the first and the second linkers comprising SEQ ID NO: 43. The polynucleotide can encode a first and a second linker comprising a sequence with at least 90% sequence identity to SEQ ID NO: 43. The polynucleotide can encode the first and the second linkers comprising SEQ ID NO: 44. The polynucleotide can encode a first and a second linker comprising a sequence with at least 90% sequence identity to SEQ ID NO: 44.

In some embodiments, the polynucleotide encodes an antibody that comprises a CH3 domain comprising the amino acid sequence of SEQ ID NO: 20. In some embodiments, the polynucleotide encodes an antibody that comprises a CH3 domain having an amino acid sequence with at least 90% sequence identity to SEQ ID NO: 20. In some embodiments, the polynucleotide encodes an antibody that comprises a CH3 domain having an amino acid sequence with at least 95% sequence identity to SEQ ID NO: 20.

In some embodiments, the polynucleotide encodes an antibody comprising a heavy chain variable region having the polypeptide sequence of SEQ ID NO: 65 and a light chain variable region having the polypeptide sequence of SEQ ID NO: 71. The polynucleotide can encode an antibody comprising a heavy chain variable region having an amino acid sequence with at least 90% sequence identity to SEQ ID NO: 65 and a light chain variable region having an amino acid sequence with at least 90% sequence identity to SEQ ID NO: 71. The polynucleotide can encode an antibody comprising a heavy chain variable region having an amino acid sequence with at least 95% sequence identity to SEQ ID NO: 65 and a light chain variable region having an amino acid sequence with at least 95% sequence identity to SEQ ID NO: 71.

In some embodiments, the polynucleotide encodes an antibody comprising a heavy chain having a polypeptide sequence of SEQ ID NO: 75 and a light chain having the polypeptide sequence of SEQ ID NO: 77. The polynucleotide can encode an antibody comprising a heavy chain having an amino acid sequence with at least 90% sequence identity to SEQ ID NO: 75 and a light chain having an amino acid sequence with at least 90% sequence identity to SEQ ID NO: 77. The polynucleotide can encode an antibody comprising a heavy chain having an amino acid sequence with at least 95% sequence identity to SEQ ID NO: 75 and a light chain having an amino acid sequence with at least 95% sequence identity to SEQ ID NO: 77. In some embodiments, in the polynucleotide encoding the heavy chain and the light chain, the polynucleotide comprises SEQ ID NO: 75 and SEQ ID NO: 77.

Disclosed herein are nucleic acid molecules comprising, consisting essentially of, or consisting of polynucleotides encoding antibodies that immunospecifically binds to FLAG-tag, wherein the antibodies comprise a heavy chain (HC) and a light chain (LC) that pair to form a binding domain comprising a heavy chain complementarity determining region 1 (HCDR1) comprising the amino acid sequence of SEQ ID NO: 163, a HCDR2 comprising the amino acid sequence of SEQ ID NO: 164, a HCDR3 comprising the amino acid sequence of SEQ ID NO: 165, a light chain complementarity determining region 1 (LCDR1) comprising the amino acid sequence of SEQ ID NO: 168, a LCDR2 comprising the amino acid sequence of YAS, and a LCDR3 comprising the amino acid sequence of SEQ ID NO: 169, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge. In some embodiments, polynucleotides further encode a CH3 domain linked to the C-terminus of the third CH2 domain.

The polynucleotide can encode a first CH2 domain, a second CH2 domain, and a third CH2 domain each comprising the amino acid sequence of SEQ ID NO: 19 or SEQ ID NO: 49. In some embodiments, the polynucleotide encodes a first CH2 domain, a second CH2 domain, and a third CH2 domain each comprising the amino acid sequence of SEQ ID NO: 19. The polynucleotide can encode a first CH2 domain, a second CH2 domain, and a third CH2 domain each comprising an amino acid sequence with at least 90% sequence identity to SEQ ID NO: 19. The polynucleotide can encode a first CH2 domain, a second CH2 domain, and a third CH2 domain each comprising an amino acid sequence with at least 95% sequence identity to SEQ ID NO: 19. In some embodiments, in the polynucleotide encoding the first CH2 domain, the second CH2 domain, and the third CH2 domain, the polynucleotide comprises SEQ ID NO: 23.

In some embodiments, the polynucleotide encodes a first CH2 domain, a second CH2 domain, and a third CH2 domain each comprising the amino acid sequence of SEQ ID NO: 49. The polynucleotide can encode a first CH2 domain, a second CH2 domain, and a third CH2 domain each comprising an amino acid sequence with at least 90% sequence identity to SEQ ID NO: 49. The polynucleotide can encode a first CH2 domain, a second CH2 domain, and a third CH2 domain each comprising an amino acid sequence with at least 95% sequence identity to SEQ ID NO: 49. In some embodiments, in the polynucleotide encoding the first CH2 domain, the second CH2 domain, and the third CH2 domain, the polynucleotide encoding each CH2 domain comprises SEQ ID NO: 62.

The polynucleotide can encode a first hinge and a second hinge each comprising the amino acid sequence of SEQ ID NO: 18 or SEQ ID NO: 48. In some embodiments, the polynucleotide encodes a first hinge and a second hinge each comprising the amino acid sequence of SEQ ID NO: 18. The polynucleotide can encode a first hinge and a second hinge each comprising an amino acid sequence with at least 90% sequence identity to SEQ ID NO: 18. The polynucleotide can encode a first hinge and a second hinge each comprising an amino acid sequence with at least 95% sequence identity to SEQ ID NO: 18.

In some embodiments, in the polynucleotide encoding the first hinge and the second hinge, the polynucleotide encoding each hinge comprises SEQ ID NO: 22.

In some embodiments, the polynucleotide encodes a first hinge and a second hinge each comprising the amino acid sequence of SEQ ID NO: 48. The polynucleotide can encode a first hinge and a second hinge each comprising an amino acid sequence with at least 90% sequence identity to SEQ ID NO: 48. The polynucleotide can encode a first hinge and a second hinge each comprising an amino acid sequence with at least 95% sequence identity to SEQ ID NO: 48. In some embodiments, in the polynucleotide encoding the first hinge and the second hinge, the polynucleotide encoding each hinge comprises SEQ ID NO: 51.

The polynucleotide can encode a first linker and a second linker comprising the amino acid sequence of GS, GGS, SEQ ID NO: 14, SEQ ID NO: 38, SEQ ID NO: 43, or SEQ ID NO: 44. The polynucleotide can encode the first and the second linkers comprising GS. The first and the second linkers can comprise GGS. The polynucleotide can encode the first and the second linkers comprising SEQ ID NO: 14. The polynucleotide can encode the first and the second linkers comprising SEQ ID NO: 38. The polynucleotide can encode the first and the second linkers comprising SEQ ID NO: 43. The polynucleotide can encode a first and a second linker comprising a sequence with at least 90% sequence identity to SEQ ID NO: 43. The polynucleotide can encode the first and the second linkers comprising SEQ ID NO: 44. The polynucleotide can encode a first and a second linker comprising a sequence with at least 90% sequence identity to SEQ ID NO: 44.

In some embodiments, the polynucleotide encodes an antibody that comprises a CH3 domain comprising the amino acid sequence of SEQ ID NO: 20. In some embodiments, the polynucleotide encodes an antibody that comprises a CH3 domain having an amino acid sequence with at least 90% sequence identity to SEQ ID NO: 20. In some embodiments, the polynucleotide encodes an antibody that comprises a CH3 domain having an amino acid sequence with at least 95% sequence identity to SEQ ID NO: 20.

In some embodiments, the polynucleotide encodes an antibody comprising a heavy chain variable region having the polypeptide sequence of SEQ ID NO: 162 and a light chain variable region having the polypeptide sequence of SEQ ID NO: 167. The polynucleotide can encode an antibody comprising a heavy chain variable region having an amino acid sequence with at least 90% sequence identity to SEQ ID NO: 162 and a light chain variable region having an amino acid sequence with at least 90% sequence identity to SEQ ID NO: 167. The polynucleotide can encode an antibody comprising a heavy chain variable region having an amino acid sequence with at least 95% sequence identity to SEQ ID NO: 162 and a light chain variable region having an amino acid sequence with at least 95% sequence identity to SEQ ID NO: 167.

In some embodiments, the polynucleotide encodes an antibody comprising a heavy chain having a polypeptide sequence of SEQ ID NO: 171 and a light chain having the polypeptide sequence of SEQ ID NO: 173. The polynucleotide can encode an antibody comprising a heavy chain having an amino acid sequence with at least 90% sequence identity to SEQ ID NO: 171 and a light chain having an amino acid sequence with at least 90% sequence identity to SEQ ID NO: 173. The polynucleotide can encode an antibody comprising a heavy chain having an amino acid sequence with at least 95% sequence identity to SEQ ID NO: 171 and a light chain having an amino acid sequence with at least 95% sequence identity to SEQ ID NO: 173. In some embodiments, in the polynucleotide encoding the heavy chain and the light chain, the polynucleotide comprises SEQ ID NO: 170 and SEQ ID NO: 172.

Disclosed herein are nucleic acid molecules comprising, consisting essentially of, or consisting of polynucleotides encoding rabbit anti-mouse IgG secondary antibodies (RAM), wherein the antibodies comprise a heavy chain (HC) and a light chain (LC) that pair to form a binding domain comprising a heavy chain complementarity determining region 1 (HCDR1) comprising the amino acid sequence of SEQ ID NO: 127, a HCDR2 comprising the amino acid sequence of SEQ ID NO: 128, a HCDR3 comprising the amino acid sequence of SEQ ID NO: 129, a light chain complementarity determining region 1 (LCDR1) comprising the amino acid sequence of SEQ ID NO: 132, a LCDR2 comprising the amino acid sequence of GAS, and a LCDR3 comprising the amino acid sequence of SEQ ID NO: 133, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge. In some embodiments, polynucleotides further encode a CH3 domain linked to the C-terminus of the third CH2 domain.

The polynucleotide can encode a first CH2 domain, a second CH2 domain, and a third CH2 domain each comprising the amino acid sequence of SEQ ID NO: 19 or SEQ ID NO: 49. In some embodiments, the polynucleotide encodes a first CH2 domain, a second CH2 domain, and a third CH2 domain each comprising the amino acid sequence of SEQ ID NO: 19. The polynucleotide can encode a first CH2 domain, a second CH2 domain, and a third CH2 domain each comprising an amino acid sequence with at least 90% sequence identity to SEQ ID NO: 19. The polynucleotide can encode a first CH2 domain, a second CH2 domain, and a third CH2 domain each comprising an amino acid sequence with at least 95% sequence identity to SEQ ID NO: 19. In some embodiments, in the polynucleotide encoding the first CH2 domain, the second CH2 domain, and the third CH2 domain, the polynucleotide comprises SEQ ID NO: 23.

In some embodiments, the polynucleotide encodes a first CH2 domain, a second CH2 domain, and a third CH2 domain each comprising the amino acid sequence of SEQ ID NO: 49. The polynucleotide can encode a first CH2 domain, a second CH2 domain, and a third CH2 domain each comprising an amino acid sequence with at least 90% sequence identity to SEQ ID NO: 49 The polynucleotide can encode a first CH2 domain, a second CH2 domain, and a third CH2 domain each comprising an amino acid sequence with at least 95% sequence identity to SEQ ID NO: 49. In some embodiments, in the polynucleotide encoding the first CH2 domain, the second CH2 domain, and the third CH2 domain, the polynucleotide encoding each CH2 domain comprises SEQ ID NO: 62.

The polynucleotide can encode a first hinge and a second hinge each comprising the amino acid sequence of SEQ ID NO: 18 or SEQ ID NO: 48. In some embodiments, the polynucleotide encodes a first hinge and a second hinge each comprising the amino acid sequence of SEQ ID NO: 18. The polynucleotide can encode a first hinge and a second hinge each comprising an amino acid sequence with at least 90% sequence identity to SEQ ID NO: 18. The polynucleotide can encode a first hinge and a second hinge each comprising an amino acid sequence with at least 95% sequence identity to SEQ ID NO: 18.

In some embodiments, in the polynucleotide encoding the first hinge and the second hinge, the polynucleotide encoding each hinge comprises SEQ ID NO: 22.

In some embodiments, the polynucleotide encodes a first hinge and a second hinge each comprising the amino acid sequence of SEQ ID NO: 48. The polynucleotide can encode a first hinge and a second hinge each comprising an amino acid sequence with at least 90% sequence identity to SEQ ID NO: 48. The polynucleotide can encode a first hinge and a second hinge each comprising an amino acid sequence with at least 95% sequence identity to SEQ ID NO: 48. In some embodiments, in the polynucleotide encoding the first hinge and the second hinge, the polynucleotide encoding each hinge comprises SEQ ID NO: 51.

The polynucleotide can encode a first linker and a second linker comprising the amino acid sequence of GS, GGS, SEQ ID NO: 14, SEQ ID NO: 38, SEQ ID NO: 43, or SEQ ID NO: 44. The polynucleotide can encode the first and the second linkers comprising GS. The first and the second linkers can comprise GGS. The polynucleotide can encode the first and the second linkers comprising SEQ ID NO: 14. The polynucleotide can encode the first and the second linkers comprising SEQ ID NO: 38. The polynucleotide can encode the first and the second linkers comprising SEQ ID NO: 43. The polynucleotide can encode a first and a second linker comprising a sequence with at least 90% sequence identity to SEQ ID NO: 43. The polynucleotide can encode the first and the second linkers comprising SEQ ID NO: 44. The polynucleotide can encode a first and a second linker comprising a sequence with at least 90% sequence identity to SEQ ID NO: 44.

In some embodiments, the polynucleotide encodes an antibody that comprises a CH3 domain comprising the amino acid sequence of SEQ ID NO: 20. In some embodiments, the polynucleotide encodes an antibody that comprises a CH3 domain having an amino acid sequence with at least 90% sequence identity to SEQ ID NO: 20. In some embodiments, the polynucleotide encodes an antibody that comprises a CH3 domain having an amino acid sequence with at least 95% sequence identity to SEQ ID NO: 20.

In some embodiments, the polynucleotide encodes an antibody comprising a heavy chain variable region having the polypeptide sequence of SEQ ID NO: 126 and a light chain variable region having the polypeptide sequence of SEQ ID NO: 131. The polynucleotide can encode an antibody comprising a heavy chain variable region having an amino acid sequence with at least 90% sequence identity to SEQ ID NO: 126 and a light chain variable region having an amino acid sequence with at least 90% sequence identity to SEQ ID NO: 131. The polynucleotide can encode an antibody comprising a heavy chain variable region having an amino acid sequence with at least 95% sequence identity to SEQ ID NO: 126 and a light chain variable region having an amino acid sequence with at least 95% sequence identity to SEQ ID NO: 131.

In some embodiments, the polynucleotide encodes an antibody comprising a heavy chain having a polypeptide sequence of SEQ ID NO: 135 and a light chain having the polypeptide sequence of SEQ ID NO: 137. The polynucleotide can encode an antibody comprising a heavy chain having an amino acid sequence with at least 90% sequence identity to SEQ ID NO: 135 and a light chain having an amino acid sequence with at least 90% sequence identity to SEQ ID NO: 137. The polynucleotide can encode an antibody comprising a heavy chain having an amino acid sequence with at least 95% sequence identity to SEQ ID NO: 135 and a light chain having an amino acid sequence with at least 95% sequence identity to SEQ ID NO: 137. In some embodiments, in the polynucleotide encoding the heavy chain and the light chain, the polynucleotide comprises SEQ ID NO: 134 and SEQ ID NO: 136.

Disclosed herein are nucleic acid molecules comprising, consisting essentially of, or consisting of polynucleotides encoding antibodies that immunospecifically binds to pERK1/2, wherein the antibodies comprise a heavy chain (HC) and a light chain (LC) that pair to form a binding domain comprising a heavy chain complementarity determining region 1 (HCDR1) comprising the amino acid sequence of SEQ ID NO: 85, a HCDR2 comprising the amino acid sequence of SEQ ID NO: 86, a HCDR3 comprising the amino acid sequence of SEQ ID NO: 87, a light chain complementarity determining region 1 (LCDR1) comprising the amino acid sequence of SEQ ID NO: 91, a LCDR2 comprising the amino acid sequence of KVS, and a LCDR3 comprising the amino acid sequence of SEQ ID NO: 92, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge. In some embodiments, polynucleotides further encode a CH3 domain linked to the C-terminus of the third CH2 domain.

The polynucleotide can encode a first CH2 domain, a second CH2 domain, and a third CH2 domain each comprising the amino acid sequence of SEQ ID NO: 104. In some embodiments, the polynucleotide encodes a first CH2 domain, a second CH2 domain, and a third CH2 domain each comprising the amino acid sequence of SEQ ID NO: 104. The polynucleotide can encode a first CH2 domain, a second CH2 domain, and a third CH2 domain each comprising an amino acid sequence with at least 90% sequence identity to SEQ ID NO: 104. The polynucleotide can encode a first CH2 domain, a second CH2 domain, and a third CH2 domain each comprising an amino acid sequence with at least 95% sequence identity to SEQ ID NO: 104. In some embodiments, in the polynucleotide encoding the first CH2 domain, the second CH2 domain, and the third CH2 domain, the polynucleotide comprises SEQ ID NO: 103.

The polynucleotide can encode a first hinge and a second hinge each comprising the amino acid sequence of SEQ ID NO: 102. In some embodiments, the polynucleotide encodes a first hinge and a second hinge each comprising the amino acid sequence of SEQ ID NO: 102. The polynucleotide can encode a first hinge and a second hinge each comprising an amino acid sequence with at least 90% sequence identity to SEQ ID NO: 102. The polynucleotide can encode a first hinge and a second hinge each comprising an amino acid sequence with at least 95% sequence identity to SEQ ID NO: 102. In some embodiments, in the polynucleotide encoding the first hinge and the second hinge, the polynucleotide encoding each hinge comprises SEQ ID NO: 101.

The polynucleotide can encode a first linker and a second linker comprising the amino acid sequence of GS, GGS, SEQ ID NO: 14, SEQ ID NO: 38, SEQ ID NO: 43, or SEQ ID NO: 44. The polynucleotide can encode the first and the second linkers comprising GS. The first and the second linkers can comprise GGS. The polynucleotide can encode the first and the second linkers comprising SEQ ID NO: 14. The polynucleotide can encode the first and the second linkers comprising SEQ ID NO: 38. The polynucleotide can encode the first and the second linkers comprising SEQ ID NO: 43. The polynucleotide can encode a first and a second linker comprising a sequence with at least 90% sequence identity to SEQ ID NO: 43. The polynucleotide can encode the first and the second linkers comprising SEQ ID NO: 44. The polynucleotide can encode a first and a second linker comprising a sequence with at least 90% sequence identity to SEQ ID NO: 44.

In some embodiments, the polynucleotide encodes an antibody that comprises a CH3 domain comprising the amino acid sequence of SEQ ID NO: 106. In some embodiments, the polynucleotide encodes an antibody that comprises a CH3 domain having an amino acid sequence with at least 90% sequence identity to SEQ ID NO: 106. In some embodiments, the polynucleotide encodes an antibody that comprises a CH3 domain having an amino acid sequence with at least 95% sequence identity to SEQ ID NO: 106.

In some embodiments, the polynucleotide encodes an antibody comprising a heavy chain variable region having the polypeptide sequence of SEQ ID NO: 84 and a light chain variable region having the polypeptide sequence of SEQ ID NO: 90. The polynucleotide can encode an antibody comprising a heavy chain variable region having an amino acid sequence with at least 90% sequence identity to SEQ ID NO: 84 and a light chain variable region having an amino acid sequence with at least 90% sequence identity to SEQ ID NO: 90. The polynucleotide can encode an antibody comprising a heavy chain variable region having an amino acid sequence with at least 95% sequence identity to SEQ ID NO: 84 and a light chain variable region having an amino acid sequence with at least 95% sequence identity to SEQ ID NO: 90.

In some embodiments, the polynucleotide encodes an antibody comprising a heavy chain having a polypeptide sequence of SEQ ID NO: 94 and a light chain having the polypeptide sequence of SEQ ID NO: 96. The polynucleotide can encode an antibody comprising a heavy chain having an amino acid sequence with at least 90% sequence identity to SEQ ID NO: 94 and a light chain having an amino acid sequence with at least 90% sequence identity to SEQ ID NO: 96. The polynucleotide can encode an antibody comprising a heavy chain having an amino acid sequence with at least 95% sequence identity to SEQ ID NO: 94 and a light chain having an amino acid sequence with at least 95% sequence identity to SEQ ID NO: 96. In some embodiments, in the polynucleotide encoding the heavy chain and the light chain, the polynucleotide comprises SEQ ID NO: 93 and SEQ ID NO: 95.

Disclosed herein are nucleic acid molecules comprising, consisting essentially of, or consisting of polynucleotides encoding antibodies that immunospecifically binds to PSD-95, wherein the antibodies comprise a heavy chain (HC) and a light chain (LC) that pair to form a binding domain comprising a heavy chain complementarity determining region 1 (HCDR1) comprising the amino acid sequence of SEQ ID NO: 111, a HCDR2 comprising the amino acid sequence of SEQ ID NO: 112, a HCDR3 comprising the amino acid sequence of SEQ ID NO: 113, a light chain complementarity determining region 1 (LCDR1) comprising the amino acid sequence of SEQ ID NO: 116, a LCDR2 comprising the amino acid sequence of DTS, and a LCDR3 comprising the amino acid sequence of SEQ ID NO: 117, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge. In some embodiments, polynucleotides further encode a CH3 domain linked to the C-terminus of the third CH2 domain.

The polynucleotide can encode a first CH2 domain, a second CH2 domain, and a third CH2 domain each comprising the amino acid sequence of SEQ ID NO: 104. In some embodiments, the polynucleotide encodes a first CH2 domain, a second CH2 domain, and a third CH2 domain each comprising the amino acid sequence of SEQ ID NO: 104. The polynucleotide can encode a first CH2 domain, a second CH2 domain, and a third CH2 domain each comprising an amino acid sequence with at least 90% sequence identity to SEQ ID NO: 104. The polynucleotide can encode a first CH2 domain, a second CH2 domain, and a third CH2 domain each comprising an amino acid sequence with at least 95% sequence identity to SEQ ID NO: 104. In some embodiments, in the polynucleotide encoding the first CH2 domain, the second CH2 domain, and the third CH2 domain, the polynucleotide comprises SEQ ID NO: 103.

The polynucleotide can encode a first hinge and a second hinge each comprising the amino acid sequence of SEQ ID NO: 102. In some embodiments, the polynucleotide encodes a first hinge and a second hinge each comprising the amino acid sequence of SEQ ID NO: 102. The polynucleotide can encode a first hinge and a second hinge each comprising an amino acid sequence with at least 90% sequence identity to SEQ ID NO: 102. The polynucleotide can encode a first hinge and a second hinge each comprising an amino acid sequence with at least 95% sequence identity to SEQ ID NO: 102. In some embodiments, in the polynucleotide encoding the first hinge and the second hinge, the polynucleotide encoding each hinge comprises SEQ ID NO: 101.

The polynucleotide can encode a first linker and a second linker comprising the amino acid sequence of GS, GGS, SEQ ID NO: 14, SEQ ID NO: 38, SEQ ID NO: 43, or SEQ ID NO: 44. The polynucleotide can encode the first and the second linkers comprising GS. The first and the second linkers can comprise GGS. The polynucleotide can encode the first and the second linkers comprising SEQ ID NO: 14. The polynucleotide can encode the first and the second linkers comprising SEQ ID NO: 38. The polynucleotide can encode the first and the second linkers comprising SEQ ID NO: 43. The polynucleotide can encode a first and a second linker comprising a sequence with at least 90% sequence identity to SEQ ID NO: 43. The polynucleotide can encode the first and the second linkers comprising SEQ ID NO: 44. The polynucleotide can encode a first and a second linker comprising a sequence with at least 90% sequence identity to SEQ ID NO: 44.

In some embodiments, the polynucleotide encodes an antibody that comprises a CH3 domain comprising the amino acid sequence of SEQ ID NO: 106. In some embodiments, the polynucleotide encodes an antibody that comprises a CH3 domain having an amino acid sequence with at least 90% sequence identity to SEQ ID NO: 106. In some embodiments, the polynucleotide encodes an antibody that comprises a CH3 domain having an amino acid sequence with at least 95% sequence identity to SEQ ID NO: 106.

In some embodiments, the polynucleotide encodes an antibody comprising a heavy chain variable region having the polypeptide sequence of SEQ ID NO: 110 and a light chain variable region having the polypeptide sequence of SEQ ID NO: 115. The polynucleotide can encode an antibody comprising a heavy chain variable region having an amino acid sequence with at least 90% sequence identity to SEQ ID NO: 110 and a light chain variable region having an amino acid sequence with at least 90% sequence identity to SEQ ID NO: 115. The polynucleotide can encode an antibody comprising a heavy chain variable region having an amino acid sequence with at least 95% sequence identity to SEQ ID NO: 110 and a light chain variable region having an amino acid sequence with at least 95% sequence identity to SEQ ID NO: 115.

In some embodiments, the polynucleotide encodes an antibody comprising a heavy chain having a polypeptide sequence of SEQ ID NO: 119 and a light chain having the polypeptide sequence of SEQ ID NO: 121. The polynucleotide can encode an antibody comprising a heavy chain having an amino acid sequence with at least 90% sequence identity to SEQ ID NO: 119 and a light chain having an amino acid sequence with at least 90% sequence identity to SEQ ID NO: 121. The polynucleotide can encode an antibody comprising a heavy chain having an amino acid sequence with at least 95% sequence identity to SEQ ID NO: 119 and a light chain having an amino acid sequence with at least 95% sequence identity to SEQ ID NO: 121. In some embodiments, in the polynucleotide encoding the heavy chain and the light chain, the polynucleotide comprises SEQ ID NO: 118 and SEQ ID NO: 120.

Disclosed herein are nucleic acid molecules comprising, consisting essentially of, or consisting of polynucleotides encoding antibodies that immunospecifically binds to TIGIT, wherein the antibodies comprise a heavy chain (HC) and a light chain (LC) that pair to form a binding domain comprising a heavy chain complementarity determining region 1 (HCDR1) comprising the amino acid sequence of SEQ ID NO: 146, a HCDR2 comprising the amino acid sequence of SEQ ID NO: 147, a HCDR3 comprising the amino acid sequence of SEQ ID NO: 148, a light chain complementarity determining region 1 (LCDR1) comprising the amino acid sequence of SEQ ID NO: 151, a LCDR2 comprising the amino acid sequence of SAS, and a LCDR3 comprising the amino acid sequence of SEQ ID NO: 152, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge. In some embodiments, polynucleotides further encode a CH3 domain linked to the C-terminus of the third CH2 domain.

The polynucleotide can encode a first CH2 domain, a second CH2 domain, and a third CH2 domain each comprising the amino acid sequence of SEQ ID NO: 19. In some embodiments, the polynucleotide encodes a first CH2 domain, a second CH2 domain, and a third CH2 domain each comprising the amino acid sequence of SEQ ID NO: 19. The polynucleotide can encode a first CH2 domain, a second CH2 domain, and a third CH2 domain each comprising an amino acid sequence with at least 90% sequence identity to SEQ ID NO: 19. The polynucleotide can encode a first CH2 domain, a second CH2 domain, and a third CH2 domain each comprising an amino acid sequence with at least 95% sequence identity to SEQ ID NO: 19. In some embodiments, in the polynucleotide encoding the first CH2 domain, the second CH2 domain, and the third CH2 domain, the polynucleotide comprises SEQ ID NO: 23.

The polynucleotide can encode a first hinge and a second hinge each comprising the amino acid sequence of SEQ ID NO: 18. In some embodiments, the polynucleotide encodes a first hinge and a second hinge each comprising the amino acid sequence of SEQ ID NO: 18. The polynucleotide can encode a first hinge and a second hinge each comprising an amino acid sequence with at least 90% sequence identity to SEQ ID NO: 18. The polynucleotide can encode a first hinge and a second hinge each comprising an amino acid sequence with at least 95% sequence identity to SEQ ID NO: 18. In some embodiments, in the polynucleotide encoding the first hinge and the second hinge, the polynucleotide encoding each hinge comprises SEQ ID NO: 22.

The polynucleotide can encode a first hinge and a second hinge each comprising the amino acid sequence of SEQ ID NO: 48. In some embodiments, the polynucleotide encodes a first hinge and a second hinge each comprising the amino acid sequence of SEQ ID NO: 48. The polynucleotide can encode a first hinge and a second hinge each comprising an amino acid sequence with at least 90% sequence identity to SEQ ID NO: 48. The polynucleotide can encode a first hinge and a second hinge each comprising an amino acid sequence with at least 95% sequence identity to SEQ ID NO: 48. In some embodiments, in the polynucleotide encoding the first hinge and the second hinge, the polynucleotide encoding each hinge comprises SEQ ID NO: 51.

The polynucleotide can encode a first linker and a second linker comprising the amino acid sequence of GS, GGS, SEQ ID NO: 14, SEQ ID NO: 38, SEQ ID NO: 43, or SEQ ID NO: 44. The polynucleotide can encode the first and the second linkers comprising GS. The first and the second linkers can comprise GGS. The polynucleotide can encode the first and the second linkers comprising SEQ ID NO: 14. The polynucleotide can encode the first and the second linkers comprising SEQ ID NO: 38. The polynucleotide can encode the first and the second linkers comprising SEQ ID NO: 43. The polynucleotide can encode a first and a second linker comprising a sequence with at least 90% sequence identity to SEQ ID NO: 43. The polynucleotide can encode the first and the second linkers comprising SEQ ID NO: 44. The polynucleotide can encode a first and a second linker comprising a sequence with at least 90% sequence identity to SEQ ID NO: 44.

In some embodiments, the polynucleotide encodes an antibody that comprises a CH3 domain comprising the amino acid sequence of SEQ ID NO: 20. In some embodiments, the polynucleotide encodes an antibody that comprises a CH3 domain having an amino acid sequence with at least 90% sequence identity to SEQ ID NO: 20. In some embodiments, the polynucleotide encodes an antibody that comprises a CH3 domain having an amino acid sequence with at least 95% sequence identity to SEQ ID NO: 20.

In some embodiments, the polynucleotide encodes an antibody comprising a heavy chain variable region having the polypeptide sequence of SEQ ID NO: 145 and a light chain variable region having the polypeptide sequence of SEQ ID NO: 150. The polynucleotide can encode an antibody comprising a heavy chain variable region having an amino acid sequence with at least 90% sequence identity to SEQ ID NO: 145 and a light chain variable region having an amino acid sequence with at least 90% sequence identity to SEQ ID NO: 150. The polynucleotide can encode an antibody comprising a heavy chain variable region having an amino acid sequence with at least 95% sequence identity to SEQ ID NO: 145 and a light chain variable region having an amino acid sequence with at least 95% sequence identity to SEQ ID NO: 150.

In some embodiments, the polynucleotide encodes an antibody comprising a heavy chain having a polypeptide sequence of SEQ ID NO: 154 and a light chain having the polypeptide sequence of SEQ ID NO: 156. The polynucleotide can encode an antibody comprising a heavy chain having an amino acid sequence with at least 90% sequence identity to SEQ ID NO: 154 and a light chain having an amino acid sequence with at least 90% sequence identity to SEQ ID NO: 156. The polynucleotide can encode an antibody comprising a heavy chain having an amino acid sequence with at least 95% sequence identity to SEQ ID NO: 154 and a light chain having an amino acid sequence with at least 95% sequence identity to SEQ ID NO: 156. In some embodiments, in the polynucleotide encoding the heavy chain and the light chain, the polynucleotide comprises SEQ ID NO: 153 and SEQ ID NO: 155.

Disclosed herein are vectors comprising any of the herein described nucleic acid molecules that comprise polynucleotides encoding antibodies. Disclosed herein are also host cells comprising any of the herein disclosed vectors. Suitable host cells include eukaryotic cells and prokaryotic cells.

An antibody or polypeptide according to the present disclosure may be produced using any known and well-established expression system and recombinant cell culturing technology, for example, by expression in bacterial hosts (prokaryotic systems) or eukaryotic systems such as yeasts, fungi, insect cells or mammalian cells. An antibody or polypeptide of the present disclosure may be produced in transgenic organisms such as a goat or a plant. An antibody or polypeptide of the present disclosure may also be produced by chemical synthesis.

Disclosed herein are methods of detecting the presence of a target protein in a sample, the methods comprising, consisting essentially of, or consisting of applying any of the herein disclosed isolated polypeptides or any of the herein disclosed antibodies to a sample containing a target protein, incubating the isolated polypeptide or the antibody in the sample under conditions permitting the isolated polypeptide or the antibody to bind to the target protein and to form a bound isolated polypeptide or a bound antibody, and determining the presence of the bound isolated polypeptide or the bound antibody, thereby detecting the presence of the target protein.

Disclosed herein are methods of detecting the presence of a target protein in a sample, the methods comprising, consisting essentially of, or consisting of applying any of the herein disclosed isolated polypeptides to a sample containing a target protein, incubating the isolated polypeptide in the sample under conditions permitting the isolated polypeptide to bind to the target protein and to form a bound isolated polypeptide, and determining the presence of the bound isolated polypeptide, thereby detecting the presence of the target protein.

Disclosed herein are methods of detecting the presence of a target protein in a sample, the methods comprising, consisting essentially of, or consisting of applying any of the herein disclosed antibodies to a sample containing a target protein, incubating the antibody in the sample under conditions permitting the antibody to bind to the target protein and to form a bound antibody, and determining the presence of the bound antibody, thereby detecting the presence of the target protein.

Disclosed herein are uses of any of the herein disclosed isolated polypeptides or any of the herein disclosed antibodies in detecting the presence of a target protein in a sample. The uses can comprise, consist essentially of, or consist of applying any of the herein disclosed isolated polypeptides or any of the herein disclosed antibodies to a sample containing a target protein, incubating the isolated polypeptide or the antibody in the sample under conditions permitting the isolated polypeptide or the antibody to bind to the target protein and to form a bound isolated polypeptide or a bound antibody, and determining the presence of the bound isolated polypeptide or the bound antibody, thereby detecting the presence of the target protein.

Disclosed herein are uses of any of the herein disclosed isolated polypeptides in detecting the presence of a target protein in a sample. The uses can comprise, consist essentially of, or consist of applying any of the herein disclosed isolated polypeptides to a sample containing a target protein, incubating the isolated polypeptide in the sample under conditions permitting the isolated polypeptide to bind to the target protein and to form a bound isolated polypeptide, and determining the presence of the bound isolated polypeptide, thereby detecting the presence of the target protein.

Disclosed herein are uses of any of the herein disclosed antibodies in detecting the presence of a target protein in a sample. The uses can comprise, consist essentially of, or consist of applying any of the herein disclosed antibodies to a sample containing a target protein, incubating the antibody in the sample under conditions permitting the antibody to bind to the target protein and to form a bound antibody, and determining the presence of the bound antibody, thereby detecting the presence of the target protein.

In some embodiments of the methods or uses, a lesser amount of herein disclosed isolated polypeptide or antibody is required relative to an amount of a wild type isolated polypeptide or wild type antibody that would be required for generating a detectable signal in an equivalent method. A lesser amount of a herein disclosed isolated polypeptide can be required relative to an amount of a wild type isolated peptide that would be required for generating a detectable signal in an equivalent method. A lesser amount of a herein disclosed antibody can be required relative to an amount of a wild type antibody that would be required for generating a detectable signal in an equivalent method. The herein disclosed isolated polypeptides and antibodies provide for enhanced sensitivity at lower concentrations which allows for robust signal to be generated at concentrations at which a wild type isolated polypeptide or wild type antibody would provide less signal.

In some embodiments of the methods or uses, a lesser quantity of sample is used (e.g., rare clinical samples) but greater signal is still generated relative to an equivalent method that uses a wild type isolated polypeptide or wild type antibody.

In some embodiments of the methods or uses, a lower concentration of target protein in the sample is able to be detected relative to an equivalent method that uses a wild type isolated polypeptide or wild type antibody.

In some embodiments of the methods or uses, the herein disclosed isolated polypeptide or antibody has less background binding to cells that express Fc gamma receptors (FcγR) relative to a wild type isolated polypeptide or a wild type antibody.

In some embodiments of the methods or uses, the target protein is OCT4, Parkin, IDO1, FLAG-tag, pERK1/2, PSD-95, or TIGIT. In some embodiments of the methods or uses, the target protein is OCT4. In some embodiments of the methods or uses, the target protein is Parkin. In some embodiments of the methods or uses, the target protein is IDO1. In some embodiments of the methods or uses, the target protein is FLAG-tag. In some embodiments of the methods or uses, the target protein is pERK1/2. In some embodiments of the methods or uses, the target protein is PSD-95. In some embodiments of the methods or uses, the target is TIGIT.

The target protein can be present in the sample in low abundance. Low abundance targets are expressed or present at low levels that fall below the limit of detection or that provide a very weak signal using a wild type antibody in standard immuno applications and immunoassays. Low abundance includes situations where there is an inability or a difficulty in detecting the target protein in a particular assay using a wild type antibody.

In some embodiments of the methods or uses, the presence of the bound isolated polypeptide or the bound antibody is determined by applying a secondary antibody to said sample, wherein the secondary antibody is labeled. In some embodiments of the methods of uses, the secondary antibody is the rabbit anti-mouse (RAM) of the present disclosure.

In some embodiments of the methods or uses, the antibody is labeled. The label can be selected from a fluorophore, a fluorescent protein, a phosphorescent dye, a tandem dye, a polymer dye, a particle, an electron transfer agent, a hapten, an enzyme, a radioisotope, a chromophore, and an oligonucleotide-based fluorescent dye. The label can be a fluorophore. The label can be a fluorescent protein. The label can be a phosphorescent dye. The label can be a tandem dye. The label can be a polymer dye. The label can be a particle. The label can be an electron transfer agent. The label can be a hapten. The label can be an enzyme. The label can be a radioisotope. The label can be a chromophore. The label can be an oligonucleotide-based fluorescent dye.

In some embodiments, the label is a fluorophore. The fluorophore can be a pyrene, an anthracene, a naphthalene, an acridine, a stilbene, an indole or benzindole, an oxazole or benzoxazole, a thiazole or benzothiazole, an indacene, a 4-amino-7-nitrobenz-2-oxa-1, 3-diazole (NBD), a cyanine, a carbocyanine, a benzocyanine, a carbostyryl, a porphyrin, a salicylate, an anthranilate, an azulene, a perylene, a pyridine, a quinoline, a borapolyazaindacene, a xanthene, a rhodamine, a silicon rhodamine, a fluorescein, a rhodol, a rosamine, an oxazine or a benzoxazine, a benzophenoxazine, a carbazine, a phenalenone, a coumarin, a furan or a benzofuran, a benzophenalenone, a benzopyrilium, a styryl, a squarine, a resorufin, an anthraquinone, a quinazolinone, an oligonucleotide-based fluorescent dyes, a conjugated polymer dye, or a tandem dye. The fluorophore can be a pyrene. The fluorophore can be an anthracene. The fluorophore can be a naphthalene. The fluorophore can be an acridine. The fluorophore can be a stilbene. The fluorophore can be an indole. The fluorophore can be a benzindole. The fluorophore can be an oxazole. The fluorophore can be a benzoxazole. The fluorophore can be a thiazole. The fluorophore can be a benzothiazole. The fluorophore can be an indacene. The fluorophore can be a 4-amino-7-nitrobenz-2-oxa-1, 3-diazole (NBD). The fluorophore can be a cyanine. The fluorophore can be a carbocyanine. The fluorophore can be a benzocyanine. The fluorophore can be a carbostyryl. The fluorophore can be a porphyrin. The fluorophore can be a salicylate. The fluorophore can be an anthranilate. The fluorophore can be an azulene. The fluorophore can be a perylene. The fluorophore can be a pyridine. The fluorophore can be a quinoline. The fluorophore can be a borapolyazaindacene. The fluorophore can be a xanthene. The fluorophore can be a rhodamine. The fluorophore can be a silicon rhodamine. The fluorophore can be a fluorescein. The fluorophore can be a rhodol. The fluorophore can be a rosamine. The fluorophore can be an oxazine. The fluorophore can be a benzoxazine. The fluorophore can be a benzophenoxazine. The fluorophore can be a carbazine. The fluorophore can be a phenalenone. The fluorophore can be a coumarin. The fluorophore can be a benzofuran. The fluorophore can be a benzophenalenone. The fluorophore can be a benzopyrilium. The fluorophore can be a styryl. The fluorophore can be a squarine. The fluorophore can be a resorufin. The fluorophore can be an anthrquinone. The fluorophore can be a quinazolinone.

The fluorophore can be an oligonucleotide-based fluorescent dye. The fluorophore can be a conjugated polymer dye. The fluorophore can be a tandem dye.

In some embodiments, the label is a particle. The particle can be a semiconductor nanocrystal.

In some embodiments, the label is a hapten. The hapten can be a biotin or a biotin derivative. The hapten can be biotin. The hapten can be a biotin derivative.

In some embodiments, the label is an enzyme. The enzyme can be a peroxidase, a phosphatase, a glycosidase, and a luciferase. The enzyme can be a peroxidase.

The enzyme can be a phosphatase. The enzyme can be a glycosidase. The enzyme can be a luciferase.

The methods or uses can further comprise adding an enzymatic substrate selected from a chromogenic, a fluorogenic, and a chemiluminescent substrate. The enzymatic substate can be a chromogenic substrate. The enzymatic substate can be a fluorogenic substrate. The enzymatic substate can be a chemiluminescent substrate.

The methods or uses can comprise detecting the labeled antibody utilizing flow cytometry, immunohistochemistry, immunocytochemistry, or western blotting. The labeled antibody can be detected utilizing flow cytometry. The labeled antibody can be detected utilizing immunohistochemistry. The labeled antibody can be detected utilizing immunocytochemistry. The labeled antibody can be detected utilizing western blotting.

Sequences

TABLE 1
Anti-Parkin Antibody Variable Domains
Anti-Parkin Antibody
Clone Name: 21H24L9
Species: Rabbit
Isotype: IgG

| DNA Leader sequence, Heavy | atggagactgggctgcgctggcttctcctggtcgctgtgctcaaaggtgtccagtgt |
|---|---|
| Chain | (SEQ ID NO: 1) |
| Heavy Chain DNA Sequence | cagtcggtggaggagtccgggggtcgcctggtcacgcctgggacacccctgacact |
| | cacttgcacagtctctggattctccctcagtagctactacatgacctgggtccgccagg |
| | ctccagggaaggggctggaatacatcggatacattgcttataatggtaacacatggta |
| | cgcgagctgggcgaaaggccgattcaccatctccaaaacctcgaccacggtggatc |
| | tcaaaatcaccagtccgacaaccgaagacacggccacctatttctgtgccagagatc |
| | ggggtgatactagtggtatcgactttaacatctggggcccaggcaccctggtcaccgt |
| | ctcgagcgggcaacctaaggctccatcagttttcccactggccccctgctgcgggga |
| | cacacccagctccacggtgaccctgggctgcctggtcaaaggctacctcccggagc |
| | cagtgaccgtgacctggaactcgggcaccctcaccaatggggtacgcaccttcccgt |
| | ccgtccggcagtcctcaggcctctactcgctgagcagcgtggtgagcgtgacctcaa |
| | gcagccagcccgtcacttgcaacgtggcccacccagccaccaacaccaaagtgga |
| | caagaccgtggcgccctcgacatgcagcaagcccacgtgcccaccccctgagttcg |
| | aggggggaccgtctgtcttcatcttccccccaaaacccaaggacaccctcatgatctc |
| | acgcacccccgaggtgacctgcgtggtggtggacgtgagccaggatgaccccgag |
| | gtgcagttcacatggtacataaacaacgagcaggtccgcaccgcccggccgccgct |
| | acgggagcagcagttcaacagcacgatccgcgtggtcagcaccctccccatcgcgc |
| | accaggactggctgaggggcaaggagttcaagtgcaaagtccacaacaaggcact |
| | cccggcctccatcgagaagaccatcagcaaggccagagggggggggcggca |
| | gcggcggcggcggcagcctagcgccctcgacatgcagcaagcccacgtgcccac |
| | cccctgagttcgaggggggaccgtctgtcttcatcttccccccaaaacccaaggaca |
| | ccctcatgatctcacgcacccccgaggtgacctgcgtggtggtggacgtgagccag |
| | gatgaccccgaggtgcagttcacatggtacataaacaacgagcaggtccgcaccgc |
| | ccggccgccgctacgggagcagcagttcaacagcacgatccgcgtggtcagcacc |
| | ctccccatcgcgcaccaggactggctgaggggcaaggagttcaagtgcaaagtcca |
| | caacaaggcactcccggcctccatcgagaagaccatcagcaaggccagaggggg |
| | cggcggcggcagcggcggcggcggcagcctagcgccctcgacatgcagcaagc |
| | ccacgtgcccaccccctgagttcgaggggggaccgtctgtcttcatcttccccccaaa |
| | acccaaggacaccctcatgatctcacgcacccccgaggtgacctgcgtggtggtgg |
| | acgtgagccaggatgaccccgaggtgcagttcacatggtacataaacaacgagcag |
| | gtccgcaccgcccggccgccgctacgggagcagcagttcaacagcacgatccgcg |
| | tggtcagcaccctccccatcgcgcaccaggactggctgaggggcaaggagttcaag |
| | tgcaaagtccacaacaaggcactcccggcctccatcgagaagaccatcagcaaggc |
| | cagagggcagcccctggagccgaaggtctacaccatgggtcctccccgggaggag |
| | ctgagcagcaggtcggtcagcctggcctgcatgatcaacggcttctacccttccgaca |
| | tctcggtggagtgggagaagaacgggaaggcagaggacaactacaagaccacgc |
| | cggccgtgctggacagcgacggctcctacttcctctacagcaagctctcagtgccca |
| | cgagtgagtggcagcggggcgacgtcttcacttgctccgtgatgcacgaggccttgc |
| | acaaccactacacgcagaagtccatctcccgctctccgggtaaa (SEQ ID NO: |
| | 2) |
| Heavy Chain Amino Acid | QSVEESGGRLVTPGTPLTLTCTVSGFSLSSYYMTWVR |
| Sequence | QAPGKGLEYIGYIAYNGNTWYASWAKGRFTISKTSTT |
| | VDLKITSPTTEDTATYFCARDRGDTSGIDFNIWGPGTL |
| | VTVSSGQPKAPSVFPLAPCCGDTPSSTVTLGCLVKGYL |
| | PEPVTVTWNSGTLTNGVRTFPSVRQSSGLYSLSSVVSV |
| | TSSSQPVTCNVAHPATNTKVDKTVAPSTCSKPTCPPPE |
| | FEGGPSVFIFPPKPKDTLMISRTPEVTCVVVDVSQDDP |
| | EVQFTWYINNEQVRTARPPLREQQFNSTIRVVSTLPIA |
| | HQDWLRGKEFKCKVHNKALPASIEKTISKARGGGGG |
| | SGGGGSLAPSTCSKPTCPPPEFEGGPSVFIFPPKPKDTL |
| | MISRTPEVTCVVVDVSQDDPEVQFTWYINNEQVRTAR |
| | PPLREQQFNSTIRVVSTLPIAHQDWLRGKEFKCKVHN |
| | KALPASIEKTISKARGGGGGSGGGGSLAPSTCSKPTCP |
| | PPEFEGGPSVFIFPPKPKDTLMISRTPEVTCVVVDVSQD |
| | DPEVQFTWYINNEQVRTARPPLREQQFNSTIRVVSTLP |
| | IAHQDWLRGKEFKCKVHNKALPASIEKTISKARGQPL |
| | EPKVYTMGPPREELSSRSVSLACMINGFYPSDISVEWE |
| | KNGKAEDNYKTTPAVLDSDGSYFLYSKLSVPTSEWQ |
| | RGDVFTCSVMHEALHNHYTQKSISRSPGK (SEQ ID |
| | NO: 3) |
| Heavy Chain CDR1 Amino Acid | GFSLSSYY (SEQ ID NO: 4) |
| Sequence | |
| (IMGT) | |
| Heavy Chain CDR2 Amino Acid | IAYNGNT (SEQ ID NO: 5) |
| Sequence | |
| (IMGT) | |
| Heavy Chain CDR3 Amino Acid | ARDRGDTSGIDFNI (SEQ ID NO: 6) |
| Sequence | |
| (IMGT) | |
| Heavy Chain Variable Region | QSVEESGGRLVTPGTPLTLTCTVSGFSLSSYYMTWVR |
| Amino Acid Sequence | QAPGKGLEYIGYIA YNGNTWYASWAKGRFTISKTSTT |
| | VDLKITSPTTEDTATYFCARDRGDTSGIDFNIWGPGTL |
| | VTVSS (SEQ ID NO: 7) |
| DNA Leader sequence, Heavy | atggagactgggctgcgctggcttctcctggtcgctgtgctcaaaggtgtccagtgt |
| Chain | (SEQ ID NO: 1) |
| Heavy Chain Fab DNA sequence | cagtcggtggaggagtccgggggtcgcctggtcacgcctgggacacccctgacact |
| | cacttgcacagtctctggattctccctcagtagctactacatgacctgggtccgccagg |
| | ctccagggaaggggctggaatacatcggatacattgcttataatggtaacacatggta |
| | cgcgagctgggcgaaaggccgattcaccatctccaaaacctcgaccacggtggatc |
| | tcaaaatcaccagtccgacaaccgaagacacggccacctatttctgtgccagagatc |
| | ggggtgatactagtggtatcgactttaacatctggggcccaggcaccctggtcaccgt |
| | ctcgagcgggcaacctaaggctccatcagttttcccactggccccctgctgcgggga |
| | cacacccagctccacggtgaccctgggctgcctggtcaaaggctacctcccggagc |
| | cagtgaccgtgacctggaactcgggcaccctcaccaatggggtacgcaccttcccgt |
| | ccgtccggcagtcctcaggcctctactcgctgagcagcgtggtgagcgtgacctcaa |
| | gcagccagcccgtcacttgcaacgtggcccacccagccaccaacaccaaagtgga |
| | caagaccgtg (SEQ ID NO: 8) |
| Heavy Chain Fab Amino Acid | QSVEESGGRLVTPGTPLTLTCTVSGFSLSSYYMTWVR |
| Sequence | QAPGKGLEYIGYIA YNGNTWYASWAK GRFTISKTSTT |
| | VDLKITSPTTEDTATYFCARDRGDTSGIDFNIWGPGTL |
| | VTVSSGQPKAPSVFPLAPCCGDTPSSTVTLGCLVKGYL |
| | PEPVTVTWNSGTLTNGVRTFPSVRQSSGLYSLSSVVSV |
| | TSSSQPVTCNVAHPATNTKVDKTV (SEQ ID NO: 9) |
| DNA Leader Sequence, Light | atggacacgagggcccccactcagctgctggggctcctgctgctctggctcccaggt |
| Chain | gccacattt (SEQ ID NO: 10) |
| Light Chain DNA Sequence | gcctatgatatgacccagactccagcctctgtggaggtagctgtgggaggcacagtc |
| | accatcaagtgccaggccagtgagagcattgatacctacttagcctggtatcagcaga |
| | aaccagggcagcctcccaagcaactgatctacagggcatccactctggcatctggg |
| | gtctcatcgcggttcaaaggcagtggagctgggacacagttcactctcaccatcagc |
| | ggcgtggagtgtgccgatgctgccacttactactgtcaacaggctactagtagtagta |
| | atattgataatgctttcggcggagggaccgaggtggtggtcaaaggtgatccagttgc |
| | acctactgtcctcatcttcccaccagctgctgatcaggtggcaactggaacagtcacc |
| | atcgtgtgtgtggcgaataaatactttcccgatgtcaccgtcacctgggaggtggatgg |
| | caccacccaaacaactggcatcgagaacagtaaaacaccgcagaattctgcagattg |
| | tacctacaacctcagcagcactctgacactgaccagcacacagtacaacagccacaa |
| | agagtacacttgcaaggtgacccagggcacgacctcagtcgtccagagcttcaatag |
| | gggtgactgt (SEQ ID NO: 11) |
| Light Chain Amino Acid | AYDMTQTPASVEVAVGGTVTIKCQASESIDTYLAWY |
| Sequence | QQKPGQPPKQLIYRASTLASGVSSRFKGSGAGTQFTLT |
| | ISGVECADAATYYCQQATSSSNIDNAFGGGTEVVVKG |
| | DPVAPTVLIFPPAADQVATGTVTIVCVANKYFPDVTV |
| | TWEVDGTTQTTGIENSKTPQNSADCTYNLSSTLTLTST |
| | QYNSHKEYTCKVTQGTTSVVQSFNRGDC (SEQ ID NO: |
| | 12) |
| Light Chain CDR1 Amino Acid | ESIDTY (SEQ ID NO: 13) |
| Sequence | |
| (IMGT) | |
| Light Chain CDR2 Amino Acid | RAS |
| Sequence | |
| (IMGT) | |
| Light Chain CDR3 Amino Acid | QQATSSSNIDNA (SEQ ID NO: 15) |
| Sequence | |
| (IMGT) | |
| Light Chain Variable Region | AYDMTQTPASVEVAVGGTVTIKCQASESIDTYLAWY |
| Amino Acid Sequence | QQKPGQPPKQLIYRASTLASGVSSRFKGSGAGTQFTLT |
| | ISGVECADAATYYCQQATSSSNIDNAFGGGTEVVVKG |
| | (SEQ ID NO: 16) |
| DNA Leader Sequence, Light | atggacacgagggcccccactcagctgctggggctcctgctgctctggctcccaggt |
| Chain | gccacattt (SEQ ID NO: 10) |
| Light Chain Full Length DNA | gcctatgatatgacccagactccagcctctgtggaggtagctgtgggaggcacagtc |
| sequence | accatcaagtgccaggccagtgagagcattgatacctacttagcctggtatcagcaga |
| | aaccagggcagcctcccaagcaactgatctacagggcatccactctggcatctggg |
| | gtctcatcgcggttcaaaggcagtggagctgggacacagttcactctcaccatcagc |
| | ggcgtggagtgtgccgatgctgccacttactactgtcaacaggctactagtagtagta |
| | atattgataatgctttcggcggagggaccgaggtggtggtcaaaggtgatccagttgc |
| | acctactgtcctcatcttcccaccagctgctgatcaggtggcaactggaacagtcacc |
| | atcgtgtgtgtggcgaataaatactttcccgatgtcaccgtcacctgggaggtggatgg |
| | caccacccaaacaactggcatcgagaacagtaaaacaccgcagaattctgcagattg |
| | tacctacaacctcagcagcactctgacactgaccagcacacagtacaacagccacaa |
| | agagtacacttgcaaggtgacccagggcacgacctcagtcgtccagagcttcaatag |
| | gggtgactgt (SEQ ID NO: 11) |
| Light Chain Full | AYDMTQTPASVEVAVGGTVTIKCQASESIDTYLAWY |
| Length Amino | QQKPGQPPKQLIYRASTLASGVSSRFKGSGAGTQFTLT |
| Acid sequence | ISGVECADAATYYCQQATSSSNIDNAFGGGTEVVVKG |
| | DPVAPTVLIFPPAADQVATGTVTIVCVANKYFPDVTV |
| | TWEVDGTTQTTGIENSKTPQNSADCTYNLSSTLTLTST |
| | QYNSHKEYTCKVTQGTTSVVQSFNRGDC (SEQ ID NO: |
| | 12) |

TABLE 2
Anti-Parkin Parental Antibody Constant Region
Anti-Parkin Parental Antibody Heavy Chain Constant Region Amino Acid Sequence
(Underlined residues are mutations in LFLEPS variants)

| | |
|---|---|
| CH1 Domain Amino Acid | GQPKAPSVFPLAPCCGDTPSSTVTLGCLVKGYLPEPVT |
| Sequence | VTWNSGTLTNGVRTFPSVRQSSGLYSLSSVVSVTSSSQ |
| | PVTCNVAHPATNTK VDKTV (SEQ ID NO: 17) |
| Hinge Amino Acid Sequence | APSTCSKPTCPPPELLGGP (SEQ ID NO: 18) |
| CH2 Domain Amino Acid | SVFIFPPKPKDTLMISRTPEVTCVVVDVSQDDPEVQFT |
| sequence | WYINNEQVRTARPPLREQQFNSTIRVVSTLPIAHQDW |
| | LRGKEFKCKVHNKALPAPIEKTISKARG (SEQ ID NO: |
| | 19) |
| CH3 Domain Amino Acid | QPLEPKVYTMGPPREELSSRSVSLACMINGFYPSDISV |
| Sequence | EWEKNGKAEDNYKTTPAVLDSDGSYFLYSKLSVPTSE |
| | WQRGDVFTCSVMHEALHNHYTQKSISRSPGK (SEQ ID |
| | NO: 20) |
| CH1 Domain DNA Sequence | gggcaacctaaggctccatcagttttcccactggccccctgctgcggggacacaccc |
| | agctccacggtgaccctgggctgcctggtcaaaggctacctcccggagccagtgac |
| | cgtgacctggaactcgggcaccctcaccaatggggtacgcaccttcccgtccgtccg |
| | gcagtcctcaggcctctactcgctgagcagcgtggtgagcgtgacctcaagcagcca |
| | gcccgtcacttgcaacgtggcccacccagccaccaacaccaaagtggacaagacc |
| | gtg (SEQ ID NO: 21) |
| Hinge DNA Sequence | gcgccctcgacatgcagcaagcccacgtgcccaccccctgagctcctggggggac |
| | cg (SEQ ID NO: 22) |
| CH2 Domain DNA sequence | tctgtcttcatcttccccccaaaacccaaggacaccctcatgatctcacgcacccccga |
| | ggtgacctgcgtggtggtggacgtgagccaggatgaccccgaggtgcagttcacat |
| | ggtacataaacaacgagcaggtccgcaccgcccggccgccgctacgggagcagc |
| | agttcaacagcacgatccgcgtggtcagcaccctccccatcgcgcaccaggactgg |
| | ctgaggggcaaggagttcaagtgcaaagtccacaacaaggcactcccggcccccat |
| | cgagaagaccatcagcaaggccagaggg (SEQ ID NO: 23) |
| CH3 Domain DNA Sequence | cagcccctggagccgaaggtctacaccatgggtcctccccgggaggagctgagca |
| | gcaggtcggtcagcctggcctgcatgatcaacggcttctacccttccgacatctcggt |
| | ggagtgggagaagaacgggaaggcagaggacaactacaagaccacgccggccg |
| | tgctggacagcgacggctcctacttcctctacagcaagctctcagtgcccacgagtga |
| | gtggcagcggggcgacgtcttcacttgctccgtgatgcacgaggccttgcacaacca |
| | ctacacgcagaagtccatctcccgctctccgggtaaa (SEQ ID NO: 24) |
| Anti-Parkin Parental Antibody | DPVAPTVLIFPPAADQVATGTVTIVCVANKYFPDVTV |
| Light Chain Constant Region | TWEVDGTTQTTGIENSKTPQNSADCTYNLSSTLTLTST |
| Amino Acid Sequence | QYNSHKEYTCKVTQGTTSVVQSFNRGDC (SEQ ID NO: |
| | 25) |
| Anti-Parkin Parental Antibody | gatccagttgcacctactgtcctcatcttcccaccagctgctgatcaggtggcaactgg |
| Light Chain Constant Region | aacagtcaccatcgtgtgtgtggcgaataaatactttcccgatgtcaccgtcacctggg |
| DNA Sequence | aggtggatggcaccacccaaacaactggcatcgagaacagtaaaacaccgcagaat |
| | tctgcagattgtacctacaacctcagcagcactctgacactgaccagcacacagtaca |
| | acagccacaaagagtacacttgcaaggtgacccagggcacgacctcagtcgtccag |
| | agcttcaataggggtgactgt (SEQ ID NO: 26) |

TABLE 3
Anti-OCT4 Antibody Variable Domains
Anti-OCT4 Antibody
Clone Name: 3H8L6
Species: Rabbit
Isotype: IgG

| | |
|---|---|
| DNA Leader sequence, Heavy | atggagactgggctgcgctggcttctcctggtcgctgtgctcaaaggtgtccagtgt |
| Chain | (SEQ ID NO: 1) |
| Heavy Chain DNA Sequence | cagtcggtggaggagtccgggggtcgcctggtcacgcctgggacacccctgacactc |
| | acttgcacagtctctggattctccctcagtaactatgcaatgagctgggtccgccagg |
| | ctccagggaaggggctggaatggatcggaatcgttagtattagtgctggcacatacta |
| | cgcgagctgggcgaaaggccgattcaccatctccaaaacctcgaccacggtggttct |
| | gagagtcaccagtccgacaaccgaggacacggccacctatttctgtgccacagggg |
| | gttatactggtaacgtctggggcccaggcaccctggtcaccgtctcgaggggcaac |
| | ctaaggctccatcagtcttcccactggccccctgctgcggggacacacccagctcca |
| | cggtgaccctgggctgcctggtcaaaggctacctcccggagccagtgaccgtgacc |
| | tggaactcgggcaccctcaccaatggggtacgcaccttcccgtccgtccggcagtcc |
| | tcaggcctctactcgctgagcagcgtggtgagcgtgacctcaagcagccagcccgtc |
| | acttgcaacgtggcccacccagccaccaacaccaaagtggacaagaccgtggcgc |
| | cctcgacatgcagcaagcccacgtgcccaccccctgagttcgaggggggaccgtct |
| | gtcttcatcttccccccaaaacccaaggacaccctcatgatctcacgcacccccgagg |
| | tgacctgcgtggtggtggacgtgagccaggatgaccccgaggtgcagttcacatggt |
| | acataaacaacgagcaggtccgcaccgcccggccgccgctacgggagcagcagtt |
| | caacagcacgatccgcgtggtcagcaccctccccatcgcgcaccaggactggctga |
| | ggggcaaggagttcaagtgcaaagtccacaacaaggcactcccggcctccatcgag |
| | aagaccatcagcaaggccagagggggcggcggcggcagcggcggcggcggca |
| | gcctagcgccctcgacatgcagcaagcccacgtgcccaccccctgagttcgagggg |
| | ggaccgtctgtcttcatcttccccccaaaacccaaggacaccctcatgatctcacgca |
| | cccccgaggtgacctgcgtggtggtggacgtgagccaggatgaccccgaggtgca |
| | gttcacatggtacataaacaacgagcaggtccgcaccgcccggccgccgctacggg |
| | agcagcagttcaacagcacgatccgcgtggtcagcaccctccccategcgcaccag |
| | gactggctgaggggcaaggagttcaagtgcaaagtccacaacaaggcactcccgg |
| | cctccatogagaagaccatcagcaaggccagagggggcggcggcggcagcggc |
| | ggcggcggcagcctagcgccctcgacatgcagcaagcccacgtgcccaccccctg |
| | agttcgaggggggaccgtctgtcttcatcttccccccaaaacccaaggacaccctcat |
| | gatctcacgcacccccgaggtgacctgcgtggtggtggacgtgagccaggatgacc |
| | ccgaggtgcagttcacatggtacataaacaacgagcaggtccgcaccgcccggccg |
| | ccgctacgggagcagcagttcaacagcacgatccgcgtggtcagcaccctccccat |
| | cgcgcaccaggactggctgaggggcaaggagttcaagtgcaaagtccacaacaag |
| | gcactcccggcctccatcgagaagaccatcagcaaggccagagggcagcccctgg |
| | agccgaaggtctacaccatgggtcctccccgggaggagctgagcagcaggtcggt |
| | cagcctggcctgcatgatcaacggcttctacccttccgacatctcggtggagtggg |
| | agaagaacgggaaggcagaggacaactacaagaccacgccggccgtgctggacagc |
| | gacggctcctacttcctctacagcaagctctcagtgcccacgagtgagtggcagcgg |
| | ggcgacgtcttcacttgctccgtgatgcacgaggccttgcacaaccactacacgcag |
| | aagtccatctcccgctctccgggtaaa (SEQ ID NO: 27) |
| Heavy Chain Amino Acid | QSVEESGGRLVTPGTPLTLTCTVSGFSLSNYAMSWVR |
| Sequence | QAPGKGLEWIGIVSISAGTYYASWAKGRFTISKTSTTV |
| | VLRVTSPTTEDTATYFCATGGYTGNVWGPGTLVTVSS |
| | GQPKAPSVFPLAPCCGDTPSSTVTLGCLVKGYLPEPVT |
| | VTWNSGTLTNGVRTFPSVRQSSGLYSLSSVVSVTSSSQ |
| | PVTCNVAHPATNTKVDKTVAPSTCSKPTCPPPEFEGGP |
| | SVFIFPPKPKDTLMISRTPEVTCVVVDVSQDDPEVQFT |
| | WYINNEQVRTARPPLREQQFNSTIRVVSTLPIAHQDW |
| | LRGKEFKCKVHNKALPASIEKTISKARGGGGGSGGGG |
| | SLAPSTCSKPTCPPPEFEGGPSVFIFPPKPKDTLMISR |
| | TPEVTCVVVDVSQDDPEVQFTWYINNEQVRTARPPLRE |
| | QQFNSTIRVVSTLPIAHQDWLRGKEFKCKVHNKALPA |
| | SIEKTISKARGGGGGSGGGGSLAPSTCSKPTCPPPEFEG |
| | GPSVFIFPPKPKDTLMISRTPEVTCVVVDVSQDDPEVQ |
| | FTWYINNEQVRTARPPLREQQFNSTIRVVSTLPIAHQD |
| | WLRGKEFKCKVHNKALPASIEKTISKARGQPLEPKVY |
| | TMGPPREELSSRSVSLACMINGFYPSDISVEWEKNGK |
| | AEDNYKTTPAVLDSDGSYFLYSKLSVPTSEWQRGDVF |
| | TCSVMHEALHNHYTQKSISRSPGK (SEQ ID NO: 28) |
| Heavy Chain CDR1 Amino Acid | GFSLSNYA (SEQ ID NO: 29) |
| Sequence (IMGT) | |
| Heavy Chain CDR2 Amino Acid | VSISAGT (SEQ ID NO: 30) |
| Sequence (IMGT) | |
| Heavy Chain CDR3 Amino Acid | ATGGYTGNV (SEQ ID NO: 31) |
| Sequence (IMGT) | |
| Heavy Chain Variable Region | QSVEESGGRLVTPGTPLTLTCTVSGFSLSNYAMSWVR |
| Amino Acid Sequence | QAPGKGLEWIGIVSISAGTYYASWAKGRFTISKTSTTV |
| | VLRVTSPTTEDTATYFCATGGYTGNVWGPGTLVTVSS |
| | (SEQ ID NO: 32) |
| DNA Leader Sequence, Heavy | atggagactgggctgcgctggcttctcctggtcgctgtgctcaaaggtgtccagtgt |
| Chain | (SEQ ID NO: 1) |
| Heavy Chain Fab DNA sequence | cagtcggtggaggagtccgggggtcgcctggtcacgcctgggacacccctgacact |
| | cacttgcacagtctctggattctccctcagtaactatgcaatgagctgggtccgccagg |
| | ctccagggaaggggctggaatggatcggaatcgttagtattagtgctggcacatacta |
| | cgcgagctgggcgaaaggccgattcaccatctccaaaacctcgaccacggtggttct |
| | gagagtcaccagtccgacaaccgaggacacggccacctatttctgtgccacagggg |
| | gttatactggtaacgtctggggcccaggcaccctggtcaccgtctcgagcgggcaac |
| | ctaaggctccatcagtcttcccactggccccctgctgcggggacacacccagctcca |
| | cggtgaccctgggctgcctggtcaaaggctacctcccggagccagtgaccgtgacc |
| | tggaactcgggcaccctcaccaatggggtacgcaccttcccgtccgtccggcagtcc |
| | tcaggcctctactcgctgagcagcgtggtgagcgtgacctcaagcagccagcccgtc |
| | acttgcaacgtggcccacccagccaccaacaccaaagtggacaagaccgtg |
| | (SEQ ID NO: 33) |
| Heavy Chain Fab Amino Acid | QSVEESGGRLVTPGTPLTLTCTVSGFSLSNYAMSWVR |
| Sequence | QAPGKGLEWIGIVSISAGTYYASWAKGRFTISKTSTTV |
| | VLRVTSPTTEDTATYFCATGGYTGNVWGPGTLVTVSS |
| | GQPKAPSVFPLAPCCGDTPSSTVTLGCLVKGYLPEPVT |
| | VTWNSGTLTNGVRTFPSVRQSSGLYSLSSVVSVTSSSQ |
| | PVTCNVAHPATNTKVDKTV (SEQ ID NO: 34) |
| DNA Leader Sequence, Light | atggacacgagggcccccactcagctgctggggctcctgctgctctggctcccaggt |
| Chain | gccacattt (SEQ ID NO: 10) |
| Light Chain DNA Sequence | gcgcaagttctgacccagactgcatcgtccgtgtctgcagctgtgggaggcacagtc |
| | accattagttgccagtccagtcagagtgtttataataagaattggttaggctggtatcag |
| | cagaaaccagggcagcctcccaggctcctgatctatggtgcatccactctggcatctg |
| | gggtcccatcgcggttcagcggcagtggatctgggacacagttcactctcaccatca |
| | gcgacgtgcagtgtgacgatgctgccacttactactgtgcaggcggttatagtaataat |
| | attgttagtttcggcggaggaaccagggtggtcgtcgaagttgctacattggcacctac |
| | tgtcctcatcttcccaccatctcctgctgagctggcaactggaacagccaccatcgtgt |
| | gcgtggcaaacaaatactttcccgatggcaccgtcacctggcaggtcgatggcaagc |
| | ccctaacaactggcatcgagaccagtaaaacaccgcagaattctgatgattgtaccta |
| | caacctcagcagtactctgacactgaaaagcgacgagtacaacagccacgacgagt |
| | acacttgccaggtggcccagggctcaggctcaccggtcgtccagagcttcaatagg |
| | ggtgactgt (SEQ ID NO: 35) |
| Light Chain Amino Acid | AQVLTQTASSVSAAVGGTVTISCQSSQSVYNKNWLG |
| Sequence | WYQQKPGQPPRLLIYGASTLASGVPSRFSGSGSGTQFT |
| | LTISDVQCDDAATYYCAGGYSNNIVSFGGGTRVVVEV |
| | ATLAPTVLIFPPSPAELATGTATIVCVANKYFPDGTVT |
| | WQVDGKPLTTGIETSKTPQNSDDCTYNLSSTLTLKSD |
| | EYNSHDEYTCQVAQGSGSPVVQSFNRGDC (SEQ ID |
| | NO: 36) |
| Light Chain CDR1 Amino Acid | QSVYNKNW (SEQ ID NO: 37) |
| Sequence (IMGT) | |
| Light Chain CDR2 Amino Acid | GAS |
| Sequence (IMGT) | |
| Light Chain CDR3 Amino Acid | AGGYSNNIVS (SEQ ID NO: 39) |
| Sequence (IMGT) | |
| Light Chain Variable Region | AQVLTQTASSVSAAVGGTVTISCQSSQSVYNKNWLG |
| Amino Acid Sequence | WYQQKPGQPPRLLIYGASTLASGVPSRFSGSGSGTQFT |
| | LTISDVQCDDAATYYCAGGYSNNIVSFGGGTRVVVE |
| | (SEQ ID NO: 40) |
| DNA Leader sequence, Light | atggacacgagggcccccactcagctgctggggctcctgctgctctggctcccaggt |
| Chain | gccacattt (SEQ ID NO: 10) |
| Light Chain DNA sequence | gcgcaagttctgacccagactgcatcgtccgtgtctgcagctgtgggaggcacagtc |
| | accattagttgccagtccagtcagagtgtttataataagaattggttaggctggtatcag |
| | cagaaaccagggcagcctcccaggctcctgatctatggtgcatccactctggcatctg |
| | gggtcccatcgcggttcagcggcagtggatctgggacacagttcactctcaccatca |
| | gcgacgtgcagtgtgacgatgctgccacttactactgtgcaggcggttatagtaataat |
| | attgttagtttcggcggaggaaccagggtggtcgtcgaagttgctacattggcacctac |
| | tgtcctcatcttcccaccatctcctgctgagctggcaactggaacagccaccatcgtgt |
| | gcgtggcaaacaaatactttcccgatggcaccgtcacctggcaggtcgatggcaagc |
| | ccctaacaactggcatcgagaccagtaaaacaccgcagaattctgatgattgtaccta |
| | caacctcagcagtactctgacactgaaaagcgacgagtacaacagccacgacgagt |
| | acacttgccaggtggcccagggctcaggctcaccggtcgtccagagcttcaatagg |
| | ggtgactgt (SEQ ID NO: 35) |
| Light Chain Amino Acid | AQVLTQTASSVSAAVGGTVTISCQSSQSVYNKNWLGW |
| sequence | YQQKPGQPPRLLIYGASTLASGVPSRFSGSGSGTQFT |
| | LTISDVQCDDAATYYCAGGYSNNIVSFGGGTRVVVEV |
| | ATLAPTVLIFPPSPAELATGTATIVCVANKYFPDGTV |
| | TWQVDGKPLTTGIETSKTPQNSDDCTYNLSSTLTLKS |
| | DEYNSHDEYTCQVAQGSGSPVVQSFNRGDC (SEQ ID |
| | NO: 36) |

TABLE 4
Anti-OCT4 Parental Antibody Constant Region
Anti-OCT4 Parental Antibody Heavy Chain Constant Region Amino Acid Sequence
(Underlined residues are mutations in LFLEPS variants)

| | |
|---|---|
| CH1 Domain Amino Acid | GQPKAPSVFPLAPCCGDTPSSTVTLGCLVKGYLPEPVT |
| Sequence | VTWNSGTLTNGVRTFPSVRQSSGLYSLSSVVSVTSSSQ |
| | PVTCNVAHPATNTKVDKTV (SEQ ID NO: 17) |
| Hinge Amino Acid Sequence | APSTCSKPTCPPPELLGGP (SEQ ID NO: 18) |
| CH2 domain Amino Acid | SVFIFPPKPKDTLMISRTPEVTCVVVDVSQDDPEVQFT |
| Sequence | WYINNEQVRTARPPLREQQFNSTIRVVSTLPIAHQDW |
| | LRGKEFKCKVHNKALPAPIEKTISKARG (SEQ ID NO: |
| | 19) |
| CH3 Domain Amino Acid | QPLEPKVYTMGPPREELSSRSVSLACMINGFYPSDISV |
| Sequence | EWEKNGKAEDNYKTTPAVLDSDGSYFLYSKLSVPTSE |
| | WQRGDVFTCSVMHEALHNHYTQKSISRSPGK (SEQ ID |
| | NO: 20) |
| CH1 Domain DNA Sequence | gggcaacctaaggctccatcagtcttcccactggccccctgctgoggggacacaccc |
| | agctccacggtgaccctgggctgcctggtcaaaggctacctcccggagccagtgac |
| | cgtgacctggaactcgggcaccctcaccaatggggtacgcaccttcccgtccgtccg |
| | gcagtcctcaggcctctactcgctgagcagcgtggtgagcgtgacctcaagcagcca |
| | gcccgtcacttgcaacgtggcccacccagccaccaacaccaaagtggacaagacc |
| | gtg (SEQ ID NO: 176) |
| Hinge DNA Sequence | gcgccctcgacatgcagcaagcccacgtgcccaccccctgagctcctggggggac |
| | cg (SEQ ID NO: 22) |
| CH2 Domain DNA sequence | tctgtcttcatcttccccccaaaacccaaggacaccctcatgatctcacgcacccccga |
| | ggtgacctgcgtggtggtggacgtgagccaggatgaccccgaggtgcagttcacat |
| | ggtacataaacaacgagcaggtccgcaccgcccggccgccgctacgggagcagc |
| | agttcaacagcacgatccgcgtggtcagcaccctccccategcgcaccaggactgg |
| | ctgaggggcaaggagttcaagtgcaaagtccacaacaaggcactcccggcccccat |
| | cgagaagaccatcagcaaggccagaggg (SEQ ID NO: 23) |
| CH3 Domain DNA Sequence | cagcccctggagccgaaggtctacaccatgggtcctccccgggaggagctgagca |
| | gcaggtcggtcagcctggcctgcatgatcaacggcttctacccttccgacatctcggt |
| | ggagtgggagaagaacgggaaggcagaggacaactacaagaccacgccggccg |
| | tgctggacagcgacggctcctacttcctctacagcaagctctcagtgcccacgagtga |
| | gtggcagcggggcgacgtcttcacttgctccgtgatgcacgaggccttgcacaacca |
| | ctacacgcagaagtccatctcccgctctccgggtaaa (SEQ ID NO: 24) |
| Anti-OCT4 Parental Antibody | VATLAPTVLIFPPSPAELATGTATIVCVANKYFPDGTV |
| Light Chain Constant Region | TWQVDGKPLTTGIETSKTPQNSDDCTYNLSSTLTLKS |
| Amino Acid Sequence | DEYNSHDEYTCQVAQGSGSPVVQSFNRGDC (SEQ ID |
| | NO: 41) |
| Anti-OCT4 Parental Antibody | gttgctacattggcacctactgtcctcatcttcccaccatctcctgctgagctggcaa |
| Light Chain Constant Region | ctggaacagccaccatcgtgtgcgtggcaaacaaatactttcccgatggcaccgtcacc |
| DNA Sequence | tggcaggtcgatggcaagcccctaacaactggcatcgagaccagtaaaacaccgcaga |
| | attctgatgattgtacctacaacctcagcagtactctgacactgaaaagcgacgagt |
| | acaacagccacgacgagtacacttgccaggtggcccagggctcaggctcaccggtc |
| | gtccagagcttcaataggggtgactgt (SEQ ID NO: 42) |

TABLE 5
Rabbit 2TdCH2 Domain Amino Acid Sequence
(Underlined residues are mutations in LFLEPS variants)

| | |
|---|---|
| Hinge 1 Amino Acid Sequence | APSTCSKPTCPPPELLGGP (SEQ ID NO: 18) |
| First CH2 Domain Amino Acid | SVFIFPPKPKDTLMISRTPEVTCVVVDVSQDDPEVQFT |
| Sequence | WYINNEQVRTARPPLREQQFNSTIRVVSTLPIAHQDW |
| | LRGKEFKCKVHNKALPAPIEKTISKARG (SEQ ID NO: |
| | 19) |
| Linker 1 Amino Acid Sequence | GGGGSGGGGSL (SEQ ID NO: 44) |
| Hinge 2 Amino Acid Sequence | APSTCSKPTCPPPELLGGP (SEQ ID NO: 18) |
| Second CH2 Domain Amino | SVFIFPPKPKDTLMISRTPEVTCVVVDVSQDDPEVQFT |
| Acid Sequence | WYINNEQVRTARPPLREQQFNSTIRVVSTLPIAHQDW |
| | LRGKEFKCKVHNKALPAPIEKTISKARG (SEQ ID NO: |
| | 19) |
| Linker 2 Amino Acid Sequence | GGGGSGGGGSL (SEQ ID NO: 44) |
| Hinge 3 Amino Acid Sequence | APSTCSKPTCPPPELLGGP (SEQ ID NO: 18) |
| Third CH2 domain Amino Acid | SVFIFPPKPKDTLMISRTPEVTCVVVDVSQDDPEVQFT |
| Sequence | WYINNEQVRTARPPLREQQFNSTIRVVSTLPIAHQDW |
| | LRGKEFKCKVHNKALPAPIEKTISKARG (SEQ ID NO: |
| | 19) |
| Fc Domain (only CH2 + CH3) | SVFIFPPKPKDTLMISRTPEVTCVVVDVSQDDPEVQFT |
| Amino Acid Sequence | WYINNEQVRTARPPLREQQFNSTIRVVSTLPIAHQDW |
| | LRGKEFKCKVHNKALPAPIEKTISKARGQPLEPKVYT |
| | MGPPREELSSRSVSLACMINGFYPSDISVEWEKNGKA |
| | EDNYKTTPAVLDSDGSYFLYSKLSVPTSEWQRGDVFT |
| | CSVMHEALHNHYTQKSISRSPGK (SEQ ID NO: 45) |
| 2TdCH2 Fc Domain | APSTCSKPTCPPPELLGGPSVFIFPPKPKDTLMISRTPE |
| | VTCVVVDVSQDDPEVQFTWYINNEQVRTARPPLREQQF |
| | NSTIRVVSTLPIAHQDWLRGKEFKCKVHNKALPAPIEK |
| | TISKARGGGGGSGGGGSLAPSTCSKPTCPPPELLGGPS |
| | VFIFPPKPKDTLMISRTPEVTCVVVDVSQDDPEVQFTW |
| | YINNEQVRTARPPLREQQFNSTIRVVSTLPIAHQDWLR |
| | GKEFKCKVHNKALPAPIEKTISKARGGGGGSGGGGSLA |
| | PSTCSKPTCPPPELLGGPSVFIFPPKPKDTLMISRTPEV |
| | TCVVVDVSQDDPEVQFTWYINNEQVRTARPPLREQQF |
| | NSTIRVVSTLPIAHQDWLRGKEFKCKVHNKALPAPIEK |
| | TISKARGQPLEPKVYTMGPPREELSSRSVSLACMINGF |
| | YPSDISVEWEKNGKAEDNYKTTPAVLDSDGSYFLYSK |
| | LSVPTSEWQRGDVFTCSVMHEALHNHYTQKSISRSPG |
| | K (SEQ ID NO: 52) |

TABLE 6
Rabbit 2TdCH2 Domain DNA Sequence
(Underlined residues are mutations in LFLEPS variants)

| | |
|---|---|
| Hinge 1 DNA Sequence | gcgccctcgacatgcagcaagcccacgtgcccaccccctgagctcctggggggac |
| | cg (SEQ ID NO: 22) |
| First CH2 Domain DNA | tctgtcttcatcttccccccaaaacccaaggacaccctcatgatctcacgcacccccga |
| Sequence | ggtgacctgcgtggtggtggacgtgagccaggatgaccccgaggtgcagttcacat |
| | ggtacataaacaacgagcaggtccgcaccgcccggccgccgctacgggagcagc |
| | agttcaacagcacgatccgcgtggtcagcaccctccccatcgcgcaccaggactgg |
| | ctgaggggcaaggagttcaagtgcaaagtccacaacaaggcactcccggcccccat |
| | cgagaagaccatcagcaaggccagaggg (SEQ ID NO: 23) |
| Linker 1 DNA Sequence | ggcggcggcggcagcggcggcggcggcagccta (SEQ ID NO: 46) |
| Hinge 2 DNA Sequence | gcgccctcgacatgcagcaagcccacgtgcccaccccctgagctcctggggggac |
| | cg (SEQ ID NO: 22) |
| Second CH2 domain DNA | tctgtcttcatcttccccccaaaacccaaggacaccctcatgatctcacgcacccccga |
| Sequence | ggtgacctgcgtggtggtggacgtgagccaggatgaccccgaggtgcagttcacat |
| | ggtacataaacaacgagcaggtccgcaccgcccggccgccgctacgggagcagc |
| | agttcaacagcacgatccgcgtggtcagcaccctccccatcgcgcaccaggactgg |
| | ctgaggggcaaggagttcaagtgcaaagtccacaacaaggcactcccggcccccat |
| | cgagaagaccatcagcaaggccagaggg (SEQ ID NO: 23) |
| Linker 2 DNA Sequence | ggcggcggcggcagcggcggcggcggcagccta (SEQ ID NO: 46) |
| Hinge 3 DNA Sequence | gcgccctcgacatgcagcaagcccacgtgcccaccccctgagctcctggggggac |
| | cg (SEQ ID NO: 22) |
| Third CH2 domain DNA | tctgtcttcatcttccccccaaaacccaaggacaccctcatgatctcacgcacccccga |
| Sequence | ggtgacctgcgtggtggtggacgtgagccaggatgaccccgaggtgcagttcacat |
| | ggtacataaacaacgagcaggtccgcaccgcccggccgccgctacgggagcagc |
| | agttcaacagcacgatccgcgtggtcagcaccctccccatcgcgcaccaggactgg |
| | ctgaggggcaaggagttcaagtgcaaagtccacaacaaggcactcccggcccccat |
| | cgagaagaccatcagcaaggccagaggg (SEQ ID NO: 23) |
| Fc Domain (only CH2 + CH3) | tctgtcttcatcttccccccaaaacccaaggacaccctcatgatctcacgcacccccga |
| DNA Sequence | ggtgacctgcgtggtggtggacgtgagccaggatgaccccgaggtgcagttcacat |
| | ggtacataaacaacgagcaggtccgcaccgcccggccgccgctacgggagcagc |
| | agttcaacagcacgatccgcgtggtcagcaccctccccatcgcgcaccaggactgg |
| | ctgaggggcaaggagttcaagtgcaaagtccacaacaaggcactcccggcccccat |
| | cgagaagaccatcagcaaggccagagggcagcccctggagccgaaggtctacac |
| | catgggtcctccccgggaggagctgagcagcaggtcggtcagcctggcctgcatga |
| | tcaacggcttctacccttccgacatctcggtggagtgggagaagaacgggaaggca |
| | gaggacaactacaagaccacgccggccgtgctggacagcgacggctcctacttcct |
| | ctacagcaagctctcagtgcccacgagtgagtggcagcggggcgacgtcttcacttg |
| | ctccgtgatgcacgaggccttgcacaaccactacacgcagaagtccatctcccgctct |
| | ccgggtaaa (SEQ ID NO: 47) |
| 2TdCH2 Fc Domain DNA | gcgccctcgacatgcagcaagcccacgtgcccaccccctgagctcctggggggac |
| Sequence | cgtctgtcttcatcttccccccaaaacccaaggacaccctcatgatctcacgcaccccc |
| | gaggtgacctgcgtggtggtggacgtgagccaggatgaccccgaggtgcagttcac |
| | atggtacataaacaacgagcaggtccgcaccgcccggccgccgctacgggagcag |
| | cagttcaacagcacgatccgcgtggtcagcaccctccccatcgcgcaccaggactg |
| | gctgaggggcaaggagttcaagtgcaaagtccacaacaaggcactcccggccccc |
| | atcgagaagaccatcagcaaggccagagggggcggcggcggcagcggcggcgg |
| | cggcagcctagcgccctcgacatgcagcaagcccacgtgcccaccccctgagctcc |
| | tggggggaccgtctgtcttcatcttccccccaaaacccaaggacaccctcatgatctc |
| | acgcacccccgaggtgacctgcgtggtggtggacgtgagccaggatgaccccgag |
| | gtgcagttcacatggtacataaacaacgagcaggtccgcaccgcccggccgccgct |
| | acgggagcagcagttcaacagcacgatccgcgtggtcagcaccctccccatcgcgc |
| | accaggactggctgaggggcaaggagttcaagtgcaaagtccacaacaaggcact |
| | cccggcccccatcgagaagaccatcagcaaggccagagggggcggcggcggca |
| | gcggcggcggcggcagcctagcgccctcgacatgcagcaagcccacgtgcccac |
| | cccctgagctcctggggggaccgtctgtcttcatcttccccccaaaacccaaggacac |
| | cctcatgatctcacgcacccccgaggtgacctgcgtggtggtggacgtgagccagg |
| | atgaccccgaggtgcagttcacatggtacataaacaacgagcaggtccgcaccgcc |
| | cggccgccgctacgggagcagcagttcaacagcacgatccgcgtggtcagcaccc |
| | tccccatcgcgcaccaggactggctgaggggcaaggagttcaagtgcaaagtccac |
| | aacaaggcactcccggcccccatcgagaagaccatcagcaaggccagagggcag |
| | cccctggagccgaaggtctacaccatgggtcctccccgggaggagctgagcagca |
| | ggtcggtcagcctggcctgcatgatcaacggcttctacccttccgacatctcggtgga |
| | gtgggagaagaacgggaaggcagaggacaactacaagaccacgccggccgtgct |
| | ggacagcgacggctcctacttcctctacagcaagctctcagtgcccacgagtgagtg |
| | gcagcggggcgacgtcttcacttgctccgtgatgcacgaggccttgcacaaccacta |
| | cacgcagaagtccatctcccgctctccgggtaaa (SEQ ID NO: 53) |

TABLE 7
Rabbit 2TdCH2 + LFLEPS Domain Amino Acid Sequence
(Underlined residues are mutations in LFLEPS variants)

| | |
|---|---|
| Hinge 1 Amino Acid Sequence | APSTCSKPTCPPPEFEGGP (SEQ ID NO: 48) |
| First CH2 Domain Amino Acid | SVFIFPPKPKDTLMISRTPEVTCVVVDVSQDDPEVQFT |
| Sequence | WYINNEQVRTARPPLREQQFNSTIRVVSTLPIAHQDW |
| | LRGKEFKCKVHNKALPASIEKTISKARG (SEQ ID NO: |
| | 49) |
| Linker 1 Amino Acid Sequence | GGGGSGGGGSL (SEQ ID NO: 44) |
| Hinge 2 Amino Acid Sequence | APSTCSKPTCPPPEFEGGP (SEQ ID NO: 48) |
| Second CH2 Domain Amino | SVFIFPPKPKDTLMISRTPEVTCVVVDVSQDDPEVQFT |
| Acid Sequence | WYINNEQVRTARPPLREQQFNSTIRVVSTLPIAHQDW |
| | LRGKEFKCKVHNKALPASIEKTISKARG (SEQ ID NO: |
| | 49) |
| Linker 2 Amino Acid Sequence | GGGGSGGGGSL (SEQ ID NO: 44) |
| Hinge 3 Amino Acid Sequence | APSTCSKPTCPPPEFEGGP (SEQ ID NO: 48) |
| Third CH2 domain Amino Acid | SVFIFPPKPKDTLMISRTPEVTCVVVDVSQDDPEVQFT |
| Sequence | WYINNEQVRTARPPLREQQFNSTIRVVSTLPIAHQDW |
| | LRGKEFKCKVHNKALPASIEKTISKARG (SEQ ID NO: |
| | 49) |
| Fc Domain (only CH2 + CH3) | SVFIFPPKPKDTLMISRTPEVTCVVVDVSQDDPEVQFT |
| Amino Acid Sequence | WYINNEQVRTARPPLREQQFNSTIRVVSTLPIAHQDW |
| | LRGKEFKCKVHNKALPASIEKTISKARGQPLEPKVYT |
| | MGPPREELSSRSVSLACMINGFYPSDISVEWEKNGKA |
| | EDNYKTTPAVLDSDGSYFLYSKLSVPTSEWQRGDVFT |
| | CSVMHEALHNHYTQKSISRSPGK (SEQ ID NO: 50) |
| 2TdCH2 + LFLEPS Fc Domain | APSTCSKPTCPPPEFEGGPSVFIFPPKPKDTLMISRTPE |
| Amino Acid Sequence | VTCVVVDVSQDDPEVQFTWYINNEQVRTARPPLREQQF |
| | NSTIRVVSTLPIAHQDWLRGKEFKCKVHNKALPASIEK |
| | TISKARGGGGGSGGGGSLAPSTCSKPTCPPPEFEGGPS |
| | VFIFPPKPKDTLMISRTPEVTCVVVDVSQDDPEVQFTW |
| | YINNEQVRTARPPLREQQFNSTIRVVSTLPIAHQDWLR |
| | GKEFKCKVHNKALPASIEKTISKARGGGGGSGGGGSL |
| | APSTCSKPTCPPPEFEGGPSVFIFPPKPKDTLMISRTPE |
| | VTCVVVDVSQDDPEVQFTWYINNEQVRTARPPLREQQF |
| | NSTIRVVSTLPIAHQDWLRGKEFKCKVHNKALPASIEK |
| | TISKARGQPLEPKVYTMGPPREELSSRSVSLACMINGF |
| | YPSDISVEWEKNGKAEDNYKTTPAVLDSDGSYFLYSK |
| | LSVPTSEWQRGDVFTCSVMHEALHNHYTQKSISRSPG |
| | K (SEQ ID NO: 54) |

TABLE 8
Rabbit 2TdCH2+LFLEPS Domain DNA Sequence
(Underlined residues are mutations in LFLEPS variants)

| | |
|---|---|
| Hinge 1 DNA Sequence | gcgccctcgacatgcagcaagcccacgtgcccaccccctgagttcgaggggggac |
| | cg (SEQ ID NO: 51) |
| First CH2 Domain DNA | tctgtcttcatcttccccccaaaacccaaggacaccctcatgatctcacgcacccccga |
| Sequence | ggtgacctgcgtggtggtggacgtgagccaggatgaccccgaggtgcagttcacat |
| | ggtacataaacaacgagcaggtccgcaccgcccggccgccgctacgggagcagc |
| | agttcaacagcacgatccgcgtggtcagcaccctccccatcgcgcaccaggactgg |
| | ctgaggggcaaggagttcaagtgcaaagtccacaacaaggcactcccggcctccat |
| | cgagaagaccatcagcaaggccagaggg (SEQ ID NO: 62) |
| Linker 1 DNA Sequence | ggcggcggcggcagcggcggcggcggcagccta (SEQ ID NO: 46) |
| Hinge 2 DNA Sequence | gcgccctcgacatgcagcaagcccacgtgcccaccccctgagttcgaggggggac |
| | cg (SEQ ID NO: 51) |
| Second CH2 domain DNA | tctgtcttcatcttccccccaaaacccaaggacaccctcatgatctcacgcaccccc |
| Sequence | gaggtgacctgcgtggtggtggacgtgagccaggatgaccccgaggtgcagttcaca |
| | tggtacataaacaacgagcaggtccgcaccgcccggccgccgctacgggagcagc |
| | agttcaacagcacgatccgcgtggtcagcaccctccccatcgcgcaccaggactgg |
| | ctgaggggcaaggagttcaagtgcaaagtccacaacaaggcactcccggcctccat |
| | cgagaagaccatcagcaaggccagaggg (SEQ ID NO: 62) |
| Linker 2 DNA Sequence | ggcggcggcggcagcggcggcggcggcagccta (SEQ ID NO: 46) |
| Hinge 3 DNA Sequence | gcgccctcgacatgcagcaagcccacgtgcccaccccctgagttcgaggggggac |
| | cg (SEQ ID NO: 51) |
| Third CH2 domain DNA | tctgtcttcatcttccccccaaaacccaaggacaccctcatgatctcacgcacccccga |
| Sequence | ggtgacctgcgtggtggtggacgtgagccaggatgaccccgaggtgcagttcacat |
| | ggtacataaacaacgagcaggtccgcaccgcccggccgccgctacgggagcagc |
| | agttcaacagcacgatccgcgtggtcagcaccctccccatcgcgcaccaggactgg |
| | ctgaggggcaaggagttcaagtgcaaagtccacaacaaggcactcccggcctccat |
| | cgagaagaccatcagcaaggccagaggg (SEQ ID NO: 62) |
| Fc Domain (only CH2 + CH3) | tctgtcttcatcttccccccaaaacccaaggacaccctcatgatctcacgcacccccga |
| DNA Sequence | ggtgacctgcgtggtggtggacgtgagccaggatgaccccgaggtgcagttcacat |
| | ggtacataaacaacgagcaggtccgcaccgcccggccgccgctacgggagcagc |
| | agttcaacagcacgatccgcgtggtcagcaccctccccatcgcgcaccaggactgg |
| | ctgaggggcaaggagttcaagtgcaaagtccacaacaaggcactcccggcctccat |
| | cgagaagaccatcagcaaggccagagggcagcccctggagccgaaggtctacac |
| | catgggtcctccccgggaggagctgagcagcaggtcggtcagcctggcctgcatga |
| | tcaacggcttctacccttccgacatctcggtggagtgggagaagaacgggaaggca |
| | gaggacaactacaagaccacgccggccgtgctggacagcgacggctcctacttcct |
| | ctacagcaagctctcagtgcccacgagtgagtggcagcggggcgacgtcttcacttg |
| | ctccgtgatgcacgaggccttgcacaaccactacacgcagaagtccatctcccgctct |
| | ccgggtaaa (SEQ ID NO: 180) |
| 2TdCH2 + LFLEPS Fc Domain | gcgccctcgacatgcagcaagcccacgtgcccaccccctgagttcgaggggggac |
| DNA Sequence | cgtctgtcttcatcttccccccaaaacccaaggacaccctcatgatctcacgcaccccc |
| | gaggtgacctgcgtggtggtggacgtgagccaggatgaccccgaggtgcagttcac |
| | atggtacataaacaacgagcaggtccgcaccgcccggccgccgctacgggagcag |
| | cagttcaacagcacgatccgcgtggtcagcaccctccccatcgcgcaccaggactg |
| | gctgaggggcaaggagttcaagtgcaaagtccacaacaaggcactcccggcctcca |
| | tcgagaagaccatcagcaaggccagagggggcggcggcggcagcggcggcggc |
| | ggcagcctagcgccctcgacatgcagcaagcccacgtgcccaccccctgagttcga |
| | ggggggaccgtctgtcttcatcttccccccaaaacccaaggacaccctcatgatctca |
| | cgcacccccgaggtgacctgcgtggtggtggacgtgagccaggatgaccccgagg |
| | tgcagttcacatggtacataaacaacgagcaggtccgcaccgcccggccgccgcta |
| | cgggagcagcagttcaacagcacgatccgcgtggtcagcaccctccccatcgcgca |
| | ccaggactggctgaggggcaaggagttcaagtgcaaagtccacaacaaggcactc |
| | ccggcctccatcgagaagaccatcagcaaggccagagggggcggcggcggcag |
| | cggcggcggcggcagcctagcgccctcgacatgcagcaagcccacgtgcccacc |
| | ccctgagttcgaggggggaccgtctgtcttcatcttccccccaaaacccaaggacac |
| | cctcatgatctcacgcacccccgaggtgacctgcgtggtggtggacgtgagccagg |
| | atgaccccgaggtgcagttcacatggtacataaacaacgagcaggtccgcaccgcc |
| | cggccgccgctacgggagcagcagttcaacagcacgatccgcgtggtcagcaccc |
| | tccccatcgcgcaccaggactggctgaggggcaaggagttcaagtgcaaagtccac |
| | aacaaggcactcccggcctccatcgagaagaccatcagcaaggccagagggcagc |
| | ccctggagccgaaggtctacaccatgggtcctccccgggaggagctgagcagcag |
| | gtcggtcagcctggcctgcatgatcaacggcttctacccttccgacatctcggtgga |
| | gtgggagaagaacgggaaggcagaggacaactacaagaccacgccggccgtgctg |
| | gacagcgacggctcctacttcctctacagcaagctctcagtgcccacgagtgagtgg |
| | cagcggggcgacgtcttcacttgctccgtgatgcacgaggccttgcacaaccactac |
| | acgcagaagtccatctcccgctctccgggtaaa (SEQ ID NO: 55) |

TABLE 9
Hinges and Linkers

| | |
|---|---|
| Rabbit Parent Hinge | APSTCSKPTCPPPELLGGP (SEQ ID NO: 18) |
| Rabbit FcR Mutated Hinge: | APSTCSKPTCPPPEFEGGP (SEQ ID NO: 48) |
| L234F + L235E | |
| Mouse IgG1 Hinge | VPRDCGCKPCICTVPEVSS (SEQ ID NO: 102) |
| Linker A | GGGGSGGGGSL (SEQ ID NO: 44) |
| Linker B | GGGGSGGGGS (SEQ ID NO: 43) |
| Linker C | GGGGS (SEQ ID NO: 38) |
| Linker D | GGGS (SEQ ID NO: 14) |
| Linker E | GGS |
| Linker F | GS |

TABLE 10
1TdFc Domain Amino Acid Sequence

| | |
|---|---|
| 1TdFc Domain Amino Acid | APSTCSKPTCPPPELLGGPSVFIFPPKPKDTLMISRTPEV |
| Sequence | TCVVVDVSQDDPEVQFTWYINNEQVRTARPPLREQQF |
| | NSTIRVVSTLPIAHQDWLRGKEFKCKVHNKALPAPIEK |
| | TISKARGQPLEPKVYTMGPPREELSSRSVSLACMINGF |
| | YPSDISVEWEKNGKAEDNYKTTPAVLDSDGSYFLYSK |
| | LSVPTSEWQRGDVFTCSVMHEALHNHYTQKSISRSPG |
| | KGGGGSGGGGSLAPSTCSKPTCPPPELLGGPSVFIFPPK |
| | PKDTLMISRTPEVTCVVVDVSQDDPEVQFTWYINNEQ |
| | VRTARPPLREQQFNSTIRVVSTLPIAHQDWLRGKEFKC |
| | KVHNKALPAPIEKTISKARGQPLEPKVYTMGPPREELS |
| | SRSVSLACMINGFYPSDISVEWEKNGKAEDNYKTTPA |
| | VLDSDGSYFLYSKLSVPTSEWQRGDVFTCSVMHEAL |
| | HNHYTQKSISRSPGK (SEQ ID NO: 56) |
| Hinge 1 Amino Acid Sequence | APSTCSKPTCPPPELLGGP (SEQ ID NO: 18) |
| Additional Fc Domain | SVFIFPPKPKDTLMISRTPEVTCVVVDVSQDDPEVQFT |
| (CH2+CH3) Amino Acid | WYINNEQVRTARPPLREQQFNSTIRVVSTLPIAHQDW |
| Sequence | LRGKEFKCKVHNKALPAPIEKTISKARGQPLEPKVYT |
| | MGPPREELSSRSVSLACMINGFYPSDISVEWEKNGKA |
| | EDNYKTTPAVLDSDGSYFLYSKLSVPTSEWQRGDVFT |
| | CSVMHEALHNHYTQKSISRSPGK (SEQ ID NO: 45) |
| Linker 1 Amino Acid Sequence | GGGGSGGGGSL (SEQ ID NO: 44) |
| Hinge 2 Amino Acid Sequence | APSTCSKPTCPPPELLGGP (SEQ ID NO: 18) |
| Fc Domain (CH2+CH3) Amino | SVFIFPPKPKDTLMISRTPEVTCVVVDVSQDDPEVQFT |
| Acid Sequence | WYINNEQVRTARPPLREQQFNSTIRVVSTLPIAHQDW |
| | LRGKEFKCKVHNKALPAPIEKTISKARGQPLEPKVYT |
| | MGPPREELSSRSVSLACMINGFYPSDISVEWEKNGKA |
| | EDNYKTTPAVLDSDGSYFLYSKLSVPTSEWQRGDVFT |
| | CSVMHEALHNHYTQKSISRSPGK (SEQ ID NO: 45) |

TABLE 11
1TdFc Domain DNA Sequence

| | |
|---|---|
| 1TdFc Domain DNA Sequence | gcgccctcgacatgcagcaagcccacgtgcccaccccctgaactcctggggggac |
| | cgtctgtcttcatcttccccccaaaacccaaggacaccctcatgatctcacgcaccccc |
| | gaggtcacatgcgtggtggtggacgtgagccaggatgaccccgaggtgcagttcac |
| | atggtacataaacaacgagcaggtgcgcaccgcccggccgccgctacgggagcag |
| | cagttcaacagcacgatccgcgtggtcagcaccctccccatcgcgcaccaggactg |
| | gctgaggggcaaggagttcaagtgcaaagtccacaacaaggcactcccggccccc |
| | atcgagaaaaccatctccaaagccagagggcagcccctggagccgaaggtctaca |
| | ccatgggccctccccgggaggagctgagcagcaggtcggtcagcctggcctgcat |
| | gatcaacggcttctacccttccgacatctcggtggagtgggagaagaacgggaagg |
| | cagaggacaactacaagaccacgccggccgtgctggacagcgacggctcctacttc |
| | ctctacagcaagctctcagtgcccacgagtgagtggcagcggggcgacgtcttcacc |
| | tgctccgtgatgcacgaggccttgcacaaccactacacgcagaagtccatctcccgct |
| | ctccgggtaaaggggcggcggcagcggggggggcagcctagcgccctcg |
| | acatgcagcaagcccacgtgcccaccccctgaactcctggggggaccgtctgtcttc |
| | atcttccccccaaaacccaaggacaccctcatgatctcacgcacccccgaggtcaca |
| | tgcgtggtggtggacgtgagccaggatgaccccgaggtgcagttcacatggtacata |
| | aacaacgagcaggtgcgcaccgcccggccgccgctacgggagcagcagttcaac |
| | agcacgatccgcgtggtcagcaccctccccatcgcgcaccaggactggctgaggg |
| | gcaaggagttcaagtgcaaagtccacaacaaggcactcccggcccccatcgagaaa |
| | accatctccaaagccagagggcagcccctggagccgaaggtctacaccatgggcc |
| | ctccccgggaggagctgagcagcaggtcggtcagcctggcctgcatgatcaacgg |
| | cttctacccttccgacatctcggtggagtgggagaagaacgggaaggcagaggaca |
| | actacaagaccacgccggccgtgctggacagcgacggctcctacttcctctacagca |
| | agctctcagtgcccacgagtgagtggcagcggggcgacgtcttcacctgctccgtga |
| | tgcacgaggccttgcacaaccactacacgcagaagtccatctcccgctctccgggta |
| | aa (SEQ ID NO: 57) |
| Hinge 1 DNA Sequence | gcgccctcgacatgcagcaagcccacgtgcccaccccctgaactcctggggggac |
| | cg (SEQ ID NO: 58) |
| Additional Fc Domain | tctgtcttcatcttccccccaaaacccaaggacaccctcatgatctcacgcacccccga |
| (CH2 + CH3) DNA Sequence | ggtcacatgcgtggtggtggacgtgagccaggatgaccccgaggtgcagttcacat |
| | ggtacataaacaacgagcaggtgcgcaccgcccggccgccgctacgggagcagc |
| | agttcaacagcacgatccgcgtggtcagcaccctccccatcgcgcaccaggactgg |
| | ctgaggggcaaggagttcaagtgcaaagtccacaacaaggcactcccggcccccat |
| | cgagaaaaccatctccaaagccagagggcagcccctggagccgaaggtctacacc |
| | atgggccctccccgggaggagctgagcagcaggtcggtcagcctggcctgcatgat |
| | caacggcttctacccttccgacatctcggtggagtgggagaagaacgggaaggcag |
| | aggacaactacaagaccacgccggccgtgctggacagcgacggctcctacttcctct |
| | acagcaagctctcagtgcccacgagtgagtggcagcggggcgacgtcttcacctgc |
| | tccgtgatgcacgaggccttgcacaaccactacacgcagaagtccatctcccgctctc |
| | cgggtaaa (SEQ ID NO: 59) |
| Linker 1 DNA Sequence | ggcggcggcggcagcggcggcggcggcagccta (SEQ ID NO: 46) |
| Hinge 2 DNA Sequence | gcgccctcgacatgcagcaagcccacgtgcccaccccctgaactcctggggggac |
| | cg (SEQ ID NO: 58) |
| Fc Domain (CH2 + CH3) DNA | tctgtcttcatcttccccccaaaacccaaggacaccctcatgatctcacgcacccccga |
| Sequence | ggtcacatgcgtggtggtggacgtgagccaggatgaccccgaggtgcagttcacat |
| | ggtacataaacaacgagcaggtgcgcaccgcccggccgccgctacgggagcagc |
| | agttcaacagcacgatccgcgtggtcagcaccctccccatcgcgcaccaggactgg |
| | ctgaggggcaaggagttcaagtgcaaagtccacaacaaggcactcccggcccccat |
| | cgagaaaaccatctccaaagccagagggcagcccctggagccgaaggtctacacc |
| | atgggccctccccgggaggagctgagcagcaggtcggtcagcctggcctgcatgat |
| | caacggcttctacccttccgacatctcggtggagtgggagaagaacgggaaggcag |
| | aggacaactacaagaccacgccggccgtgctggacagcgacggctcctacttcctct |
| | acagcaagctctcagtgcccacgagtgagtggcagcggggcgacgtcttcacctgc |
| | tccgtgatgcacgaggccttgcacaaccactacacgcagaagtccatctcccgctctc |
| | cgggtaaa (SEQ ID NO: 59) |

TABLE 12
2TdFc Domain Amino Acid Sequence

| | |
|---|---|
| 2TdFc Domain Amino Acid | APSTCSKPTCPPPELLGGPSVFIFPPKPKDTLMISRTPEV |
| Sequence | TCVVVDVSQDDPEVQFTWYINNEQVRTARPPLREQQF |
| | NSTIRVVSTLPIAHQDWLRGKEFKCKVHNKALPAPIEK |
| | TISKARGQPLEPKVYTMGPPREELSSRSVSLACMINGF |
| | YPSDISVEWEKNGKAEDNYKTTPAVLDSDGSYFLYSK |
| | LSVPTSEWQRGDVFTCSVMHEALHNHYTQKSISRSPG |
| | KGGGGSGGGGSLAPSTCSKPTCPPPELLGGPSVFIFPPK |
| | PKDTLMISRTPEVTCVVVDVSQDDPEVQFTWYINNEQ |
| | VRTARPPLREQQFNSTIRVVSTLPIAHQDWLRGKEFKC |
| | KVHNKALPAPIEKTISKARGQPLEPKVYTMGPPREELS |
| | SRSVSLACMINGFYPSDISVEWEKNGKAEDNYKTTPA |
| | VLDSDGSYFLYSKLSVPTSEWQRGDVFTCSVMHEAL |
| | HNHYTQKSISRSPGKGGGGSGGGGSLAPSTCSKPTCPP |
| | PELLGGPSVFIFPPKPKDTLMISRTPEVTCVVVDVSQD |
| | DPEVQFTWYINNEQVRTARPPLREQQFNSTIRVVSTLP |
| | IAHQDWLRGKEFKCKVHNKALPAPIEKTISKARGQPL |
| | EPKVYTMGPPREELSSRSVSLACMINGFYPSDISVEWE |
| | KNGKAEDNYKTTPAVLDSDGSYFLYSKLSVPTSEWQ |
| | RGDVFTCSVMHEALHNHYTQKSISRSPGK (SEQ ID |
| | NO: 60) |
| Hinge 1 Amino Acid Sequence | APSTCSKPTCPPPELLGGP (SEQ ID NO: 18) |
| Additional Fc Domain 1 | SVFIFPPKPKDTLMISRTPEVTCVVVDVSQDDPEVQFT |
| (CH2+CH3) Amino Acid | WYINNEQVRTARPPLREQQFNSTIRVVSTLPIAHQDW |
| Sequence | LRGKEFKCKVHNKALPAPIEKTISKARGQPLEPKVYT |
| | MGPPREELSSRSVSLACMINGFYPSDISVEWEKNGKA |
| | EDNYKTTPAVLDSDGSYFLYSKLSVPTSEWQRGDVFT |
| | CSVMHEALHNHYTQKSISRSPGK (SEQ ID NO: 45) |
| Linker 1 Amino Acid Sequence | GGGGSGGGGSL (SEQ ID NO: 44) |
| Hinge 2 Amino Acid Sequence | APSTCSKPTCPPPELLGGP (SEQ ID NO: 18) |
| Additional Fc Domain 2 | SVFIFPPKPKDTLMISRTPEVTCVVVDVSQDDPEVQFT |
| (CH2+CH3) Amino Acid | WYINNEQVRTARPPLREQQFNSTIRVVSTLPIAHQDW |
| Sequence | LRGKEFKCKVHNKALPAPIEKTISKARGQPLEPKVYT |
| | MGPPREELSSRSVSLACMINGFYPSDISVEWEKNGKA |
| | EDNYKTTPAVLDSDGSYFLYSKLSVPTSEWQRGDVFT |
| | CSVMHEALHNHYTQKSISRSPGK (SEQ ID NO: 45) |
| Linker 2 Amino Acid Sequence | GGGGSGGGGSL (SEQ ID NO: 44) |
| Hinge 3 Amino Acid Sequence | APSTCSKPTCPPPELLGGP (SEQ ID NO: 18) |
| Fc Domain (CH2+CH3) Amino | SVFIFPPKPKDTLMISRTPEVTCVVVDVSQDDPEVQFT |
| Acid Sequence | WYINNEQVRTARPPLREQQFNSTIRVVSTLPIAHQDW |
| | LRGKEFKCKVHNKALPAPIEKTISKARGQPLEPKVYT |
| | MGPPREELSSRSVSLACMINGFYPSDISVEWEKNGKA |
| | EDNYKTTPAVLDSDGSYFLYSKLSVPTSEWQRGDVFT |
| | CSVMHEALHNHYTQKSISRSPGK (SEQ ID NO: 45) |

TABLE 13
2TdFc Domain DNA Sequence

| | |
|---|---|
| 2TdFc Domain DNA Sequence | gcgccctcgacatgcagcaagcccacgtgcccaccccctgaactcctggggggac |
| | cgtctgtcttcatcttccccccaaaacccaaggacaccctcatgatctcacgcaccccc |
| | gaggtcacatgcgtggtggtggacgtgagccaggatgaccccgaggtgcagttcac |
| | atggtacataaacaacgagcaggtgcgcaccgcccggccgccgctacgggagcag |
| | cagttcaacagcacgatccgcgtggtcagcaccctccccategcgcaccaggactg |
| | gctgaggggcaaggagttcaagtgcaaagtccacaacaaggcactcccggccccc |
| | atcgagaaaaccatctccaaagccagagggcagcccctggagccgaaggtctaca |
| | ccatgggccctccccgggaggagctgagcagcaggtcggtcagcctggcctgcat |
| | gatcaacggcttctacccttccgacatctcggtggagtgggagaagaacgggaagg |
| | cagaggacaactacaagaccacgccggccgtgctggacagcgacggctcctacttc |
| | ctctacagcaagctctcagtgcccacgagtgagtggcagcggggcgacgtcttcacc |
| | tgctccgtgatgcacgaggccttgcacaaccactacacgcagaagtccatctcccgct |
| | ctccgggtaaaggcggcggcggcagcggcggcggeggcagcctagcgccctcg |
| | acatgcagcaagcccacgtgcccaccccctgaactcctggggggaccgtctgtcttc |
| | atcttccccccaaaacccaaggacaccctcatgatctcacgcacccccgaggtcaca |
| | tgcgtggtggtggacgtgagccaggatgaccccgaggtgcagttcacatggtacata |
| | aacaacgagcaggtgcgcaccgcccggccgccgctacgggagcagcagttcaac |
| | agcacgatccgcgtggtcagcaccctccccatcgcgcaccaggactggctgaggg |
| | gcaaggagttcaagtgcaaagtccacaacaaggcactcccggcccccatcgagaaa |
| | accatctccaaagccagagggcagcccctggagccgaaggtctacaccatgggcc |
| | ctccccgggaggagctgagcagcaggtcggtcagcctggcctgcatgatcaacgg |
| | cttctacccttccgacatctcggtggagtgggagaagaacgggaaggcagaggaca |
| | actacaagaccacgccggccgtgctggacagcgacggctcctacttcctctacagca |
| | agctctcagtgcccacgagtgagtggcagcggggcgacgtcttcacctgctccgtga |
| | tgcacgaggccttgcacaaccactacacgcagaagtccatctcccgctctccgggta |
| | aaggcggcggcggcagcggcggcggcggcagcctagcgccctcgacatgcagc |
| | aagcccacgtgcccaccccctgaactcctggggggaccgtctgtcttcatcttccccc |
| | caaaacccaaggacaccctcatgatctcacgcacccccgaggtcacatgcgtggtg |
| | gtggacgtgagccaggatgaccccgaggtgcagttcacatggtacataaacaacga |
| | gcaggtgcgcaccgcccggccgccgctacgggagcagcagttcaacagcacgatc |
| | cgcgtggtcagcaccctccccatcgcgcaccaggactggctgaggggcaaggagtt |
| | caagtgcaaagtccacaacaaggcactcccggcccccatcgagaaaaccatctcca |
| | aagccagagggcagcccctggagccgaaggtctacaccatgggccctccccggga |
| | ggagctgagcagcaggtcggtcagcctggcctgcatgatcaacggcttctacccttc |
| | cgacatctcggtggagtgggagaagaacgggaaggcagaggacaactacaagac |
| | cacgccggccgtgctggacagcgacggctcctacttcctctacagcaagctctcagt |
| | gcccacgagtgagtggcagcggggcgacgtcttcacctgctccgtgatgcacgagg |
| | ccttgcacaaccactacacgcagaagtccatctcccgctctcogggtaaa (SEQ |
| | ID NO: 61) |
| Hinge 1 DNA Sequence | gcgccctcgacatgcagcaagcccacgtgcccaccccctgaactcctggggggac |
| | cg (SEQ ID NO: 58) |
| Additional Fc Domain 1 | tctgtcttcatcttccccccaaaacccaaggacaccctcatgatctcacgcacccccga |
| (CH2 + CH3) DNA Sequence | ggtcacatgcgtggtggtggacgtgagccaggatgaccccgaggtgcagttcacat |
| | ggtacataaacaacgagcaggtgcgcaccgcccggccgccgctacgggagcagc |
| | agttcaacagcacgatccgcgtggtcagcaccctccccatcgcgcaccaggactgg |
| | ctgaggggcaaggagttcaagtgcaaagtccacaacaaggcactcccggcccccat |
| | cgagaaaaccatctccaaagccagagggcagcccctggagccgaaggtctacacc |
| | atgggccctccccgggaggagctgagcagcaggtcggtcagcctggcctgcatgat |
| | caacggcttctacccttccgacatctcggtggagtgggagaagaacgggaaggcag |
| | aggacaactacaagaccacgccggccgtgctggacagcgacggctcctacttcctct |
| | acagcaagctctcagtgcccacgagtgagtggcagcggggcgacgtcttcacctgc |
| | tccgtgatgcacgaggccttgcacaaccactacacgcagaagtccatctcccgctctc |
| | cgggtaaa (SEQ ID NO: 59) |
| Linker 1 DNA Sequence | ggcggcggcggcagcggcggcggcggcagccta (SEQ ID NO: 46) |
| Hinge 2 DNA Sequence | gcgccctcgacatgcagcaagcccacgtgcccaccccctgaactcctggggggac |
| | cg (SEQ ID NO: 58) |
| Additional Fc Domain 2 | tctgtcttcatcttccccccaaaacccaaggacaccctcatgatctcacgcacccccga |
| (CH2+CH3) DNA Sequence | ggtcacatgcgtggtggtggacgtgagccaggatgaccccgaggtgcagttcacat |
| | ggtacataaacaacgagcaggtgcgcaccgcccggccgccgctacgggagcagc |
| | agttcaacagcacgatccgcgtggtcagcaccctccccatcgcgcaccaggactgg |
| | ctgaggggcaaggagttcaagtgcaaagtccacaacaaggcactcccggcccccat |
| | cgagaaaaccatctccaaagccagagggcagcccctggagccgaaggtctacacc |
| | atgggccctccccgggaggagctgagcagcaggtcggtcagcctggcctgcatgat |
| | caacggcttctacccttccgacatctcggtggagtgggagaagaacgggaaggcag |
| | aggacaactacaagaccacgccggccgtgctggacagcgacggctcctacttcctct |
| | acagcaagctctcagtgcccacgagtgagtggcagcggggcgacgtcttcacctgc |
| | tccgtgatgcacgaggccttgcacaaccactacacgcagaagtccatctcccgetctc |
| | cgggtaaa (SEQ ID NO: 59) |
| Linker 2 DNA Sequence | ggcggcggcggcagcggcggeggcggcagccta (SEQ ID NO: 46) |
| Hinge 3 DNA Sequence | gcgccctcgacatgcagcaagcccacgtgcccaccccctgaactcctggggggac |
| | cg (SEQ ID NO: 58) |
| Fc Domain (CH2 + CH3) DNA | tctgtcttcatcttccccccaaaacccaaggacaccctcatgatctcacgcacccccga |
| Sequence | ggtcacatgcgtggtggtggacgtgagccaggatgaccccgaggtgcagttcacat |
| | ggtacataaacaacgagcaggtgcgcaccgcccggccgccgctacgggagcagc |
| | agttcaacagcacgatccgcgtggtcagcaccctccccatcgcgcaccaggactgg |
| | ctgaggggcaaggagttcaagtgcaaagtccacaacaaggcactcccggcccccat |
| | cgagaaaaccatctccaaagccagagggcagcccctggagccgaaggtctacacc |
| | atgggccctccccgggaggagctgagcagcaggtcggtcagcctggcctgcatgat |
| | caacggcttctacccttccgacatctcggtggagtgggagaagaacgggaaggcag |
| | aggacaactacaagaccacgccggccgtgctggacagcgacggctcctacttcctct |
| | acagcaagctctcagtgcccacgagtgagtggcagcggggcgacgtcttcacctgc |
| | tccgtgatgcacgaggccttgcacaaccactacacgcagaagtccatctcccgctctc |
| | cgggtaaa (SEQ ID NO: 59) |

TABLE 14
Anti-IDO1 Antibody Variable Domains
Anti-IDO1 Antibody
Clone Name: 7H8L17
Species: Rabbit
Isotype: IgG

| | |
|---|---|
| Heavy Chain Leader DNA | atggagactgggctgcgctggcttctcctggtcgctgtgctcaaaggtgtccagtgt |
| Sequence | (SEQ ID NO: 1) |
| Heavy Chain Leader Amino acid | METGLRWLLLVAVLKGVQC (SEQ ID NO: 63) |
| Sequence | |
| Heavy Chain Variable Domain | cagtcgctggaggagtccgggggtcgcctggtcacgcctgggacacccctgacact |
| DNA Sequence | cacctgcacagtctctggaatcgacctcagtacctatccaatgggctgggtccgccag |
| | gcgacagggaaggggctggaatacatcggaatcattagtagtagtggtagcacatac |
| | tacgcgagctggacaaaaggccgattcaccatctccaaaacctcgaccacggtggat |
| | ctgaaaatgaccactctgacaacttcagacacggccacctatttctgtaccagaccatg |
| | gggatacggcatggacccctggggcccagggaccctcgtcaccgtctcttca |
| | (SEQ ID NO: 64) |
| Heavy Chain Variable Domain | QSLEESGGRLVTPGTPLTLTCTVSGIDLSTYPMGWVR |
| Amino Acid Sequence | QATGKGLEYIGIISSSGSTYYASWTKGRFTISKTSTTVD |
| | LKMTTLTTSDTATYFCTRPWGYGMDPWGPGTLVTVS |
| | S (SEQ ID NO: 65) |
| Heavy Chain CDR1 Amino Acid | GIDLSTYP (SEQ ID NO: 66) |
| Sequence | |
| (IMGT) | |
| Heavy Chain CDR2 Amino Acid | ISSSGST (SEQ ID NO: 67) |
| Sequence | |
| (IMGT) | |
| Heavy Chain CDR3 Amino Acid | TRPWGYGMDP (SEQ ID NO: 68) |
| Sequence | |
| (IMGT) | |
| Light Chain Leader DNA | atggacacgagggcccccactcagctgctggggctcctgctgctctggctcccaggt |
| Sequence | gccacattt (SEQ ID NO: 10) |
| Light Chain Leader Amino acid | MDTRAPTQLLGLLLLWLPGATF (SEQ ID NO: 69) |
| Sequence | |
| Light Chain Variable Domain | gcgcaagtgctgacccagactccatcccctgtgtctgcagctgtgggaggcacagtc |
| DNA Sequence | accatcagttgccaggccagtcagagtgtttataatgacaacgccttatcctggtatca |
| | gcagaaaccagggcagcctcccaaagtcctgatctactatgcatccactctggcatct |
| | ggggtcccatcgcggttcaaaggcagtggatctgggacacagttcactctcaccatc |
| | agcgaagtacagtgtgacgatgctgccacttactattgtcaaggctattatagtggtgct |
| | atttatgttttcggcggagggaccgaggtggtgctcaaaggt (SEQ ID NO: 70) |
| Light Chain Variable Domain | AQVLTQTPSPVSAAVGGTVTISCQASQSVYNDNALSW |
| Amino Acid Sequence | YQQKPGQPPKVLIYYASTLASGVPSRFKGSGSGTQFTL |
| | TISEVQCDDAATYYCQGYYSGAIYVFGGGTEVVLKG |
| | (SEQ ID NO: 71) |
| Light Chain CDR1 Amino Acid | QSVYNDNA (SEQ ID NO: 72) |
| Sequence | |
| (IMGT) | |
| Light Chain CDR2 Amino Acid | YAS |
| Sequence | |
| (IMGT) | |
| Light Chain CDR3 Amino Acid | QGYYSGAIYV (SEQ ID NO: 73) |
| Sequence | |
| (IMGT) | |
| Heavy Chain DNA Sequence | cagtcgctggaggagtccgggggtcgcctggtcacgcctgggacacccctgacact |
| | cacctgcacagtctctggaatcgacctcagtacctatccaatgggctgggtccgccag |
| | gcgacagggaaggggctggaatacatcggaatcattagtagtagtggtagcacatac |
| | tacgcgagctggacaaaaggccgattcaccatctccaaaacctcgaccacggtggat |
| | ctgaaaatgaccactctgacaacttcagacacggccacctatttctgtaccagaccatg |
| | gggatacggcatggacccctggggcccagggaccctcgtcaccgtctcttcagggc |
| | aacctaaggctccatcagtcttcccactggccccctgctgcggggacacacccagct |
| | ccacggtgaccctgggctgcctggtcaaaggctacctcccggagccagtgaccgtg |
| | acctggaactcgggcaccctcaccaatggggtacgcaccttcccgtccgtccggca |
| | gtcctcaggcctctactcgctgagcagcgtggtgagcgtgacctcaagcagccagcc |
| | cgtcacctgcaacgtggcccacccagccaccaacaccaaagtggacaagaccgtg |
| | gcgccctcgacatgcagcaagcccacgtgcccaccccctgaactcctggggggac |
| | cgtctgtcttcatcttccccccaaaacccaaggacaccctcatgatctcacgcaccccc |
| | gaggtcacatgcgtggtggtggacgtgagccaggatgaccccgaggtgcagttcac |
| | atggtacataaacaacgagcaggtgcgcaccgcccggccgccgctacgggagcag |
| | cagttcaacagcacgatccgcgtggtcagcaccctccccatcgcgcaccaggactg |
| | gctgaggggcaaggagttcaagtgcaaagtccacaacaaggcactcccggccccc |
| | atcgagaaaaccatctccaaagccagagggcagcccctggagccgaaggtctaca |
| | ccatgggccctccccgggaggagctgagcagcaggtcggtcagcctggcctgcat |
| | gatcaacggcttctacccttccgacatctcggtggagtgggagaagaacgggaagg |
| | cagaggacaactacaagaccacgccggccgtgctggacagcgacggctcctacttc |
| | ctctacagcaagctctcagtgcccacgagtgagtggcagcggggcgacgtcttcacc |
| | tgctccgtgatgcacgaggccttgcacaaccactacacgcagaagtccatctcccgct |
| | ctccgggtaaa (SEQ ID NO: 74) |
| Heavy Chain Amino Acid | QSLEESGGRLVTPGTPLTLTCTVSGIDLSTYPMGWVR |
| Sequence | QATGKGLEYIGIISSSGSTYYASWTKGRFTISKTSTTVD |
| | LKMTTLTTSDTATYFCTRPWGYGMDPWGPGTLVTVS |
| | SGQPKAPSVFPLAPCCGDTPSSTVTLGCLVKGYLPEPV |
| | TVTWNSGTLTNGVRTFPSVRQSSGLYSLSSVVSVTSSS |
| | QPVTCNVAHPATNTKVDKTVAPSTCSKPTCPPPELLG |
| | GPSVFIFPPKPKDTLMISRTPEVTCVVVDVSQDDPEVQ |
| | FTWYINNEQVRTARPPLREQQFNSTIRVVSTLPIAHQD |
| | WLRGKEFKCKVHNKALPAPIEKTISKARGQPLEPKVY |
| | TMGPPREELSSRSVSLACMINGFYPSDISVEWEKNGK |
| | AEDNYKTTPAVLDSDGSYFLYSKLSVPTSEWQRGDVF |
| | TCSVMHEALHNHYTQKSISRSPGK (SEQ ID NO: 75) |
| Light Chain DNA Sequence | gcgcaagtgctgacccagactccatcccctgtgtctgcagctgtgggaggcacagtc |
| | accatcagttgccaggccagtcagagtgtttataatgacaacgccttatcctggtatca |
| | gcagaaaccagggcagcctcccaaagtcctgatctactatgcatccactctggcatct |
| | ggggtcccatcgcggttcaaaggcagtggatctgggacacagttcactctcaccatc |
| | agcgaagtacagtgtgacgatgctgccacttactattgtcaaggctattatagtggtgct |
| | atttatgttttcggcggagggaccgaggtggtgctcaaaggtgatccagttgcacctac |
| | tgtcctcatcttcccaccagctgctgatcaggtggcaactggaacagtcaccatcgtgt |
| | gtgtggcgaataaatactttcccgatgtcaccgtcacctgggaggtggatggcaccac |
| | ccaaacaactggcatcgagaacagtaaaacaccgcagaattctgcagattgtaccta |
| | caacctcagcagcactctgacactgaccagcacacagtacaacagccacaaagagt |
| | acacctgcaaggtgacccagggcacgacctcagtcgtccagagcttcaataggggt |
| | gactgt (SEQ ID NO: 76) |
| Light Chain Amino Acid | AQVLTQTPSPVSAAVGGTVTISCQASQSVYNDNALSW |
| Sequence | YQQKPGQPPKVLIYYASTLASGVPSRFKGSGSGTQFTL |
| | TISEVQCDDAATYYCQGYYSGAIYVFGGGTEVVLKG |
| | DPVAPTVLIFPPAADQVATGTVTIVCVANKYFPDVTV |
| | TWEVDGTTQTTGIENSKTPQNSADCTYNLSSTLTLTST |
| | QYNSHKEYTCKVTQGTTSVVQSFNRGDC (SEQ ID NO: 77) |
| Heavy Chain Fab DNA sequence | cagtcgctggaggagtccgggggtcgcctggtcacgcctgggacacccctgacact |
| | cacctgcacagtctctggaatcgacctcagtacctatccaatgggctgggtccgccag |
| | gcgacagggaaggggctggaatacatcggaatcattagtagtagtggtagcacatac |
| | tacgcgagctggacaaaaggccgattcaccatctccaaaacctcgaccacggtggat |
| | ctgaaaatgaccactctgacaacttcagacacggccacctatttctgtaccagaccatg |
| | gggatacggcatggacccctggggcccagggaccctcgtcaccgtctcttcagggc |
| | aacctaaggctccatcagtcttcccactggccccctgctgcggggacacacccagct |
| | ccacggtgaccctgggctgcctggtcaaaggctacctcccggagccagtgaccgtg |
| | acctggaactcgggcaccctcaccaatggggtacgcaccttcccgtccgtccggca |
| | gtcctcaggcctctactcgctgagcagcgtggtgagcgtgacctcaagcagccagcc |
| | cgtcacctgcaacgtggcccacccagccaccaacaccaaagtggacaagaccgtg |
| | (SEQ ID NO: 78) |
| Heavy Chain Fab amino acid | QSLEESGGRLVTPGTPLTLTCTVSGIDLSTYPMGWVR |
| sequence | QATGKGLEYIGIISSSGSTYYASWTKGRFTISKTSTTVD |
| | LKMTTLTTSDTATYFCTRPWGYGMDPWGPGTLVTVS |
| | SGQPKAPSVFPLAPCCGDTPSSTVTLGCLVKGYLPEPV |
| | TVTWNSGTLINGVRTFPSVRQSSGLYSLSSVVSVTSSS |
| | QPVTCNVAHPATNTKVDKTV (SEQ ID NO: 79) |

TABLE 15
Anti-IDO1 Parental Antibody Constant Domains
Anti-IDO1 Antibody
Clone Name: 7H8L17
Species: Rabbit
Isotype: IgG

| | |
|---|---|
| Heavy Chain CH1 DNA | gggcaacctaaggctccatcagtcttcccactggccccctgctgcggggacacaccc |
| sequence | agctccacggtgaccctgggctgcctggtcaaaggctacctcccggagccagtgac |
| | cgtgacctggaactcgggcaccctcaccaatggggtacgcaccttcccgtccgtccg |
| | gcagtcctcaggcctctactcgctgagcagcgtggtgagcgtgacctcaagcagcca |
| | gcccgtcacctgcaacgtggcccacccagccaccaacaccaaagtggacaagacc |
| | gtg (SEQ ID NO: 177) |
| Heavy Chain CH1 Amino acid | GQPKAPSVFPLAPCCGDTPSSTVTLGCLVKGYLPEPVT |
| Sequence | VTWNSGTLTNGVRTFPSVRQSSGLYSLSSVVSVTSSSQ |
| | PVTCNVAHPATNTKVDKTV (SEQ ID NO: 17) |
| Heavy Chain Hinge DNA | gcgccctcgacatgcagcaagcccacgtgcccaccccctgaactcctggggggac |
| Sequence | cg (SEQ ID NO: 22) |
| Heavy Chain Hinge Amino Acid | APSTCSKPTCPPPELLGGP (SEQ ID NO: 18) |
| Sequence | |
| Heavy Chain CH2 DNA | tctgtcttcatcttccccccaaaacccaaggacaccctcatgatctcacgcacccccga |
| Sequence | ggtcacatgcgtggtggtggacgtgagccaggatgaccccgaggtgcagttcacat |
| | ggtacataaacaacgagcaggtgcgcaccgcccggccgccgctacgggagcagc |
| | agttcaacagcacgatccgcgtggtcagcaccctccccatcgcgcaccaggactgg |
| | ctgaggggcaaggagttcaagtgcaaagtccacaacaaggcactcccggcccccat |
| | cgagaaaaccatctccaaagccagaggg (SEQ ID NO: 178) |
| Heavy Chain CH2 Amino Acid | SVFIFPPKPKDTLMISRTPEVTCVVVDVSQDDPEVQFT |
| Sequence | WYINNEQVRTARPPLREQQFNSTIRVVSTLPIAHQDW |
| | LRGKEFKCKVHNKALPAPIEKTISKARG (SEQ ID NO: 19) |
| Heavy Chain CH3 DNA | cagcccctggagccgaaggtctacaccatgggccctccccgggaggagctgagca |
| Sequence | gcaggtcggtcagcctggcctgcatgatcaacggcttctacccttccgacatctcggt |
| | ggagtgggagaagaacgggaaggcagaggacaactacaagaccacgccggccg |
| | tgctggacagcgacggctcctacttcctctacagcaagctctcagtgcccacgagtga |
| | gtggcagcggggcgacgtcttcacctgctccgtgatgcacgaggccttgcacaacc |
| | actacacgcagaagtccatctcccgctctccgggtaaa (SEQ ID NO: 179) |
| Heavy Chain CH3 Amino Acid | QPLEPKVYTMGPPREELSSRSVSLACMINGFYPSDISV |
| Sequence | EWEKNGKAEDNYKTTPAVLDSDGSYFLYSKLSVPTSE |
| | WQRGDVFTCSVMHEALHNHYTQKSISRSPGK (SEQ ID NO: 20) |
| Light Chain Constant Domain | gatccagttgcacctactgtcctcatcttcccaccagctgctgatcaggtggcaactgg |
| DNA sequence | aacagtcaccatcgtgtgtgtggcgaataaatactttcccgatgtcaccgtcacctggg |
| | aggtggatggcaccacccaaacaactggcatcgagaacagtaaaacaccgcagaat |
| | tctgcagattgtacctacaacctcagcagcactctgacactgaccagcacacagtaca |
| | acagccacaaagagtacacctgcaaggtgacccagggcacgacctcagtcgtcca |
| | gagcttcaataggggtgactgt (SEQ ID NO: 80) |
| Light Chain Constant Domain | DPVAPTVLIFPPAADQVATGTVTIVCVANKYFPDVTV |
| Amino acid Sequence | TWEVDGTTQTTGIENSKTPQNSADCTYNLSSTLTLTST |
| | QYNSHKEYTCKVTQGTTSVVQSFNRGDC (SEQ ID NO: 25) |

TABLE 16
Anti-Phospho-ERK1/2 (Thr202, Tyr204) Antibody Variable Domains
(Anti-pERK1/2 Antibody)
Anti-Phospho-ERK1/2 (Thr202, Tyr204) Antibody
Clone Name: MILAN8R
Species: Mouse
Isotype: IgG1, kappa

| | |
|---|---|
| Heavy Chain Leader DNA | atggacatgagggtgcccgctcaactgctgggtctgctgcttctttggctgcgcgggg |
| Sequence | cccgatgt (SEQ ID NO: 81) |
| Heavy Chain Leader Amino acid | MDMRVPAQLLGLLLLWLRGARC (SEQ ID NO: 82) |
| Sequence | |
| Heavy Chain Variable Domain | gaagtgcacctggtggaaacaggcggaggactggttcagcctaagggcagcctga |
| DNA Sequence | cactgtcttgtgccgccagcggcttcaccttcaacatcaacgccatgaactgggtccg |
| | acaggcccctggcaaaggcctggaatggattgcccggatccgcagcatcatcaaca |
| | acaatgccatctactacgccgacagcgtgaaggaccggttcaccatcagcagagatg |
| | acagccagagcatggtgtacctgcagatgaacgacgtgaagtccgaggacaccgc |
| | catctattactgcgtgcggggactgcctcacgctctggattattggggccagggcaca |
| | agcgtgaccgtctcgagc (SEQ ID NO: 83) |
| Heavy Chain Variable Domain | EVHLVETGGGLVQPKGSLTLSCAASGFTFNINAMNW |
| Amino Acid Sequence | VRQAPGKGLEWIARIRSIINNNAIYYADSVKDRFTISR |
| | DDSQSMVYLQMNDVKSEDTAIYYCVRGLPHALDYW |
| | GQGTSVTVSS (SEQ ID NO: 84) |
| Heavy Chain CDR1 Amino Acid | GFTFNINA (SEQ ID NO: 85) |
| Sequence | |
| (IMGT) | |
| Heavy Chain CDR2 Amino Acid | IRSIINNNAI (SEQ ID NO: 86) |
| Sequence | |
| (IMGT) | |
| Heavy Chain CDR3 Amino Acid | VRGLPHALDY (SEQ ID NO: 87) |
| Sequence | |
| (IMGT) | |
| Light Chain Leader DNA | atggacatgagggtgcccgctcaactgctgggtctgctgcttctttggctgcgcgggg |
| Sequence | cccgatgc (SEQ ID NO: 88) |
| Light Chain Leader Amino acid | MDMRVPAQLLGLLLLWLRGARC (SEQ ID NO: 82) |
| Sequence | |
| Light Chain Variable Domain | gacgttgtgctgacccagacaccactgagcctgcctgtgtctctgggagatcaggcc |
| DNA Sequence | agcatcagctgcagatctagccagagcctggtgcacagcaacggcaacacctacct |
| | gtactggtatctgcagaagcccggacagagccccaagctgctgatctacaaggtgtc |
| | caaccggttcagcggcgtgcccgatagattttctggcagcggctctggcaccgacttc |
| | accctgaagatcagctccgtggaagccgaggatctgggcgtgtacttctgcagccag |
| | agcacacacgtgccaccttacacatttggcggaggtaccaagctggaaatcaag |
| | (SEQ ID NO: 89) |
| Light Chain Variable Domain | DVVLTQTPLSLPVSLGDQASISCRSSQSLVHSNGNTYL |
| Amino Acid Sequence | YWYLQKPGQSPKLLIYKVSNRFSGVPDRFSGSGSGTD |
| | FTLKISSVEAEDLGVYFCSQSTHVPPYTFGGGTKLEIK |
| | (SEQ ID NO: 90) |
| Light Chain CDR1 Amino Acid | QSLVHSNGNTY (SEQ ID NO: 91) |
| Sequence | |
| (IMGT) | |
| Light Chain CDR2 Amino Acid | KVS |
| Sequence | |
| (IMGT) | |
| Light Chain CDR3 Amino Acid | SQSTHVPPYT (SEQ ID NO: 92) |
| Sequence | |
| (IMGT) | |
| Heavy Chain DNA Sequence | gaagtgcacctggtggaaacaggcggaggactggttcagcctaagggcagcctga |
| | cactgtcttgtgccgccagcggcttcaccttcaacatcaacgccatgaactgggtccg |
| | acaggcccctggcaaaggcctggaatggattgcccggatccgcagcatcatcaaca |
| | acaatgccatctactacgccgacagcgtgaaggaccggttcaccatcagcagagatg |
| | acagccagagcatggtgtacctgcagatgaacgacgtgaagtccgaggacaccgc |
| | catctattactgcgtgcggggactgcctcacgctctggattattggggccagggcaca |
| | agcgtgaccgtctcgagcgctaagacaacacctcctagcgtgtaccctctggctcctg |
| | gatctgccgctcagaccaatagcatggtcaccctgggctgtctggtcaagggctacttt |
| | cctgagcctgtgaccgtgacctggaacagcggatctctgtctagcggcgtgcacacc |
| | tttccagccgtgctgcagagcgatctgtacaccctgagcagcagcgtgacagtgcct |
| | agctctacctggcctagcgagacagtgacctgcaacgtggcccatcctgccagcag |
| | caccaaggtggacaagaaaatcggcgccgtgcccagagactgcggctgcaagccc |
| | tgcatctgtaccgtgcctgaagtgtccagcgtgttcatcttcccacctaagcctaagga |
| | cgtgctgaccatcacactgacccctaaagtgacctgtgtggtggtggacatcagcaa |
| | ggacgaccccgaggtgcagttcagttggttcgtggacgacgtggaagtgcacacag |
| | cccagacacagcctagagaggaacagttcaacagcaccttcagaagcgtgtccgag |
| | ctgcccatcatgcaccaggattggctgaacggcaaagaattcaagtgcagagtgaac |
| | agcgccgcctttcctgctcctatcgagaaaaccatctccaagaccaagggcagaccc |
| | aaggctccccaggtgtacacaatccctccacctaaagaacagatggccaaggacaa |
| | ggtgtccctgacctgcatgatcaccgatttcttcccagaggacatcaccgtggaatgg |
| | cagtggaatggacagcccgccgagaactacaagaacacccagcctatcatggacac |
| | cgacggcagctacttcgtgtacagcaagctgaacgtgcagaagtccaactgggagg |
| | ccggcaacaccttcacctgttctgtgctgcacgagggcctgcacaatcaccacaccg |
| | agaagtccctgagccactctcctggaaag (SEQ ID NO: 93) |
| Heavy Chain Amino Acid | EVHLVETGGGLVQPKGSLTLSCAASGFTFNINAMNW |
| Sequence | VRQAPGKGLEWIARIRSIINNNAIYYADSVKDRFTISR |
| | DDSQSMVYLQMNDVKSEDTAIYYCVRGLPHALDYW |
| | GQGTSVTVSSAKTTPPSVYPLAPGSAAQTNSMVTLGC |
| | LVKGYFPEPVTVTWNSGSLSSGVHTFPAVLQSDLYTL |
| | SSSVTVPSSTWPSETVTCNVAHPASSTKVDKKIGA |
| | VPRDCGCKPCICTVPEVSSVFIFPPKPKDVLTITLTPKV |
| | TCVVVDISKDDPEVQFSWFVDDVEVHTAQTQPREEQF |
| | NSTFRSVSELPIMHQDWLNGKEFKCRVNSAAFPAPIEK |
| | TISKTKGRPKAPQVYTIPPPKEQMAKDKVSLTCMITDF |
| | FPEDITVEWQWNGQPAENYKNTQPIMDTDGSYFVYS |
| | KLNVQKSNWEAGNTFTCSVLHEGLHNHHTEKSLSHS |
| | PGK (SEQ ID NO: 94) |
| Light Chain DNA Sequence | gacgttgtgctgacccagacaccactgagcctgcctgtgtctctgggagatcaggcc |
| | agcatcagctgcagatctagccagagcctggtgcacagcaacggcaacacctacct |
| | gtactggtatctgcagaagcccggacagagccccaagctgctgatctacaaggtgtc |
| | caaccggttcagcggcgtgcccgatagattttctggcagcggctctggcaccgacttc |
| | accctgaagatcagctccgtggaagccgaggatctgggcgtgtacttctgcagccag |
| | agcacacacgtgccaccttacacatttggcggaggtaccaagctggaaatcaagcg |
| | ggccgacgcggcgcccaccgtgagcatcttcccccccagcagcgagcagctgaca |
| | agcggcggagctagcgtcgtgtgcttcctgaacaacttctaccccaaggacatcaac |
| | gtgaagtggaagatcgacggcagcgagagacagaacggcgtgctgaatagctgga |
| | ccgaccaggacagcaaggactccacctacagcatgtccagcacactgaccctgacc |
| | aaggacgagtacgagcggcacaacagctacacatgcgaggccacacacaagacc |
| | agcacaagccccatcgtgaagtccttcaaccggaacgagtgc (SEQ ID NO: 95) |
| Light Chain Amino Acid | DVVLTQTPLSLPVSLGDQASISCRSSQSLVHSNGNTYL |
| Sequence | YWYLQKPGQSPKLLIYKVSNRFSGVPDRFSGSGSGTD |
| | FTLKISSVEAEDLGVYFCSQSTHVPPYTFGGGTKLEIK |
| | RADAAPTVSIFPPSSEQLTSGGASVVCFLNNFYPKDIN |
| | VKWKIDGSERQNGVLNSWTDQDSKDSTYSMSSTLTL |
| | TKDEYERHNSYTCEATHKTSTSPIVKSFNRNEC (SEQ ID NO: 96) |
| Heavy Chain Fab DNA sequence | gaagtgcacctggtggaaacaggcggaggactggttcagcctaagggcagcctga |
| | cactgtcttgtgccgccagcggcttcaccttcaacatcaacgccatgaactgggtccg |
| | acaggcccctggcaaaggcctggaatggattgcccggatccgcagcatcatcaaca |
| | acaatgccatctactacgccgacagcgtgaaggaccggttcaccatcagcagagatg |
| | acagccagagcatggtgtacctgcagatgaacgacgtgaagtccgaggacaccgc |
| | catctattactgcgtgcggggactgcctcacgctctggattattggggccagggcaca |
| | agcgtgaccgtctcgagcgctaagacaacacctcctagcgtgtaccctctggctcctg |
| | gatctgccgctcagaccaatagcatggtcaccctgggctgtctggtcaagggctacttt |
| | cctgagcctgtgaccgtgacctggaacagcggatctctgtctagcggcgtgcacacc |
| | tttccagccgtgctgcagagcgatctgtacaccctgagcagcagcgtgacagtgcct |
| | agctctacctggcctagcgagacagtgacctgcaacgtggcccatcctgccagcag |
| | caccaaggtggacaagaaaatcggcgcc (SEQ ID NO: 97) |
| Heavy Chain Fab amino acid | EVHLVETGGGLVQPKGSLTLSCAASGFTFNINAMNW |
| sequence | VRQAPGKGLEWIARIRSIINNNAIYYADSVKDRFTISR |
| | DDSQSMVYLQMNDVKSEDTAIYYCVRGLPHALDYW |
| | GQGTSVTVSSAKTTPPSVYPLAPGSAAQTNSMVTLGC |
| | LVKGYFPEPVTVTWNSGSLSSGVHTFPAVLQSDLYTL |
| | SSSVTVPSSTWPSETVTCNVAHPASSTKVDKKIGA |
| | (SEQ ID NO: 98) |

TABLE 17
Anti-Phospho-ERK1/2 Parental Antibody Constant Domains
(Anti-pERK1/2 Antibody)
Anti-Phospho-ERK1/2 (Thr202, Tyr204) Antibody
Clone Name: MILAN8R
Species: Mouse
Isotype: IgG1, kappa

| | |
|---|---|
| Heavy Chain CH1 DNA | gctaagacaacacctcctagcgtgtaccctctggctcctggatctgccgctcagacca |
| Sequence | atagcatggtcaccctgggctgtctggtcaagggctactttcctgagcctgtgaccgtg |
| | acctggaacagcggatctctgtctagcggcgtgcacacctttccagccgtgctgcag |
| | agcgatctgtacaccctgagcagcagcgtgacagtgcctagctctacctggcctagc |
| | gagacagtgacctgcaacgtggcccatcctgccagcagcaccaaggtggacaaga |
| | aaatcggcgcc (SEQ ID NO: 99) |
| Heavy Chain CH1 Amino acid | AKTTPPSVYPLAPGSAAQTNSMVTLGCLVKGYFPEPV |
| Sequence | TVTWNSGSLSSGVHTFPAVLQSDLYTLSSSVTVPSST |
| | WPSETVTCNVAHPASSTKVDKKIGA (SEQ ID NO: 100) |
| Heavy Chain Hinge DNA | gtgcccagagactgcggctgcaagccctgcatctgtaccgtgcctgaagtgtccagc |
| Sequence | (SEQ ID NO: 101) |
| Heavy Chain Hinge Amino Acid | VPRDCGCKPCICTVPEVSS (SEQ ID NO: 102) |
| Sequence | |
| Heavy Chain CH2 DNA | gtgttcatcttcccacctaagcctaaggacgtgctgaccatcacactgacccctaaagt |
| Sequence | gacctgtgtggtggtggacatcagcaaggacgaccccgaggtgcagttcagttggtt |
| | cgtggacgacgtggaagtgcacacagcccagacacagcctagagaggaacagttc |
| | aacagcaccttcagaagcgtgtccgagctgcccatcatgcaccaggattggctgaac |
| | ggcaaagaattcaagtgcagagtgaacagcgccgcctttcctgctcctatcgagaaa |
| | accatctccaagaccaag (SEQ ID NO: 103) |
| Heavy Chain CH2 Amino Acid | VFIFPPKPKDVLTITLTPKVTCVVVDISKDDPEVQFSWF |
| Sequence | VDDVEVHTAQTQPREEQFNSTFRSVSELPIMHQDWLN |
| | GKEFKCRVNSAAFPAPIEKTISKTK (SEQ ID NO: 104) |
| Heavy Chain CH3 DNA | ggcagacccaaggctccccaggtgtacacaatccctccacctaaagaacagatggc |
| Sequence | caaggacaaggtgtccctgacctgcatgatcaccgatttcttcccagaggacatcacc |
| | gtggaatggcagtggaatggacagcccgccgagaactacaagaacacccagcctat |
| | catggacaccgacggcagctacttcgtgtacagcaagctgaacgtgcagaagtcca |
| | actgggaggccggcaacaccttcacctgttctgtgctgcacgagggcctgcacaatc |
| | accacaccgagaagtccctgagccactctcctggaaag (SEQ ID NO: 105) |
| Heavy Chain CH3 Amino Acid | GRPKAPQVYTIPPPKEQMAKDKVSLTCMITDFFPEDIT |
| Sequence | VEWQWNGQPAENYKNTQPIMDTDGSYFVYSKLNVQ |
| | KSNWEAGNTFTCSVLHEGLHNHHTEKSLSHSPGK |
| | (SEQ ID NO: 106) |
| Light Chain Constant DNA | cgggccgacgcggcgcccaccgtgagcatcttcccccccagcagcgagcagctga |
| Sequence | caagcggcggagctagcgtcgtgtgcttcctgaacaacttctaccccaaggacatca |
| | acgtgaagtggaagatcgacggcagcgagagacagaacggcgtgctgaatagctg |
| | gaccgaccaggacagcaaggactccacctacagcatgtccagcacactgaccctga |
| | ccaaggacgagtacgagcggcacaacagctacacatgcgaggccacacacaaga |
| | ccagcacaagccccatcgtgaagtccttcaaccggaacgagtgc (SEQ ID NO: 107) |
| Light Chain Constant Amino | RADAAPTVSIFPPSSEQLTSGGASVVCFLNNFYPKDIN |
| Acid sequence | VKWKIDGSERQNGVLNSWTDQDSKDSTYSMSSTLTL |
| | TKDEYERHNSYTCEATHKTSTSPIVKSFNRNEC (SEQ ID NO: 108) |

TABLE 18
Anti PSD-95 Antibody Variable Domains
Anti-PSD-95 Antibody
Clone Name: 7E3-1B8
Species: Mouse
Isotype: IgG1, kappa

| | |
|---|---|
| Heavy Chain Leader DNA | atggacatgagggtgcccgctcaactgctgggtctgctgcttctttggctgcgcgggg |
| Sequence | cccgatgt (SEQ ID NO: 81) |
| Heavy Chain Leader Amino acid | MDMRVPAQLLGLLLLWLRGARC (SEQ ID NO: 82) |
| Sequence | |
| Heavy Chain Variable Domain | gaaggacagctgcagcagtctggacctgagctgcttaaacctggagcctccgtgaa |
| DNA Sequence | gatgagctgcaaggccagcggctacaccttcaccagctacgtgatgcactgggtcaa |
| | gcagaagccaggccagggccttgagtggatcggctacatcaaccccttcaacgact |
| | acaccaactacaacgagaagttccgcggcaaggccacactgaccagcgacaagtct |
| | agcaacaccgcctacatggacctgtccagcctgacctctgaggacagcgccgtgtac |
| | tactgtgccagacaaggtggcagagccctggattattggggccagggcacaagcgtt |
| | accgtctcgagc (SEQ ID NO: 109) |
| Heavy Chain Variable Domain | EGQLQQSGPELLKPGASVKMSCKASGYTFTSYVMHW |
| Amino Acid Sequence | VKQKPGQGLEWIGYINPFNDYTNYNEKFRGKATLTSD |
| | KSSNTAYMDLSSLTSEDSAVYYCARQGGRALDYWGQ |
| | GTSVTVSS (SEQ ID NO: 110) |
| Heavy Chain CDR1 Amino Acid | GYTFTSYV (SEQ ID NO: 111) |
| Sequence | |
| (IMGT) | |
| Heavy Chain CDR2 Amino Acid | INPFNDYT (SEQ ID NO: 112) |
| Sequence | |
| (IMGT) | |
| Heavy Chain CDR3 Amino Acid | ARQGGRALDY (SEQ ID NO: 113) |
| Sequence | |
| (IMGT) | |
| Light Chain Leader DNA | atggacatgagggtgcccgctcaactgctgggtctgctgcttctttggctgcgcgggg |
| Sequence | cccgatgc (SEQ ID NO: 88) |
| Light Chain Leader Amino Acid | MDMRVPAQLLGLLLLWLRGARC (SEQ ID NO: 82) |
| Sequence | |
| Light Chain Variable Domain | cagatcgtgctgacacagagccctgccatcatgtctgctagccctggcgagaaagtg |
| DNA Sequence | accatgacctgtagcgccagcagcagcgtgacctacatccactggtatcagctgaag |
| | tccggcacaagccccaagcggtggatctacgatacaagcaagctggcctctggcgt |
| | gcccgctagattttctggctctggcagcggcaccagctacagcctgacaatcagcag |
| | catggaagccgaggatgccgccacctactactgccagcagtggtccagcaatccctt |
| | cacatttggctccggcaccaagctggaaatcaag (SEQ ID NO: 114) |
| Light Chain Variable Domain | QIVLTQSPAIMSASPGEKVTMTCSASSSVTYIHWYQLK |
| Amino Acid Sequence | SGTSPKRWIYDTSKLASGVPARFSGSGSGTSYSLTISS |
| | MEAEDAATYYCQQWSSNPFTFGSGTKLEIK (SEQ ID NO: 115) |
| Light Chain CDR1 Amino Acid | SSVTY (SEQ ID NO: 116) |
| Sequence | |
| (IMGT) | |
| Light Chain CDR2 Amino Acid | DTS |
| Sequence | |
| (IMGT) | |
| Light Chain CDR3 Amino Acid | QQWSSNPFT (SEQ ID NO: 117) |
| Sequence | |
| (IMGT) | |
| Heavy Chain DNA Sequence | gaaggacagctgcagcagtctggacctgagctgcttaaacctggagcctccgtgaa |
| | gatgagctgcaaggccagcggctacaccttcaccagctacgtgatgcactgggtcaa |
| | gcagaagccaggccagggccttgagtggatcggctacatcaaccccttcaacgact |
| | acaccaactacaacgagaagttccgcggcaaggccacactgaccagcgacaagtct |
| | agcaacaccgcctacatggacctgtccagcctgacctctgaggacagcgccgtgtac |
| | tactgtgccagacaaggtggcagagccctggattattggggccagggcacaagcgtt |
| | accgtctcgagcgctaagacaacacctcctagcgtgtaccctctggctcctggatctg |
| | ccgctcagaccaatagcatggtcaccctgggctgtctggtcaagggctactttcctga |
| | gcctgtgaccgtgacctggaacagcggatctctgtctagcggcgtgcacacctttcca |
| | gccgtgctgcagagcgatctgtacaccctgagcagcagcgtgacagtgcctagctct |
| | acctggcctagcgagacagtgacctgcaacgtggcccatcctgccagcagcaccaa |
| | ggtggacaagaaaatcggcgccgtgcccagagactgcggctgcaagccctgcatct |
| | gtaccgtgcctgaagtgtccagcgtgttcatcttcccacctaagcctaaggacgtgctg |
| | accatcacactgacccctaaagtgacctgtgtggtggtggacatcagcaaggacgac |
| | cccgaggtgcagttcagttggttcgtggacgacgtggaagtgcacacagcccagac |
| | acagcctagagaggaacagttcaacagcaccttcagaagcgtgtccgagctgcccat |
| | catgcaccaggattggctgaacggcaaagaattcaagtgcagagtgaacagcgccg |
| | cctttcctgctcctatcgagaaaaccatctccaagaccaagggcagacccaaggctc |
| | cccaggtgtacacaatccctccacctaaagaacagatggccaaggacaaggtgtcc |
| | ctgacctgcatgatcaccgatttcttcccagaggacatcaccgtggaatggcagtgga |
| | atggacagcccgccgagaactacaagaacacccagcctatcatggacaccgacgg |
| | cagctacttcgtgtacagcaagctgaacgtgcagaagtccaactgggaggccggca |
| | acaccttcacctgttctgtgctgcacgagggcctgcacaatcaccacaccgagaagtc |
| | cctgagccactctcctggaaag (SEQ ID NO: 118) |
| Heavy Chain Amino Acid | EGQLQQSGPELLKPGASVKMSCKASGYTFTSYVMHW |
| Sequence | VKQKPGQGLEWIGYINPFNDYTNYNEKFRGKATLTSD |
| | KSSNTAYMDLSSLTSEDSAVYYCARQGGRALDYWGQ |
| | GTSVTVSSAKTTPPSVYPLAPGSAAQTNSMVTLGCLV |
| | KGYFPEPVTVTWNSGSLSSGVHTFPAVLQSDLYTLSSS |
| | VTVPSSTWPSETVTCNVAHPASSTKVDKKIGAVPRDC |
| | GCKPCICTVPEVSSVFIFPPKPKDVLTITLTPKVTCVVV |
| | DISKDDPEVQFSWFVDDVEVHTAQTQPREEQFNSTFR |
| | SVSELPIMHQDWLNGKEFKCRVNSAAFPAPIEKTISKT |
| | KGRPKAPQVYTIPPPKEQMAKDKVSLTCMITDFFPEDI |
| | TVEWQWNGQPAENYKNTQPIMDTDGSYFVYSKLNV |
| | QKSNWEAGNTFTCSVLHEGLHNHHTEKSLSHSPGK |
| | (SEQ ID NO: 119) |
| Light Chain DNA Sequence | cagatcgtgctgacacagagccctgccatcatgtctgctagccctggcgagaaagtg |
| | accatgacctgtagcgccagcagcagcgtgacctacatccactggtatcagctgaag |
| | tccggcacaagccccaagcggtggatctacgatacaagcaagctggcctctggcgt |
| | gcccgctagattttctggctctggcagcggcaccagctacagcctgacaatcagcag |
| | catggaagccgaggatgccgccacctactactgccagcagtggtccagcaatccctt |
| | cacatttggctccggcaccaagctggaaatcaagcgggccgacgcggcgcccacc |
| | gtgagcatcttcccccccagcagcgagcagctgacaagcggcggagctagcgtcgt |
| | gtgcttcctgaacaacttctaccccaaggacatcaacgtgaagtggaagatcgacgg |
| | cagcgagagacagaacggcgtgctgaatagctggaccgaccaggacagcaagga |
| | ctccacctacagcatgtccagcacactgaccctgaccaaggacgagtacgagcggc |
| | acaacagctacacatgcgaggccacacacaagaccagcacaagccccatcgtgaa |
| | gtccttcaaccggaacgagtgc (SEQ ID NO: 120) |
| Light Chain Amino Acid | QIVLTQSPAIMSASPGEKVTMTCSASSSVTYIHWYQLK |
| Sequence | SGTSPKRWIYDTSKLASGVPARFSGSGSGTSYSLTISS |
| | MEAEDAATYYCQQWSSNPFTFGSGTKLEIKRADAAPT |
| | VSIFPPSSEQLTSGGASVVCFLNNFYPKDINVKWKIDG |
| | SERQNGVLNSWTDQDSKDSTYSMSSTLTLTKDEYER |
| | HNSYTCEATHKTSTSPIVKSFNRNEC (SEQ ID NO: 121) |
| Heavy chain Fab DNA sequence | gaaggacagctgcagcagtctggacctgagctgcttaaacctggagcctccgtgaa |
| | gatgagctgcaaggccagcggctacaccttcaccagctacgtgatgcactgggtcaa |
| | gcagaagccaggccagggccttgagtggatcggctacatcaaccccttcaacgact |
| | acaccaactacaacgagaagttccgcggcaaggccacactgaccagcgacaagtct |
| | agcaacaccgcctacatggacctgtccagcctgacctctgaggacagcgccgtgtac |
| | tactgtgccagacaaggtggcagagccctggattattggggccagggcacaagcgtt |
| | accgtctcgagcgctaagacaacacctcctagcgtgtaccctctggctcctggatctg |
| | ccgctcagaccaatagcatggtcaccctgggctgtctggtcaagggctactttcctga |
| | gcctgtgaccgtgacctggaacagcggatctctgtctagcggcgtgcacacctttcca |
| | gccgtgctgcagagcgatctgtacaccctgagcagcagcgtgacagtgcctagctct |
| | acctggcctagcgagacagtgacctgcaacgtggcccatcctgccagcagcaccaa |
| | ggtggacaagaaaatcggcgcc (SEQ ID NO: 122) |
| Heavy chain Fab amino acid | EGQLQQSGPELLKPGASVKMSCKASGYTFTSYVMHW |
| sequence | VKQKPGQGLEWIGYINPFNDYTNYNEKFRGKATLTSD |
| | KSSNTAYMDLSSLTSEDSAVYYCARQGGRALDYWGQ |
| | GTSVTVSSAKTTPPSVYPLAPGSAAQTNSMVTLGCLV |
| | KGYFPEPVTVTWNSGSLSSGVHTFPAVLQSDLYTLSSS |
| | VTVPSSTWPSETVTCNVAHPASSTKVDKKIGA (SEQ ID NO: 123) |

TABLE 19
Anti PSD-95 Antibody Constant Domains
Anti-PSD-95 Antibody
Clone Name: 7E3-1B8
Species: Mouse
Isotype: IgG1, kappa

| | |
|---|---|
| Heavy Chain CH1 DNA | gctaagacaacacctcctagcgtgtaccctctggctcctggatctgccgctcagacca |
| Sequence | atagcatggtcaccctgggctgtctggtcaagggctactttcctgagcctgtgaccgtg |
| | acctggaacagcggatctctgtctagcggcgtgcacacctttccagccgtgctgcag |
| | agcgatctgtacaccctgagcagcagcgtgacagtgcctagctctacctggcctagc |
| | gagacagtgacctgcaacgtggcccatcctgccagcagcaccaaggtggacaaga |
| | aaatcggcgcc (SEQ ID NO: 99) |
| Heavy Chain CH1 Amino acid | AKTTPPSVYPLAPGSAAQTNSMVTLGCLVKGYFPEPV |
| Sequence | TVTWNSGSLSSGVHTFPAVLQSDLYTLSSSVTVPSST |
| | WPSETVTCNVAHPASSTKVDKKIGA (SEQ ID NO: 100) |
| Heavy Chain Hinge DNA | gtgcccagagactgcggctgcaagccctgcatctgtaccgtgcctgaagtgtccagc |
| Sequence | (SEQ ID NO: 101) |
| Heavy Chain Hinge Amino Acid | VPRDCGCKPCICTVPEVSS (SEQ ID NO: 102) |
| Sequence | |
| Heavy Chain CH2 DNA | gtgttcatcttcccacctaagcctaaggacgtgctgaccatcacactgacccctaaagt |
| Sequence | gacctgtgtggtggtggacatcagcaaggacgaccccgaggtgcagttcagttggtt |
| | cgtggacgacgtggaagtgcacacagcccagacacagcctagagaggaacagttc |
| | aacagcaccttcagaagcgtgtccgagctgcccatcatgcaccaggattggctgaac |
| | ggcaaagaattcaagtgcagagtgaacagcgccgcctttcctgctcctatcgagaaa |
| | accatctccaagaccaag (SEQ ID NO: 103) |
| Heavy Chain CH2 Amino Acid | VFIFPPKPKDVLTITLTPKVTCVVVDISKDDPEVQFSWF |
| Sequence | VDDVEVHTAQTQPREEQFNSTFRSVSELPIMHQDWLN |
| | GKEFKCRVNSAAFPAPIEKTISKTK (SEQ ID NO: 104) |
| Heavy Chain CH3 DNA | ggcagacccaaggctccccaggtgtacacaatccctccacctaaagaacagatggc |
| Sequence | caaggacaaggtgtccctgacctgcatgatcaccgatttcttcccagaggacatcacc |
| | gtggaatggcagtggaatggacagcccgccgagaactacaagaacacccagcctat |
| | catggacaccgacggcagctacttcgtgtacagcaagctgaacgtgcagaagtcca |
| | actgggaggccggcaacaccttcacctgttctgtgctgcacgagggcctgcacaatc |
| | accacaccgagaagtccctgagccactctcctggaaag (SEQ ID NO: 105) |
| Heavy Chain CH3 Amino Acid | GRPKAPQVYTIPPPKEQMAKDKVSLTCMITDFFPEDIT |
| Sequence | VEWQWNGQPAENYKNTQPIMDTDGSYFVYSKLNVQ |
| | KSNWEAGNTFTCSVLHEGLHNHHTEKSLSHSPGK |
| | (SEQ ID NO: 106) |
| Light Chain Constant DNA | cgggccgacgcggcgcccaccgtgagcatcttcccccccagcagcgagcagctga |
| Sequence | caagcggcggagctagcgtcgtgtgcttcctgaacaacttctaccccaaggacatca |
| | acgtgaagtggaagatcgacggcagcgagagacagaacggcgtgctgaatagctg |
| | gaccgaccaggacagcaaggactccacctacagcatgtccagcacactgaccctga |
| | ccaaggacgagtacgagcggcacaacagctacacatgcgaggccacacacaaga |
| | ccagcacaagccccatcgtgaagtccttcaaccggaacgagtgc (SEQ ID NO: 107) |
| Light Chain Constant Amino | RADAAPTVSIFPPSSEQLTSGGASVVCFLNNFYPKDIN |
| Acid sequence | VKWKIDGSERQNGVLNSWTDQDSKDSTYSMSSTLTL |
| | TKDEYERHNSYTCEATHKTSTSPIVKSFNRNEC (SEQ ID NO: 108) |

TABLE 20
Rabbit anti-Mouse IgG Secondary Antibody (RAM) Variable Domains
Rabbit anti-Mouse IgG Secondary Antibody
Clone Name: RAM 35HC35LC37
Species: Rabbit
Isotype: IgG

| | |
|---|---|
| Heavy Chain Leader DNA | atggagactgggctgcgctggctgctcctggtcgctgtgctcaaaggtgtccagtgt |
| Sequence | (SEQ ID NO: 124) |
| Heavy Chain Leader Amino acid | METGLRWLLLVAVLKGVQC (SEQ ID NO: 63) |
| Sequence | |
| Heavy Chain Variable Domain | cagtcgctggaggagtccgggggtcgcctggtggcgcctgggacacccctgacact |
| DNA Sequence | cacctgcacagtctctggaatcgacctcagtagctatgcaatgggctgggtccgcca |
| | ggctccagggaaggggctggaatacatcggatacataagcagtagtggtagcacag |
| | actacgcgagctgggcgagaggccgattcaccatctccaaaacctcgtcgaccacg |
| | gtggatctggaaatgaccagtctgacaaccgaggacacggccacctatttctgtgcca |
| | gggacacgaattataatggttatcgatttaagatctggggcccaggcaccctggtcac |
| | cgtctcctca (SEQ ID NO: 125) |
| Heavy Chain Variable Domain | QSLEESGGRLVAPGTPLTLTCTVSGIDLSSYAMGWVR |
| Amino Acid Sequence | QAPGKGLEYIGYISSSGSTDYASWARGRFTISKTSSTT |
| | VDLEMTSLTTEDTATYFCARDTNYNGYRFKIWGPGTL |
| | VTVSS (SEQ ID NO: 126) |
| Heavy Chain CDR1 Amino Acid | GIDLSSYA (SEQ ID NO: 127) |
| Sequence | |
| (IMGT) | |
| Heavy Chain CDR2 Amino Acid | ISSSGST (SEQ ID NO: 128) |
| Sequence | |
| (IMGT) | |
| Heavy Chain CDR3 Amino Acid | ARDTNYNGYRFKI (SEQ ID NO: 129) |
| Sequence | |
| (IMGT) | |
| Light Chain Leader DNA | atggacacgagggcccccactcagctgctggggctcctgctgctctggctcccaggt |
| Sequence | gccacattt (SEQ ID NO: 10) |
| Light Chain Leader Amino acid | MDTRAPTQLLGLLLLWLPGATF (SEQ ID NO: 69) |
| Sequence | |
| Light Chain Variable Domain | gccgccgtgctgacccagactccagcctccgtgtctgcagctgtgggaggcacagt |
| DNA Sequence | caccatcaattgccagcccagtcagagtgtttacaataatcactacttagcctggtatca |
| | acagaaatcagggcagcctcccaagctcctgatctatggtgcatcttatttggaatctg |
| | gggtcccatcaaggttcaaaggcagtggatctgggacacagttcactctcaccatcag |
| | cgacgtacagtgtgacgatgctgccacttactactgtgtgggcgcttatgatagttatag |
| | tgatactgctttcggcggcgggaccgaggtggtggtcaaaggt (SEQ ID NO: 130) |
| Light Chain Variable Domain | AAVLTQTPASVSAAVGGTVTINCQPSQSVYNNHYLA |
| Amino Acid Sequence | WYQQKSGQPPKLLIYGASYLESGVPSRFKGSGSGTQF |
| | TLTISDVQCDDAATYYCVGAYDSYSDTAFGGGTEVV |
| | VKG (SEQ ID NO: 131) |
| Light Chain CDR1 Amino Acid | QSVYNNHY (SEQ ID NO: 132) |
| Sequence | |
| (IMGT) | |
| Light Chain CDR2 Amino Acid | GAS |
| Sequence | |
| (IMGT) | |
| Light Chain CDR3 Amino Acid | VGAYDSYSDTA (SEQ ID NO: 133) |
| Sequence | |
| (IMGT) | |
| Heavy Chain DNA Sequence | cagtcgctggaggagtccgggggtcgcctggtggcgcctgggacacccctgacact |
| | cacctgcacagtctctggaatcgacctcagtagctatgcaatgggctgggtccgcca |
| | ggctccagggaaggggctggaatacatcggatacataagcagtagtggtagcacag |
| | actacgcgagctgggcgagaggccgattcaccatctccaaaacctcgtcgaccacg |
| | gtggatctggaaatgaccagtctgacaaccgaggacacggccacctatttctgtgcca |
| | gggacacgaattataatggttatcgatttaagatctggggcccaggcaccctggtcac |
| | cgtctcctcagggcaacctaaggctccatcagtcttcccactggccccctgctgcggg |
| | gacacacccagctccacggtgaccctgggctgcctggtcaaaggctacctcccgga |
| | gccagtgaccgtgacctggaactcgggcaccctcaccaatggggtacgcaccttcc |
| | cgtccgtccggcagtcctcaggcctctactcgctgagcagcgtggtgagcgtgacct |
| | caagcagccagcccgtcacctgcaacgtggcccacccagccaccaacaccaaagt |
| | ggacaagaccgtggcgccctcgacatgcagcaagcccacgtgcccaccccctgaa |
| | ctcctggggggaccgtctgtcttcatcttccccccaaaacccaaggacaccctcatga |
| | tctcacgcacccccgaggtcacatgcgtggtggtggacgtgagccaggatgacccc |
| | gaggtgcagttcacatggtacataaacaacgagcaggtgcgcaccgcccggccgc |
| | cgctacgggagcagcagttcaacagcacgatccgcgtggtcagcaccctccccatc |
| | gcgcaccaggactggctgaggggcaaggagttcaagtgcaaagtccacaacaagg |
| | cactcccggcccccatcgagaaaaccatctccaaagccagaggg (SEQ ID NO: 134) |
| Heavy Chain Amino Acid | QSLEESGGRLVAPGTPLTLTCTVSGIDLSSYAMGWVR |
| Sequence | QAPGKGLEYIGYISSSGSTDYASWARGRFTISKTSSTT |
| | VDLEMTSLTTEDTATYFCARDTNYNGYRFKIWGPGTL |
| | VTVSSGQPKAPSVFPLAPCCGDTPSSTVTLGCLVKGYL |
| | PEPVTVTWNSGTLTNGVRTFPSVRQSSGLYSLSSVVSV |
| | TSSSQPVTCNVAHPATNTKVDKTVAPSTCSKPTCPPPE |
| | LLGGPSVFIFPPKPKDTLMISRTPEVTCVVVDVSQDDP |
| | EVQFTWYINNEQVRTARPPLREQQFNSTIRVVSTLPIA |
| | HQDWLRGKEFKCKVHNKALPAPIEKTISKARGQPLEP |
| | KVYTMGPPREELSSRSVSLTCMINGFYPSDISVEWEKN |
| | GKAEDNYKTTPAVLDSDGSYFLYSKLSVPTSEWQRG |
| | DVFTCSVMHEALHNHYTQKSISRSPGK (SEQ ID NO: 135) |
| Light Chain DNA Sequence | gccgccgtgctgacccagactccagcctccgtgtctgcagctgtgggaggcacagt |
| | caccatcaattgccagcccagtcagagtgtttacaataatcactacttagcctggtatca |
| | acagaaatcagggcagcctcccaagctcctgatctatggtgcatcttatttggaatctg |
| | gggtcccatcaaggttcaaaggcagtggatctgggacacagttcactctcaccatcag |
| | cgacgtacagtgtgacgatgctgccacttactactgtgtgggcgcttatgatagttatag |
| | tgatactgctttcggcggcgggaccgaggtggtggtcaaaggtgatccagttgcacct |
| | actgtcctcatcttcccaccagctgctgatcaggtggcaactggaacagtcaccatcgt |
| | gtgtgtggcgaataaatactttcccgatgtcaccgtcacctgggaggtggatggcacc |
| | acccaaacaactggcatcgagaacagtaaaacaccgcagaattctgcagattgtacc |
| | tacaacctcagcagcactctgacactgaccagcacacagtacaacagccacaaaga |
| | gtacacctgcaaggtgacccagggcacgacctcagtcgtccagagcttcaataggg |
| | gtgactgt (SEQ ID NO: 136) |
| Light Chain Amino Acid | AAVLTQTPASVSAAVGGTVTINCQPSQSVYNNHYLA |
| Sequence | WYQQKSGQPPKLLIYGASYLESGVPSRFKGSGSGTQF |
| | TLTISDVQCDDAATYYCVGAYDSYSDTAFGGGTEVV |
| | VKGDPVAPTVLIFPPAADQVATGTVTIVCVANKYFPD |
| | VTVTWEVDGTTQTTGIENSKTPQNSADCTYNLSSTLT |
| | LTSTQYNSHKEYTCKVTQGTTSVVQSFNRGDC (SEQ ID NO: 137) |
| Heavy Chain Fab DNA sequence | cagtcgctggaggagtccgggggtcgcctggtggcgcctgggacacccctgacact |
| | cacctgcacagtctctggaatcgacctcagtagctatgcaatgggctgggtccgcca |
| | ggctccagggaaggggctggaatacatcggatacataagcagtagtggtagcacag |
| | actacgcgagctgggcgagaggccgattcaccatctccaaaacctcgtcgaccacg |
| | gtggatctggaaatgaccagtctgacaaccgaggacacggccacctatttctgtgcca |
| | gggacacgaattataatggttatcgatttaagatctggggcccaggcaccctggtcac |
| | cgtctcctcagggcaacctaaggctccatcagtcttcccactggccccctgctgcggg |
| | gacacacccagctccacggtgaccctgggctgcctggtcaaaggctacctcccgga |
| | gccagtgaccgtgacctggaactcgggcaccctcaccaatggggtacgcaccttcc |
| | cgtccgtccggcagtcctcaggcctctactcgctgagcagcgtggtgagcgtgacct |
| | caagcagccagcccgtcacctgcaacgtggcccacccagccaccaacaccaaagt |
| | ggacaagaccgtgcagcccctggagccgaaggtctacaccatgggccctccccgg |
| | gaggagctgagcagcaggtcggtcagcctgacctgcatgatcaacggcttctaccct |
| | tccgacatctcggtggagtgggagaagaacgggaaggcagaggacaactacaaga |
| | ccacgccggccgtgctggacagcgacggctcctacttcctctacagcaagctctcag |
| | tgcccacgagtgagtggcagcggggcgacgtcttcacctgctccgtgatgcacgag |
| | gccttgcacaaccactacacgcagaagtccatctcccgctctccgggtaaa (SEQ |
| | ID NO: 138) |
| Heavy Chain Fab amino acid | QSLEESGGRLVAPGTPLTLTCTVSGIDLSSYAMGWVR |
| sequence | QAPGKGLEYIGYISSSGSTDYASWARGRFTISKTSSTT |
| | VDLEMTSLTTEDTATYFCARDTNYNGYRFKIWGPGTL |
| | VTVSSGQPKAPSVFPLAPCCGDTPSSTVTLGCLVKGYL |
| | PEPVTVTWNSGTLTNGVRTFPSVRQSSGLYSLSSVVSV |
| | TSSSQPVTCNVAHPATNTKVDKTV (SEQ ID NO: 139) |

TABLE 21
Rabbit anti-Mouse IgG Secondary Parental Antibody (RAM) Constant Domains
Rabbit anti-Mouse IgG Secondary Antibody
Clone Name: RAM 35HC35LC37
Species: Rabbit
Isotype: IgG

| | |
|---|---|
| Heavy Chain CH1 DNA | gggcaacctaaggctccatcagtcttcccactggccccctgctgggggacacaccc |
| sequence | agctccacggtgaccctgggctgcctggtcaaaggctacctcccggagccagtgac |
| | cgtgacctggaactcgggcaccctcaccaatggggtacgcaccttcccgtccgtccg |
| | gcagtcctcaggcctctactcgctgagcagcgtggtgagcgtgacctcaagcagcca |
| | gcccgtcacctgcaacgtggcccacccagccaccaacaccaaagtggacaagacc |
| | gtg (SEQ ID NO: 177) |
| Heavy Chain CH1 Amino acid | GQPKAPSVFPLAPCCGDTPSSTVTLGCLVKGYLPEPVT |
| Sequence | VTWNSGTLTNGVRTFPSVRQSSGLYSLSSVVSVTSSSQ |
| | PVTCNVAHPATNTKVDKTV (SEQ ID NO: 17) |
| Heavy Chain Hinge DNA | gcgccctcgacatgcagcaagcccacgtgcccaccccctgaactcctggggggac |
| Sequence | cg (SEQ ID NO: 22) |
| Heavy Chain Hinge Amino Acid | APSTCSKPTCPPPELLGGP (SEQ ID NO: 18) |
| Sequence | |
| Heavy Chain CH2 DNA | tctgtcttcatcttccccccaaaacccaaggacaccctcatgatctcacgcacccccga |
| Sequence | ggtcacatgcgtggtggtggacgtgagccaggatgaccccgaggtgcagttcacat |
| | ggtacataaacaacgagcaggtgcgcaccgcccggccgccgctacgggagcagc |
| | agttcaacagcacgatccgcgtggtcagcaccctccccatcgcgcaccaggactgg |
| | ctgaggggcaaggagttcaagtgcaaagtccacaacaaggcactcccggcccccat |
| | cgagaaaaccatctccaaagccagaggg (SEQ ID NO: 23) |
| Heavy Chain CH2 Amino Acid | SVFIFPPKPKDTLMISRTPEVTCVVVDVSQDDPEVQFT |
| Sequence | WYINNEQVRTARPPLREQQFNSTIRVVSTLPIAHQDW |
| | LRGKEFKCKVHNKALPAPIEKTISKARG (SEQ ID NO: 19) |
| Heavy Chain CH3 DNA | cagcccctggagccgaaggtctacaccatgggccctccccgggaggagctgagca |
| Sequence | gcaggtcggtcagcctgacctgcatgatcaacggcttctacccttccgacatctcggt |
| | ggagtgggagaagaacgggaaggcagaggacaactacaagaccacgccggccg |
| | tgctggacagcgacggctcctacttcctctacagcaagctctcagtgcccacgagtga |
| | gtggcagcggggcgacgtcttcacctgctccgtgatgcacgaggccttgcacaacc |
| | actacacgcagaagtccatctcccgctctccgggtaaa (SEQ ID NO: 140) |
| Heavy Chain CH3 Amino Acid | QPLEPKVYTMGPPREELSSRSVSLTCMINGFYPSDISVE |
| Sequence | WEKNGKAEDNYKTTPAVLDSDGSYFLYSKLSVPTSE |
| | WQRGDVFTCSVMHEALHNHYTQKSISRSPGK (SEQ ID NO: 141) |
| Light Chain Constant DNA | gatccagttgcacctactgtcctcatcttcccaccagctgctgatcaggtggcaactgg |
| Sequence | aacagtcaccatcgtgtgtgtggcgaataaatactttcccgatgtcaccgtcacctggg |
| | aggtggatggcaccacccaaacaactggcatcgagaacagtaaaacaccgcagaat |
| | tctgcagattgtacctacaacctcagcagcactctgacactgaccagcacacagtaca |
| | acagccacaaagagtacacctgcaaggtgacccagggcacgacctcagtcgtcca |
| | gagcttcaataggggtgactgt (SEQ ID NO: 80) |
| Light Chain Constant Amino | DPVAPTVLIFPPAADQVATGTVTIVCVANKYFPDVTV |
| Acid sequence | TWEVDGTTQTTGIENSKTPQNSADCTYNLSSTLTLTST |
| | QYNSHKEYTCKVTQGTTSVVQSFNRGDC (SEQ ID NO: 25) |

TABLE 22
Mouse IgG1 2TdCH2 Domain DNA Sequence

| | |
|---|---|
| Hinge 1 DNA Sequence | gtgcccagagactgcggctgcaagccctgcatctgtaccgtgcctgaagtgtccagc |
| | (SEQ ID NO: 101) |
| First CH2 domain DNA | gtgttcatcttcccacctaagcctaaggacgtgctgaccatcacactgacccctaaagt |
| Sequence | gacctgtgtggtggtggacatcagcaaggacgaccccgaggtgcagttcagttggtt |
| | cgtggacgacgtggaagtgcacacagcccagacacagcctagagaggaacagttc |
| | aacagcaccttcagaagcgtgtccgagctgcccatcatgcaccaggattggctgaac |
| | ggcaaagaattcaagtgcagagtgaacagcgccgcctttcctgctcctatcgagaaa |
| | accatctccaagaccaag (SEQ ID NO: 103) |
| Linker 1 DNA sequence | ggcggcggcggcagcggcggcggcggcagccta (SEQ ID NO: 46) |
| Hinge 2 DNA Sequence | gtgcccagagactgcggctgcaagccctgcatctgtaccgtgcctgaagtgtccagc |
| | (SEQ ID NO: 101) |
| Second CH2 domain DNA | gtgttcatcttcccacctaagcctaaggacgtgctgaccatcacactgacccctaaagt |
| Sequence | gacctgtgtggtggtggacatcagcaaggacgaccccgaggtgcagttcagttggtt |
| | cgtggacgacgtggaagtgcacacagcccagacacagcctagagaggaacagttc |
| | aacagcaccttcagaagcgtgtccgagctgcccatcatgcaccaggattggctgaac |
| | ggcaaagaattcaagtgcagagtgaacagcgccgcctttcctgctcctatcgagaaa |
| | accatctccaagaccaag (SE ID NO: 103) |
| Linker 2 DNA Sequence | ggcggcggcggcagcggcggcggcggcagccta (SEQ ID NO: 46) |
| Hinge 3 DNA Sequence | gtgcccagagactgcggctgcaagccctgcatctgtaccgtgcctgaagtgtccagc |
| | (SEQ ID NO: 101) |
| Fc Domain (CH2 + CH3) | gtgttcatcttcccacctaagcctaaggacgtgctgaccatcacactgacccctaaagt |
| DNA Sequence | gacctgtgtggtggtggacatcagcaaggacgaccccgaggtgcagttcagttggtt |
| | cgtggacgacgtggaagtgcacacagcccagacacagcctagagaggaacagttc |
| | aacagcaccttcagaagcgtgtccgagctgcccatcatgcaccaggattggctgaac |
| | ggcaaagaattcaagtgcagagtgaacagcgccgcctttcctgctcctatcgagaaa |
| | accatctccaagaccaagggcagacccaaggctccccaggtgtacacaatccctcc |
| | acctaaagaacagatggccaaggacaaggtgtccctgacctgcatgatcaccgattt |
| | cttcccagaggacatcaccgtggaatggcagtggaatggacagcccgccgagaact |
| | acaagaacacccagcctatcatggacaccgacggcagctacttcgtgtacagcaag |
| | ctgaacgtgcagaagtccaactgggaggccggcaacaccttcacctgttctgtgctg |
| | cacgagggcctgcacaatcaccacaccgagaagtccctgagccactctcctggaaa |
| | g (SEQ ID NO: 142) |

TABLE 23
Mouse IgG1 2TdCH2 Domain Amino Acid Sequence

| | |
|---|---|
| Hinge 1 Amino Acid Sequence | VPRDCGCKPCICTVPEVSS (SEQ ID NO: 102) |
| First CH2 domain Amino Acid | VFIFPPKPKDVLTITLTPKVTCVVVDISKDDPEVQFSWF |
| Sequence | VDDVEVHTAQTQPREEQFNSTFRSVSELPIMHQDWLN |
| | GKEFKCRVNSAAFPAPIEKTISKTK (SEQ ID NO: 104) |
| Linker 1 Amino Acid Sequence | GGGGSGGGGSL (SEQ ID NO: 44) |
| Hinge 2 Amino Acid Sequence | VPRDCGCKPCICTVPEVSS (SEQ ID NO: 102) |
| Second CH2 domain Amino | VFIFPPKPKDVLTITLTPKVTCVVVDISKDDPEVQFSWF |
| Acid Sequence | VDDVEVHTAQTQPREEQFNSTFRSVSELPIMHQDWLN |
| | GKEFKCRVNSAAFPAPIEKTISKTK (SEQ ID NO: 104) |
| Linker 2 Amino Acid Sequence | GGGGSGGGGSL (SEQ ID NO; 44) |
| Hinge 3 Amino Acid Sequence | VPRDCGCKPCICTVPEVSS (SEQ ID NO: 102) |
| Fc Domain (CH2 + CH3) | VFIFPPKPKDVLTITLTPKVTCVVVDISKDDPEVQFSWF |
| Amino Acid Sequence | VDDVEVHTAQTQPREEQFNSTFRSVSELPIMHQDWLN |
| | GKEFKCRVNSAAFPAPIEKTISKTKGRPKAPQVYTIPPP |
| | KEQMAKDKVSLTCMITDFFPEDITVEWQWNGQPAEN |
| | YKNTQPIMDTDGSYFVYSKLNVQKSNWEAGNTFTCS |
| | VLHEGLHNHHTEKSLSHSPGK (SEQ ID NO: 143) |

TABLE 24
Anti TIGIT Chimeric Antibody Variable Domains
Anti-TIGIT Antibody
Clone Name: MBSA43
Species: Mouse Fab-Rabbit Fc Chimera
Isotype: Rabbit IgG

| | |
|---|---|
| Heavy Chain Leader DNA | atggacatgagggtgcccgctcaactgctgggtctgctgcttctttggctgcgcgggg |
| Sequence | cccgatgc (SEQ ID NO: 88) |
| Heavy Chain Leader Amino acid | MDMRVPAQLLGLLLLWLRGARC (SEQ ID NO: 82) |
| Sequence | |
| Heavy Chain Variable Domain | gatgtgcagattcaggagtcgggacctggcctggtgaaaccttctcagtctctgtccct |
| DNA Sequence | cacttgcactgtcactggctactcaatcaccagtgattatgcctggaactggatacggc |
| | agtttccaggaaacaaactggagtggatgggctacataagctacagtggtagcactta |
| | ctacaacccatctctcaaaagtcgaatctcattcactcgggacacatccaagaatcagt |
| | tcttcctgcagctgaactctgtgactactgaggacacagccacatattactgtgcaaga |
| | tgggagagggacttctatgctatggactactggggtcaaggaacctcagtcaccgtct |
| | cgagc (SEQ ID NO: 144) |
| Heavy Chain Variable Domain | DVQIQESGPGLVKPSQSLSLTCTVTGYSITSDYAWNWI |
| Amino Acid Sequence | RQFPGNKLEWMGYISYSGSTYYNPSLKSRISFTRDTSK |
| | NQFFLQLNSVTTEDTATYYCARWERDFYAMDYWGQ |
| | GTSVTVSS (SEQ ID NO: 145) |
| Heavy Chain CDR1 Amino Acid | GYSITSDYA (SEQ ID NO: 146) |
| Sequence | |
| (IMGT) | |
| Heavy Chain CDR2 Amino Acid | ISYSGST (SEQ ID NO: 147) |
| Sequence | |
| (IMGT) | |
| Heavy Chain CDR3 Amino Acid | ARWERDFYAMDY (SEQ ID NO: 148) |
| Sequence | |
| (IMGT) | |
| Light Chain Leader DNA | atggacatgagggtgcccgctcaactgctgggtctgctgcttctttggctgcgcgggg |
| Sequence | cccgatgc (SEQ ID NO: 88) |
| Light Chain Leader Amino Acid | MDMRVPAQLLGLLLLWLRGARC (SEQ ID NO: 82) |
| Sequence | |
| Light Chain Variable Domain | gacattgtgatgacccagtctcacaaattcatgtccacatcaataggagacagggtca |
| DNA Sequence | gcatcacttgcaatgccagtcagggtgtgactactgctgtagcctggtatcaacagaa |
| | accaggacagtctcctaaactactgatttcctcggcatcctaccggtacactggagtcc |
| | ctgatcgcttcactggcagtggatctgggacggatttcactttcaccatcagcagtgtg |
| | caggctgaagacctggcactttattactgtcagcaatattatagtcctacgtggacgttc |
| | ggtggaggcaccaagctggaaatcaaa (SEQ ID NO: 149) |
| Light Chain Variable Domain | DIVMTQSHKFMSTSIGDRVSITCNASQGVTTAVAWYQ |
| Amino Acid Sequence | QKPGQSPKLLISSASYRYTGVPDRFTGSGSGTDFTFTIS |
| | SVQAEDLALYYCQQYYSPTWTFGGGTKLEIK (SEQ ID NO: 150) |
| Light Chain CDR1 Amino Acid | QGVTTA (SEQ ID NO: 151) |
| Sequence | |
| (IMGT) | |
| Light Chain CDR2 Amino Acid | SAS |
| Sequence | |
| (IMGT) | |
| Light Chain CDR3 Amino Acid | QQYYSPTWT (SEQ ID NO: 152) |
| Sequence | |
| (IMGT) | |
| Heavy Chain DNA Sequence | gatgtgcagattcaggagtcgggacctggcctggtgaaaccttctcagtctctgtccct |
| | cacttgcactgtcactggctactcaatcaccagtgattatgcctggaactggatacggc |
| | agtttccaggaaacaaactggagtggatgggctacataagctacagtggtagcactta |
| | ctacaacccatctctcaaaagtcgaatctcattcactcgggacacatccaagaatcagt |
| | tcttcctgcagctgaactctgtgactactgaggacacagccacatattactgtgcaaga |
| | tgggagagggacttctatgctatggactactggggtcaaggaacctcagtcaccgtct |
| | cgagcgctaagaccaccccccccagcgtgtaccccctggcccccggcagcgccgc |
| | ccagaccaacagcatggtgaccctgggctgcctggtgaagggctacttccccgagc |
| | ccgtgaccgtgacctggaacagcggcagcctgagcagcggcgtgcacaccttccc |
| | cgccgtgctgcagagcgacctgtacaccctgagcagcagcgtgaccgtgcccagc |
| | agcacctggcccagcgagaccgtgacctgcaacgtggcccaccccgccagcagca |
| | ccaaggtggacaagaagatcgcgccctcgacatgcagcaagcccacgtgcccacc |
| | ccctgaactcctggggggaccgtctgtcttcatcttccccccaaaacccaaggacacc |
| | ctcatgatctcacgcacccccgaggtcacatgcgtggtggtggacgtgagccaggat |
| | gaccccgaggtgcagttcacatggtacataaacaacgagcaggtgcgcaccgcccg |
| | gccgccgctacgggagcagcagttcaacagcacgatccgcgtggtcagcaccctcc |
| | ccatcgcgcaccaggactggctgaggggcaaggagttcaagtgcaaagtccacaa |
| | caaggcactcccggcccccatcgagaaaaccatctccaaagccagagggcagccc |
| | ctggagccgaaggtctacaccatgggccctccccgggaggagctgagcagcaggt |
| | cggtcagcctggcctgcatgatcaacggcttctacccttccgacatctcggtggagtg |
| | ggagaagaacgggaaggcagaggacaactacaagaccacgccggccgtgctgga |
| | cagcgacggctcctacttcctctacagcaagctctcagtgcccacgagtgagtggca |
| | gcggggcgacgtcttcacctgctccgtgatgcacgaggccttgcacaaccactacac |
| | gcagaagtccatctcccgctctccgggtaaa (SEQ ID NO: 153) |
| Heavy Chain Amino Acid | DVQIQESGPGLVKPSQSLSLTCTVTGYSITSDYAWNWI |
| Sequence | RQFPGNKLEWMGYISYSGSTYYNPSLKSRISFTRDTSK |
| | NQFFLQLNSVTTEDTATYYCARWERDFYAMDYWGQ |
| | GTSVTVSSAKTTPPSVYPLAPGSAAQTNSMVTLGCLV |
| | KGYFPEPVTVTWNSGSLSSGVHTFPAVLQSDLYTLSSS |
| | VTVPSSTWPSETVTCNVAHPASSTKVDKKIAPSTCSKP |
| | TCPPPELLGGPSVFIFPPKPKDTLMISRTPEVTCVVVDV |
| | SQDDPEVQFTWYINNEQVRTARPPLREQQFNSTIRVVS |
| | TLPIAHQDWLRGKEFKCKVHNKALPAPIEKTISKARG |
| | QPLEPKVYTMGPPREELSSRSVSLACMINGFYPSDISV |
| | EWEKNGKAEDNYKTTPAVLDSDGSYFLYSKLSVPTSE |
| | WQRGDVFTCSVMHEALHNHYTQKSISRSPGK (SEQ ID NO: 154) |
| Light Chain DNA Sequence | gacattgtgatgacccagtctcacaaattcatgtccacatcaataggagacagggtca |
| | gcatcacttgcaatgccagtcagggtgtgactactgctgtagcctggtatcaacagaa |
| | accaggacagtctcctaaactactgatttcctcggcatcctaccggtacactggagtcc |
| | ctgatcgcttcactggcagtggatctgggacggatttcactttcaccatcagcagtgtg |
| | caggctgaagacctggcactttattactgtcagcaatattatagtcctacgtggacgttc |
| | ggtggaggcaccaagctggaaatcaaacgggccgacgcggcgcccaccgtgagc |
| | atcttcccccccagcagcgagcagctgaccagcggcggcgcgagcgtggtgtgctt |
| | cctgaacaacttctaccccaaggacatcaacgtgaagtggaagatcgacggcagcg |
| | agaggcagaacggcgtgctgaacagctggaccgaccaggacagcaaggacagca |
| | cctacagcatgagcagcaccctgaccctgaccaaggacgagtacgagaggcacaa |
| | cagctacacatgtgaggccacccacaagaccagcaccagccccatcgtgaagagct |
| | tcaacaggaacgagtgc (SEQ ID NO: 155) |
| Light Chain Amino Acid | DIVMTQSHKFMSTSIGDRVSITCNASQGVTTAVAWYQ |
| Sequence | QKPGQSPKLLISSASYRYTGVPDRFTGSGSGTDFTFTIS |
| | SVQAEDLALYYCQQYYSPTWTFGGGTKLEIKRADAA |
| | PTVSIFPPSSEQLTSGGASVVCFLNNFYPKDINVKWKI |
| | DGSERQNGVLNSWTDQDSKDSTYSMSSTLTLTKDEY |
| | ERHNSYTCEATHKTSTSPIVKSFNRNEC (SEQ ID NO: 156) |
| Heavy Chain Fab DNA sequence | gatgtgcagattcaggagtcgggacctggcctggtgaaaccttctcagtctctgtccct |
| | cacttgcactgtcactggctactcaatcaccagtgattatgcctggaactggatacggc |
| | agtttccaggaaacaaactggagtggatgggctacataagctacagtggtagcactta |
| | ctacaacccatctctcaaaagtcgaatctcattcactogggacacatccaagaatcagt |
| | tcttcctgcagctgaactctgtgactactgaggacacagccacatattactgtgcaaga |
| | tgggagagggacttctatgctatggactactggggtcaaggaacctcagtcaccgtct |
| | cgagcgctaagaccaccccccccagcgtgtaccccctggcccccggcagcgccgc |
| | ccagaccaacagcatggtgaccctgggctgcctggtgaagggctacttccccgagc |
| | ccgtgaccgtgacctggaacagcggcagcctgagcagcggcgtgcacaccttccc |
| | cgccgtgctgcagagcgacctgtacaccctgagcagcagcgtgaccgtgcccagc |
| | agcacctggcccagcgagaccgtgacctgcaacgtggcccaccccgccagcagca |
| | ccaaggtggacaagaagatc (SEQ ID NO: 157) |
| Heavy Chain Fab amino acid | DVQIQESGPGLVKPSQSLSLTCTVTGYSITSDYAWNWI |
| sequence | RQFPGNKLEWMGYISYSGSTYYNPSLKSRISFTRDTSK |
| | NQFFLQLNSVTTEDTATYYCARWERDFYAMDYWGQ |
| | GTSVTVSSAKTTPPSVYPLAPGSAAQTNSMVTLGCLV |
| | KGYFPEPVTVTWNSGSLSSGVHTFPAVLQSDLYTLSSS |
| | VTVPSSTWPSETVTCNVAHPASSTKVDKKI (SEQ ID NO: 158) |

TABLE 25
Anti TIGIT Chimeric Antibody Constant Domains
Species: Anti-TIGIT Antibody
Clone Name: MBSA43
Mouse Fab-Rabbit Fc Chimera
Isotype: Rabbit IgG

| | |
|---|---|
| Heavy Chain CH1 DNA | gctaagaccaccccccccagcgtgtaccccctggcccccggcagcgccgcccaga |
| Sequence | ccaacagcatggtgaccctgggctgcctggtgaagggctacttccccgagcccgtg |
| | accgtgacctggaacagcggcagcctgagcagcggcgtgcacaccttccccgccg |
| | tgctgcagagcgacctgtacaccctgagcagcagcgtgaccgtgcccagcagcacc |
| | tggcccagcgagaccgtgacctgcaacgtggcccaccccgccagcagcaccaag |
| | gtggacaagaagatc (SEQ ID NO: 159) |
| Heavy Chain CH1 Amino acid | AKTTPPSVYPLAPGSAAQTNSMVTLGCLVKGYFPEPV |
| Sequence | TVTWNSGSLSSGVHTFPAVLQSDLYTLSSSVTVPSST |
| | WPSETVTCNVAHPASSTKVDKKI (SEQ ID NO: 160) |
| Heavy Chain Hinge DNA | gcgccctcgacatgcagcaagcccacgtgcccaccccctgaactcctggggggac |
| Sequence | cg (SEQ ID NO: 22) |
| Heavy Chain Hinge Amino Acid | APSTCSKPTCPPPELLGGP (SEQ ID NO: 18) |
| Sequence | |
| Heavy Chain CH2 DNA | tctgtcttcatcttccccccaaaacccaaggacaccctcatgatctcacgcacccccga |
| Sequence | ggtcacatgcgtggtggtggacgtgagccaggatgaccccgaggtgcagttcacat |
| | ggtacataaacaacgagcaggtgcgcaccgcccggccgccgctacgggagcagc |
| | agttcaacagcacgatccgcgtggtcagcaccctccccatcgcgcaccaggactgg |
| | ctgaggggcaaggagttcaagtgcaaagtccacaacaaggcactcccggcccccat |
| | cgagaaaaccatctccaaagccagaggg (SEQ ID NO: 23) |
| Heavy Chain CH2 Amino Acid | SVFIFPPKPKDTLMISRTPEVTCVVVDVSQDDPEVQFT |
| Sequence | WYINNEQVRTARPPLREQQFNSTIRVVSTLPIAHQDW |
| | LRGKEFKCKVHNKALPAPIEKTISKARG (SEQ ID NO: 19) |
| Heavy Chain CH3 DNA | cagcccctggagccgaaggtctacaccatgggccctccccgggaggagctgagca |
| Sequence | gcaggtcggtcagcctggcctgcatgatcaacggcttctacccttccgacatctcggt |
| | ggagtgggagaagaacgggaaggcagaggacaactacaagaccacgccggccg |
| | tgctggacagcgacggctcctacttcctctacagcaagctctcagtgcccacgagtga |
| | gtggcagcggggcgacgtcttcacctgctccgtgatgcacgaggccttgcacaacc |
| | actacacgcagaagtccatctcccgctctccgggtaaa (SEQ ID NO: 179) |
| Heavy Chain CH3 Amino Acid | QPLEPKVYTMGPPREELSSRSVSLACMINGFYPSDISV |
| Sequence | EWEKNGKAEDNYKTTPAVLDSDGSYFLYSKLSVPTSE |
| | WQRGDVFTCSVMHEALHNHYTQKSISRSPGK (SEQ ID NO: 20) |
| Light Chain Constant DNA | cgggccgacgcggcgcccaccgtgagcatcttcccccccagcagcgagcagctga |
| Sequence | ccagcggcggcgcgagcgtggtgtgcttcctgaacaacttctaccccaaggacatca |
| | acgtgaagtggaagatcgacggcagcgagaggcagaacggcgtgctgaacagct |
| | ggaccgaccaggacagcaaggacagcacctacagcatgagcagcaccctgaccct |
| | gaccaaggacgagtacgagaggcacaacagctacacatgtgaggccacccacaag |
| | accagcaccagccccatcgtgaagagcttcaacaggaacgagtgc (SEQ ID NO: 161) |
| Light Chain Constant Amino | RADAAPTVSIFPPSSEQLTSGGASVVCFLNNFYPKDIN |
| Acid sequence | VKWKIDGSERQNGVLNSWTDQDSKDSTYSMSSTLTL |
| | TKDEYERHNSYTCEATHKTSTSPIVKSFNRNEC (SEQ ID NO: 108) |

TABLE 26
Anti-FLAG-tag Antibody Variable Domains
Anti-FLAG-tag Antibody
Clone Name: 8H8L17
Species: Rabbit
Isotype: IgG

| | |
|---|---|
| Heavy Chain Leader DNA | atggagactgggctgcgctggcttctcctggtcgctgtgctcaaaggtgtccagtgt |
| Sequence | (SEQ ID NO: 1) |
| Heavy Chain Leader Amino acid | METGLRWLLLVAVLKGVQC (SEQ ID NO: 63) |
| Sequence | |
| Heavy Chain Variable Domain | cagtcggtggaggagtccgggggtcgcctggtcacgcctgggacacccctgacact |
| DNA Sequence | cacctgcaccgtctctggattctccctcaataaccatgcaatgggctgggtccgccag |
| | gctcctaggaaggggctggaatatatcggatacattagtattagtggtaccacatacta |
| | cgcgagctgggcgaaaggccgattcaccatctccaagacctcgaccacggtggatc |
| | tgaaaatcaccagtccgacaaccgaggacacgggcacctatttctgtggcagaggtt |
| | gggctagtgataatttgtacttgtggggccaaggcaccctggtcaccgtctcctca |
| | (SEQ ID NO: 181) |
| Heavy Chain Variable Domain | QSVEESGGRLVTPGTPLTLTCTVSGFSLNNHAMGWVR |
| Amino Acid Sequence | QAPRKGLEYIGYISISGTTYYASWAKGRFTISKTSTTV |
| | DLKITSPTTEDTGTYFCGRGWASDNLYLWGQGTLVT |
| | VSS (SEQ ID NO: 162) |
| Heavy Chain CDR1 Amino Acid | GFSLNNHA (SEQ ID NO: 163) |
| Sequence | |
| (IMGT) | |
| Heavy Chain CDR2 Amino Acid | ISISGTT (SEQ ID NO: 164) |
| Sequence | |
| (IMGT) | |
| Heavy Chain CDR3 Amino Acid | GRGWASDNLYL (SEQ ID NO: 165) |
| Sequence | |
| (IMGT) | |
| Light Chain Leader DNA | atggacacgagggcccccactcagctgctggggctcctgctgctctggctcccaggt |
| Sequence | gccacattt (SEQ ID NO: 10) |
| Light Chain Leader Amino acid | MDTRAPTQLLGLLLLWLPGATF (SEQ ID NO: 69) |
| Sequence | |
| Light Chain Variable Domain | gcccaagttctgacaacccagactccatcgtccgtgtctgcagctgtgggaggcaca |
| DNA Sequence | gtcaccatcaactgccaggccagtcagagtctttataataagaaaaatttagcctggttt |
| | cagcagaaaccagggcagcctcccaagcgcctgatctattatgcatccactctggcat |
| | ctggggtctcatcgcggttcaaaggcagtggatctgggacacagttcactctcaccat |
| | cagtgacgtgcagtgtgacgatactgccacttactactgtctaggcgactttgattgtag |
| | tagtgctgattgttatagtttcggcggagggaccgaggtggtcgtcaaaggt (SEQ |
| | ID NO: 166) |
| Light Chain Variable Domain | AQVLTTQTPSSVSAAVGGTVTINCQASQSLYNKKNLA |
| Amino Acid Sequence | WFQQKPGQPPKRLIYYASTLASGVSSRFKGSGSGTQF |
| | TLTISDVQCDDTATYYCLGDFDCSSADCYSFGGGTEV |
| | VVKG (SEQ ID NO: 167) |
| Light Chain CDR1 Amino Acid | QSLYNKKN (SEQ ID NO: 168) |
| Sequence | |
| (IMGT) | |
| Light Chain CDR2 Amino Acid | YAS |
| Sequence | |
| (IMGT) | |
| Light Chain CDR3 Amino Acid | LGDFDCSSADCYS (SEQ ID NO: 169) |
| Sequence | |
| (IMGT) | |
| Heavy Chain DNA Sequence | cagtcggtggaggagtccgggggtcgcctggtcacgcctgggacacccctgacact |
| | cacctgcaccgtctctggattctccctcaataaccatgcaatgggctgggtccgccag |
| | gctcctaggaaggggctggaatatatcggatacattagtattagtggtaccacatacta |
| | cgcgagctgggcgaaaggccgattcaccatctccaagacctcgaccacggtggatc |
| | tgaaaatcaccagtccgacaaccgaggacacgggcacctatttctgtggcagaggtt |
| | gggctagtgataatttgtacttgtggggccaaggcaccctggtcaccgtctcctcagg |
| | gcaacctaaggctccatcagtcttcccactggccccctgctgcggggacacacccag |
| | ctccacggtgaccctgggctgcctggtcaaaggctacctcccggagccagtgaccgt |
| | gacctggaactcgggcaccctcaccaatggggtacgcaccttcccgtccgtccggc |
| | agtcctcaggcctctactcgctgagcagcgtggtgagcgtgacctcaagcagccagc |
| | ccgtcacctgcaacgtggcccacccagccaccaacaccaaagtggacaagaccgt |
| | ggcgccctcgacatgcagcaagcccacgtgcccaccccctgaactcctgggggga |
| | ccgtctgtcttcatcttccccccaaaacccaaggacaccctcatgatctcacgcacccc |
| | cgaggtcacatgcgtggtggtggacgtgagccaggatgaccccgaggtgcagttca |
| | catggtacataaacaacgagcaggtgcgcaccgcccggccgccgctacgggagca |
| | gcagttcaacagcacgatccgcgtggtcagcaccctccccatcgcgcaccaggact |
| | ggctgaggggcaaggagttcaagtgcaaagtccacaacaaggcactcccggcccc |
| | catcgagaaaaccatctccaaagccagagggcagcccctggagccgaaggtctac |
| | accatgggccctccccgggaggagctgagcagcaggtcggtcagcctggcctgca |
| | tgatcaacggcttctacccttccgacatctcggtggagtgggagaagaacgggaagg |
| | cagaggacaactacaagaccacgccggccgtgctggacagcgacggctcctacttc |
| | ctctacagcaagctctcagtgcccacgagtgagtggcagcggggcgacgtcttcacc |
| | tgctccgtgatgcacgaggccttgcacaaccactacacgcagaagtccatctcccgct |
| | ctccgggtaaa (SEQ ID NO: 170) |
| Heavy Chain Amino Acid | QSVEESGGRLVTPGTPLTLTCTVSGFSLNNHAMGWVR |
| Sequence | QAPRKGLEYIGYISISGTTYYASWAKGRFTISKTSTTV |
| | DLKITSPTTEDTGTYFCGRGWASDNLYLWGQGTLVT |
| | VSSGQPKAPSVFPLAPCCGDTPSSTVTLGCLVKGYLPE |
| | PVTVTWNSGTLTNGVRTFPSVRQSSGLYSLSSVVSVTS |
| | SSQPVTCNVAHPATNTKVDKTVAPSTCSKPTCPPPELL |
| | GGPSVFIFPPKPKDTLMISRTPEVTCVVVDVSQDDPEV |
| | QFTWYINNEQVRTARPPLREQQFNSTIRVVSTLPIAHQ |
| | DWLRGKEFKCKVHNKALPAPIEKTISKARGQPLEPKV |
| | YTMGPPREELSSRSVSLACMINGFYPSDISVEWEKNG |
| | KAEDNYKTTPAVLDSDGSYFLYSKLSVPTSEWQRGD |
| | VFTCSVMHEALHNHYTQKSISRSPGK (SEQ ID NO: 171) |
| Light Chain DNA Sequence | gcccaagttctgacaacccagactccatcgtccgtgtctgcagctgtgggaggcaca |
| | gtcaccatcaactgccaggccagtcagagtctttataataagaaaaatttagcctggttt |
| | cagcagaaaccagggcagcctcccaagcgcctgatctattatgcatccactctggcat |
| | ctggggtctcatcgcggttcaaaggcagtggatctgggacacagttcactctcaccat |
| | cagtgacgtgcagtgtgacgatactgccacttactactgtctaggcgactttgattgtag |
| | tagtgctgattgttatagtttcggcggagggaccgaggtggtcgtcaaaggtgatcca |
| | gttgcacctactgtcctcatcttcccaccagctgctgatcaggtggcaactggaacagt |
| | caccatcgtgtgtgtggcgaataaatactttcccgatgtcaccgtcacctgggaggtg |
| | gatggcaccacccaaacaactggcatcgagaacagtaaaacaccgcagaattctgc |
| | agattgtacctacaacctcagcagcactctgacactgaccagcacacagtacaacag |
| | ccacaaagagtacacctgcaaggtgacccagggcacgacctcagtcgtccagagct |
| | tcaataggggtgactgt (SEQ ID NO: 172) |
| Light Chain Amino Acid | AQVLTTQTPSSVSAAVGGTVTINCQASQSLYNKKNLA |
| Squence | WFQQKPGQPPKRLIYYASTLASGVSSRFKGSGSGTQF |
| | TLTISDVQCDDTATYYCLGDFDCSSADCYSFGGGTEV |
| | VVKGDPVAPTVLIFPPAADQVATGTVTIVCVANKYFP |
| | DVTVTWEVDGTTQTTGIENSKTPQNSADCTYNLSSTL |
| | TLTSTQYNSHKEYTCKVTQGTTSVVQSFNRGDC (SEQ ID NO: 173) |
| Heavy Chain Fab DNA sequence | cagtcggtggaggagtccgggggtcgcctggtcacgcctgggacacccctgacact |
| | cacctgcaccgtctctggattctccctcaataaccatgcaatgggctgggtccgccag |
| | gctcctaggaaggggctggaatatatcggatacattagtattagtggtaccacatacta |
| | cgcgagctgggcgaaaggccgattcaccatctccaagacctcgaccacggtggatc |
| | tgaaaatcaccagtccgacaaccgaggacacgggcacctatttctgtggcagaggtt |
| | gggctagtgataatttgtacttgtggggccaaggcaccctggtcaccgtctcctcagg |
| | gcaacctaaggctccatcagtcttcccactggccccctgctgcggggacacacccag |
| | ctccacggtgaccctgggctgcctggtcaaaggctacctcccggagccagtgaccgt |
| | gacctggaactcgggcaccctcaccaatggggtacgcaccttcccgtccgtccggc |
| | agtcctcaggcctctactcgctgagcagcgtggtgagcgtgacctcaagcagccagc |
| | ccgtcacctgcaacgtggcccacccagccaccaacaccaaagtggacaagaccgt |
| | g (SEQ ID NO: 174) |
| Heavy Chain Fab amino acid | QSVEESGGRLVTPGTPLTLTCTVSGFSLNNHAMGWVR |
| sequence | QAPRKGLEYIGYISISGTTYYASWAKGRFTISKTSTTV |
| | DLKITSPTTEDTGTYFCGRGWASDNLYLWGQGTLVT |
| | VSSGQPKAPSVFPLAPCCGDTPSSTVTLGCLVKGYLPE |
| | PVTVTWNSGTLTNGVRTFPSVRQSSGLYSLSSVVSVTS |
| | SSQPVTCNVAHPATNTKVDKTV (SEQ ID NO: 175) |

### EXAMPLES

These examples are provided for illustrative purposes only and not to limit the scope of the claims provided herein.

### Example 1: Design, Expression and Analysis of 2TDCH2-LFLEPS Design of Recombinant Antibodies

### Materials and Methods

All materials were obtained from Thermo Fisher Scientific (Waltham, MA) unless otherwise noted.

Generation of anti-OCT4-2TdCH2-LFLEPS, anti-Parkin-2TdCH2-LFLEPS, anti-IDO1-2TdCH2-LFLEPS, anti-FLAG-tag-2TdCH2-LFLEPS, anti-pERK]2-2TdCH2, anti-PSD-95-2TdCH2, anti-TIGIT 2TdCH2-LFLEPS, and Rabbit-anti-mouse IgG-2TdCH2-LFLEPS Antibody Overexpression Constructs

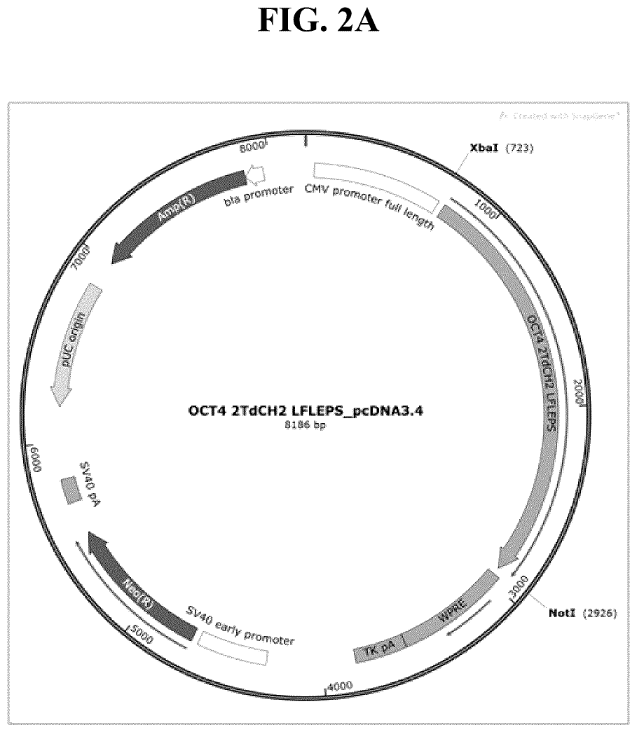

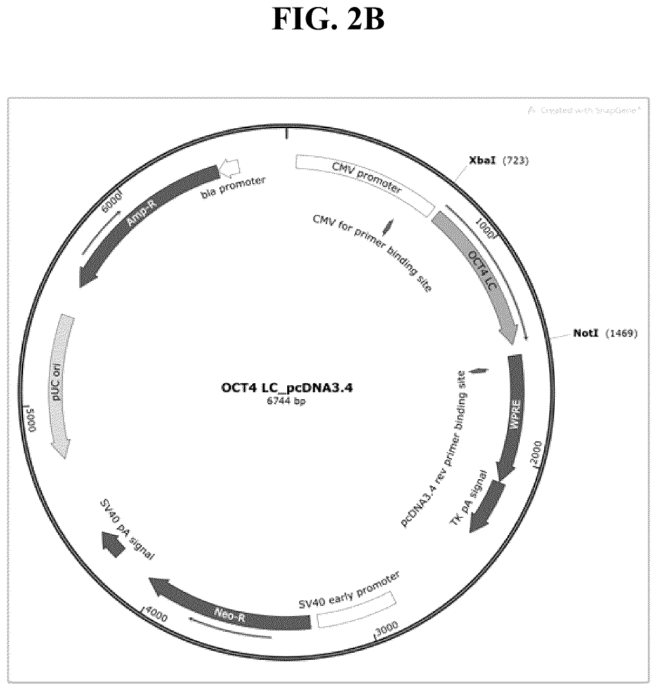

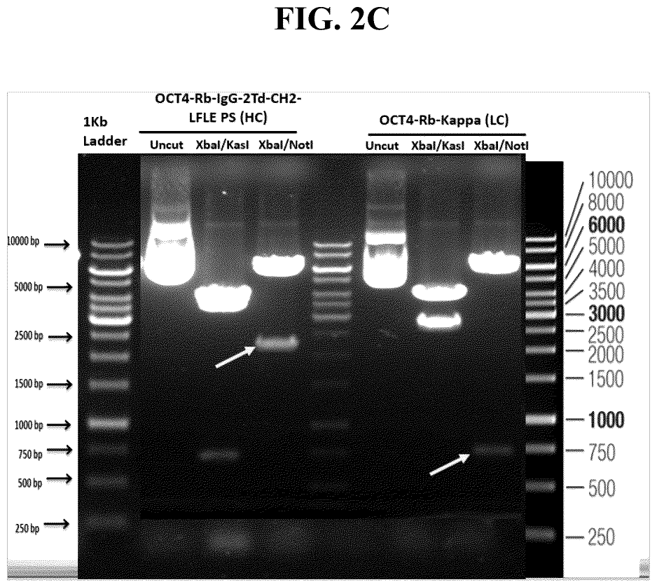

Heavy chain expression construct: The backbone vector containing a rabbit 2TdCH2-LFLEPS antibody backbone was synthesized in pCDNA3.4 with the annotated restriction sites as illustrated in FIG. 2A.

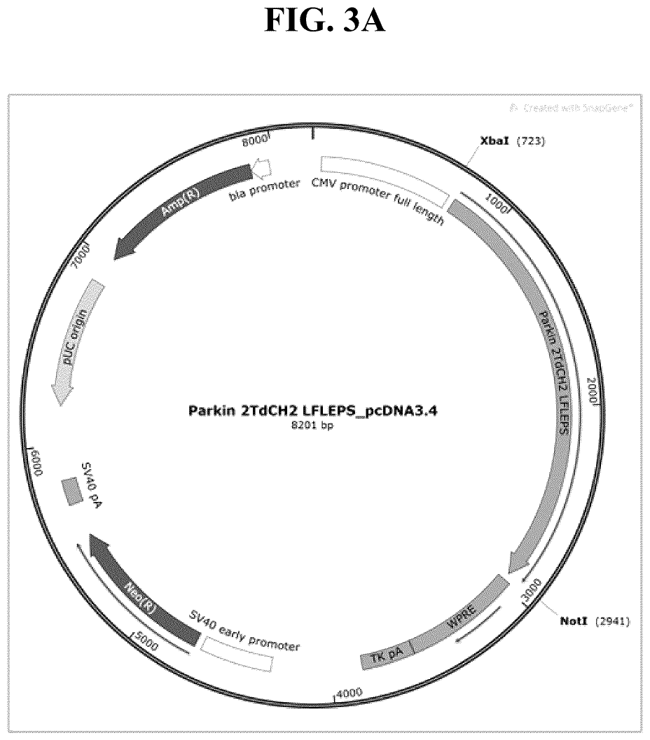

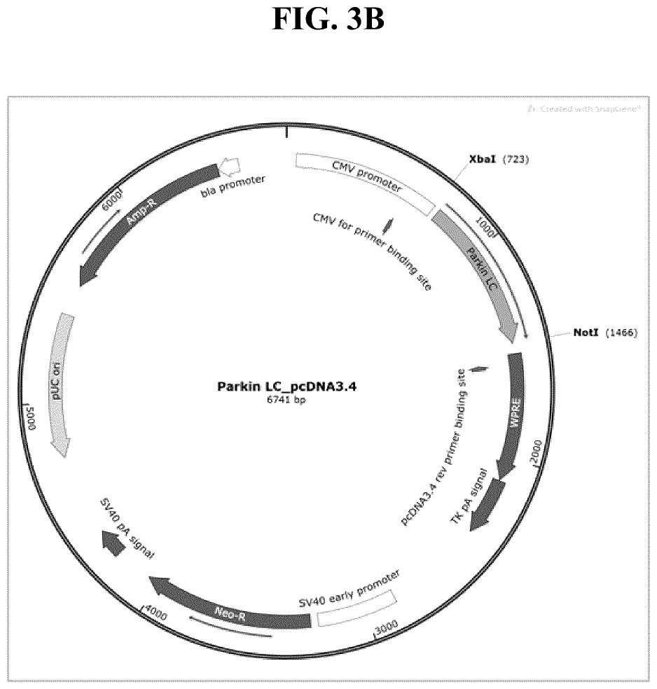

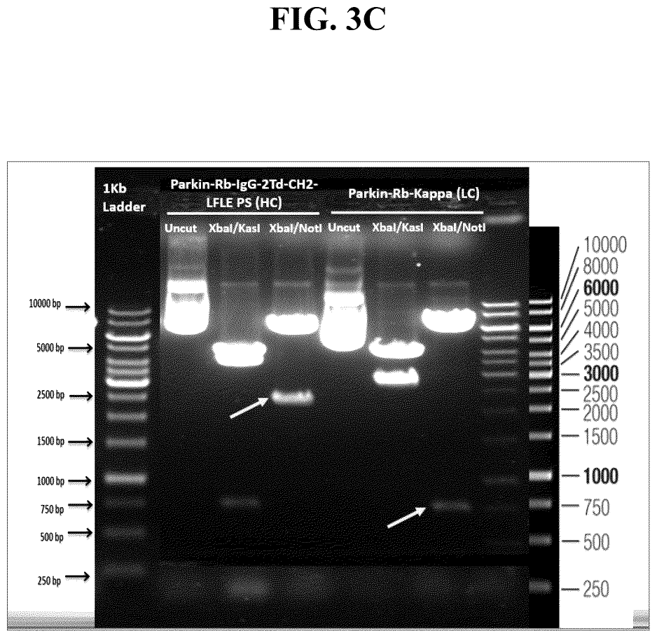

To generate the expression construct for the OCT4 and Parkin antibodies with the 2TdCH2-LFLEPS backbone, the Fab regions of the parental OCT4 (Clone ID: 3H8L6) and Parkin antibodies (Clone ID: 21H24L9) were cloned into the above vector between the restriction sites XBA1 and KAS 1. Restriction analysis of the construct confirmed the accuracy of the expression construct (FIG. 2C & FIG. 3C).

Light chain expression construct: The constructs for light chain for rabbit OCT4 (Clone ID: 3H8L6) and Parkin (Clone ID: 21H24L9) are illustrated in (FIG. 2B & FIG. 31B). Similarly, other targets were cloned into the rabbit 2TdCH2-LFLEPS or mouse 2TdCH2 backbone as shown in Table 27 below.

TABLE 27
Targets Engineered into the 2TdCH2-LFLEPS Antibody Backbone

| | | | Engineered |
|---|---|---|---|
| | | Host/ | backbone |
| Target | Antibody Name/Clone | Isotype | used |
| Parkin | Parkin Recombinant Rabbit | Rabbit/IgG | Rabbit |
| | Monoclonal Antibody (Clone: | | 2TdCH2- |
| | 21H24L9) | | LFLEPS |
| OCT4 | OCT4 Recombinant Rabbit | Rabbit/IgG | Rabbit |
| | Monoclonal Antibody (Clone: | | 2TdCH2- |
| | 3H8L6) | | LFLEPS |
| IDO1 | IDO Recombinant Rabbit | Rabbit/IgG | Rabbit |
| | Monoclonal Antibody (Clone: | | 2TdCH2- |
| | 7H8L17) | | LFLEPS |
| FLAG- | FLAG tag Recombinant Rabbit | Rabbit/IgG | Rabbit |
| tag | Monoclonal Antibody (Clone: | | 2TdCH2- |
| | 8H8L17) | | LFLEPS |
| pERK1/2 | Phospho-ERK1/2 (Thr202, | Mouse/ | Mouse IgG1 |
| | Tyr204) Monoclonal Antibody | IgG1, kappa | 2TdCH2 |
| | (Clone: MILAN8R) | | |
| PSD-95 | PSD-95 Monoclonal Antibody | Mouse/ | Mouse IgG1 |
| | (Clone: 7E3-1B8) | IgG1, kappa | 2TdCH2 |
| RAM | Rabbit anti-Mouse IgG | Rabbit/IgG | Rabbit |
| | Secondary Antibody (Clone: | | 2TdCH2- |
| | RAM35HC35LC37) | | LFLEPS |
| TIGIT | TIGIT Recombinant Chimeric | Rabbit/IgG | Rabbit |
| | Monoclonal Antibody (Clone: | | 2TdCH2- |
| | MBSA43), Mouse Fab-Rabbit | | LFLEPS |
| | Fc Chimera | | |

### Expression and Production of 2TdCH2-LFLEPS Recombinant Antibodies

Heavy chain and light chain for the targets listed in Table 27 were transfected into Expi293F™ cells at a ratio of 1:1 using ExpiFectamine™ 293 Transfection Kit (Product #A14524, Protocol Manual: MAN0007814) or ExpiFectamine™ CHO Transfection (Product #A29129, Protocol Manual: MAN0014337). Five days or eight days post transfection, the supernatants were collected and purified over POROS™ MabCapture™ A/POROS™ MabCapture™ G/CaptureSelect™ IgG-Fc (Multispecies) Affinity Matrix column and formulated in PBS pH 7.4. The antibodies were run through HPLC column (BioSep™ 5 μm SEC-s3000 290 Å, LC Column 300×7.8 mm, Ea) to check for the monomer content.

### Immunoassays:

Immunocytochemistry: Immunofluorescence analysis was performed using 70% confluent cells of the desired cell lines. The cells were fixed with 4% paraformaldehyde for 10 minutes, permeabilized with 0.5% Triton X-100 for 15 minutes and blocked with 2% BSA for 1 hour at room temperature. The cells were stained with the appropriate antibodies. Imaging was performed either at 60× or 40× or 20× magnification. Nuclei were stained using ProLong™ Diamond Antifade Mountant with DAPI (Product #P36962) or Hoescht (Product #H1399) and cytoskeletal F-actin staining was performed using Rhodamine Phalloidin (Product #R415).

Western Blotting: Known quantities of protein samples were electrophoresed using Novex® NuPAGE@4-12% Bis-Tris gel (Product #NPO322BOX), XCell SureLock™ Electrophoresis System (Product #E10002) and Novex® Sharp Pre-Stained Protein Standard (Product #LC5800). Resolved proteins were then transferred onto a nitrocellulose membrane with iBlot® Dry Blotting System (Product #IB21001) and detected by probing with the indicated primary and secondary antibodies. Detection was performed using Novex® ECL Chemiluminescent Substrate Reagent Kit (Product #WP20005) or Chemiluminescent SuperSignal™ West Dura Extended Duration Substrate (Product #34075) and detected using the iBright™ FL 1500 (Product #A44115).

Immunohistochemistry: To expose the target protein on the tissue section, heat-induced epitope retrieval was performed on de-paraffinized sections using eBioscience™ IHC Antigen Retrieval Solution—High pH (10×) (Product #00-4956-58) diluted to 1× solution in water in a decloaking chamber at 110° C. for 15 minutes. Following antigen retrieval, the sections were blocked with 2% normal goat serum in 1×PBS for 45 minutes at room temperature and then probed with or without the indicated primary antibody at a 1:100 dilution in 0.1% normal goat serum overnight at 4 degrees Celsius in a humidified chamber. Detection was performed using Goat anti-Rabbit IgG (H+L) Highly Cross-Adsorbed Secondary Antibody, Alexa Fluor™ Plus 488 (Product #A32731) at a dilution of 1:2,000 in 0.1% normal goat serum for 1 hour at room temperature. ReadyProbes™ Tissue Autofluorescence Quenching Kit (Product #R37630) was used to quench autofluorescence from the tissues. Nuclei were stained with DAPI (Product #D1306) and the sections were mounted using ProLong™ Glass Antifade Mountant (Product #P36984). The images were captured on EVOS™ M7000 Imaging System (Product #AMF7000) at 20× magnification and externally deconvoluted.

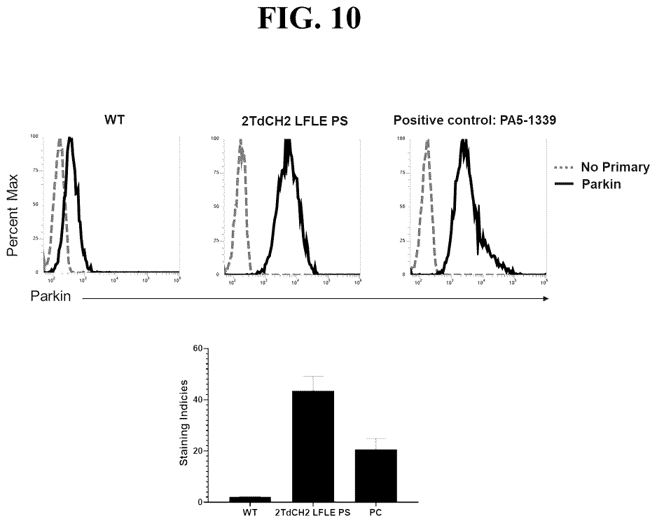

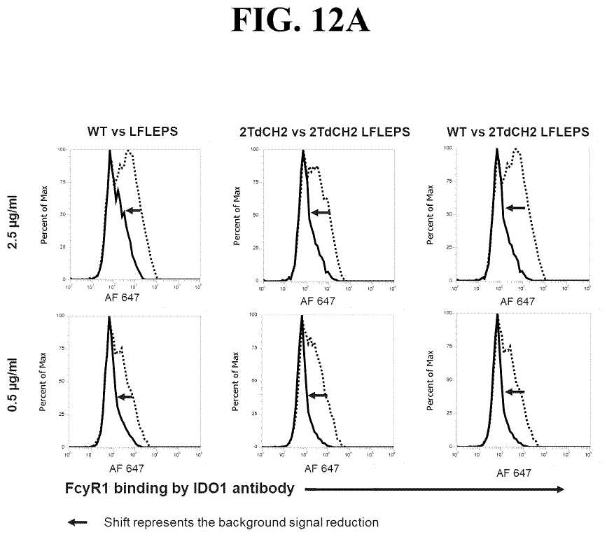

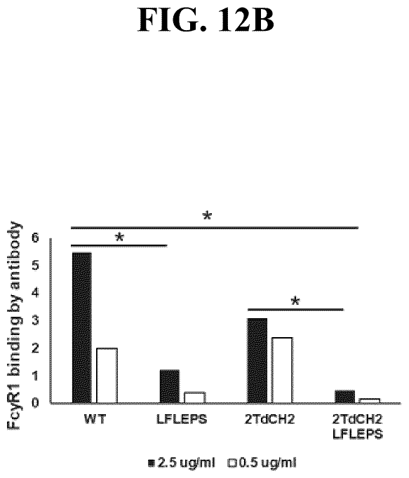

Flow cytometry: Cells were fixed and permeabilized using either the Intracellular Fixation & Permeabilization Buffer Set (Product #88-8824-00) (For Parkin) or the Foxp3/Transcription Factor Staining Buffer Set (Product #00-5523-00) (For OCT4) and stained with the respective antibodies. Protein of interest staining is represented as solid line histogram and unstained population as dashed line histogram (FIG. 10 for Parkin, FIGS. 12A-12B for IDO1). Viable cells were used for analysis, as determined by Fixable Viability Dye eFluor™ 450 (Product #65-0863-14) or Violet fluorescent reactive dye (L34964 Å). The flow cytometry data was acquired using Attune™ N×T Flow Cytometer (Product #A29004).

Conjugation: For Alexa Fluor™ Plus 555 dye conjugation, the antibody was dialyzed against bicarbonate buffer (0.05 M; pH=8.5) overnight at 4° C. 15-fold molar excess of dye was mixed with antibody and incuibated for 1 hour at room temperature protected from light. Excess dye was removed by passing the sample through a Dowex^{T}M column 1×4-400, 50% slurry (Product #22858). The antibody conjugate was formulated in StabilZyme® Protein-Free Stabilizer (pH 6 3-73) with EDTA at a final concentration of 0 1 mm.

For Alexa Fluor™ Plus 647 dye conjugation, the antibody was dialyzed against bicarbonate buffer (0.05 M; pH=8.5) overnight at 4° C. 11--fold molar excess of dye was mixed with antibody and incubated for 1 hour at room temperature protected from light. Excess dye was removed by passing the sample through a Dowex™ column 1×4-400, 50% slurry (Product #22858). The antibody conjugate was formulated in StabidZyne® Protein-Free Stabilizer (pH 63-7.3) with EDTA at a final concentration of 0 1 mM.

For Alexa Fluor™ 488 dye SiteClick™ conjugation, first the carbohydrate domain of the antibody was modified, and azide attachment was performed using SiteClick™ Antibody Azido Modification Kit (Product #S10901). Then an sDIBO Alkyne label (Alexa Fluor™ 647-sDIBO) dye was conjugated to the azide modified antibody using SiteClick™ sDIBO0 Alkyne Kits for Antibody Labeling Alexa Fluor 647 (Product #S10906). Excess dye was removed by passing the sample through Zeba™ Dye and Biotin Removal Spin Columns and Filter Plates, 2 ml, (Product #A44299). The antibody conjugate was formulated in 1× Tris pH 7.0 (or TBS) with 0.02% sodium azide.

List of antibodies used for immunoassays: All antibodies were obtained from Thermo Fisher Scientific unless otherwise noted. Parental Parkin Recombinant Rabbit Monoclonal Antibody (21H24L9) (Product #702785); Parental OCT4 Recombinant Rabbit Monoclonal Antibody (3H8L6) (Product #701756); Parental FLAG Tag Recombinant Rabbit Monoclonal Antibody (81-8L1,7) Recombinant Rabbit Monoclonal Antibody (8H8L17), Product #701629), Parental IDO1 Recombinant Rabbit Monoclonal Antibody (71-8L17), (Product #702743), Parental TIGIT Recombinant Mouse Monoclonal Antibody (MBSA43), (Product #740010M), Parental PSD-95 Recombinant Mouse Monoclonal Antibody (7E3-1B8), (Product #MA1-046); Goat anti-Rabbit IgG (Heavy chain), Superclonal™ Recombinant Secondary Antibody, Alexa Fluor™ 647, (Product #A27040); Goat anti-Rabbit IgG (H+L) Poly-HRP Secondary Antibody, HRP (Product #32260); Goat anti-Rabbit IgG (Heavy chain), Superclonal™ Recombinant Secondary Antibody, HRP (Product #A27036); Goat anti-Rabbit IgG (H+L) Polyclonal Secondary Antibody, HRP (Catalog #31460); Donkey anti-Rabbit IgG (H+L) Secondary Antibody, Alexa Fluor™ Plus 488 (Product #A32790), Donkey anti-Rabbit IgG (1-1+L) Secondary Antibody, Alexa Fluor™ Plus 488 (Product #A32795), Goat anti-Mouse IgG (H+L) Superclonal™ Secondary antibody (Product #A28177).

List of reagents used for immunoassays: All reagents were obtained from Thermo Fisher Scientific unless otherwise noted. Flow Cytometry Staining Buffer, (Product #00-4222-26), IC Fixation Buffer, (Product #00-8222-49), Foxp3/Transcription Factor Staining Buffer Set, (ProdLuct #00-5523-00), Fixable Viability Dye eFluor™ 450, (Product #65-0863-14), Violet fluorescent reactive dye, for 405 nm excitation (Product #L34964 Å), DAPI, (Product #D1306), Hoescht 33342, trihydrochloride trihydrate, (Product #H1399), ProLong™ Glass Andtfade Mountant, (Product #P36984).

siRNAs: OCT4 specific siRNA (Silencer® select Product #s10871, s10873), Parkin specific siRNA (Silencer® select Product #s530941, s530942).

### Results

Generation of Rabbit and Mouse Recombinant Antibodies with Fc Engineered Backbone

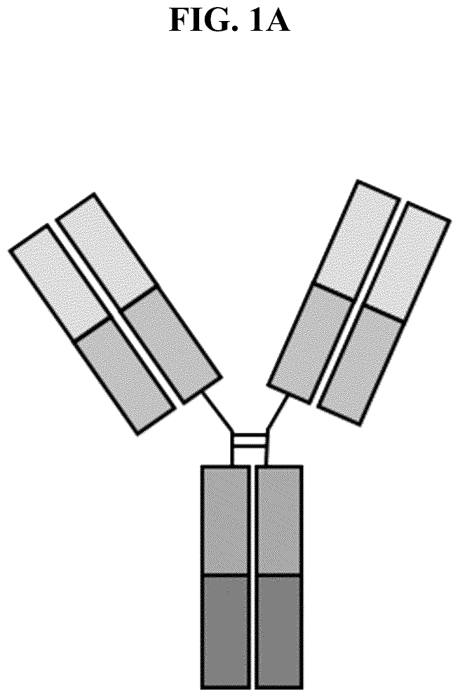

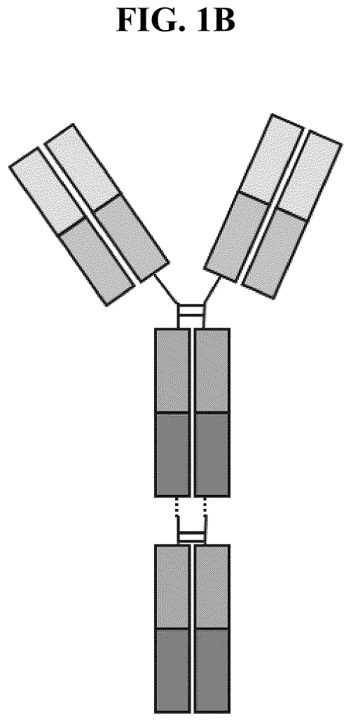

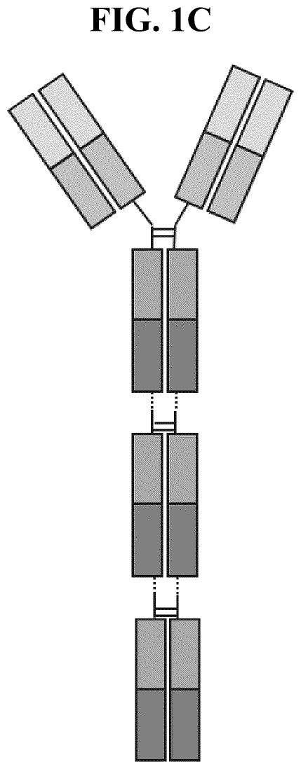

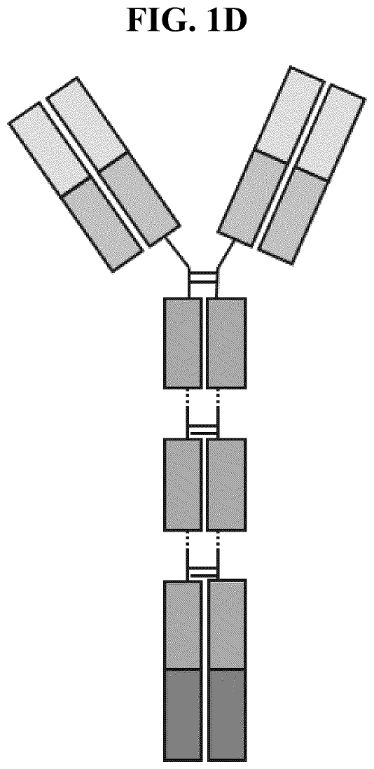

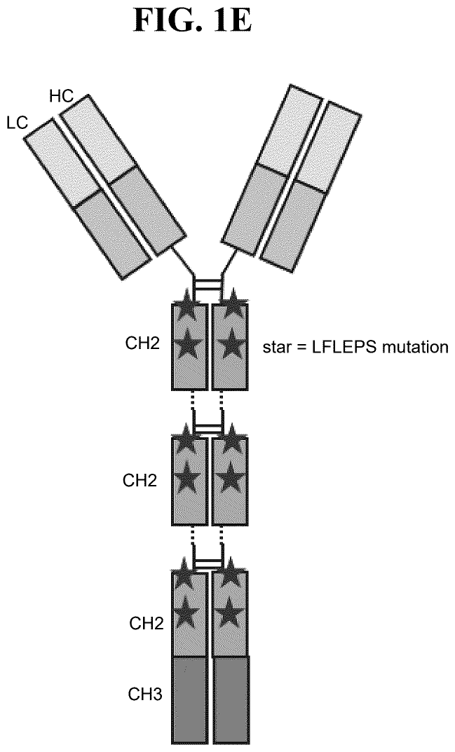

To evaluate the effect of the 2TdCH2 Fc engineered backbone, three rabbit antibodies were generated in which the heavy chain (HC) Fab region (SEQ ID NO: 8) and light chain (LC) Fab region (SEQ ID NO: 11) were taken from Parkin (Clone ID: 21H24L9, Product #702785), or the HC Fab region (SEQ ID NO: 33) and LC Fab region (SEQ ID NO: 35) were taken from OCT4 (Clone ID: 3H8L6, Product #701756), or the HC Fab region (SEQ ID NO: 78) and LC region (SEQ ID NO: 76) were taken from IDO1 (Clone ID: 7H8L17, Product #702743) rabbit recombinant antibodies, and the HC Fab regions were cloned into vectors containing Rabbit 2TdCH2 with LFLEPS mutations (SEQ ID NO: 55) or without LFLEPS mutations (SEQ ID NO: 53). Antibodies in which these Fabs were cloned into vectors containing one additional Fc unit (1TdFc) comprising a nucleic acid having SEQ ID NO: 57 (encoding amino acid SEQ ID NO: 56) or two additional Fc units (2TdFc) comprising a nucleic acid having SEQ ID NO: 61 (encoding amino acid SEQ ID NO: 60) also were generated. Schematic representations of the different engineering designs are provided in FIGS. 1A-1E. The yield, purity, and stability of these constructs were compared (Tables 28 and 29). The antibodies with 2TdCH2 or 2TdCH2-LFLEPS backbone were comparable to the parental wild type antibodies with respect to yield, purity, and stability (Tables 28 and 29). In contrast, antibodies with 1TdFc or 2TdFc backbones had poor yield, lower monomer purity, and stability compared to parental wild type antibodies (Tables 28 and 29).

To assess if the engineered rabbit Fc backbone was able to confer a benefit in chimeric antibodies with a rabbit Fc, an anti-TIGIT mouse-rabbit chimeric recombinant antibody was generated in which the HC Fab region (SEQ ID NO: 157) and LC region (SEQ ID NO: 155) was taken from Mouse TIGIT antibody (Clone ID: MBSA43, Product #16-9500-82), and cloned into vectors containing Rabbit HC 2TdCH2 with LFLEPS mutations (SEQ ID NO: 55) or without LFLEPS mutations (SEQ ID NO: 53). Schematic representation of the engineering design is provided in FIG. 1E. To evaluate the effect of 2TdCH2+LFLEPS Fc engineered backbone for rabbit secondary antibodies, a rabbit anti-mouse IgG (RAM) secondary antibody in which the HC Fab region (SEQ ID NO: 138) was taken from Rabbit anti-Mouse secondary (Clone ID: RAM35HC35LC37) and cloned into vectors containing Rabbit 2TdCH2 with LFLEPS mutations (SEQ ID NO: 55) or without LFLEPS mutations (SEQ ID NO: 53). Schematic representations of the engineering designs are provided in FIGS. 1D and 1E, respectively.

Additionally, the effect of the 2TdCH2 Fc design was investigated for mouse antibodies. Mouse IgG1 Fc domain lacks the amino acids that mediate interaction with FcγR1, therefore LFLEPS mutations would confer no additional benefits to background reduction for this Fc isotype. Therefore, mouse antibodies were generated in which the HC Fab region (SEQ ID NO: 97) was taken from Mouse pERK1/2 (Clone ID: MILAN8R, Product #14-9109-82) or the HC Fab region (SEQ ID NO: 122) was taken from Mouse PSD-95 hybridoma antibody (Clone ID: 7E3-1B8, Product #MA1-046), and cloned into vectors containing Mouse IgG1 2TdCH2. Schematic representation of the engineering design is provided in FIG. 1D.

TABLE 28
Summary of yield, purity, sensitivity, and stability of engineered constructs for OCT4.

| | | | Purity | Sensitivity | | | |
|---|---|---|---|---|---|---|---|
| Backbone | Yield | Assembly | (% Monomer) | WB | ICC | FLOW | Stability |
| WT | +++ | Complete | High | +++ | +++ | + | Stable |
| 1TdFc | ++ | Partial | Med | ++++++ | ++++ | ++ | Precipitation observed |
| 2TdFc | + | Complete | High* | ++++++ | +++ | ++ | Precipitation observed |
| 2TdCH2 | +++ | Complete | High | ++++++ | ++++ | +++ | Stable* |
| 2TdCH2- | +++ | Complete | High | ++++++ | ++++ | +++ | Stable* |
| LFLEPS | | | | | | | |
| Comments/ | ++++ >130 | * Partial: | High Purity | ++++++ | +++++ | ++ | *Accelerated stability |
| Legend | ug/ml | High aggregation/ | (>75%) | indicates > | indicates > | indicates >1 | studies indicate |
| | +++: 80-130 | half molecules/ | Medium Purity | or = 2-fold | or = 2 -fold | but <1.5-fold | stability compared to |
| | ug/ml | fragments | (50-75%) | enhancement | enhancement | increase in | wild type antibodies |
| | ++: 50-80 | # Incomplete: | Low Purity | ++++ | ++++ | staining index | |
| | ug/ml | No assembly | (<50%) | indicates | indicates | +++ | |
| | + <50 ug/ml | | *High purity | 1-2 fold | 1-2 fold | indicates >2- | |
| | | | before SEC | enhancement | enhancement | fold increase | |
| | | | (>70%) | | | in staining | |
| | | | & after SEC | | | index | |
| | | | (>90%) | | | | |

TABLE 29
Summary of yield, purity, sensitivity, and stability of engineered constructs for Parkin.

| | | | Purity (% | Sensitivity | |
|---|---|---|---|---|---|
| Backbone | Yield | Assembly | monomer) | WB | Stability |
| WT | +++ | Complete | High | +++ | Stable |
| 1TdFc | ++ | Partial | Med | ++++++ | Precipitation |
| | | | | | observed |
| 2TdFc | + | Complete | High* | ++++++ | Precipitation |
| | | | | | observed |
| 2TdCH2 | +++ | Complete | High | ++++++ | Stable* |
| 2TdCH2-LFLEPS | +++ | Complete | High | ++++++ | Stable* |
| Comments/Legend | ++++ >130 | * Partial: High | High | ++++++ | *Accelerated |
| | ug/ml | aggregation/half | Purity | indicates > | stability |
| | +++: | molecules/fragments | (>75%) | or = 2-fold | studies |
| | 80-130 | # Incomplete: No | Medium | enhancement | indicate |
| | ug/ml | assembly | Purity | ++++ | stability |
| | ++: 50- | | (50-75%) | indicates 1-2 | compared to |
| | 80 ug/ml | | Low | fold | wild type |
| | + <50 | | Purity | enhancement | antibodies |
| | ug/ml | | (<50%) | | |
| | | | *High | | |
| | | | purity | | |
| | | | before | | |
| | | | SEC | | |
| | | | (>70%) & | | |
| | | | after SEC | | |
| | | | (>90%) | | |

Parkin and OCT4 Antibodies with 2TdCH2 Backbone Demonstrate Increased Sensitivity in Western Blotting and Immunocytochemistry

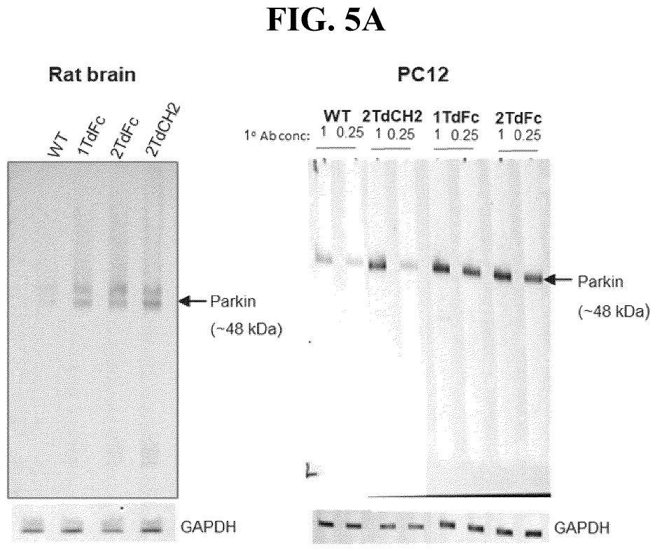

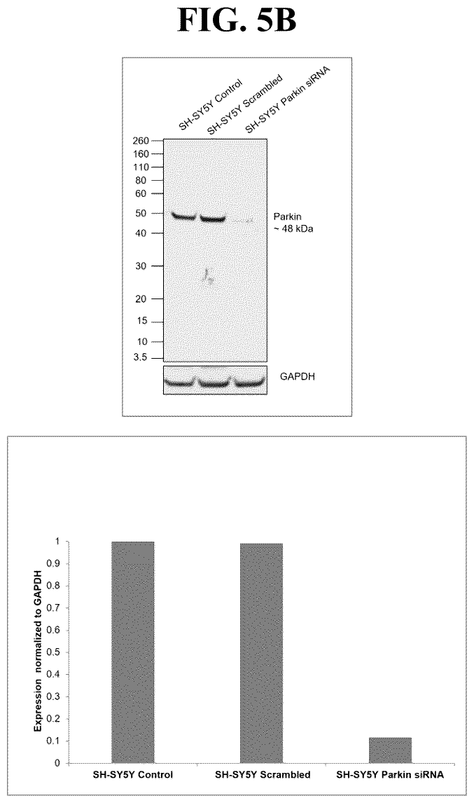

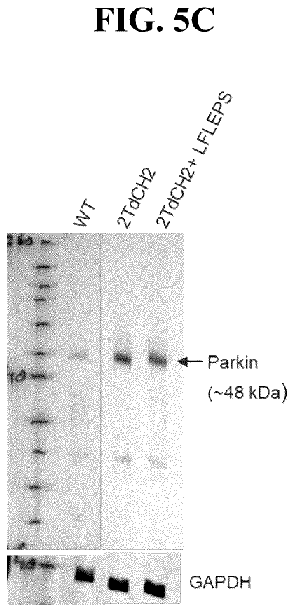

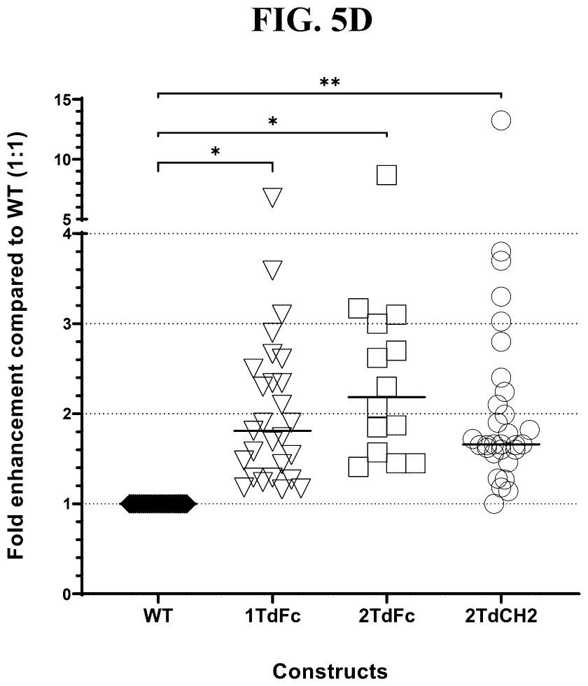

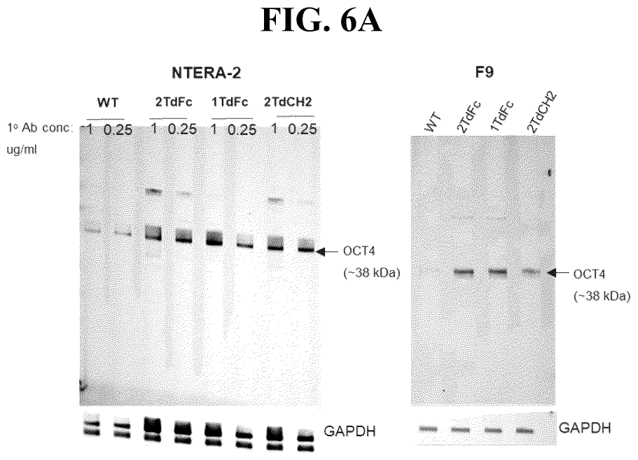

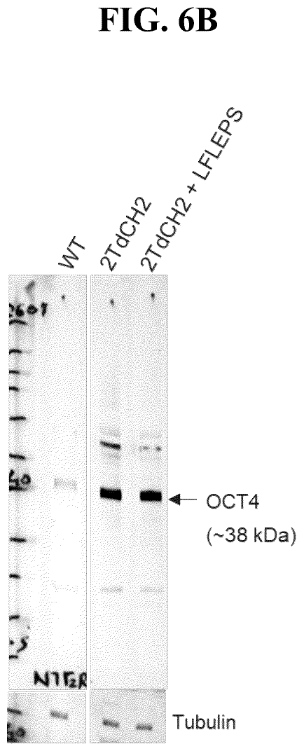

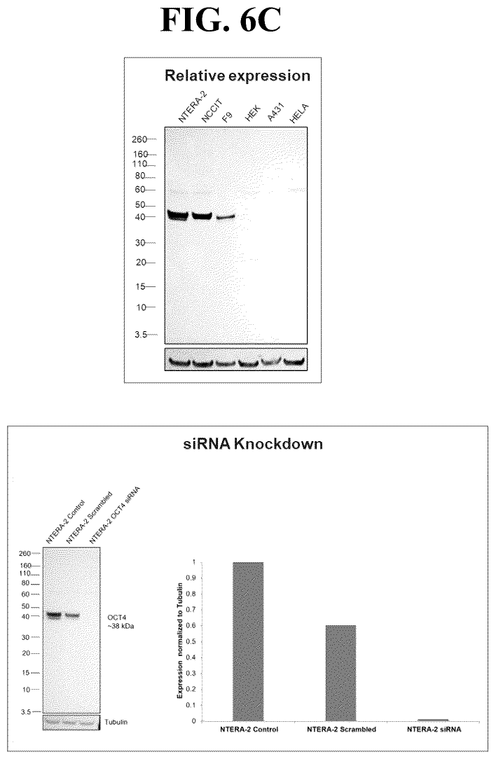

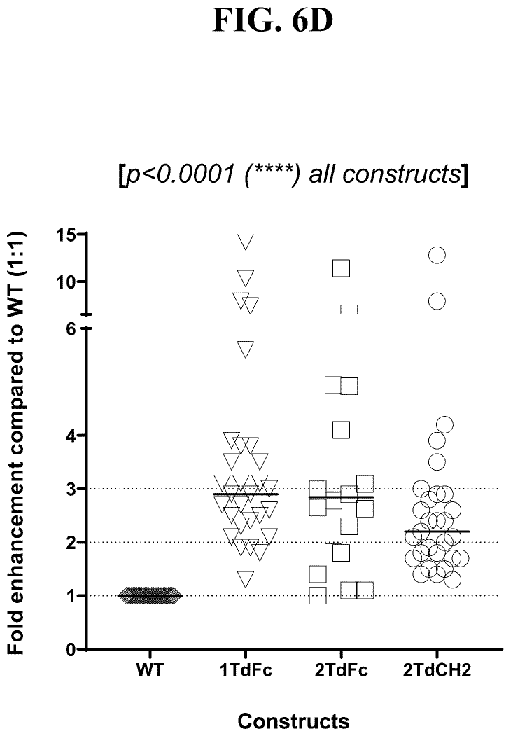

The performance of the 2TdCH2 engineered antibodies was first compared with the parental antibodies. In western blotting across different cell and/or tissue models, both Parkin 2TdCH2 and OCT4 2TdCH2 antibodies demonstrated a significant sensitivity enhancement of ~2 fold over the wildtype parental antibodies (FIG. 5A, FIG. 6A).

Table 30 illustrates the fold change normalized to WT for the rat brain blot in FIG. 5A and Table 31 illustrates the fold change normalized to WT for the PC12 blot in FIG. 5A.

TABLE 30

| Samples | Fold change normalized to WT |
|---|---|
| 1TdFc | 2.9 |
| 2TdFc | 3.1 |
| 2TdCH2 | 3.8 |

TABLE 31

| Samples | Fold change normalized to WT |
|---|---|
| 2TdCH2 1 ug/ml | 4 |
| 2TdCH2 0.25 ug/ml | 3.4 |
| 1TdFC 1 ug/ml | 4.4 |
| 1TdFC 0.25 ug/ml | 10 |
| 2TdFC 1 ug/ml | 4.7 |
| 2TdFC 0.25 ug/ml | 9.4 |

Table 32 illustrates the fold change normalized to WT for the NTERA-2 blot in FIG. 6A and Table 33 illustrates the fold change normalized to WT for the F9 blot in FIG. 6A.

TABLE 32

| Samples | Fold change normalized to WT |
|---|---|
| OCT4 1TdFC 1 ug/ml | 4.1 |
| OCT4 1TdFC 0.25 ug/ml | 3.99 |
| OCT4 2TdFC 1 ug/ml | 5.54 |
| OCT4 2TdFC 0.25 ug/ml | 2.4 |
| OCT4 2TdCH2 1 ug/ml | 4.27 |
| OCT4 2TdCH2 0.25 ug/ml | 3.31 |

TABLE 33

| Samples | Fold change normalized to WT |
|---|---|
| OCT4 1TdFC 0.25 ug/ml | 3.089 |
| OCT4 2TdFC 0.25 ug/ml | 3.096 |
| OCT4 2TdCH2 0.25 ug/ml | 1.522 |

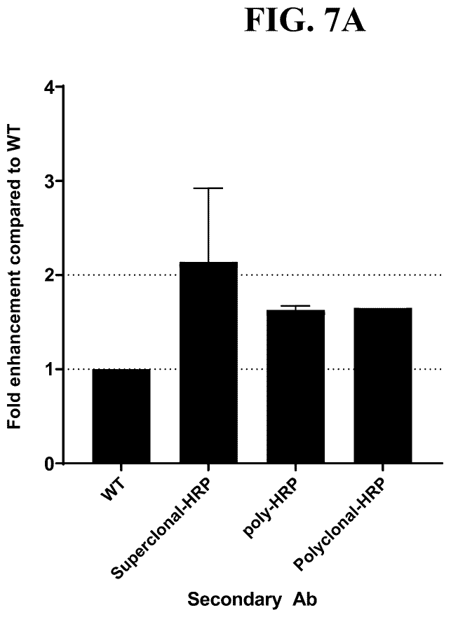

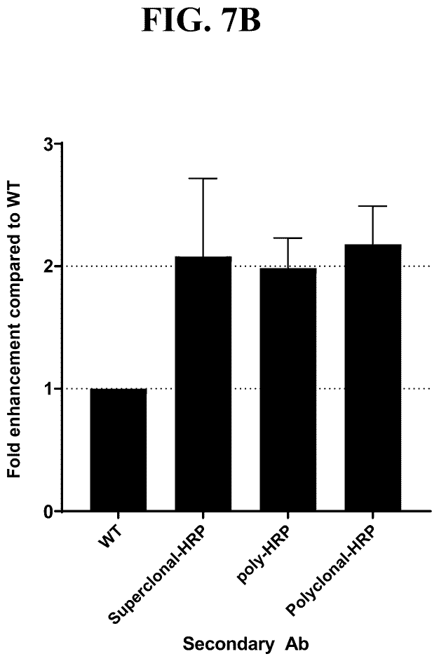

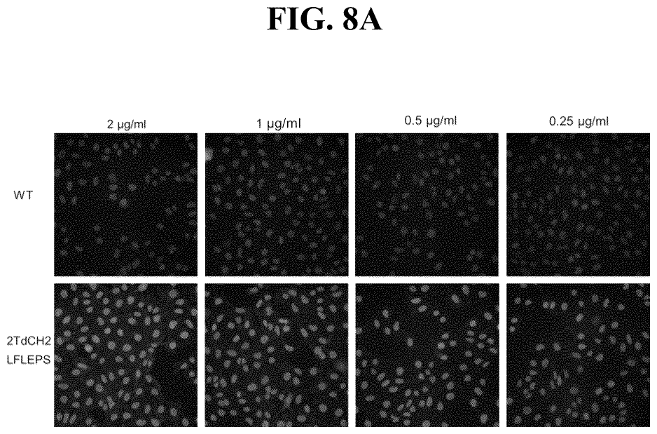

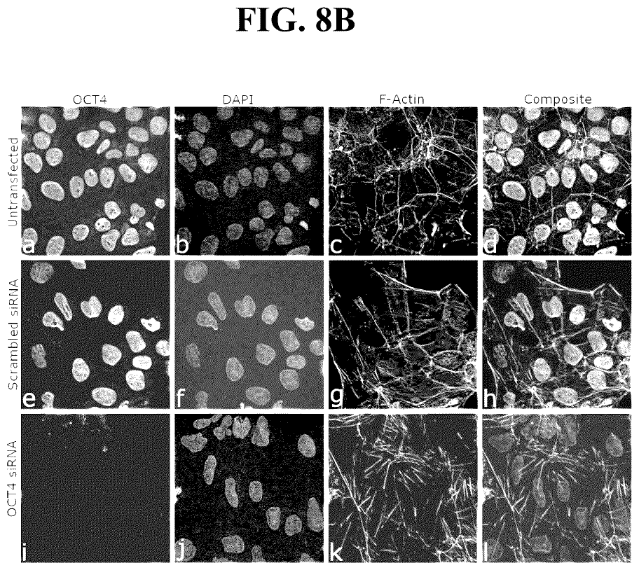

While the actual fold change might vary between experiments, cell/tissue models, and the antibody concentrations used, the results were significant as determined by the independent student's T test. Furthermore, along with the increased sensitivity, there was no compromise on the specificity of the engineered antibodies, as demonstrated by a reduced signal in samples that were transfected with siRNA directed against Parkin or OCT4 and probed by the respective engineered antibodies (FIG. 5B, FIG. 6C). Testing was performed to determine if the benefits of the 2TdCH2 backbone were limited to a particular secondary antibody. It was found that the 2TdCH2 backbone provided a significant and comparable increase in sensitivity irrespective of the secondary antibody used, further underscoring the wide applicability of this design (FIGS. 7A and 7B). Testing was then performed to determine whether mutating residues responsible for Fc receptor binding (LFLEPS) would affect secondary antibody recognition. For both Parkin and OCT4, the sensitivity enhancements were comparable for 2TdCH2 and 2TdCH2-LFLEPS backbones (FIG. 5C, FIG. 6B) indicating that the 2TdCH2-LFLEPS backbone can be a universal backbone for different applications. Next, the OCT4 2TdCH2 antibody was tested and compared with the parental OCT4 antibody in immunocytochemistry. It was found that engineered OCT4 2TdCH2 antibody demonstrated a −1.5 fold increase in signal to noise over parental antibody even when used at ⅛th the concentration of the parental antibody (FIG. 8A). It was also confirmed that the strong nuclear signal produced by the engineered antibody is specific using cells transfected with siRNA that are specific to OCT4 (FIG. 8B).

Parkin Antibody with 2TdCH2 Backbone has Increased Application Coverage as Compared to Its Parental Antibody

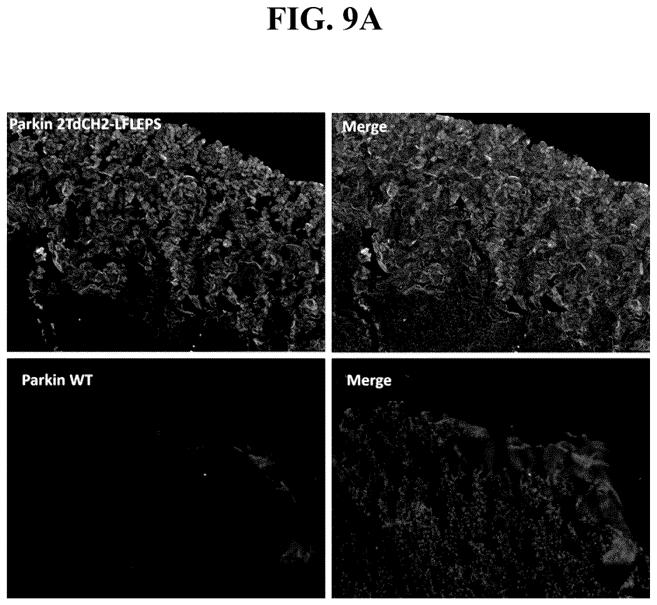

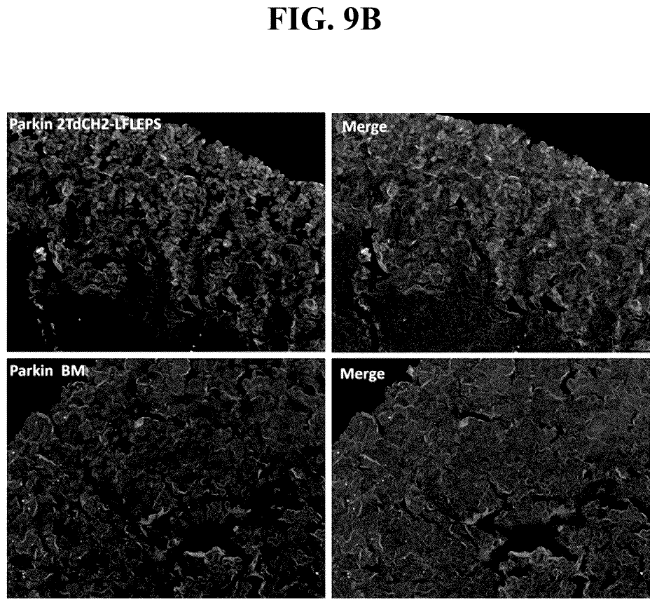

The parental Parkin rabbit recombinant antibody is only sufficiently sensitive in Western blotting. To test if the engineered Parkin 2TdCH2-LFLEPS antibody was sufficiently sensitive for a wider range of applications, this antibody was tested in additional applications. In immunohistochemistry, it was found that, while the parental antibody was not sensitive enough to be detectable in that application, the engineered Parkin 2TdCH2-LFLEPS antibody produced a strong signal and a comparable staining pattern to a benchmark rabbit polyclonal Parkin antibody (Thermo Fisher Scientific, cat. no. PA5-13399) (FIG. 9A and FIG. 9B). In flow cytometry, while the parental Parkin antibody showed only a basal level staining, the engineered antibody demonstrated almost 40-fold increase in staining index (FIG. 10). Table 34 shows the staining indices for the indicated antibodies for the flow cytometry analysis of FIG. 10.

TABLE 34

| Antibody (1:50 dilution, 2 ug/test) | Staining indices |
|---|---|
| WT | 2.13301 |
| WT | 2.106384 |
| WT | 1.884504 |
| 2TdCH2 LFLE PS | 46.69546 |
| 2TdCH2 LFLE PS | 46.80196 |
| 2TdCH2 LFLE PS | 37.11023 |
| Positive control (PC) | 24.47192 |
| Positive control (PC) | 21.04609 |
| Positive control (PC) | 16.00497 |

Idol Antibody with 2TdCH2+LFLEPS Backbone has Decreased Background as Compared to Its Parental Antibody

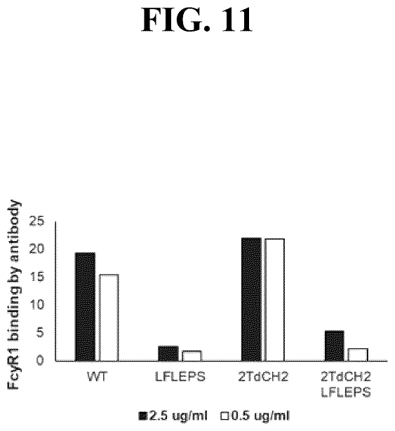

To study the effect of LFLEPS mutations on binding of rabbit antibodies to FcR receptors, a system was employed in which FcγR1 (CD64) fused to GFP was overexpressed in HEK293 cells. Immunofluorescence staining was carried out with wild type, engineered IDO1 with LFLEPS mutations, and engineered IDO1 with 2TdCH2+LFLEPS mutations. Since there is no endogenous expression of IDO1 in CD64 overexpressing HEK293 cells, this experiment purely tracks the binding of the rabbit Fc to the FcγR1, which represents background staining in immunoassays. As can be seen from FIG. 11, there was significant background binding of the IDO1 antibody to the cells in the case of wild type and 2TdCH2 engineered antibodies. However, the introduction of LFLEPS mutations within the context of either wild type (“LFLEPS”) or 2TdCH2 (“2TdCH2 LFLEPS”) rabbit Fc dramatically abolished this background binding. Similar results were observed in flow cytometry staining of CD64 overexpressing HEK293 cells with the different IDO1 recombinant antibodies (FIGS. 12A-12B). As in the immunofluorescence experiment, the staining index (FcγRI binding by antibody) in FIG. 12B denotes background binding since there is no endogenous expression of IDO1 in this model.

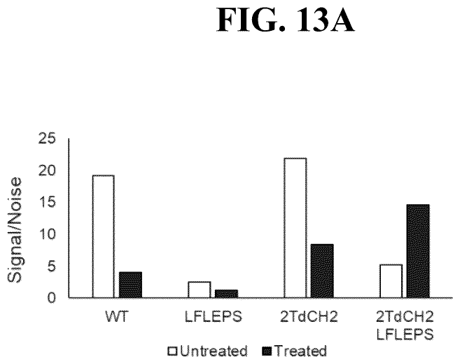

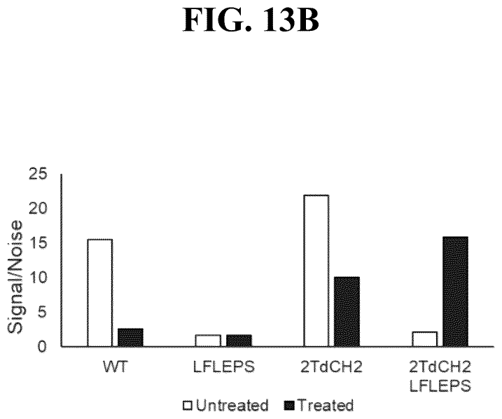

To assess the effect of signal enhancement along with background reduction of the 2TdCH2+LFLEPS backbone, CD64-GFP overexpressing HEK293 cells were subjected to interferon-gamma (IFNγ) treatment which has been demonstrated to increase endogenous IDO1 expression in these cells (FIGS. 13A-13B). In this model, binding of anti-Idol antibodies will result from both the antibody recognizing its target antigen Idol (signal), as well as any non-specific binding of the anti-Idol 1 Fc to CD64 (background). An increased staining of anti-Idol wild type antibody to CD64 transfected cells was observed compared to untransfected cells. Upon treatment of transfected cells with IFNγ, there was no appreciable change in the average intensity (Signal/Noise) for the wild type, LFLEPS, or 2TdCH2 anti-Idol antibodies (FIGS 13A and 13B). (Tverage circ intensity or average intensity of signal divided by noise is equal to signal/noise.) This is likely attributed to low signal (wild type and LFLEPS), and high background (wild type and 2TdCH2) when these antibodies are used. However, when these cells were stained with the anti-Idol antibody engineered with the 2TdCH2+LFLEPS backbone, a sharp increase in staining index (Signal/Noise) was observed both when compared to wild type staining in treated cells (2X), as well as 2TdCH2+LFLEPS staining in untreated cells (~3×) (FIGS. 13A and 13B). These results indicate that engineering for increased sensitivity (2TdCH2) combined with engineering for decreased background (LFLEPS mutations) provide a powerful increase in signal to background.

TIGIT Mouse-Rabbit Chimeric Antibody with Rabbit 2TdCH2+LFLEPS Backbone Demonstrates Increased Sensitivity

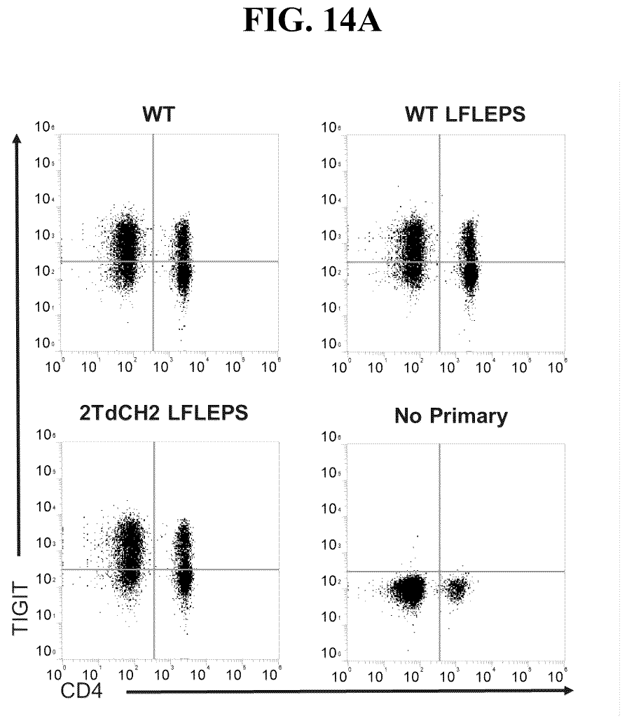

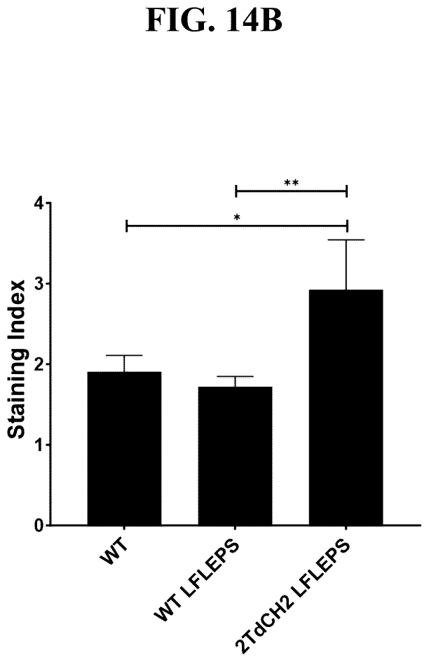

In order to study the utility of the rabbit 2TdCH2+LFLEPS as a universal rabbit backbone with increased sensitivity in species switched antibodies, a chimeric antibody was tested in which the Fab region of Mouse TIGIT IgG1 recombinant antibody was fused with a rabbit 2TdCH2+LFLEPS backbone. In flow cytometry analysis of human PBMC cells for expression of TIGIT, a significant increase in sensitivity was observed for the anti-TIGIT Mouse-Rabbit chimeric antibody with the 2TdCH2+LFLEPS backbone as compared to the chimeric antibody with rabbit Fe with only the LFLEPS mutations (FIGS. 14A-14B), indicating the broad utility of this design.

Rabbit Anti-Mouse (RAM) Secondary Antibody with Rabbit 2TdCH2+LFLEPS Backbone Demonstrates Decreased Background as Compared to its Parental Antibody

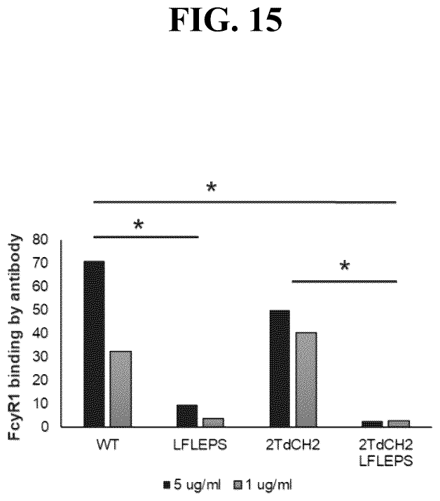

To test whether the 2TdCH2+LFLEPS backbone would have an effect on secondary antibodies, a secondary antibody (Rabbit Anti-Mouse; RAM) was created with LFLEPS and 2TdCH2 with or without LFLEPS and tested for background binding in immunofluorescence in HEK293 cells overexpressing CD64-GFP. The staining observed for the parental antibody was indicative of background binding of secondary antibody to the cells since this model does not have an antigen detecting primary antibody. Consistent with the reduced background observed with the engineered primary antibody (IDO1), a reduction in background staining was observed for the RAM containing both the LFLEPS backbone and the 2TdCH2+LFLEPS backbone (FIG. 15).

Phospho-ERK]2 and PSD-95 Antibodies with Mouse 2TdCH2 Backbone Demonstrate Increased Sensitivity as Compared to their Parental Antibodies

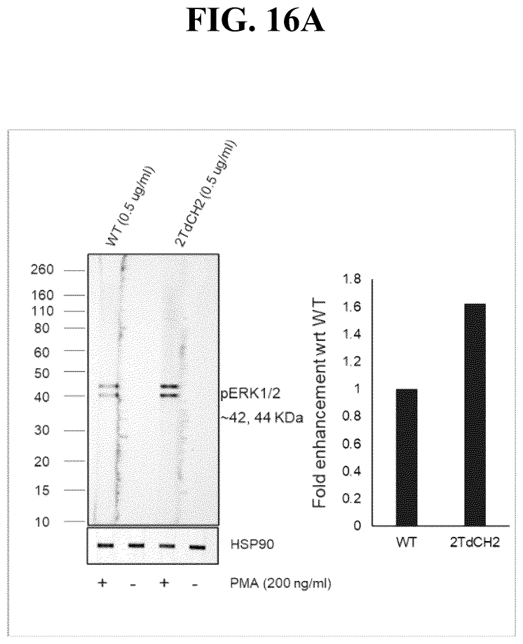

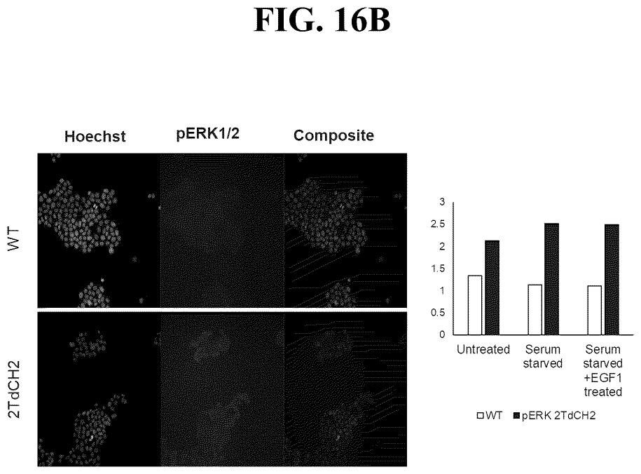

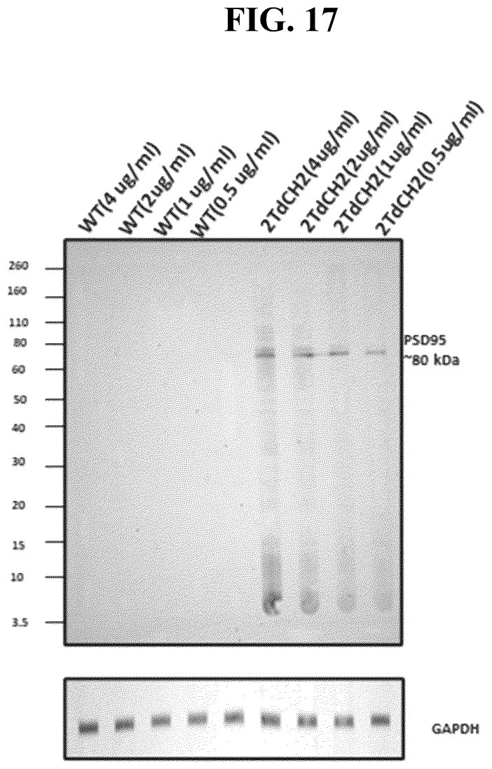

Mouse IgG1 Fc domain lacks the amino acids that mediate interaction with FcγR1, therefore LFLEPS mutations would confer no additional benefits to background reduction for this Fc isotype. In order to assess the 2TdCH2 engineered mouse IgG1 antibodies for increased sensitivity, the performance of 2TdCH2 engineered pERK1/2 and PSD-95 antibodies were compared with the parental wildtype antibodies in western blotting (FIGS. 16A and 17). In the case of the anti-phospho-ERK1/2-2TdCH2 antibody, a 1.6 fold increase in sensitivity was observed compared to the parental (wild type) antibody in Jurkat cells treated with PMA, which is known to induce the phosphorylation of ERK1/2. Although Neuro2A are known to express PSD-95, it is a low abundance protein; however, an increased sensitivity of the anti-PSD-95-2TdCH2 antibody was observed compared to the parental (wild type) antibody. In addition, the pERK1/2 engineered antibody also demonstrated increased sensitivity in immunofluorescence application (two fold) compared to wild type antibody (FIG. 16B). These data confirm the utility of 2TdCH2 design for recombinant mouse IgG1 antibodies.

### 2TdCH2 Engineered Backbone Increases Sensitivity of Primary Conjugated Antibodies Using Amine Chemistry

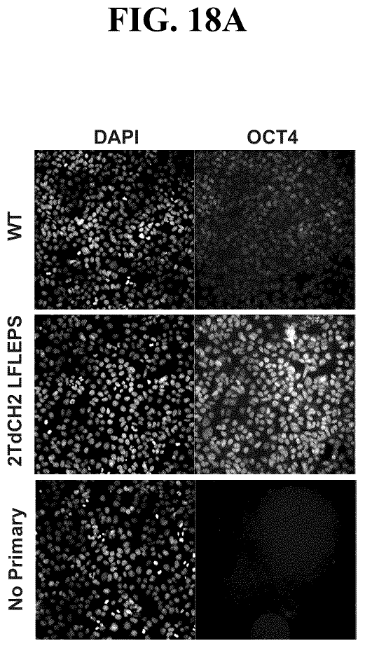

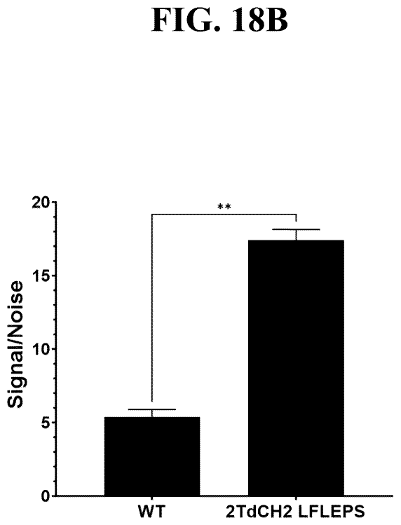

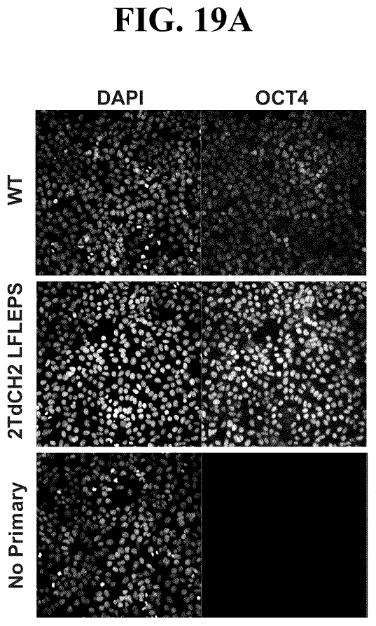

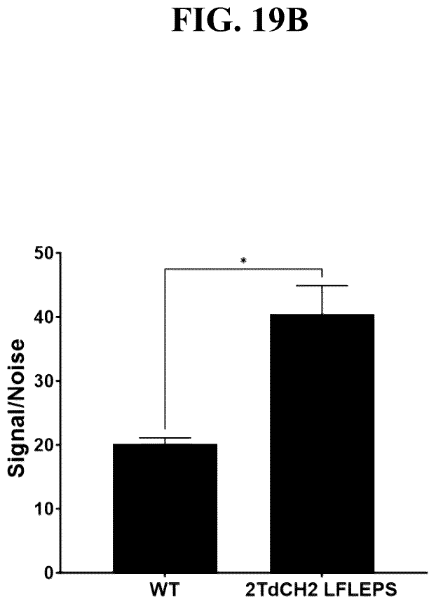

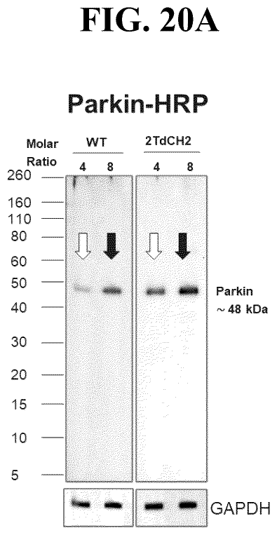

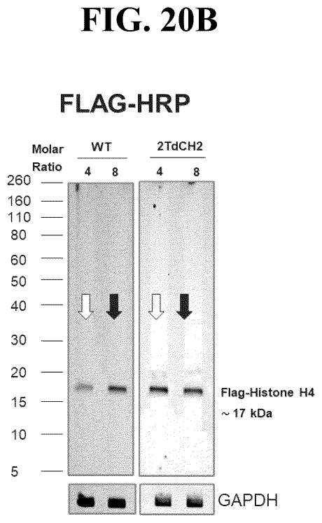

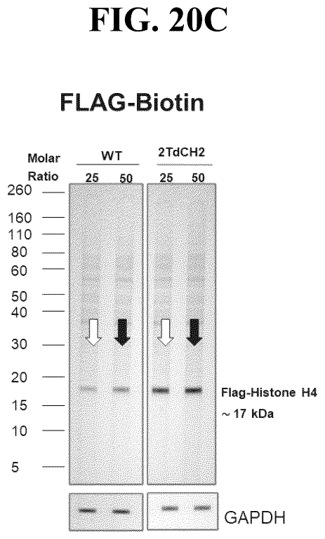

The 2TdCH2 engineered Fc backbone contains additional lysine residues due to the presence of additional CH2 domains. To determine if this results in a higher degree of labeling and sensitivity in immuno applications, the use of lysines as conjugation sites was investigated. First, the anti-OCT4-2TdCH2-LFLEPS engineered antibody was conjugated with Alexa Fluor™ Plus 555 (AFP555) dye and compared the sensitivity to wild type anti-OCT4 antibody conjugated to Alexa Fluor Plus 555 dye in an immunofluorescence assay. A 3-fold increase in signal intensity was observed (FIGS. 18A-18B) in the engineered anti-OCT4-2TdCH2-LFLEPS-AFP555 conjugated antibody. A similar increase in sensitivity was observed with an engineered anti-OCT4-2TdCH2-LFLEPS antibody conjugated to Alexa Fluor™ Plus 647 (FIGS. 19A-19B). Next, an anti-Parkin-2TdCH2-HRP antibody conjugate was compared with wild type anti-Parkin-RP antibody conjugate in a western blot analysis. An increase in sensitivity was observed for the engineered anti-Parkin-2TdCH2-RP antibody conjugate at both 4 and 8 molar ratios tested (FIG. 20A). Finally, an anti-FLAG Tag-2TdCH2 antibody conjugated with either HRP or Biotin at different molar ratios was compared with the corresponding parental (wild type) anti-FLAG Tag antibody (Product #701629) conjugates. A greater than 2-fold increase in sensitivity was observed for the anti-FLAG Tag-2TdCH2-RP antibody conjugate (4 molar excess of HRP) as compared to the parental (wild type) HRP conjugate (FIG. 20B). A 1.6 fold and 2 fold increase in sensitivity was observed for the FLAG Tag-2TdCH2-biotin antibody conjugated with 25 and 50M excess biotin compared to the corresponding wildtype (FIG. 20C).

### 2TdCH2 Engineered Backbone Increases Sensitivity of Primary Antibodies Conjugated Using SiteClick™ Chemistry

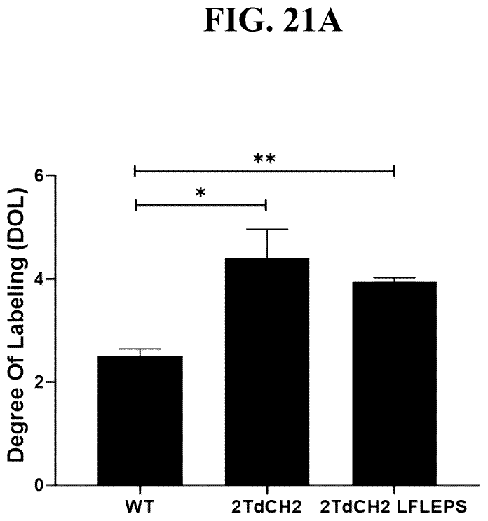

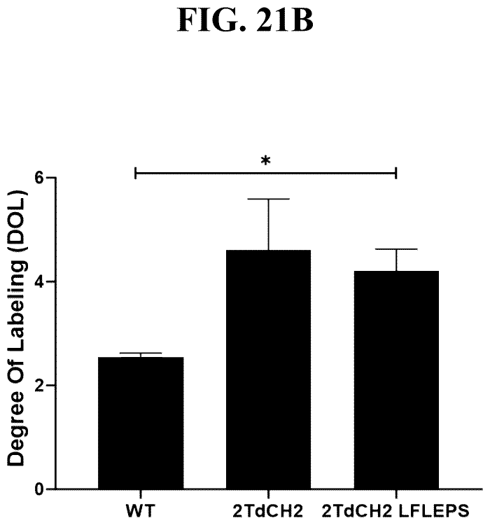

A site-specific conjugation chemistry, SiteClick™ chemistry, was used to evaluate whether the degree of labeling (DOL) is increased in the engineered backbone when conjugation occurs via a glycan residue. For this purpose, Parkin-2TdCH2 and OCT4-2TdCH2 antibodies with or without the LFLEPS mutations were conjugated to Alexa Fluor™ 488 (AF488) dye using SiteClick™ chemistry and the degree of labeling was compared to the corresponding parental (wild type) antibodies. A significant 2 fold increase in DOL was observed in all the engineered antibodies compared to the corresponding Parkin parental antibody-AF488 conjugate (FIG. 21A) and OCT4 parental antibody-AF488 conjugate (FIG. 21B).

Together, these results demonstrate that rabbit 2TdCH2 engineered backbone with or without LFLEPS mutations can increase the sensitivity of primary conjugates through increased degree of labeling and this is applicable to both lysine based and glycan-based conjugation chemistries.

### DISCUSSION

The above results provide a rational design to circumvent the disadvantages of prior studies by selectively adding additional CH2 domains (2TdCH2) instead of the entire Fc region. It was discovered that the 2TdCH2 engineered antibodies provided comparable yields, monomer purity, and stability to wildtype antibodies. In addition, the 2TdCH2 engineered antibodies provided a significant increase in sensitivity in multiple applications such as western blotting and immunocytochemistry and broadened the application coverage for the antibodies. In addition to the benefits for the unconjugated antibodies, 2TdCH2 engineering also provides additional binding sites for different conjugation chemistries (lysine residues for amine-based chemistry or glycan residues for SiteClick™ based chemistry). It was also discovered that the 2TdCH2 engineered backbone increased the degree of labeling (DOL) and consequently increased the signal for conjugated versions of these antibodies. Thus, 2TdCH2 Fc engineering provides many advantages to sensitivity enhancement for both unconjugated and conjugated antibodies and this effect can be observed across different species (rabbit, mouse) as well as primary and secondary antibodies.

FcγR1 receptors are expressed on the surface of certain cell types such as monocytes, neutrophils and eosinophils. Certain Fc domains such as those from rabbit and human IgG contain residues that bind to FcγR1 and this is important for mediating effector functions such as Antibody Dependent Cellular Cytotoxicity (ADCC). While this interaction has proven useful for therapeutic antibodies, it results in background binding and thus increased noise and lower sensitivity in immunoassays such as immunofluorescence and flow cytometry using these cell models. Immunostaining applications of targets in these cell types with rabbit antibodies result in increased background staining that is usually reduced by an additional Fc-block step in the protocol. It has also been demonstrated that mutations of certain amino acids in the lower hinge and CH2 domain of certain IgGs, such as human IgG1, Rabbit IgG, Mouse IgG2a and Rat IgG2b, reduce or abolish the background binding of these antibodies to Fc receptors, thus increasing antibody sensitivity. In amplifying the lower hinge and CH2 domains in the 2TdCH2 design, the number of binding sites for Fc receptors increase, and hence this design when used for antibodies in flow cytometry analysis of cells expressing the Fc receptors will increase background staining. To overcome this undesired result, the LFLEPS mutations were introduced into the 2TdCH2 design to reduce to reduce or abolish Fc receptor binding. Other Fc domains such as that of Mouse IgG1 do not bind FcγR1 since their Fc domains lack these residues; therefore, 2TdCH2 engineering alone is able to confer the benefits of increased sensitivity without LFLEPS mutations. It has been demonstrated herein that introduction of LFLEPS mutations into the 2TdCH2 backbone does not disrupt any secondary antibody binding sites for western blotting. This enables the development of the 2TdCH2-LFLEPS backbone as a universal Fc engineered backbone which retains the benefits of the CH2 amplification while abolishing the increased binding sites for Fc receptors contained within this domain.

One of the advantages of the engineered antibodies of the present disclosure is the ease of scalability since it can be applied to any antibody once the engineered backbone for that particular species/isotype has been developed. Additionally, because the engineering is far away from the antigen binding site, there is no change to the paratope and the antibody clone can be maintained. This disclosure has demonstrated the utility of this engineered backbone in species-switched chimeric antibodies. For these species-switched chimeric antibodies, antigen binding sites can be grafted onto the engineered Fc backbone which can provide the same benefits of increased sensitivity in the new species. Another advantage is that the engineered backbone is translatable across species as it has been demonstrated for both rabbit and mouse IgG1. Furthermore, the ease of use of the instant engineered backbone enables its usage with any commonly available relevant secondary antibody to realize its benefit. When combined with other technology for sensitivity enhancement, such as developing reagent chemistry, the 2TdCH2 backbone with or without the LFLEPS mutations will have an additive effect to lower the limit of detection.

### Embodiments

The following list of embodiments is intended to complement, rather than displace or supersede, the previous descriptions.

Embodiment 1. An isolated polypeptide comprising the amino acid sequence of Formula I: Z-(X-Yₙ-Zᵣ)ₚ-X, wherein
Z is a hinge,
X is a CH2 domain,
Y is a linker,
n is an integer of 1 or greater,
p is an integer of 2 or greater, and
r is an integer of 0 or 1.

Embodiment 2. The isolated polypeptide of embodiment 1, wherein
Z comprises an amino acid sequence comprising at least 90% identity to SEQ ID NO: 18, SEQ ID NO: 48, or SEQ ID NO: 102,
X comprises an amino acid sequence comprising at least 90% identity to SEQ ID NO: 19, SEQ ID NO: 49, or SEQ ID NO: 104,
Y is a linker,
n is an integer of 1 or greater,
p is an integer of 2 or greater, and
r is an integer of 0 or 1.

Embodiment 3. The isolated polypeptide of embodiment 1 or embodiment 2, wherein Y comprises an amino acid sequence of GS, GGS, at least 90% identity to SEQ ID NO: 14, at least 90% identity to SEQ ID NO: 38, at least 90% identity to SEQ ID NO: 43, or at least 90% identity to SEQ ID NO: 44.

Embodiment 4. The isolated polypeptide of any one of embodiments 1 to 3, wherein Z comprises SEQ ID NO: 18, SEQ ID NO: 48, or SEQ ID NO: 102; X comprises SEQ ID NO: 19, SEQ ID NO: 49, or SEQ ID NO: 104; Y comprises GS, GGS, SEQ ID NO: 14, SEQ ID NO: 38, SEQ ID NO: 43, or SEQ ID NO: 44; n is an integer of 1 or greater; p is an integer of 2 or greater; and r is an integer of 0 or 1.

Embodiment 5. The isolated polypeptide of any one of embodiments 1 to 4, wherein Z comprises the amino acid sequence of SEQ ID NO: 18, X comprises the amino acid sequence of SEQ ID NO: 19, Y comprises the amino acid sequence of SEQ ID NO: 44, n is 1, and p is 2.

Embodiment 6. The isolated polypeptide of any one of embodiments 1 to 4, wherein Z comprises the amino acid sequence of SEQ ID NO: 48, X comprises the amino acid sequence of SEQ ID NO: 49, Y comprises the amino acid sequence of SEQ ID NO: 44, n is 1, and p is 2.

Embodiment 7. The isolated polypeptide of any one of embodiments 1 to 4, wherein Z comprises the amino acid sequence of SEQ ID NO: 102, X comprises the amino acid sequence of SEQ ID NO: 104, Y comprises the amino acid sequence of SEQ ID NO: 44, n is 1, and p is 2.

Embodiment 8. The isolated polypeptide of any one of the preceding embodiments, wherein the isolated polypeptide is linked to a second polypeptide by one or more disulfide bonds, wherein the isolated polypeptide and the second polypeptide share the same amino acid sequence.

Embodiment 9. The isolated polypeptide of any one of the preceding embodiments, wherein the isolated polypeptide comprises the amino acid sequence of SEQ ID NO: 53 or SEQ ID NO: 55.

Embodiment 10. The isolated polypeptide of embodiment 8, wherein the one or more disulfide bonds occur between the hinge of the isolated polypeptide and the hinge of the second polypeptide.

Embodiment 11. The isolated polypeptide of any one of the preceding embodiments, wherein the isolated polypeptide further comprises a CH3 domain.

Embodiment 12. The isolated polypeptide of embodiment 9, wherein the CH3 domain comprises the amino acid sequence of SEQ ID NO: 20, or SEQ ID NO: 106.

Embodiment 13. An antibody comprising the isolated polypeptide of any one of the preceding embodiments linked to a Fab domain.

Embodiment 14. The antibody of embodiment 13, wherein the antibody has a reduction of background signal of at least 1.5-fold compared to a corresponding wildtype antibody without the isolated polypeptide.

Embodiment 15. The antibody of embodiment 13 or 14, wherein the antibody has an increase in sensitivity of at least 1.5-fold compared to a corresponding wildtype antibody without the isolated polypeptide.

Embodiment 16. The antibody of embodiment 13, wherein the Fab comprises a CH1 domain.

Embodiment 17. The antibody of embodiment 16, wherein the CH1 domain comprises the amino acid sequence of SEQ ID NO: 17, SEQ ID NO: 100, or SEQ ID NO: 160.

Embodiment 18. The antibody of embodiment 13, wherein the Fab comprises a Fab heavy chain and a Fab light chain that pair to form a binding domain.

Embodiment 19. The antibody of any one of embodiments 13 to 18, wherein the Fab immunospecifically binds to Parkin.

Embodiment 20. The antibody of embodiment 19, wherein the Fab heavy chain comprises a heavy chain complementarity determining region 1 (HCDR1) comprising the amino acid sequence of SEQ ID NO: 4, a HCDR2 comprising the amino acid sequence of SEQ ID NO: 5, and a HCDR3 comprising the amino acid sequence of SEQ ID NO: 6.

Embodiment 21. The antibody of embodiment 19 or 20, wherein the Fab heavy chain comprises a variable region comprising the amino acid sequence of SEQ ID NO: 7.

Embodiment 22. The antibody of any one of embodiments 19 to 21, wherein the Fab heavy chain comprises the amino acid sequence of SEQ ID NO: 9.

Embodiment 23. The antibody of any one of embodiments 19 to 22, wherein the Fab light chain comprises a light chain complementarity determining region 1 (LCDR1) comprising the amino acid sequence of SEQ ID NO: 13, a LCDR2 comprising the amino acid sequence RAS, and a LCDR3 comprising the amino acid sequence of SEQ ID NO: 15.

Embodiment 24. The antibody of any one of embodiments 19 to 23, wherein the Fab light chain comprises a variable region comprising the amino acid sequence of SEQ ID NO: 16.

Embodiment 25. The antibody of any one of embodiments 19 to 24, wherein the Fab light chain comprises the amino acid sequence of SEQ ID NO: 12.

Embodiment 26. The antibody of any one of embodiments 19 to 25, wherein the isolated polypeptide comprises the amino acid sequence of SEQ ID NO: 3.

Embodiment 27. The antibody of any one of embodiments 13 to 18, wherein the Fab immunospecifically binds to OCT4.

Embodiment 28. The antibody of embodiment 27, wherein the Fab heavy chain comprises a heavy chain complementarity determining region 1 (HCDR1) comprising the amino acid sequence of SEQ ID NO: 29, a HCDR2 comprising the amino acid sequence of SEQ ID NO: 30, and a HCDR3 comprising the amino acid sequence of SEQ ID NO: 31.

Embodiment 29. The antibody of embodiment 27 or embodiment 28, wherein the Fab heavy chain comprises a variable region comprising the amino acid sequence of SEQ ID NO: 32.

Embodiment 30. The antibody of any one of embodiments 27 to 29, wherein the Fab heavy chain comprises the amino acid sequence of SEQ ID NO: 34.

Embodiment 31. The antibody of any one of embodiments 27 to 30, wherein the Fab light chain comprises a light chain complementarity determining region 1 (LCDR1) comprising the amino acid sequence of SEQ ID NO: 37, a LCDR2 comprising the amino acid sequence GAS, and a LCDR3 comprising the amino acid sequence of SEQ ID NO: 39.

Embodiment 32. The antibody of any one of embodiments 27 to 31, wherein the Fab light chain comprises a variable region comprising the amino acid sequence of SEQ ID NO: 40.

Embodiment 33. The antibody of any one of embodiments 27 to 32, wherein the Fab light chain comprises the amino acid sequence of SEQ ID NO: 36.

Embodiment 34. The antibody of any one of embodiments 27 to 33, wherein the isolated polypeptide comprises the amino acid sequence of SEQ ID NO: 28.

Embodiment 35. The antibody of any one of embodiments 13 to 18, wherein the Fab immunospecifically binds to IDO1.

Embodiment 36. The antibody of embodiment 35, wherein the Fab heavy chain comprises a heavy chain complementarity determining region 1 (HCDR1) comprising the amino acid sequence of SEQ ID NO: 66, a HCDR2 comprising the amino acid sequence of SEQ ID NO: 67, and a HCDR3 comprising the amino acid sequence of SEQ ID NO: 68.

Embodiment 37. The antibody of embodiment 35 or 36, wherein the Fab heavy chain comprises a variable region comprising the amino acid sequence of SEQ ID NO: 65.

Embodiment 38. The antibody of any one of embodiments 35 to 37, wherein the Fab heavy chain comprises the amino acid sequence of SEQ ID NO: 75.

Embodiment 39. The antibody of any one of embodiments 35 to 38, wherein the Fab light chain comprises a light chain complementarity determining region 1 (LCDR1) comprising the amino acid sequence of SEQ ID NO: 72, a LCDR2 comprising the amino acid sequence of YAS, and a LCDR3 comprising the amino acid sequence of SEQ ID NO: 73.

Embodiment 40. The antibody of any one of embodiments 35 to 39, wherein the Fab light chain comprises a variable region comprising the amino acid sequence of SEQ ID NO: 71.

Embodiment 41. The antibody of any one of embodiments 35 to 40, wherein the Fab light chain comprises the amino acid sequence of SEQ ID NO: 77.

Embodiment 42. The antibody of any one of embodiments 35 to 41, wherein the isolated polypeptide comprises the amino acid sequence of SEQ ID NO: 79.

Embodiment 43. The antibody of any one of embodiments 13 to 18, wherein the Fab immunospecifically binds to FLAG-tag.

Embodiment 44. The antibody of embodiment 43, wherein the Fab heavy chain comprises a heavy chain complementarity determining region 1 (HCDR1) comprising the amino acid sequence of SEQ ID NO: 163, a HCDR2 comprising the amino acid sequence of SEQ ID NO: 164, and a HCDR3 comprising the amino acid sequence of SEQ ID NO: 165.

Embodiment 45. The antibody of embodiment 43 or 44, wherein the Fab heavy chain comprises a variable region comprising the amino acid sequence of SEQ ID NO: 162.

Embodiment 46. The antibody of any one of embodiments 43 to 45, wherein the Fab heavy chain comprises the amino acid sequence of SEQ ID NO: 171.

Embodiment 47. The antibody of any one of embodiments 43 to 46, wherein the Fab light chain comprises a light chain complementarity determining region 1 (LCDR1) comprising the amino acid sequence of SEQ ID NO: 168, a LCDR2 comprising the amino acid sequence of YAS, and a LCDR3 comprising the amino acid sequence of SEQ ID NO: 169.

Embodiment 48. The antibody of any one of embodiments 43 to 47, wherein the Fab light chain comprises a variable region comprising the amino acid sequence of SEQ ID NO: 167.

Embodiment 49. The antibody of any one of embodiments 43 to 48, wherein the Fab light chain comprises the amino acid sequence of SEQ ID NO: 172.

Embodiment 50. The antibody of any one of embodiments 43 to 49, wherein the isolated polypeptide comprises the amino acid sequence of SEQ ID NO: 175.

Embodiment 51. The antibody of any one of embodiments 13 to 18, wherein the Fab is a Rabbit anti-Mouse IgG secondary antibody.

Embodiment 52. The antibody of embodiment 51, wherein the Fab heavy chain comprises a heavy chain complementarity determining region 1 (HCDR1) comprising the amino acid sequence of SEQ ID NO: 127, a HCDR2 comprising the amino acid sequence of SEQ ID NO: 128, and a HCDR3 comprising the amino acid sequence of SEQ ID NO: 129.

Embodiment 53. The antibody of embodiment 51 or 52, wherein the Fab heavy chain comprises a variable region comprising the amino acid sequence of SEQ ID NO: 126.

Embodiment 54. The antibody of any one of embodiments 51 to 53, wherein the Fab heavy chain comprises the amino acid sequence of SEQ ID NO: 135.

Embodiment 55. The antibody of any one of embodiments 51 to 54, wherein the Fab light chain comprises a light chain complementarity determining region 1 (LCDR1) comprising the amino acid sequence of SEQ ID NO: 132, a LCDR2 comprising the amino acid sequence GAS, and a LCDR3 comprising the amino acid sequence of SEQ ID NO: 133.

Embodiment 56. The antibody of any one of embodiments 51 to 55, wherein the Fab light chain comprises a variable domain comprising the amino acid sequence of SEQ ID NO: 130.

Embodiment 57. The antibody of any one of embodiments 51 to 56, wherein the Fab light chain comprises the amino acid sequence of SEQ ID NO: 137.

Embodiment 58. The antibody of any one of embodiments 51 to 57, wherein the isolated polypeptide comprises the amino acid sequence of SEQ ID NO: 139.

Embodiment 59. The antibody of any one of embodiments 13 to 18, wherein the Fab immunospecifically binds to pERK1/2.

Embodiment 60. The antibody of embodiment 59, wherein the Fab heavy chain comprises a heavy chain complementarity determining region 1 (HCDR1) comprising the amino acid sequence of SEQ ID NO: 85, a HCDR2 comprising the amino acid sequence of SEQ ID NO: 86, and a HCDR3 comprising the amino acid sequence of SEQ ID NO: 87.

Embodiment 61. The antibody of embodiment 59 or 60, wherein the Fab heavy chain comprises a variable region comprising the amino acid sequence of SEQ ID NO: 84.

Embodiment 62. The antibody of any one of embodiments 59 to 61, wherein the Fab heavy chain comprises the amino acid sequence of SEQ ID NO: 94.

Embodiment 63. The antibody of any one of embodiments 59 to 62, wherein the Fab light chain comprises a light chain complementarity determining region 1 (LCDR1) comprising the amino acid sequence of SEQ ID NO: 91, a LCDR2 comprising the amino acid sequence of KVS, and a LCDR3 comprising the amino acid sequence of SEQ ID NO: 92.

Embodiment 64. The antibody of any one of embodiments 59 to 63, wherein the Fab light chain comprises a variable domain comprising the amino acid sequence of SEQ ID NO: 90.

Embodiment 65. The antibody of any one of embodiments 59 to 64, wherein the Fab light chain comprises the amino acid sequence of SEQ ID NO: 96.

Embodiment 66. The antibody of any one of embodiments 59 to 65, wherein the isolated polypeptide comprises the amino acid sequence of SEQ ID NO: 98.

Embodiment 67. The antibody of any one of embodiments 13 to 18, wherein the Fab immunospecifically binds to PSD-95.

Embodiment 68. The antibody of embodiment 67, wherein the Fab heavy chain comprises a heavy chain complementarity determining region 1 (HCDR1) comprising the amino acid sequence of SEQ ID NO: 111, a HCDR2 comprising the amino acid sequence of SEQ ID NO: 112, and a HCDR3 comprising the amino acid sequence of SEQ ID NO: 113.

Embodiment 69. The antibody of embodiment 67 or embodiment 68, wherein the Fab heavy chain comprises a variable region comprising the amino acid sequence of SEQ ID NO: 110.

Embodiment 70. The antibody of any one of embodiments 67 to 69, wherein the Fab heavy chain comprises the amino acid sequence of SEQ ID NO: 119.

Embodiment 71. The antibody of any one of embodiments 67 to 70, wherein the Fab light chain comprises a light chain complementarity determining region 1 (LCDR1) comprising the amino acid sequence of SEQ ID NO: 116, a LCDR2 comprising the amino acid sequence of DTS, and a LCDR3 comprising the amino acid sequence of SEQ ID NO: 117.

Embodiment 72. The antibody of any one of embodiments 67 to 71, wherein the Fab light chain comprises a variable domain comprising the amino acid sequence of SEQ ID NO: 115.

Embodiment 73. The antibody of any one of embodiments 67 to 72, wherein the Fab light chain comprises the amino acid sequence of SEQ ID NO: 121.

Embodiment 74. The antibody of any one of embodiments 67 to 73, wherein the isolated polypeptide comprises the amino acid sequence of SEQ ID NO: 123.

Embodiment 75. The antibody of any one of embodiments 13 to 18, wherein the Fab immunospecifically binds to TIGIT.

Embodiment 76. The antibody of embodiment 75, wherein the Fab heavy chain comprises a heavy chain complementarity determining region 1 (HCDR1) comprising the amino acid sequence of SEQ ID NO: 146, a HCDR2 comprising the amino acid sequence of SEQ ID NO: 147, and a HCDR3 comprising the amino acid sequence of SEQ ID NO: 148.

Embodiment 77. The antibody of embodiment 75 or embodiment 76, wherein the Fab heavy chain comprises a variable region comprising the amino acid sequence of SEQ ID NO: 145.

Embodiment 78. The antibody of any one of embodiments 75 to 77, wherein the Fab heavy chain comprises the amino acid sequence of SEQ ID NO: 154.

Embodiment 79. The antibody of any one of embodiments 75 to 78, wherein the Fab light chain comprises a light chain complementarity determining region 1 (LCDR1) comprising the amino acid sequence of SEQ ID NO: 151, a LCDR2 comprising the amino acid sequence of SAS, and a LCDR3 comprising the amino acid sequence of SEQ ID NO: 152.

Embodiment 80. The antibody of any one of embodiments 75 to 79, wherein the Fab light chain comprises a variable domain comprising the amino acid sequence of SEQ ID NO: 150.

Embodiment 81. The antibody of any one of embodiments 75 to 80, wherein the Fab light chain comprises the amino acid sequence of SEQ ID NO: 156.

Embodiment 82. The antibody of any one of embodiments 75 to 81, wherein the isolated polypeptide comprises the amino acid sequence of SEQ ID NO: 158.

Embodiment 83. The antibody of any one of embodiments 13 to 82, wherein the isolated polypeptide is an antibody with the isotype IgG.

Embodiment 84. The isolated polypeptide of any one of embodiments 1-12 or the antibody of any one of embodiments 13-83, further comprising a label.

Embodiment 85. A nucleic acid encoding the isolated polypeptide of any one of embodiments 1-10 or the antibody of any one of embodiments 13-83.

Embodiment 86. An antibody that immunospecifically binds to Parkin, wherein the antibody comprises a heavy chain (HC) and a light chain (LC) that pair to form a binding domain comprising a heavy chain complementarity determining region 1 (HCDR1) comprising the amino acid sequence of SEQ ID NO: 4, a HCDR2 comprising the amino acid sequence of SEQ ID NO: 5, a HCDR3 comprising the amino acid sequence of SEQ ID NO: 6, a light chain complementarity determining region 1 (LCDR1) comprising the amino acid sequence of SEQ ID NO: 13, a LCDR2 comprising the amino acid sequence RAS, and a LCDR3 comprising the amino acid sequence of SEQ ID NO: 15, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

Embodiment 87. The antibody of embodiment 86, wherein the first CH2 domain, the second CH2 domain, and the third CH2 domain each comprises the amino acid sequence of SEQ ID NO: 19 or SEQ ID NO: 49.

Embodiment 88. The antibody of embodiment 86 or embodiment 87, wherein the first hinge and the second hinge each comprises the amino acid sequence of SEQ ID NO: 18 or SEQ ID NO: 48.

Embodiment 89. The antibody of any one of embodiments 86 to 88, wherein the first linker and the second linker each comprises the amino acid sequence of GS, GGS, SEQ ID NO: 14, SEQ ID NO: 38, SEQ ID NO: 43, or SEQ ID NO: 44.

Embodiment 90. The antibody of any one of embodiments 86 to 89, wherein the CH3 domain comprises the amino acid sequence of SEQ ID NO: 20.

Embodiment 91. The antibody of any one of embodiments 86 to 90, wherein the antibody comprises a heavy chain variable region having the polypeptide sequence of SEQ ID NO: 7 and a light chain variable region having the polypeptide sequence of SEQ ID NO: 16.

Embodiment 92. The antibody of any one of embodiments 86 to 91, wherein the antibody comprises a heavy chain having a polypeptide sequence of SEQ ID NO: 3 and a light chain having the polypeptide sequence of SEQ ID NO: 12.

Embodiment 93. The antibody of any one of embodiments 86 to 92, wherein the antibody comprises a heavy chain that is encoded by a polynucleotide sequence of SEQ ID NO: 2 and a light chain that is encoded by a polynucleotide sequence of SEQ ID NO: 11.

Embodiment 94. An antibody that immunospecifically binds to OCT4, wherein the antibody comprises a heavy chain (HC) and a light chain (LC) that pair to form a binding domain comprising a heavy chain complementarity determining region 1 (HCDR1) comprising the amino acid sequence of SEQ ID NO: 29, a HCDR2 comprising the amino acid sequence of SEQ ID NO: 30, and a HCDR3 comprising the amino acid sequence of SEQ ID NO: 31, a light chain complementarity determining region 1 (LCDR1) comprising the amino acid sequence of SEQ ID NO: 37, a LCDR2 comprising the amino acid sequence GAS, and a LCDR3 comprising the amino acid sequence of SEQ ID NO: 39, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

Embodiment 95. The antibody of embodiment 94, wherein the first CH2 domain, the second CH2 domain, and the third CH2 domain each comprises the amino acid sequence of SEQ ID NO: 19 or SEQ ID NO: 49.

Embodiment 96. The antibody of embodiment 94 or embodiment 95, wherein the first hinge and the second hinge each comprises the amino acid sequence of SEQ ID NO: 18 or SEQ ID NO: 48.

Embodiment 97. The antibody of any one of embodiments 94 to 96, wherein the first linker and the second linker each comprises the amino acid sequence of GS, GGS, SEQ ID NO: 14, SEQ ID NO: 38, SEQ ID NO: 43, or SEQ ID NO: 44.

Embodiment 98. The antibody of any one of embodiments 94 to 97, wherein the CH3 domain comprises the amino acid sequence of SEQ ID NO: 20.

Embodiment 99. The antibody of any one of embodiments 94 to 98, wherein the antibody comprises a heavy chain variable region having the polypeptide sequence of SEQ ID NO: 32 and a light chain variable region having the polypeptide sequence of SEQ ID NO: 40.

Embodiment 100. The antibody of any one of embodiments 94 to 99, wherein the antibody comprises a heavy chain having a polypeptide sequence of SEQ ID NO: 28 and a light chain having the polypeptide sequence of SEQ ID NO: 36.

Embodiment 101. The antibody of any one of embodiments 94 to 100, wherein the antibody comprises a heavy chain that is encoded by a polynucleotide sequence of SEQ ID NO: 27 and a light chain that is encoded by a polynucleotide sequence of SEQ ID NO: 35.

Embodiment 102. An antibody that immunospecifically binds to IDO1, wherein the antibody comprises a heavy chain (HC) and a light chain (LC) that pair to form a binding domain comprising a heavy chain complementarity determining region 1 (HCDR1) comprising the amino acid sequence of SEQ ID NO: 66, a HCDR2 comprising the amino acid sequence of SEQ ID NO: 67, a HCDR3 comprising the amino acid sequence of SEQ ID NO: 68, a light chain complementarity determining region 1 (LCDR1) comprising the amino acid sequence of SEQ ID NO: 72, a LCDR2 comprising the amino acid sequence of YAS, and a LCDR3 comprising the amino acid sequence of SEQ ID NO: 73, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

Embodiment 103. The antibody of embodiment 102, wherein the first CH2 domain, the second CH2 domain, and the third CH2 domain each comprises the amino acid sequence of SEQ ID NO: 19 or SEQ ID NO: 49.

Embodiment 104. The antibody of embodiment 102 or embodiment 103, wherein the first hinge and the second hinge each comprises the amino acid sequence of SEQ ID NO: 18 or SEQ ID NO: 48.

Embodiment 105. The antibody of any one of embodiments 102 to 104, wherein the first linker and the second linker each comprises the amino acid sequence of GS, GGS, SEQ ID NO: 14, SEQ ID NO: 38, SEQ ID NO: 43, or SEQ ID NO: 44.

Embodiment 106. The antibody of any one of embodiments 102 to 105, wherein the CH3 domain comprises the amino acid sequence of SEQ ID NO: 20.

Embodiment 107. The antibody of any one of embodiments 102 to 106, wherein the antibody comprises a heavy chain variable region having the polypeptide sequence of SEQ ID NO: 65 and a light chain variable region having the polypeptide sequence of SEQ ID NO: 71.

Embodiment 108. The antibody of any one of embodiments 102 to 107, wherein the antibody comprises a heavy chain having a polypeptide sequence of SEQ ID NO: 75 and a light chain having the polypeptide sequence of SEQ ID NO: 77.

Embodiment 109. The antibody of any one of embodiments 102 to 108, wherein the antibody comprises a heavy chain that is encoded by a polynucleotide sequence of SEQ ID NO: 74 and a light chain that is encoded by a polynucleotide sequence of SEQ ID NO: 76.

Embodiment 110. An antibody that immunospecifically binds to FLAG-tag, wherein the antibody comprises a heavy chain (HC) and a light chain (LC) that pair to form a binding domain comprising a heavy chain complementarity determining region 1 (HCDR1) comprising the amino acid sequence of SEQ ID NO: 163, a HCDR2 comprising the amino acid sequence of SEQ ID NO: 164, a HCDR3 comprising the amino acid sequence of SEQ ID NO: 165, a light chain complementarity determining region 1 (LCDR1) comprising the amino acid sequence of SEQ ID NO: 168, a LCDR2 comprising the amino acid sequence of YAS, and a LCDR3 comprising the amino acid sequence of SEQ ID NO: 169, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

Embodiment 111. The antibody of embodiment 110, wherein the first CH2 domain, the second CH2 domain, and the third CH2 domain each comprises the amino acid sequence of SEQ ID NO: 19 or SEQ ID NO: 49.

Embodiment 112. The antibody of embodiment 110 or embodiment 111, wherein the first hinge and the second hinge each comprises the amino acid sequence of SEQ ID NO: 18 or SEQ ID NO: 48.

Embodiment 113. The antibody of any one of embodiments 110 to 112, wherein the first linker and the second linker each comprises the amino acid sequence of GS, GGS, SEQ ID NO: 14, SEQ ID NO: 38, SEQ ID NO: 43, or SEQ ID NO: 44.

Embodiment 114. The antibody of any one of embodiments 110 to 113, wherein the CH3 domain comprises the amino acid sequence of SEQ ID NO: 20.

Embodiment 115. The antibody of any one of embodiments 110 to 114, wherein the antibody comprises a heavy chain variable region having the polypeptide sequence of SEQ ID NO: 162 and a light chain variable region having the polypeptide sequence of SEQ ID NO: 167.

Embodiment 116. The antibody of any one of embodiments 110 to 115, wherein the antibody comprises a heavy chain having a polypeptide sequence of SEQ ID NO: 171 and a light chain having the polypeptide sequence of SEQ ID NO: 173.

Embodiment 117. The antibody of any one of embodiments 110 to 116, wherein the antibody comprises a heavy chain that is encoded by a polynucleotide sequence of SEQ ID NO: 170 and a light chain that is encoded by a polynucleotide sequence of SEQ ID NO: 172.

Embodiment 118. An antibody that is a Rabbit anti-Mouse IgG secondary antibody, wherein the antibody comprises a heavy chain (HC) and a light chain (LC) that pair to form a binding domain comprising a heavy chain complementarity determining region 1 (HCDR1) comprising the amino acid sequence of SEQ ID NO: 127, a HCDR2 comprising the amino acid sequence of SEQ ID NO: 128, a HCDR3 comprising the amino acid sequence of SEQ ID NO: 129, a light chain complementarity determining region 1 (LCDR1) comprising the amino acid sequence of SEQ ID NO: 132, a LCDR2 comprising the amino acid sequence of GAS, and a LCDR3 comprising the amino acid sequence of SEQ ID NO: 133, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

Embodiment 119. The antibody of embodiment 118, wherein the first CH2 domain, the second CH2 domain, and the third CH2 domain each comprises the amino acid sequence of SEQ ID NO: 19 or SEQ ID NO: 49.

Embodiment 120. The antibody of embodiment 118 or embodiment 119, wherein the first hinge and the second hinge each comprises the amino acid sequence of SEQ ID NO: 18 or SEQ ID NO: 48.

Embodiment 121. The antibody of any one of embodiments 118 to 120, wherein the first linker and the second linker each comprises the amino acid sequence of GS, GGS, SEQ ID NO: 14, SEQ ID NO: 38, SEQ ID NO: 43, or SEQ ID NO: 44.

Embodiment 122. The antibody of any one of embodiments 118 to 121, wherein the CH3 domain comprises the amino acid sequence of SEQ ID NO: 20.

Embodiment 123. The antibody of any one of embodiments 118 to 122, wherein the antibody comprises a heavy chain variable region having the polypeptide sequence of SEQ ID NO: 126 and a light chain variable region having the polypeptide sequence of SEQ ID NO: 131.

Embodiment 124. The antibody of any one of embodiments 118 to 123, wherein the antibody comprises a heavy chain having a polypeptide sequence of SEQ ID NO: 135 and a light chain having the polypeptide sequence of SEQ ID NO: 137.

Embodiment 125. The antibody of any one of embodiments 118 to 124, wherein the antibody comprises a heavy chain that is encoded by a polynucleotide sequence of SEQ ID NO: 134 and a light chain that is encoded by a polynucleotide sequence of SEQ ID NO: 136.

Embodiment 126. An antibody that immunospecifically binds to pERK1/2, wherein the antibody comprises a heavy chain (HC) and a light chain (LC) that pair to form a binding domain comprising a heavy chain complementarity determining region 1 (HCDR1) comprising the amino acid sequence of SEQ ID NO: 85, a HCDR2 comprising the amino acid sequence of SEQ ID NO: 86, a HCDR3 comprising the amino acid sequence of SEQ ID NO: 87, a light chain complementarity determining region 1 (LCDR1) comprising the amino acid sequence of SEQ ID NO: 91, a LCDR2 comprising the amino acid sequence of KVS, and a LCDR3 comprising the amino acid sequence of SEQ ID NO: 92, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

Embodiment 127. The antibody of embodiment 126, wherein the first CH2 domain, the second CH2 domain, and the third CH2 domain each comprises the amino acid sequence of SEQ ID NO: 104.

Embodiment 128. The antibody of embodiment 126 or embodiment 127, wherein the first hinge and the second hinge each comprises the amino acid sequence of SEQ ID NO: 102.

Embodiment 129. The antibody of any one of embodiments 126 to 128, wherein the first linker and the second linker each comprises the amino acid sequence of GS, GGS, SEQ ID NO: 14, SEQ ID NO: 38, SEQ ID NO: 43, or SEQ ID NO: 44.

Embodiment 130. The antibody of any one of embodiments 126 to 129, wherein the CH3 domain comprises the amino acid sequence of SEQ ID NO: 106.

Embodiment 131. The antibody of any one of embodiments 126 to 130, wherein the antibody comprises a heavy chain variable region having the polypeptide sequence of SEQ ID NO: 83 and a light chain variable region having the polypeptide sequence of SEQ ID NO: 90.

Embodiment 132. The antibody of any one of embodiments 126 to 131, wherein the antibody comprises a heavy chain having a polypeptide sequence of SEQ ID NO: 94 and a light chain having the polypeptide sequence of SEQ ID NO: 96.

Embodiment 133. The antibody of any one of embodiments 126 to 132, wherein the antibody comprises a heavy chain that is encoded by a polynucleotide sequence of SEQ ID NO: 93 and a light chain that is encoded by a polynucleotide sequence of SEQ ID NO: 95.

Embodiment 134. An antibody that immunospecifically binds to PSD-95, wherein the antibody comprises a heavy chain (HC) and a light chain (LC) that pair to form a binding domain comprising a heavy chain complementarity determining region 1 (HCDR1) comprising the amino acid sequence of SEQ ID NO: 111, a HCDR2 comprising the amino acid sequence of SEQ ID NO: 112, a HCDR3 comprising the amino acid sequence of SEQ ID NO: 113, a light chain complementarity determining region 1 (LCDR1) comprising the amino acid sequence of SEQ ID NO: 116, a LCDR2 comprising the amino acid sequence of DTS, and a LCDR3 comprising the amino acid sequence of SEQ ID NO: 117, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

Embodiment 135. The antibody of embodiment 134, wherein the first CH2 domain, the second CH2 domain, and the third CH2 domain each comprises the amino acid sequence of SEQ ID NO: 104.

Embodiment 136. The antibody of embodiment 134 or embodiment 135, wherein the first hinge and the second hinge each comprises the amino acid sequence of SEQ ID NO: 102.

Embodiment 137. The antibody of any one of embodiments 134 to 136, wherein the first linker and the second linker each comprises the amino acid sequence of GS, GGS, SEQ ID NO: 14, SEQ ID NO: 38, SEQ ID NO: 43, or SEQ ID NO: 44.

Embodiment 138. The antibody of any one of embodiments 134 to 137, wherein the CH3 domain comprises the amino acid sequence of SEQ ID NO: 106.

Embodiment 139. The antibody of any one of embodiments 134 to 138, wherein the antibody comprises a heavy chain variable region having the polypeptide sequence of SEQ ID NO: 110 and a light chain variable region having the polypeptide sequence of SEQ ID NO: 115.

Embodiment 140. The antibody of any one of embodiments 134 to 139, wherein the antibody comprises a heavy chain having a polypeptide sequence of SEQ ID NO: 119 and a light chain having the polypeptide sequence of SEQ ID NO: 121.

Embodiment 141. The antibody of any one of embodiments 134 to 140, wherein the antibody comprises a heavy chain that is encoded by a polynucleotide sequence of SEQ ID NO: 118 and a light chain that is encoded by a polynucleotide sequence of SEQ ID NO: 120.

Embodiment 142. An antibody that immunospecifically binds to TIGIT, wherein the antibody comprises a heavy chain (HC) and a light chain (LC) that pair to form a binding domain comprising a heavy chain complementarity determining region 1 (HCDR1) comprising the amino acid sequence of SEQ ID NO: 146, a HCDR2 comprising the amino acid sequence of SEQ ID NO: 147, a HCDR3 comprising the amino acid sequence of SEQ ID NO: 148, a light chain complementarity determining region 1 (LCDR1) comprising the amino acid sequence of SEQ ID NO: 151, a LCDR2 comprising the amino acid sequence of SAS, and a LCDR3 comprising the amino acid sequence of SEQ ID NO: 152, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

Embodiment 143. The antibody of embodiment 1420, wherein the first CH2 domain, the second CH2 domain, and the third CH2 domain each comprises the amino acid sequence of SEQ ID NO: 19.

Embodiment 144. The antibody of embodiment 142 or embodiment 143, wherein the first hinge and the second hinge each comprises the amino acid sequence of SEQ ID NO: 18.

Embodiment 145. The antibody of any one of embodiments 142 to 144, wherein the first linker and the second linker each comprises the amino acid sequence of GS, GGS, SEQ ID NO: 14, SEQ ID NO: 38, SEQ ID NO: 43, or SEQ ID NO: 44.

Embodiment 146. The antibody of any one of embodiments 142 to 145, wherein the CH3 domain comprises the amino acid sequence of SEQ ID NO: 20.

Embodiment 147. The antibody of any one of embodiments 142 to 146, wherein the antibody comprises a heavy chain variable region having the polypeptide sequence of SEQ ID NO: 145 and a light chain variable region having the polypeptide sequence of SEQ ID NO: 150.

Embodiment 148. The antibody of any one of embodiments 142 to 147, wherein the antibody comprises a heavy chain having a polypeptide sequence of SEQ ID NO: 154 and a light chain having the polypeptide sequence of SEQ ID NO: 156.

Embodiment 149. The antibody of any one of embodiments 142 to 148, wherein the antibody comprises a heavy chain that is encoded by a polynucleotide sequence of SEQ ID NO: 153 and a light chain that is encoded by a polynucleotide sequence of SEQ ID NO: 155.

Embodiment 150. The antibody of any one of embodiments 13 to 84 or 86 to 149, wherein the antibody provides at least a 1.5-fold increase in signal enhancement compared to a corresponding wild type antibody.

Embodiment 151. The antibody of any one of embodiments 13 to 84 or 86 to 150, wherein the antibody provides at least a 1.5-fold reduction in background signal compared to a corresponding wild type antibody.

Embodiment 152. A method of detecting the presence of a target protein in a sample, said method comprising: Q applying the isolated polypeptide of any one of embodiments 1 to 12 or the antibody of any one of embodiments 13 to 84 or embodiments 86 to 151 to a sample containing a target protein, Q incubating the isolated polypeptide or the antibody in the sample under conditions permitting the isolated polypeptide or the antibody to bind to the target protein and to form a bound isolated polypeptide or a bound antibody, and D determining the presence of the bound isolated polypeptide or the bound antibody, thereby detecting the presence of the target protein.

Embodiment 153. The method of embodiment 152, wherein the isolated polypeptide or the antibody have less background binding to cells that express Fc gamma receptors relative to a wild type isolated polypeptide or a wild type antibody.

Embodiment 154. The method of embodiment 152 or embodiment 153, wherein the target protein is OCT4, Parkin, IDO1, FLAG-tag, pERK1/2, PSD-95 or TIGIT.

Embodiment 155. The method of any one of embodiments 152 to 154, wherein the target protein is present in the sample in low abundance.

Embodiment 156. The method of any one of embodiments 152 to 155, wherein the presence of the bound isolated polypeptide or the bound antibody is determined by applying a secondary antibody to said sample, wherein the secondary antibody is labeled.

Embodiment 157. The method of any one of embodiments 152 to 155, wherein the antibody is labeled.

Embodiment 158. The method of embodiment 156 or embodiment 157, wherein the label is selected from a fluorophore, a fluorescent protein, a phosphorescent dye, a tandem dye, a polymer dye, a particle, an electron transfer agent, a hapten, an enzyme, a radioisotope, a chromophore, and an oligonucleotide-based fluorescent dye.

Embodiment 159. The method of embodiment 154, wherein the fluorophore is a pyrene, an anthracene, a naphthalene, an acridine, a stilbene, an indole or benzindole, an oxazole or benzoxazole, a thiazole or benzothiazole, an indacene, a 4-amino-7-nitrobenz-2-oxa-1,3-diazole (NBD), a cyanine, a carbocyanine, a benzocyanine, a carbostyryl, a porphyrin, a salicylate, an anthranilate, an azulene, a perylene, a pyridine, a quinoline, a borapolyazaindacene, a xanthene, a rhodamine, a silicon rhodamine, a fluorescein, a rhodol, a rosamine, an oxazine or a benzoxazine, a benzophenoxazine, a carbazine, a phenalenone, a coumarin, a benzofuran, a benzophenalenone, a benzopyrilium, a styryl, a squarine, a resorufin, an anthraquinone, or a quinazolinone, or a conjugated polymer dye.

Embodiment 160. The method of embodiment 154, wherein the particle is a semiconductor nanocrystal.

Embodiment 161. The method of embodiment 154, wherein the hapten is a biotin or a biotin derivative.

Embodiment 162. The method of embodiment 154, wherein the enzyme is selected from a peroxidase, a phosphatase, a glycosidase, and a luciferase.

Embodiment 163. The method of embodiment 162, wherein the method further comprises adding an enzymatic substrate selected from a chromogenic, a fluorogenic, and a chemiluminescent substrate.

Embodiment 164. The method of any one of embodiments 152 to 163, wherein detecting the labeled antibody utilizes flow cytometry, immunohistochemistry, immunocytochemistry, or Western blotting.

Embodiment 165. The method of any one of embodiments 152 to 164, wherein Y comprises the amino acid sequence of GS, GGS, SEQ ID NO: 14, SEQ ID NO: 38, SEQ ID NO: 43, or SEQ ID NO: 44.

Embodiment 166. The method of any one of embodiments 152 to 165, wherein Z comprises the amino acid sequence of SEQ ID NO: 18, X comprises the amino acid sequence of SEQ ID NO: 19, Y comprises the amino acid sequence of SEQ ID NO: 44, n is 1, and p is 2.

Embodiment 167. The method of any one of embodiments 152 to 165, wherein Z comprises the amino acid sequence of SEQ ID NO: 48, X comprises the amino acid sequence of SEQ ID NO: 49, Y comprises the amino acid sequence of SEQ ID NO: 45, n is 1, and p is 2.

Embodiment 168. The method of any one of embodiments 152 to 165, wherein Z comprises the amino acid sequence of SEQ ID NO: 102, X comprises the amino acid sequence of SEQ ID NO: 104, Y comprises the amino acid sequence of SEQ ID NO: 44, n is 1, and p is 2.

Embodiment 169. The method of any one of embodiments 152 to 168, wherein the isolated polypeptide is linked to a second polypeptide by one or more disulfide bonds, wherein the isolated polypeptide and the second polypeptide share the same amino acid sequence.

Embodiment 170. The method of embodiment 169, wherein the one or more disulfide bonds occur between the hinge of the isolated polypeptide and the hinge of the second polypeptide.

Embodiment 171. The method of embodiment 169 or embodiment 170, wherein the isolated polypeptide comprises the amino acid sequence of SEQ ID NO: 53, or SEQ ID NO: 55.

Embodiment 172. The method of any one of embodiments 152 to 171, wherein the isolated polypeptide or antibody further comprises a CH3 domain.

Embodiment 173. The method of embodiment 172, wherein the CH3 domain comprises the amino acid sequence of SEQ ID NO: 20, SEQ ID NO: 106, or SEQ ID NO: 141.

Embodiment 174. The method of any one of embodiments 152 to 173, wherein the isolated polypeptide is linked to a Fab domain.

Embodiment 175. The method of any one of embodiments 152 to 174, wherein the Fab comprises a CH1 domain.

Embodiment 176. The method of embodiment 175, wherein the CH1 domain comprises the amino acid sequence of SEQ ID NO: 17 or SEQ ID NO: 100.

Embodiment 177. The method of any one of embodiments 174 to 176, wherein the Fab comprises a Fab heavy chain and a Fab light chain that pair to form a binding domain.

Embodiment 178. The method of any one of embodiments 174 to 177, wherein the Fab immunospecifically binds to Parkin.

Embodiment 179. The method of embodiment 178, wherein the Fab heavy chain comprises a heavy chain complementarity determining region 1 (HCDR1) comprising the amino acid sequence of SEQ ID NO: 4, a HCDR2 comprising the amino acid sequence of SEQ ID NO: 5, and a HCDR3 comprising the amino acid sequence of SEQ ID NO: 6.

Embodiment 180. The method of embodiment 178 or embodiment 179, wherein the Fab heavy chain comprises a variable region comprising the amino acid sequence of SEQ ID NO: 7.

Embodiment 181. The method of any one of embodiments 178 to 180, wherein the Fab heavy chain comprises the amino acid sequence of SEQ ID NO: 9.

Embodiment 182. The method of any one of embodiments 178 to 181, wherein the Fab light chain comprises a light chain complementarity determining region 1 (LCDR1) comprising the amino acid sequence of SEQ ID NO: 13, a LCDR2 comprising the amino acid sequence RAS, and a LCDR3 comprising the amino acid sequence of SEQ ID NO: 15.

Embodiment 183. The method of any one of embodiments 178 to 182, wherein the Fab light chain comprises a variable region comprising the amino acid sequence of SEQ ID NO: 16.

Embodiment 184. The method of any one of embodiments 178 to 183, wherein the Fab light chain comprises the amino acid sequence of SEQ ID NO: 12.

Embodiment 185. The method of any one of embodiments 178 to 184, wherein the isolated polypeptide comprises the amino acid sequence of SEQ ID NO: 3.

Embodiment 186. The method of any one of embodiments 174 to 177, wherein the Fab immunospecifically binds to OCT4.

Embodiment 187. The method of embodiment 186, wherein the Fab heavy chain comprises a heavy chain complementarity determining region 1 (HCDR1) comprising the amino acid sequence of SEQ ID NO: 29, a HCDR2 comprising the amino acid sequence of SEQ ID NO: 30, and a HCDR3 comprising the amino acid sequence of SEQ ID NO: 31.

Embodiment 188. The method of embodiment 186 or embodiment 187, wherein the Fab heavy chain comprises a variable region comprising the amino acid sequence of SEQ ID NO: 32.

Embodiment 189. The method of any one of embodiments 186 to 188, wherein the Fab heavy chain comprises the amino acid sequence of SEQ ID NO: 34.

Embodiment 190. The method of any one of embodiments 186 to 189, wherein the Fab light chain comprises a light chain complementarity determining region 1 (LCDR1) comprising the amino acid sequence of SEQ ID NO: 37, a LCDR2 comprising the amino acid sequence GAS, and a LCDR3 comprising the amino acid sequence of SEQ ID NO: 39.

Embodiment 191. The method of any one of embodiments 186 to 190, wherein the Fab light chain comprises a variable region comprising the amino acid sequence of SEQ ID NO: 40.

Embodiment 192. The method of any one of embodiments 186 to 191, wherein the Fab light chain comprises the amino acid sequence of SEQ ID NO: 36.

Embodiment 193. The method of any one of embodiments 186 to 192, wherein the isolated polypeptide comprises the amino acid sequence of SEQ ID NO: 28.

Embodiment 194. The method of any one of embodiments 174 to 177, wherein the Fab immunospecifically binds to IDO1.

Embodiment 195. The method of embodiment 194, wherein the Fab heavy chain comprises a heavy chain complementarity determining region 1 (HCDR1) comprising the amino acid sequence of SEQ ID NO: 66, a HCDR2 comprising the amino acid sequence of SEQ ID NO: 67, and a HCDR3 comprising the amino acid sequence of SEQ ID NO: 68.

Embodiment 196. The method of embodiment 194 or embodiment 195, wherein the Fab heavy chain comprises a variable region comprising the amino acid sequence of SEQ ID NO: 65.

Embodiment 197. The method of any one of embodiments 194 to 196, wherein the Fab heavy chain comprises the amino acid sequence of SEQ ID NO: 75.

Embodiment 198. The method of any one of embodiments 194 to 197, wherein the Fab light chain comprises a light chain complementarity determining region 1 (LCDR1) comprising the amino acid sequence of SEQ ID NO: 72, a LCDR2 comprising the amino acid sequence of YAS, and a LCDR3 comprising the amino acid sequence of SEQ ID NO: 73.

Embodiment 199. The method of any one of embodiments 194 to 198, wherein the Fab light chain comprises a variable region comprising the amino acid sequence of SEQ ID NO: 71.

Embodiment 200. The method of any one of embodiments 194 to 199 wherein the Fab light chain comprises the amino acid sequence of SEQ ID NO: 77.

Embodiment 201. The method of any one of embodiments 194 to 200, wherein the isolated polypeptide comprises the amino acid sequence of SEQ ID NO: 79.

Embodiment 202. The method of any one of embodiments 174 to 177, wherein the Fab immunospecifically binds to FLAG-tag.

Embodiment 203. The method of embodiment 202, wherein the Fab heavy chain comprises a heavy chain complementarity determining region 1 (HCDR1) comprising the amino acid sequence of SEQ ID NO: 163, a HCDR2 comprising the amino acid sequence of SEQ ID NO: 164, and a HCDR3 comprising the amino acid sequence of SEQ ID NO: 165.

Embodiment 204. The method of embodiment 202 or embodiment 203, wherein the Fab heavy chain comprises a variable region comprising the amino acid sequence of SEQ ID NO: 162.

Embodiment 205. The method of any one of embodiments 202 to 204, wherein the Fab heavy chain comprises the amino acid sequence of SEQ ID NO: 171.

Embodiment 206. The method of any one of embodiments 202 to 205, wherein the Fab light chain comprises a light chain complementarity determining region 1 (LCDR1) comprising the amino acid sequence of SEQ ID NO: 168, a LCDR2 comprising the amino acid sequence of YAS, and a LCDR3 comprising the amino acid sequence of SEQ ID NO: 169.

Embodiment 207. The method of any one of embodiments 202 to 206, wherein the Fab light chain comprises a variable region comprising the amino acid sequence of SEQ ID NO: 167.

Embodiment 208. The method of any one of embodiments 202 to 207 wherein the Fab light chain comprises the amino acid sequence of SEQ ID NO: 173.

Embodiment 209. The method of any one of embodiments 202 to 208, wherein the isolated polypeptide comprises the amino acid sequence of SEQ ID NO: 175.

Embodiment 210. The method of any one of embodiments 174 to 177, wherein the Fab is a Rabbit anti-Mouse IgG secondary antibody.

Embodiment 211. The method of embodiment 210, wherein the Fab heavy chain comprises a heavy chain complementarity determining region 1 (HCDR1) comprising the amino acid sequence of SEQ ID NO: 127, a HCDR2 comprising the amino acid sequence of SEQ ID NO: 128, and a HCDR3 comprising the amino acid sequence of SEQ ID NO: 129.

Embodiment 212. The method of embodiment 210 or embodiment 211, wherein the Fab heavy chain comprises a variable region comprising the amino acid sequence of SEQ ID NO: 126.

Embodiment 213. The method of any one of embodiments 210 to 212, wherein the Fab heavy chain comprises the amino acid sequence of SEQ ID NO: 135.

Embodiment 214. The method of any one of embodiments 210 to 213, wherein the Fab light chain comprises a light chain complementarity determining region 1 (LCDR1) comprising the amino acid sequence of SEQ ID NO: 132, a LCDR2 comprising the amino acid sequence GAS, and a LCDR3 comprising the amino acid sequence of SEQ ID NO: 133.

Embodiment 215. The method of any one of embodiments 210 to 214, wherein the Fab light chain comprises a variable region comprising the amino acid sequence of SEQ ID NO: 131.

Embodiment 216. The method of any one of embodiments 210 to 215, wherein the Fab light chain comprises the amino acid sequence of SEQ ID NO: 137.

Embodiment 217. The method of any one of embodiments 210 to 216, wherein the isolated polypeptide comprises the amino acid sequence of SEQ ID NO: 139.

Embodiment 218. The method of any one of embodiments 174 to 177, wherein the Fab immunospecifically binds to pERK1/2.

Embodiment 219. The method of embodiment 218, wherein the Fab heavy chain comprises a heavy chain complementarity determining region 1 (HCDR1) comprising the amino acid sequence of SEQ ID NO: 85, a HCDR2 comprising the amino acid sequence of SEQ ID NO: 86, and a HCDR3 comprising the amino acid sequence of SEQ ID NO: 87.

Embodiment 220. The method of embodiment 218 or embodiment 219, wherein the Fab heavy chain comprises a variable region comprising the amino acid sequence of SEQ ID NO: 84.

Embodiment 221. The method of any one of embodiments 218 to 220, wherein the Fab heavy chain comprises the amino acid sequence of SEQ ID NO: 94.

Embodiment 222. The method of any one of embodiments 218 to 220, wherein the Fab light chain comprises a light chain complementarity determining region 1 (LCDR1) comprising the amino acid sequence of SEQ ID NO: 91, a LCDR2 comprising the amino acid sequence of KVS, and a LCDR3 comprising the amino acid sequence of SEQ ID NO: 92.

Embodiment 223. The method of any one of embodiments 218 to 222, wherein the Fab light chain comprises a variable region comprising the amino acid sequence of SEQ ID NO: 90.

Embodiment 224. The method of any one of embodiments 218 to 223, wherein the Fab light chain comprises the amino acid sequence of SEQ ID NO: 96.

Embodiment 225. The method of any one of embodiments 218 to 224, wherein the isolated polypeptide comprises the amino acid sequence of SEQ ID NO: 98.

Embodiment 226. The method of any one of embodiments 174 to 177, wherein the Fab immunospecifically binds to PSD-95.

Embodiment 227. The method of embodiment 226, wherein the Fab heavy chain comprises a heavy chain complementarity determining region 1 (HCDR1) comprising the amino acid sequence of SEQ ID NO: 111, a HCDR2 comprising the amino acid sequence of SEQ ID NO: 112, and a HCDR3 comprising the amino acid sequence of SEQ ID NO: 113.

Embodiment 228. The method of embodiment 226 or embodiment 227, wherein the Fab heavy chain comprises a variable region comprising the amino acid sequence of SEQ ID NO: 110.

Embodiment 229. The method of any one of embodiments 226 to 228, wherein the Fab heavy chain comprises the amino acid sequence of SEQ ID NO: 119.

Embodiment 230. The method of any one of embodiments 226 to 229, wherein the Fab light chain comprises a light chain complementarity determining region 1 (LCDR1) comprising the amino acid sequence of SEQ ID NO: 116, a LCDR2 comprising the amino acid sequence of DTS, and a LCDR3 comprising the amino acid sequence of SEQ ID NO: 117.

Embodiment 231. The method of any one of embodiments 226 to 230, wherein the Fab light chain comprises a variable region comprising the amino acid sequence of SEQ ID NO: 115.

Embodiment 232. The method of any one of embodiments 226 to 231, wherein the Fab light chain comprises the amino acid sequence of SEQ ID NO: 121.

Embodiment 233. The method of any one of embodiments 226 to 232, wherein the isolated polypeptide comprises the amino acid sequence of SEQ ID NO: 123.

Embodiment 234. The method of any one of embodiments 174 to 177, wherein the Fab immunospecifically binds to TIGIT.

Embodiment 235. The method of embodiment 234, wherein the Fab heavy chain comprises a heavy chain complementarity determining region 1 (HCDR1) comprising the amino acid sequence of SEQ ID NO: 146, a HCDR2 comprising the amino acid sequence of SEQ ID NO: 147, and a HCDR3 comprising the amino acid sequence of SEQ ID NO: 148.

Embodiment 236. The method of embodiment 234 or embodiment 235, wherein the Fab heavy chain comprises a variable region comprising the amino acid sequence of SEQ ID NO: 145.

Embodiment 237. The method of any one of embodiments 234 to 236, wherein the Fab heavy chain comprises the amino acid sequence of SEQ ID NO: 154.

Embodiment 238. The method of any one of embodiments 234 to 237, wherein the Fab light chain comprises a light chain complementarity determining region 1 (LCDR1) comprising the amino acid sequence of SEQ ID NO: 151, a LCDR2 comprising the amino acid sequence of SAS, and a LCDR3 comprising the amino acid sequence of SEQ ID NO: 152.

Embodiment 239. The method of any one of embodiments 234 to 238, wherein the Fab light chain comprises a variable region comprising the amino acid sequence of SEQ ID NO: 150.

Embodiment 240. The method of any one of embodiments 234 to 239, wherein the Fab light chain comprises the amino acid sequence of SEQ ID NO: 156.

Embodiment 241. The method of any one of embodiments 234 to 240, wherein the isolated polypeptide comprises the amino acid sequence of SEQ ID NO: 158.

Embodiment 242. The method of any one of embodiments 152 to 241, wherein the isolated polypeptide is an antibody with the isotype IgG.

Embodiment 243. A method of increasing sensitivity of an antibody, the method comprising: introducing into a heavy chain of the antibody a polypeptide comprising amino acid sequence of Formula I: Z-(X-Yₙ-Zᵣ)ₚ-X, wherein
Z is a hinge,
X is a CH2 domain,
Y is a linker,
n is an integer of 1 or greater,
p is an integer of 2 or greater, and
r is an integer of 0 or 1,
wherein the sensitivity of the antibody is increased by at least 1.5-fold compared to a corresponding wild type antibody.

Embodiment 244. The method of embodiment 243, further comprising introducing LFLEPS mutations into the amino acid sequence of X, wherein the LFLEPS mutations comprise L234F, L235E and P331S substitutions.

Embodiment 245. The method of embodiment 243 or embodiment 244, wherein X comprises an amino acid sequence at least 90% identical to SEQ ID NO: 19, at least 90% identical to SEQ ID NO: 49, or at least 90% identical to SEQ ID NO: 104.

Embodiment 246. The method of any one of embodiments 243 to 245, wherein Z comprises an amino acid sequence at least 90% identical to SEQ ID NO: 18, at least 90% identical to SEQ ID NO: 48, or at least 90% identical to SEQ ID NO: 102.

Embodiment 247. The method of any one of embodiments 243 to 246, wherein Y comprises an amino acid sequence at least 90% identical to the amino acid sequence of GS, GGS, SEQ ID NO: 14, SEQ ID NO: 38, SEQ ID NO: 43, or SEQ ID NO: 44.

Embodiment 248. A method of reducing background signal of an antibody, the method comprising: introducing into a heavy chain of the antibody a polypeptide comprising amino acid sequence of Formula I: Z-(X-Yₙ-Zᵣ)ₚ-X, wherein
Z is a hinge,
X is a CH2 domain,
Y is a linker,
n is an integer of 1 or greater,
p is an integer of 2 or greater, and
r is an integer of 0 or 1,
wherein the background signal of the antibody is decreased by at least 1.5-fold compared to a corresponding wild type antibody.

Embodiment 249. The method of embodiment 248, further comprising introducing LFLEPS mutations into the amino acid sequence of X, wherein the LFLEPS mutations comprise L234F, L235E and P331S substitutions.

Embodiment 250. The method of embodiment 248 or embodiment 249, wherein X comprises an amino acid sequence at least 90% identical to SEQ ID NO: 19, at least 90% identical to SEQ ID NO: 49, or at least 90% identical to SEQ ID NO: 104.

Embodiment 251. The method of any one of embodiments 248 to embodiment 250, wherein Z comprises an amino acid sequence at least 90% identical to SEQ ID NO: 18, at least 90% identical to SEQ ID NO: 48, or at least 90% identical to SEQ ID NO: 102.

Embodiment 252. The method of any one of embodiments 248 to 251, wherein Y comprises an amino acid sequence at least 90% identical to the amino acid sequence of GS, GGS, SEQ ID NO: 14, SEQ ID NO: 38, SEQ ID NO: 43, or SEQ ID NO: 44.

What is claimed:

1. An isolated polypeptide comprising the amino acid sequence of Formula I: Z-(X-Yₙ-Zᵣ)ₚ-X, wherein
Z is a hinge;
X is a CH2 domain;
Y is a linker;
n is an integer of 1 or greater;
p is an integer of 2 or greater; and
r is an integer of 0 or 1.

2. The isolated polypeptide of claim 1, wherein
Z comprises an amino acid sequence comprising at least 90% identity to SEQ ID NO: 18, SEQ ID NO: 48, or SEQ ID NO: 102;
X comprises an amino acid sequence comprising at least 90% identity to SEQ ID NO: 19, SEQ ID NO: 49, or SEQ ID NO: 104;
Y is a linker;
n is an integer of 1 or greater;
p is an integer of 2 or greater; and
r is an integer of 0 or 1.

3. The isolated polypeptide of claim 1 or claim 2, wherein Y comprises an amino acid sequence of GS, GGS, at least 90% identity to SEQ ID NO: 14, at least 90% identity to SEQ ID NO: 38, at least 90% identity to SEQ ID NO: 43, or at least 90% identity to SEQ ID NO: 44.

4. The isolated polypeptide of any one of claims 1 to 3, wherein Z comprises SEQ ID NO: 18, SEQ ID NO: 48, or SEQ ID NO: 102; X comprises SEQ ID NO: 19, SEQ ID NO: 49, or SEQ ID NO: 104; Y comprises GS, GGS, SEQ ID NO: 14, SEQ ID NO: 38, SEQ ID NO: 43, or SEQ ID NO: 44; n is an integer of 1 or greater; p is an integer of 2 or greater; and r is an integer of 0 or 1.

5. The isolated polypeptide of any one of claims 1 to 4, wherein Z comprises the amino acid sequence of SEQ ID NO: 18, X comprises the amino acid sequence of SEQ ID NO: 19, Y comprises the amino acid sequence of SEQ ID NO: 44, n is 1, and p is 2.

6. The isolated polypeptide of any one of claims 1 to 4, wherein Z comprises the amino acid sequence of SEQ ID NO: 48, X comprises the amino acid sequence of SEQ ID NO: 49, Y comprises the amino acid sequence of SEQ ID NO: 44, n is 1, and p is 2.

7. The isolated polypeptide of any one of claims 1 to 4, wherein Z comprises the amino acid sequence of SEQ ID NO: 102, X comprises the amino acid sequence of SEQ ID NO: 104, Y comprises the amino acid sequence of SEQ ID NO: 44, n is 1, and p is 2.

8. The isolated polypeptide of any one of the preceding claims, wherein the isolated polypeptide is linked to a second polypeptide by one or more disulfide bonds, wherein the isolated polypeptide and the second polypeptide share the same amino acid sequence.

9. The isolated polypeptide of any one of the preceding claims, wherein the isolated polypeptide comprises the amino acid sequence of SEQ ID NO: 53 or SEQ ID NO: 55.

10. The isolated polypeptide of claim 8, wherein the one or more disulfide bonds occur between the hinge of the isolated polypeptide and the hinge of the second polypeptide.

11. The isolated polypeptide of any one of the preceding claims, wherein the isolated polypeptide further comprises a CH3 domain.

12. The isolated polypeptide of claim 9, wherein the CH3 domain comprises the amino acid sequence of SEQ ID NO: 20, or SEQ ID NO: 106.

13. An antibody comprising the isolated polypeptide of any one of the preceding claims linked to a Fab domain.

14. The antibody of claim 13, wherein the Fab comprises a CH1 domain.

15. The antibody of claim 14, wherein the CH1 domain comprises the amino acid sequence of SEQ ID NO: 17, SEQ ID NO: 100, or SEQ ID NO: 160.

16. The antibody of claim 13, wherein the Fab comprises a Fab heavy chain and a Fab light chain that pair to form a binding domain.

17. The antibody of any one of claims 13 to 16, wherein the Fab immunospecifically binds to Parkin, OCT4, IDO1, FLAG-tag, pERK1/2, PSD-95, or TIGIT.

18. The antibody of any one of claims 13 to 17, wherein the isolated polypeptide is an antibody with the isotype IgG.

19. An antibody comprising a polypeptide comprising the amino acid sequence of Formula I: Z-(X-Yₙ-Zᵣ)ₚ-X, wherein
Z is a hinge;
X is a CH2 domain;
Y is a linker;
n is an integer of 1 or greater;
p is an integer of 2 or greater; and
r is an integer of 0 or 1.

20. The antibody of claim 19, wherein
comprises an amino acid sequence comprising at least 90% identity to SEQ ID NO: 18, SEQ ID NO: 48, or SEQ ID NO: 102;
X comprises an amino acid sequence comprising at least 90% identity to SEQ ID NO: 19, SEQ ID NO: 49, or SEQ ID NO: 104;
Y is a linker;
n is an integer of 1 or greater;
p is an integer of 2 or greater; and
r is an integer of 0 or 1.

21. The antibody of claim 19 or claim 20, wherein Y comprises an amino acid sequence of GS, GGS, at least 90% identity to SEQ ID NO: 14, at least 90% identity to SEQ ID NO: 38, at least 90% identity to SEQ ID NO: 43, or at least 90% identity to SEQ ID NO: 44.

22. The antibody of any one of claims 19 to 21, wherein Z comprises SEQ ID NO: 18, SEQ ID NO: 48, or SEQ ID NO: 102; X comprises SEQ ID NO: 19, SEQ ID NO: 49, or SEQ ID NO: 104; Y comprises GS, GGS, SEQ ID NO: 14, SEQ ID NO: 38, SEQ ID NO: 43, or SEQ ID NO: 44; n is an integer of 1 or greater; p is an integer of 2 or greater; and r is an integer of 0 or 1.

23. The antibody of any one of claims 19 to 22, wherein Z comprises the amino acid sequence of SEQ ID NO: 18, X comprises the amino acid sequence of SEQ ID NO: 19, Y comprises the amino acid sequence of SEQ ID NO: 44, n is 1, and p is 2.

24. The antibody of any one of claims 19 to 22, wherein Z comprises the amino acid sequence of SEQ ID NO: 48, X comprises the amino acid sequence of SEQ ID NO: 49, Y comprises the amino acid sequence of SEQ ID NO: 44, n is 1, and p is 2.

25. The antibody of any one of claims 19 to 22, wherein Z comprises the amino acid sequence of SEQ ID NO: 102, X comprises the amino acid sequence of SEQ ID NO: 104, Y comprises the amino acid sequence of SEQ ID NO: 44, n is 1, and p is 2.

26. The antibody of any one of claims 19 to 25, wherein the polypeptide is linked to a second polypeptide by one or more disulfide bonds, wherein the polypeptide and the second polypeptide share the same amino acid sequence.

27. The antibody of any one of claims 19 to 26, wherein the polypeptide comprises the amino acid sequence of SEQ ID NO: 53 or SEQ ID NO: 55.

28. The antibody of claim 26, wherein the one or more disulfide bonds occur between the hinge of the polypeptide and the hinge of the second polypeptide.

29. The antibody of any one of claims 19 to 28, further comprising a CH3 domain.

30. The antibody of claim 29, wherein the CH3 domain comprises the amino acid sequence of SEQ ID NO: 20, or SEQ ID NO: 106.

31. The antibody of any one of claims 13 to 30, wherein the antibody has a reduction of background signal of at least 1.5-fold compared to a corresponding wildtype antibody without the isolated polypeptide.

32. The antibody of any one of claims 13 to 31, wherein the antibody has an increase in sensitivity of at least 1.5-fold compared to a corresponding wildtype antibody without the isolated polypeptide.

33. The isolated polypeptide of any one of claims 1-12 or the antibody of any one of claims 13-32, further comprising a label.

34. A nucleic acid encoding the isolated polypeptide of any one of claims 1-12 or the antibody of any one of claims 13-32.

35. An antibody that immunospecifically binds to Parkin, wherein the antibody comprises a heavy chain (HC) and a light chain (LC) that pair to form a binding domain comprising a heavy chain complementarity determining region 1 (HCDR1) comprising the amino acid sequence of SEQ ID NO: 4, a HCDR2 comprising the amino acid sequence of SEQ ID NO: 5, a HCDR3 comprising the amino acid sequence of SEQ ID NO: 6, a light chain complementarity determining region 1 (LCDR1) comprising the amino acid sequence of SEQ ID NO: 13, a LCDR2 comprising the amino acid sequence RAS, and a LCDR3 comprising the amino acid sequence of SEQ ID NO: 15, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

36. The antibody of claim 35, wherein the first CH2 domain, the second CH2 domain, and the third CH2 domain each comprises the amino acid sequence of SEQ ID NO: 19 or SEQ ID NO: 49.

37. The antibody of claim 35 or claim 36, wherein the first hinge and the second hinge each comprises the amino acid sequence of SEQ ID NO: 18 or SEQ ID NO: 48.

38. The antibody of any one of claims 35 to 37, wherein the first linker and the second linker each comprises the amino acid sequence of GS, GGS, SEQ ID NO: 14, SEQ ID NO: 38, SEQ ID NO: 43, or SEQ ID NO: 44.

39. The antibody of any one of claims 35 to 38, wherein the CH3 domain comprises the amino acid sequence of SEQ ID NO: 20.

40. The antibody of any one of claims 35 to 39, wherein the antibody comprises a heavy chain variable region having the polypeptide sequence of SEQ ID NO: 7 and a light chain variable region having the polypeptide sequence of SEQ ID NO: 16.

41. The antibody of any one of claims 35 to 40, wherein the antibody comprises a heavy chain having a polypeptide sequence of SEQ ID NO: 3 and a light chain having the polypeptide sequence of SEQ ID NO: 12.

42. The antibody of any one of claims 35 to 41, wherein the antibody comprises a heavy chain that is encoded by a polynucleotide sequence of SEQ ID NO: 2 and a light chain that is encoded by a polynucleotide sequence of SEQ ID NO: 11.

43. An antibody that immunospecifically binds to OCT4, wherein the antibody comprises a heavy chain (HC) and a light chain (LC) that pair to form a binding domain comprising a heavy chain complementarity determining region 1 (HCDR1) comprising the amino acid sequence of SEQ ID NO: 29, a HCDR2 comprising the amino acid sequence of SEQ ID NO: 30, and a HCDR3 comprising the amino acid sequence of SEQ ID NO: 31, a light chain complementarity determining region 1 (LCDR1) comprising the amino acid sequence of SEQ ID NO: 37, a LCDR2 comprising the amino acid sequence GAS, and a LCDR3 comprising the amino acid sequence of SEQ ID NO: 39, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

44. The antibody of claim 43, wherein the first CH2 domain, the second CH2 domain, and the third CH2 domain each comprises the amino acid sequence of SEQ ID NO: 19 or SEQ ID NO: 49.

45. The antibody of claim 43 or claim 44, wherein the first hinge and the second hinge each comprises the amino acid sequence of SEQ ID NO: 18 or SEQ ID NO: 48.

46. The antibody of any one of claims 43 to 45, wherein the first linker and the second linker each comprises the amino acid sequence of GS, GGS, SEQ ID NO: 14, SEQ ID NO: 38, SEQ ID NO: 43, or SEQ ID NO: 44.

47. The antibody of any one of claims 43 to 46, wherein the CH3 domain comprises the amino acid sequence of SEQ ID NO: 20.

48. The antibody of any one of claims 43 to 47, wherein the antibody comprises a heavy chain variable region having the polypeptide sequence of SEQ ID NO: 32 and a light chain variable region having the polypeptide sequence of SEQ ID NO: 40.

49. The antibody of any one of claims 43 to 48, wherein the antibody comprises a heavy chain having a polypeptide sequence of SEQ ID NO: 28 and a light chain having the polypeptide sequence of SEQ ID NO: 36.

50. The antibody of any one of claims 43 to 49, wherein the antibody comprises a heavy chain that is encoded by a polynucleotide sequence of SEQ ID NO: 27 and a light chain that is encoded by a polynucleotide sequence of SEQ ID NO: 35.

51. An antibody that immunospecifically binds to IDO1, wherein the antibody comprises a heavy chain (HC) and a light chain (LC) that pair to form a binding domain comprising a heavy chain complementarity determining region 1 (HCDR1) comprising the amino acid sequence of SEQ ID NO: 66, a HCDR2 comprising the amino acid sequence of SEQ ID NO: 67, a HCDR3 comprising the amino acid sequence of SEQ ID NO: 68, a light chain complementarity determining region 1 (LCDR1) comprising the amino acid sequence of SEQ ID NO: 72, a LCDR2 comprising the amino acid sequence of YAS, and a LCDR3 comprising the amino acid sequence of SEQ ID NO: 73, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

52. The antibody of claim 51, wherein the first CH2 domain, the second CH2 domain, and the third CH2 domain each comprises the amino acid sequence of SEQ ID NO: 19 or SEQ ID NO: 49.

53. The antibody of claim 51 or claim 52, wherein the first hinge and the second hinge each comprises the amino acid sequence of SEQ ID NO: 18 or SEQ ID NO: 48.

54. The antibody of any one of claims 51 to 53, wherein the first linker and the second linker each comprises the amino acid sequence of GS, GGS, SEQ ID NO: 14, SEQ ID NO: 38, SEQ ID NO: 43, or SEQ ID NO: 44.

55. The antibody of any one of claims 51 to 54, wherein the CH3 domain comprises the amino acid sequence of SEQ ID NO: 20.

56. The antibody of any one of claims 51 to 55, wherein the antibody comprises a heavy chain variable region having the polypeptide sequence of SEQ ID NO: 65 and a light chain variable region having the polypeptide sequence of SEQ ID NO: 71.

57. The antibody of any one of claims 51 to 56, wherein the antibody comprises a heavy chain having a polypeptide sequence of SEQ ID NO: 75 and a light chain having the polypeptide sequence of SEQ ID NO: 77.

58. The antibody of any one of claims 51 to 57, wherein the antibody comprises a heavy chain that is encoded by a polynucleotide sequence of SEQ ID NO: 74 and a light chain that is encoded by a polynucleotide sequence of SEQ ID NO: 76.

59. An antibody that immunospecifically binds to FLAG-tag, wherein the antibody comprises a heavy chain (HC) and a light chain (LC) that pair to form a binding domain comprising a heavy chain complementarity determining region 1 (HCDR1) comprising the amino acid sequence of SEQ ID NO: 163, a HCDR2 comprising the amino acid sequence of SEQ ID NO: 164, a HCDR3 comprising the amino acid sequence of SEQ ID NO: 165, a light chain complementarity determining region 1 (LCDR1) comprising the amino acid sequence of SEQ ID NO: 168, a LCDR2 comprising the amino acid sequence of YAS, and a LCDR3 comprising the amino acid sequence of SEQ ID NO: 169, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

60. The antibody of claim 59, wherein the first CH2 domain, the second CH2 domain, and the third CH2 domain each comprises the amino acid sequence of SEQ ID NO: 19 or SEQ ID NO: 49.

61. The antibody of claim 59 or claim 60, wherein the first hinge and the second hinge each comprises the amino acid sequence of SEQ ID NO: 18 or SEQ ID NO: 48.

62. The antibody of any one of claims 59 to 61, wherein the first linker and the second linker each comprises the amino acid sequence of GS, GGS, SEQ ID NO: 14, SEQ ID NO: 38, SEQ ID NO: 43, or SEQ ID NO: 44.

63. The antibody of any one of claims 59 to 62, wherein the CH3 domain comprises the amino acid sequence of SEQ ID NO: 20.

64. The antibody of any one of claims 59 to 63, wherein the antibody comprises a heavy chain variable region having the polypeptide sequence of SEQ ID NO: 162 and a light chain variable region having the polypeptide sequence of SEQ ID NO: 167.

65. The antibody of any one of claims 59 to 64, wherein the antibody comprises a heavy chain having a polypeptide sequence of SEQ ID NO: 171 and a light chain having the polypeptide sequence of SEQ ID NO: 173.

66. The antibody of any one of claims 59 to 65, wherein the antibody comprises a heavy chain that is encoded by a polynucleotide sequence of SEQ ID NO: 170 and a light chain that is encoded by a polynucleotide sequence of SEQ ID NO: 172.

67. An antibody that is a Rabbit anti-Mouse IgG secondary antibody, wherein the antibody comprises a heavy chain (HC) and a light chain (LC) that pair to form a binding domain comprising a heavy chain complementarity determining region 1 (HCDR1) comprising the amino acid sequence of SEQ ID NO: 127, a HCDR2 comprising the amino acid sequence of SEQ ID NO: 128, a HCDR3 comprising the amino acid sequence of SEQ ID NO: 129, a light chain complementarity determining region 1 (LCDR1) comprising the amino acid sequence of SEQ ID NO: 132, a LCDR2 comprising the amino acid sequence of GAS, and a LCDR3 comprising the amino acid sequence of SEQ ID NO: 133, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

68. The antibody of claim 67, wherein the first CH2 domain, the second CH2 domain, and the third CH2 domain each comprises the amino acid sequence of SEQ ID NO: 19 or SEQ ID NO: 49.

69. The antibody of claim 67 or claim 68, wherein the first hinge and the second hinge each comprises the amino acid sequence of SEQ ID NO: 18 or SEQ ID NO: 48.

70. The antibody of any one of claims 67 to 69, wherein the first linker and the second linker each comprises the amino acid sequence of GS, GGS, SEQ ID NO: 14, SEQ ID NO: 38, SEQ ID NO: 43, or SEQ ID NO: 44.

71. The antibody of any one of claims 67 to 70, wherein the CH3 domain comprises the amino acid sequence of SEQ ID NO: 20.

72. The antibody of any one of claims 67 to 71, wherein the antibody comprises a heavy chain variable region having the polypeptide sequence of SEQ ID NO: 126 and a light chain variable region having the polypeptide sequence of SEQ ID NO: 131.

73. The antibody of any one of claims 67 to 72, wherein the antibody comprises a heavy chain having a polypeptide sequence of SEQ ID NO: 135 and a light chain having the polypeptide sequence of SEQ ID NO: 137.

74. The antibody of any one of claims 67 to 73, wherein the antibody comprises a heavy chain that is encoded by a polynucleotide sequence of SEQ ID NO: 134 and a light chain that is encoded by a polynucleotide sequence of SEQ ID NO: 136.

75. An antibody that immunospecifically binds to pERK1/2, wherein the antibody comprises a heavy chain (HC) and a light chain (LC) that pair to form a binding domain comprising a heavy chain complementarity determining region 1 (HCDR1) comprising the amino acid sequence of SEQ ID NO: 85, a HCDR2 comprising the amino acid sequence of SEQ ID NO: 86, a HCDR3 comprising the amino acid sequence of SEQ ID NO: 87, a light chain complementarity determining region 1 (LCDR1) comprising the amino acid sequence of SEQ ID NO: 91, a LCDR2 comprising the amino acid sequence of KVS, and a LCDR3 comprising the amino acid sequence of SEQ ID NO: 92, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

76. The antibody of claim 75, wherein the first CH2 domain, the second CH2 domain, and the third CH2 domain each comprises the amino acid sequence of SEQ ID NO: 104.

77. The antibody of claim 75 or claim 76, wherein the first hinge and the second hinge each comprises the amino acid sequence of SEQ ID NO: 102.

78. The antibody of any one of claims 75 to 77, wherein the first linker and the second linker each comprises the amino acid sequence of GS, GGS, SEQ ID NO: 14, SEQ ID NO: 38, SEQ ID NO: 43, or SEQ ID NO: 44.

79. The antibody of any one of claims 75 to 78, wherein the CH3 domain comprises the amino acid sequence of SEQ ID NO: 106.

80. The antibody of any one of claims 75 to 79, wherein the antibody comprises a heavy chain variable region having the polypeptide sequence of SEQ ID NO: 83 and a light chain variable region having the polypeptide sequence of SEQ ID NO: 90.

81. The antibody of any one of claims 75 to 80, wherein the antibody comprises a heavy chain having a polypeptide sequence of SEQ ID NO: 94 and a light chain having the polypeptide sequence of SEQ ID NO: 96.

82. The antibody of any one of claims 75 to 81, wherein the antibody comprises a heavy chain that is encoded by a polynucleotide sequence of SEQ ID NO: 93 and a light chain that is encoded by a polynucleotide sequence of SEQ ID NO: 95.

83. An antibody that immunospecifically binds to PSD-95, wherein the antibody comprises a heavy chain (HC) and a light chain (LC) that pair to form a binding domain comprising a heavy chain complementarity determining region 1 (HCDR1) comprising the amino acid sequence of SEQ ID NO: 111, a HCDR2 comprising the amino acid sequence of SEQ ID NO: 112, a HCDR3 comprising the amino acid sequence of SEQ ID NO: 113, a light chain complementarity determining region 1 (LCDR1) comprising the amino acid sequence of SEQ ID NO: 116, a LCDR2 comprising the amino acid sequence of DTS, and a LCDR3 comprising the amino acid sequence of SEQ ID NO: 117, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

84. The antibody of claim 83, wherein the first CH2 domain, the second CH2 domain, and the third CH2 domain each comprises the amino acid sequence of SEQ ID NO: 104.

85. The antibody of claim 83 or claim 84, wherein the first hinge and the second hinge each comprises the amino acid sequence of SEQ ID NO: 102.

86. The antibody of any one of claims 83 to 85 wherein the first linker and the second linker each comprises the amino acid sequence of GS, GGS, SEQ ID NO: 14, SEQ ID NO: 38, SEQ ID NO: 43, or SEQ ID NO: 44.

87. The antibody of any one of claims 83 to 86, wherein the CH3 domain comprises the amino acid sequence of SEQ ID NO: 106.

88. The antibody of any one of claims 83 to 87, wherein the antibody comprises a heavy chain variable region having the polypeptide sequence of SEQ ID NO: 110 and a light chain variable region having the polypeptide sequence of SEQ ID NO: 115.

89. The antibody of any one of claims 83 to 88, wherein the antibody comprises a heavy chain having a polypeptide sequence of SEQ ID NO: 119 and a light chain having the polypeptide sequence of SEQ ID NO: 121.

90. The antibody of any one of claims 83 to 89, wherein the antibody comprises a heavy chain that is encoded by a polynucleotide sequence of SEQ ID NO: 118 and a light chain that is encoded by a polynucleotide sequence of SEQ ID NO: 120.

91. An antibody that immunospecifically binds to TIGIT, wherein the antibody comprises a heavy chain (HC) and a light chain (LC) that pair to form a binding domain comprising a heavy chain complementarity determining region 1 (HCDR1) comprising the amino acid sequence of SEQ ID NO: 146, a HCDR2 comprising the amino acid sequence of SEQ ID NO: 147, a HCDR3 comprising the amino acid sequence of SEQ ID NO: 148, a light chain complementarity determining region 1 (LCDR1) comprising the amino acid sequence of SEQ ID NO: 151, a LCDR2 comprising the amino acid sequence of SAS, and a LCDR3 comprising the amino acid sequence of SEQ ID NO: 152, and wherein the heavy chain further comprises a first CH2 domain, a second CH2 domain, and a third CH2 domain, wherein the first CH2 domain and the second CH2 domain are connected by a first linker and an optional first hinge, and wherein the second CH2 domain and the third CH2 domain are connected by a second linker and an optional second hinge, and wherein the heavy chain further comprises a CH3 domain.

92. The antibody of claim 91, wherein the first CH2 domain, the second CH2 domain, and the third CH2 domain each comprises the amino acid sequence of SEQ ID NO: 19.

93. The antibody of claim 91 or claim 92, wherein the first hinge and the second hinge each comprises the amino acid sequence of SEQ ID NO: 18.

94. The antibody of any one of claims 91 to 93, wherein the first linker and the second linker each comprises the amino acid sequence of GS, GGS, SEQ ID NO: 14, SEQ ID NO: 38, SEQ ID NO: 43, or SEQ ID NO: 44.

95. The antibody of any one of claims 91 to 94, wherein the CH3 domain comprises the amino acid sequence of SEQ ID NO: 20.

96. The antibody of any one of claims 91 to 95, wherein the antibody comprises a heavy chain variable region having the polypeptide sequence of SEQ ID NO: 145 and a light chain variable region having the polypeptide sequence of SEQ ID NO: 150.

97. The antibody of any one of claims 91 to 96, wherein the antibody comprises a heavy chain having a polypeptide sequence of SEQ ID NO: 154 and a light chain having the polypeptide sequence of SEQ ID NO: 156.

98. The antibody of any one of claims 91 to 97, wherein the antibody comprises a heavy chain that is encoded by a polynucleotide sequence of SEQ ID NO: 153 and a light chain that is encoded by a polynucleotide sequence of SEQ ID NO: 155.

99. The antibody of any one of claims 13 to 32 or 35 to 98, wherein the antibody provides at least a 1.5-fold increase in signal enhancement compared to a corresponding wild type antibody.

100. The antibody of any one of claims 13 to 33 or 35 to 99, wherein the antibody provides at least a 1.5-fold reduction in background signal compared to a corresponding wild type antibody.

101. A method of detecting the presence of a target protein in a sample, said method comprising:
applying the isolated polypeptide of any one of claims 1 to 12 or the antibody of any one of claims 13 to 33 or claims 35 to 100 to a sample containing a target protein,
incubating the isolated polypeptide or the antibody in the sample under conditions permitting the isolated polypeptide or the antibody to bind to the target protein and to form a bound isolated polypeptide or a bound antibody, and
determining the presence of the bound isolated polypeptide or the bound antibody, thereby detecting the presence of the target protein.

102. The method of claim 101, wherein the isolated polypeptide or the antibody has less background binding to cells that express Fc gamma receptors relative to a wild type isolated polypeptide or a wild type antibody.

103. The method of claim 101 or claim 102, wherein the target protein is OCT4, Parkin, IDO1, FLAG-tag, pERK1/2, PSD-95 or TIGIT.

104. The method of any one of claims 101 to 103, wherein the target protein is present in the sample in low abundance.

105. The method of any one of claims 101 to 104, wherein the presence of the bound isolated polypeptide or the bound antibody is determined by applying a secondary antibody to said sample, wherein the secondary antibody is labeled.

106. The method of any one of claims 101 to 105, wherein the antibody is labeled.

107. The method of claim 105 or claim 106, wherein the label is selected from a fluorophore, a fluorescent protein, a phosphorescent dye, a tandem dye, a polymer dye, a particle, an electron transfer agent, a hapten, an enzyme, a radioisotope, a chromophore, and an oligonucleotide-based fluorescent dye.

108. The method of claim 107, wherein the fluorophore is a pyrene, an anthracene, a naphthalene, an acridine, a stilbene, an indole or benzindole, an oxazole or benzoxazole, a thiazole or benzothiazole, an indacene, a 4-amino-7-nitrobenz-2-oxa-1,3-diazole (NBD), a cyanine, a carbocyanine, a benzocyanine, a carbostyryl, a porphyrin, a salicylate, an anthranilate, an azulene, a perylene, a pyridine, a quinoline, a borapolyazaindacene, a xanthene, a rhodamine, a silicon rhodamine, a fluorescein, a rhodol, a rosamine, an oxazine or a benzoxazine, a benzophenoxazine, a carbazine, a phenalenone, a coumarin, a benzofuran, a benzophenalenone, a benzopyrilium, a styryl, a squarine, a resorufin, an anthraquinone, or a quinazolinone, or a conjugated polymer dye.

109. The method of claim 107 wherein the particle is a semiconductor nanocrystal.

110. The method of claim 107, wherein the hapten is a biotin or a biotin derivative.

111. The method of claim 107, wherein the enzyme is selected from a peroxidase, a phosphatase, a glycosidase, and a luciferase.

112. The method of claim 111, wherein the method further comprises adding an enzymatic substrate selected from a chromogenic, a fluorogenic, and a chemiluminescent substrate.

113. The method of any one of claims 106 to 112, wherein detecting the labeled antibody utilizes flow cytometry, immunohistochemistry, immunocytochemistry, or Western blotting.

114. The method of any one of claims 101 to 113, wherein the isolated polypeptide is an antibody with the isotype IgG.

115. A method of increasing sensitivity of an antibody, the method comprising: introducing into a heavy chain of the antibody a polypeptide comprising amino acid sequence of Formula I: Z-(X-Yₙ-Zᵣ)ₚ-X, wherein
is a hinge;
X is a CH2 domain;
Y is a linker;
n is an integer of 1 or greater;
p is an integer of 2 or greater; and
r is an integer of 0 or 1,
wherein the sensitivity of the antibody is increased by at least 1.5-fold compared to a corresponding wild type antibody.

116. The method of claim 115, further comprising introducing LFLEPS mutations into the amino acid sequence of X, wherein the LFLEPS mutations comprise L234F, L235E and P331S substitutions.

117. The method of claim 115 or claim 116, wherein X comprises an amino acid sequence at least 90% identical to SEQ ID NO: 19, at least 90% identical to SEQ ID NO: 49, or at least 90% identical to SEQ ID NO: 104.

118. The method of any one of claims 115 to 117, wherein Z comprises an amino acid sequence at least 90% identical to SEQ ID NO: 18, at least 90% identical to SEQ ID NO: 48, or at least 90% identical to SEQ ID NO: 102.

119. The method of any one of claims 115 to 118, wherein Y comprises an amino acid sequence at least 90% identical to the amino acid sequence of GS, GGS, SEQ ID NO: 14, SEQ ID NO: 38, SEQ ID NO: 43, or SEQ ID NO: 44.

120. A method of reducing background signal of an antibody, the method comprising: introducing into a heavy chain of the antibody a polypeptide comprising amino acid sequence of Formula I: Z-(X-Yₙ-Zᵣ)ₚ-X, wherein
Z is a hinge;
X is a CH2 domain;
Y is a linker;
n is an integer of 1 or greater;
p is an integer of 2 or greater; and
r is an integer of 0 or 1;
wherein the background signal of the antibody is decreased by at least 1.5-fold compared to a corresponding wild type antibody.

121. The method of claim 120, further comprising introducing LFLEPS mutations into the amino acid sequence of X, wherein the LFLEPS mutations comprise L234F, L235E and P331S substitutions.

122. The method of claim 120 or claim 121, wherein X comprises an amino acid sequence at least 90% identical to SEQ ID NO: 19, at least 90% identical to SEQ ID NO: 49, or at least 90% identical to SEQ ID NO: 104.

123. The method of any one of claim 120 to claim 122, wherein Z comprises an amino acid sequence at least 90% identical to SEQ ID NO: 18, at least 90% identical to SEQ ID NO: 48, or at least 90% identical to SEQ ID NO: 102.

124. The method of any one of claims 120 to 123, wherein Y comprises an amino acid sequence at least 90% identical to the amino acid sequence of GS, GGS, SEQ ID NO: 14, SEQ ID NO: 38, SEQ ID NO: 43, or SEQ ID NO: 44.
